SEQUENCE LISTING (84 sequences)

SEQ ID NO: 1 - DNA, 3195 bp, Staphylococcus aureus
atggtggcatatttaaatattcatacggcttatgatttgttaaattcaagcttaaaaatagaagatgccgtaagacttgctgtgtctgaaaatgttgatgcacttgccataactgacaccaatgtattgtatggttttcctaaattttatgatgcatgtatagcaaataacattaaaccgatttttggtatgacaatatatgtgacaaatggattaaatacagtcgaaacagttgttctagctaaaaataatgatggattaaaagatttgtatcaactatcatcggaaataaaaatgaatgcattagaacatgtgtcgtttgaattattaaaacgattttctaacaatatgattatcatttttaaaaaagtcggtgatcaacatcgtgatattgtacaagtgtttgaaacccataatgacacatatatggaccaccttagtatttcgattcaaggtagaaaacatgtttggattcaaaatgtttgttaccaaacacgtcaagatgccgatacgatttctgcattagcagctattagagacaatacaaaattagacttaattcatgatcaagaagattttggtgcacattttttaactgaaaaggaaattaatcaattagatattaaccaagaatatttaacgcaggttgatgttatagctcaaaagtgtgatgcagaattaaaatatcatcaatctctacttcctcaatatgagacacctaatgatgaatcagctaaaaaatatttgtggcgtgtcttagttacacaattgaaaaaattagaacttaattatgacgtctatttagagcgattgaaatatgagtataaagttattactaatatgggttttgaagattatttcttaatagtaagtgatttaatccattatgcgaaaacgaatgatgtgatggtaggtcctggtcgtggttcttcagctggctcactggtcagttatttattgggaattacaacgattgatcctattaaattcaatctattatttgaacgttttttaaacccagaacgtgtaacaatgcctgatattgatattgactttgaagatacacgccgagaaagggtcattcagtacgtccaagaaaaatatggcgagctacatgtatctggaattgtgactttcggtcatctgcttgcaagagcagttgctagagatgttggaagaattatggggtttgatgaagttacattaaatgaaatttcaagtttaatcccacataaattaggaattacacttgatgaagcatatcaaattgacgattttaaagagtttgtacatcgaaaccatcgacatgaacgctggttcagtatttgtaaaaagttagaaggtttaccaagacatacatctacacatgcggcaggaattattattaatgaccatccattatatgaatatgcccctttaacgaaaggggatacaggattattaacgcaatggacaatgactgaagccgaacgtattgggttattaaaaatagattttctagggttgagaaacttatcgattattcatcaaatcttaacacaagtcaaaaaagatttaggtattaatattgatatcgaaaagattccgtttgatgatcaaaaagtgtttgaattgttgtcgcaaggagatacgactggcatattccaattagagtctgacggtgtaagaagtgtattaaaaaaattaaagccggaacactttgaagatattgttgctgtaacttctttgtatagaccaggtccaatggaagaaattccaacttacattacaagaagacatgatccaagcaaagttcaatatttacatccgcatttagaacctatattaaaaaatacttacggtgttattatttatcaagagcaaattatgcaaatagcgagcacatttgcaaacttcagttatggtgaagcggatattttaagaagagcaatgagtaaaaaaaatagagctgttcttgaaagtgagcgtcaacattttatagaaggtgcaaagcaaaatggttatcacgaagacattagtaagcaaatatttgatttgattctgaaatttgctgattatggttttcctagagcacatgctgtcagctattctaaaattgcatacattatgagctttttaaaagtccattatccaaattatttttacgcaaatattttaagtaatgttattggaagtgagaagaaaactgctcaaatgatagaagaagcaaaaaaacaaggtatcactatattgccaccgaacattaacgaaagtcattggttttataaaccttcccaagaaggcatttatttatcaattggtacaattaaaggtgttggttatcaaagtgtgaaagtgattgttgatgaacgttatcagaacggcaaatttaaagatttctttgattttgctagacgtataccgaagagagtcaaaacgagaaagttacttgaagcactgattttagtgggagcgtttgatgcttttggtaaaacacgttcaacgttgttgcaagctattgatcaagtgttggatggcgatttaaacattgaacaagatggttttttatttgatattttaacgccaaaacagatgtatgaagataaagaagaattgcctgatgcacttattagtcagtacgaaaaagaatatttaggattttatgtttcgcaacacccagtagataaaaagtttgttgccaaacaatatttaacgatatttaaattgagtaacgcgcagaattataaacctatattagtacagtttgataaagttaaacaaattcgaactaaaaatggtcaaaatatggcattcgtcacattaaatgatggcattgaaactttagatggtgtgattttccctaatcagtttaaaaagtacgaagagttgttatcacataatgacttgtttatagttagcgggaaatttgaccatagaaagcaacaacgtcaactaattataaatgagattcagacattagccacttttgaagaacaaaaattagcatttgccaaacaaattataattagaaataaatcacaaatagatatgtttgaagagatgattaaagctacgaaagagaatgctaatgatgttgtgttatccttttatgatgaaacgattaaacaaatgactactttaggctatattaatcaaaaagatagtatgtttaataattttatacaatcctttaaccctagtgatattaggcttata

SEQ ID NO: 2 - protein, 1065 aa, Staphylococcus aureus
MVAYLNIHTAYDLLNSSLKIEDAVRLAVSENVDALAITDTNVLYGFPKFYDACIANNIKPIFGMTIYVTNGLNTVETVVLAKNNDGLKDLYQLSSEIKMNALEHVSFELLKRFSNNMIIIFKKVGDQHRDIVQVFETHNDTYMDHLSISIQGRKHVWIQNVCYQTRQDADTISALAAIRDNTKLDLIHDQEDFGAHFLTEKEINQLDINQEYLTQVDVIAQKCDAELKYHQSLLPQYETPNDESAKKYLWRVLVTQLKKLELNYDVYLERLKYEYKVITNMGFEDYFLIVSDLIHYAKTNDVMVGPGRGSSAGSLVSYLLGITTIDPIKFNLLFERFLNPERVTMPDIDIDFEDTRRERVIQYVQEKYGELHVSGIVTFGHLLARAVARDVGRIMGFDEVTLNEISSLIPHKLGITLDEAYQIDDFKEFVHRNHRHERWFSICKKLEGLPRHTSTHAAGIIINDHPLYEYAPLTKGDTGLLTQWTMTEAERIGLLKIDFLGLRNLSIIHQILTQVKKDLGINIDIEKIPFDDQKVFELLSQGDTTGIFQLESDGVRSVLKKLKPEHFEDIVAVTSLYRPGPMEEIPTYITRRHDPSKVQYLHPHLEPILKNTYGVIIYQEQIMQIASTFANFSYGEADILRRAMSKKNRAVLESERQHFIEGAKQNGYHEDISKQIFDLILKFADYGFPRAHAVSYSKIAYIMSFLKVHYPNYFYANILSNVIGSEKKTAQMIEEAKKQGITILPPNINESHWFYKPSQEGIYLSIGTIKGVGYQSVKVIVDERYQNGKFKDFFDFARRIPKRVKTRKLLEALILVGAFDAFGKTRSTLLQAIDQVLDGDLNIEQDGFLFDILTPKQMYEDKEELPDALISQYEKEYLGFYVSQHPVDKKFVAKQYLTIFKLSNAQNYKPILVQFDKVKQIRTKNGQNMAFVTLNDGIETLDGVIFPNQFKKYEELLSHNDLFIVSGKFDHRKQQRQLIINEIQTLATFEEQKLAFAKQIIIRNKSQIDMFEEMIKATKENANDVVLSFYDETIKQMTTLGYINQKDSMFNNFIQSFNPSDIRLI

SEQ ID NO: 3 - DNA, 1698 bp, Staphylococcus aureus
ttgaattatcaagccttatatcgtatgtacagaccccaaagtttcgaggatgtcgtcggacaagaacatgtcacgaagacattgcgcaatgcgatttcgaaagaaaaacagtcgcatgcatatatttttagtggtccgagaggtacggggaaaacgagtattgccaaagtgtttgctaaagcaatcaactgtttaaatagcactgatggagaaccttgtaatgaatgtcatatttgtaaaggcattacgcaggggactaattcagatgtgatagaaattgatgctgctagtaataatggcgttgatgaaataagaaatattagagacaaagttaaatatgcaccaagtgaatcgaaatataaagtttatattatagatgaggtgcacatgctaacaacaggtgcttttaatgcccttttaaagacgttagaagaacctccagcacacgctatttttatattggcaacgacagaaccacataaaatccctccaacaatcatttctagggcacaacgttttgattttaaagcaattagcctagatcaaattgttgaacgtttaaaatttgtagcagatgcacaacaaattgaatgtgaagatgaagccttggcatttatcgctaaagcgtctgaagggggtatgcgtgatgcattaagtattatggatcaggctattgctttcggcgatggcacattgacattacaagatgccctaaatgttacgggtagcgttcatgatgaagcgttggatcacttgtttgatgatattgtacaaggtgacgtacaagcatcttttaaaaaataccatcagtttataacagaaggtaaagaagtgaatcgcctaataaatgatatgatttattttgtcagagatacgattatgaataaaacatctgagaaagatactgagtatcgagcactgatgaacttagaattagatatgttatatcaaatgattgatcttattaatgatacattagtgtcgattcgttttagtgtgaatcaaaacgttcattttgaagtattgttagtaaaattagctgagcagattaagggtcaaccacaagtgattgcgaatgtagctgaaccagcacaaattgcttcatcgccaaacacagatgtattgttgcaacgtatggaacagttagagcaagaactaaaaacactaaaagcacaaggagtgagtgttgctcctactcaaaaatcttcgaaaaagcctgcgagaggtatacaaaaatctaaaaatgcattttcaatgcaacaaattgcaaaagtgctagataaagcgaataaggcagatatcaaattgttgaaagatcattggcaagaagtgattgaccatgcccaaaacaatgataaaaaatcactcgttagtttattgcaaaattcggaacctgtggcggcaagtgaagatcacgtccttgtgaaatttgaggaagagatccattgtgaaatcgtcaataaagacgacgagaaacgtagtagtatagaaagtgttgtatgtaatatcgttaataaaaacgttaaagttgttggtgtaccatcagatcaatggcaaagagttcgaacggagtatttacaaaatcgtaaaaacgaaggcgatgatatgccaaagcaacaagcacaacaaacagatattgctcaaaaagcaaaagatcttttcggtgaagaaactgtacatgtgatagatgaagagtga

SEQ ID NO: 4 - protein, 566 aa, Staphylococcus aureus
LNYQALYRMYRPQSFEDVVGQEHVTKTLRNAISKEKQSHAYIFSGPRGTGKTSIAKVFAKAINCLNSTDGEPCNECHICKGITQGTNSDVIEIDAASNNGVDEIRNIRDKVKYAPSESKYKVYIIDEVHMLTTGAFNALLKTLEEPPAHAIFILATTEPHKIPPTIISRAQRFDFKAISLDQIVERLKFVADAQQIECEDEALAFIAKASEGGMRDALSIMDQAIAFGDGTLTLQDALNVTGSVHDEALDHLFDDIVQGDVQASFKKYHQFITEGKEVNRLINDMIYFVRDTIMNKTSEKDTEYRALMNLELDMLYQMIDLINDTLVSIRFSVNQNVHFEVLLVKLAEQIKGQPQVIANVAEPAQIASSPNTDVLLQRMEQLEQELKTLKAQGVSVAPTQKSSKKPARGIQKSKNAFSMQQIAKVLDKANKADIKLLKDHWQEVIDHAQNNDKKSLVSLLQNSEPVAASEDHVLVKFEEEIHCEIVNKDDEKRSSIESVVCNIVNKNVKVVGVPSDQWQRVRTEYLQNRKNEGDDMPKQQAQQTDIAQKAKDLFGEETVHVIDEEZ

SEQ ID NO: 5 - DNA, 1398 bp, Staphylococcus aureus
atggatagaatgtatgagcaaaatcaaatgccgcataacaatgaagctgaacagtctgtcttaggttcaattattatagatccagaattgattaatactactcaggaagttttgcttcctgagtcgttttataggggtgcccatcaacatattttccgtgcaatgatgcacttaaatgaagataataaagaaattgatgttgtaacattgatggatcaattatcgacggaaggtacgttgaatgaagcgggtggcccgcaatatcttgcagagttatctacaaatgtaccaacgacgcgaaatgttcagtattatactgatatcgtttctaagcatgcattaaaacgtagattgattcaaactgcagatagtattgccaatgatggatataatgatgaacttgaactagatgcgattttaagtgatgcagaacgtcgaattttagagctatcatcttctcgtgaaagcgatggctttaaagacattcgagacgtcttaggacaagtgtatgaaacagctgaagagcttgatcaaaatagtggtcaaacaccaggtatacctacaggatatcgagatttagaccaaatgacagcagggttcaaccgaaatgatttaattatccttgcagcgcgtccatctgtaggtaagactgcgttcgcacttaatattgcacaaaaagttgcaacgcatgaagatatgtatacagttggtattttctcgctagagatgggtgctgatcagttagccacacgtatgatttgtagttctggaaatgttgactcaaaccgcttaagaacgggtactatgactgaggaagattggagtcgttttactatagcggtaggtaaattatcacgtacgaagatttttattgatgatacaccgggtattcgaattaatgatttacgttctaaatgtcgtcgattaaagcaagaacatggcttagacatgattgtgattgactacttacagttgattcaaggtagtggttcacgtgcgtccgataacagacaacaggaagtttctgaaatctctcgtacattaaaagcattagcccgtgaattaaaatgtccagttatcgcattaagtcagttatctcgtggtgttgaacaacgacaagataaacgtccaatgatgagtgatattcgtgaatctggttcgattgagcaagatgccgatatcgttgcattcttataccgtgatgattactataaccgtggcggcgatgaagatgatgacgatgatggtggtttcgagccacaaacgaatgatgaaaacggtgaaattgaaattatcattgctaagcaacgtaacggtccaacaggcacagttaagttacattttatgaaacaatataataaatttaccgatatcgattatgcacatgcagatatgatg

SEQ ID NO: 6 - protein, 466 aa, Staphylococcus aureus
MDRMYEQNQMPHNNEAEQSVLGSIIIDPELINTTQEVLLPESFYRGAHQHIFRAMMHLNEDNKEIDVVTLMDQLSTEGTLNEAGGPQYLAELSTNVPTTRNVQYYTDIVSKHALKRRLIQTADSIANDGYNDELELDAILSDAERRILELSSSRESDGFKDIRDVLGQVYETAEELDQNSGQTPGIPTGYRDLDQMTAGFNRNDLIILAARPSVGKTAFALNIAQKVATHEDMYTVGIFSLEMGADQLATRMICSSGNVDSNRLRTGTMTEEDWSRFTIAVGKLSRTKIFIDDTPGIRINDLRSKCRRLKQEHGLDMIVIDYLQLIQGSGSRASDNRQQEVSEISRTLKALARELKCPVIALSQLSRGVEQRQDKRPMMSDIRESGSIEQDADIVAFLYRDDYYNRGGDEDDDDDGGFEPQTNDENGEIEIIIAKQRNGPTGTVKLHFMKQYNKFTDIDYAHADMM

SEQ ID NO: 7 - DNA, 4308 bp, Staphylococcus aureus
atgacagagcaacaaaaatttaaagtgcttgctgatcaaattaaaatttcaaatcaattagatgctgaaattttaaattcaggtgaactgacacgtatagatgtttctaacaaaaacagaacatgggaatttcatattacattaccacaattcttagctcatgaagattatttattatttataaatgcaatagagcaagagtttaaagatatcgccaacgttacatgtcgttttacggtaacaaatggcacgaatcaagatgaacatgcaattaaatactttgggcactgtattgaccaaacagctttatctccaaaagttaaaggtcaattgaaacagaaaaagcttattatgtctggaaaagtattaaaagtaatggtatcaaatgacattgaacgtaatcattttgataaggcatgtaatggaagtcttatcaaagcgtttagaaattgtggttttgatatcgataaaatcatattcgaaacaaatgataatgatcaagaacaaaacttagcttctttagaagcacatattcaagaagaagacgaacaaagtgcacgattggcaacagagaaacttgaaaaaatgaaagctgaaaaagcgaaacaacaagataacaacgaaagtgctgtcgataagtgtcaaattggtaagccgattcaaattgaaaatattaaaccaattgaatctattattgaggaagagtttaaagttgcaatagagggtgtcatttttgatataaacttaaaagaacttaaaagtggtcgccatatcgtagaaattaaagtgactgactatacggactctttagttttaaaaatgtttactcgtaaaaacaaagatgatttagaacattttaaagcgctaagtgttggtaaatgggttagggctcaaggtcgtattgaagaagatacatttattagagatttagttatgatgatgtctgatattgaagagattaaaaaagcgacaaaaaaagataaggctgaagaaaagcgtgtagaattccacttgcatactgcaatgagccaaatggatggtatacccaatattggtgcgtatgttaaacaggcagcagactggggacatccagccattgcggttacagaccataatgttgtgcaagcatttccagatgctcacgcagcagcggaaaaacatggcattaaaatgatatacggtatggaaggtatgttagttgatgatggtgttccgattgcatacaaaccacaagatgtcgtattaaaagatgctacttatgttgtgttcgacgttgagacaactggtttatcaaatcagtatgataaaatcatcgagcttgcagctgtgaaagttcataacggtgaaatcatcgataagtttgaaaggtttagtaatccgcatgaacgattatcggaaacgattatcaatttgacgcatattactgatgatatgttagtagatgcccctgagattgaagaagtacttacagagtttaaagaatgggttggcgatgcgatattcgtagcgcataatgcttcgtttgatatgggcttcatcgatacgggatatgaacgtcttgggtttggaccatcaacgaatggtgttatcgatactttagaattatctcgtacgattaatactgaatatggtaaacatggtttgaatttcttggctaaaaaatatggcgtagaattaacgcaacatcaccgtgccatttatgatacagaagcaacagcttacattttcataaaaatggttcaacaaatgaaagaattaggcgtattaaatcataacgaaatcaacaaaaaactcagtaatgaagatgcatataaacgtgcaagacctagtcatgtcacattaattgtacaaaaccaacaaggtcttaaaaatctatttaaaattgtaagtgcatcattggtgaagtatttctaccgtacacctcgaattccacgttcattgttagatgaatatcgtgagggattattggtaggtacagcgtgtgatgaaggtgaattatttacggcagttatgcagaaggaccagagtcaagttgaaaaaattgccaaatattatgattttattgaaattcaaccaccggcactttatcaagatttaattgatagagagcttattagagatactgaaacattacatgaaatttatcaacgtttaatacatgcaggtgacacagcgggtatacctgttattgcgacaggaaatgcacactatttgtttgaacatgatggtatcgcacgtaaaattttaatagcatcacaacccggcaatccacttaatcgctcaactttaccggaagcacattttagaactacagatgaaatgttaaacgagtttcattttttaggtgaagaaaaagcgcatgaaattgttgtgaaaaatacaaacgaattagcagatcgaattgaacgtgttgttcctattaaagatgaattatacacaccgcgtatggaaggtgctaacgaagaaattagagaactaagttatgcaaatgcgcgtaaactgtatggtgaagacctgcctcaaatcgtaattgatcgattagaaaaagaattaaaaagtattatcggtaatggatttgcggtaatttacttaatttcgcaacgtttagttaaaaaatcattagatgatggatacttagttggttcccgtggttcagtaggttctagttttgtagcgacaatgactgagattactgaagtaaacccgttaccgccacactatatttgtccgaactgtaaaacgagtgaatttttcaatgatggttcagtaggatcaggatttgatttacctgataagacgtgtgaaacttgtggagcgccacttattaaagaaggacaagatattccgtttgaaacatttttaggatttaagggagataaagttcctgatatcgacttaaactttagtggtgaatatcaaccgaatgcccataactacacaaaagtattatttggtgaggataaagtattccgtgcaggtacaattggtactgttgctgaaaagactgcttttggttatgttaaaggttatttgaatgatcaaggtatccacaaaagaggtgctgaaatagatcgactcgttaaaggatgtacaggtgttaaacgtacaactggacagcatccagggggtattattgtagtacctgattacatggatatttatgattttacgccgatacaatatcctgccgatgatcaaaattcagcatggatgacgacacattttgatttccattctattcatgataatgtattaaaacttgatatacttggacacgatgatccaacaatgattcgtatgcttcaagatttatcaggaattgatccaaaaacaatacctgtagatgataaagaagttatgcagatatttagtacacctgaaagtttgggtgttactgaagatgaaattttatgtaaaacaggtacatttggggtaccagaattcggtacaggattcgtgcgtcaaatgttagaagatacaaagccaacaacattttctgaattagttcaaatctcaggattatctcatggtacagatgtgtggttaggcaatgctcaagaattaattaaaaccggtatatgtgatttatcaagtgtaattggttgtcgtgatgatatcatggtttatttaatgtatgctggtttagaaccatcaatggcttttaaaataatggagtcagtacgtaaaggtaaaggtttaactgaagaaatgattgaaacgatgaaagaaaatgaagtgccagattggtatttagattcatgtcttaaaattaagtacatgttccctaaagcccatgcagcagcatacgttttaatggcagtacgtatcgcatatttcaaagtacatcatccactttattactatgcatcttactttacaattcgtgcgtcagactttgatttaatcacgatgattaaagataaaacaagcattcgaaatactgtaaaagacatgtattctcgctatatggatctaggtaaaaaagaaaaagacgtattaacagtcttggaaattatgaatgaaatggcgcatcgaggttatcgaatgcaaccgattagtttagaaaagagtcaggcgttcgaatttatcattgaaggcgatacacttattccgccgttcatatcagtgcctgggcttggcgaaaacgttgcgaaacgaattgttgaagctcgtgacgatggcccatttttatcaaaagaagatttaaacaaaaaagctggattatctcagaaaattattgagtatttagatgagttaggctcattaccgaatttaccagataaagctcaactttcgatatttgatatg

SEQ ID NO: 8 - protein, 1435 aa, Staphylococcus aureus
MTEQQKFKVLADQIKISNQLDAEILNSGELTRIDVSNKNRTWEFHITLPQFLAHEDYLLFINAIEQEFKDIANVTCRFTVTNGTNQDEHAIKYFGHCIDQTALSPKVKGQLKQKKLIMSGKVLKVMVSNDIERNHFDKACNGSLIKAFRNCGFDIDKIIFETNDNDQEQNLASLEAHIQEEDEQSARLATEKLEKMKAEKAKQQDNKQSAVDKCQIGKPIQIENIKPIESIIEEEFKVAIEGVIFDINLKELKSGRHIVEIKVTDYTDSLVLKMFTRKNKDDLEHFKALSVGKWVRAQGRIEEDTFIRDLVMMMSDIEEIKKATKKDKAEEKRVEFHLHTAMSQMDGIPNIGAYVKQAADWGHPAIAVTDHNVVQAFPDAHAAAEKHGIKMIYGMEGMLVDDGVPIAYKPQDVVLKDATYVVFDVETTGLSNQYDKIIELAAVKVHNGEIIDKFERFSNPHERLSETIINLTHITDDMLVDAPEIEEVLTEFKEWVGDAIFVAHNASFDMGFIDTGYERLGFGPSTNGVIDTLELSRTINTEYGKHGLNFLAKKYGVELTQHHRAIYDTEATAYIFIKMVQQMKELGVLNHNEINKKLSNEDAYKRARPSHVTLIVQNQQGLKNLFKIVSASLVKYFYRTPRIPRSLLDEYREGLLVGTACDEGELFTAVMQKDQSQVEKIAKYYDFIEIQPPALYQDLIDRELIRDTETLHEIYQRLIHAGDTAGIPVIATGNAHYLFEHDGIARKILIASQPGNPLNRSTLPEAHFRTTDEMLNEFHFLGEEKAHEIVVKNTNELADRIERVVPIKDELYTPRMEGANEEIRELSYANARKLYGEDLPQIVIDRLEKELKSIIGNGFAVIYLISQRLVKKSLDDGYLVGSRGSVGSSFVATMTEITEVNPLPPHYICPNCKTSEFFNDGSVGSGFDLPDKTCETCGAPLIKEGQDIPFEKFLGFKGDKVPDIDLNFSGEYQPNAHNYTKVLFGEDKVFRAGTIGTVAEKTAFGYVKGYLNDQGIHKRGAEIDRLVKGCTGVKATTGQHPGGIIVVPDYMDIYDFTPIQYPADDQNSAWMTTHFDFHSIHDNVLKLDILGHDDPTMIRMLQDLSGIDPKTIPVDDKEVMQIFSTPESLGVTEDEILCKTGTFGVPNSDRIRRQMLEDTKPTTFSELVQISGLSHGTDVWLGNAQELIKTGICDLSSVIGCRDDIMVYLMYAGLEPSMAFKIMESVRKGKGLTEEMIETMKENEVPDWYLDSCLKIKYIFPKAHAAAYVLMAVRIAYFKVHHPLYYYASYFTIRASDFDLITMIKDKTSIRNTVKDMYSRYMDLGKKEKDVLTVLEIMNEMAHRGYRMQPISLEKSQAFEFIIEGDTLIPPFISVPGLGENVAKRIVEARDDGPFLSKEDLNKKAGLYQKIIEYLDELGSLPNLPDKAQLSIFDM

SEQ ID NO: 9 - DNA, 1134 bp, Staphylococcus aureus
atgatggaattcactattaaaagagattattttattacacaattaaatgacacattaaaagctatttcaccaagaacaacattacctatattaactggtatcaaaatcgatgcgaaagaacatgaagttatattaactggttcagactctgaaatttcaatagaaatcactattcctaaaactgtagatggcgaagatattgtcaatatttcagaaacaggctcagtagtacttcctggacgattctttgttgatattataaaaaaattacctggtaaagatgttaaattatctacaaatgaacaattccagacattaattacatcaggtcattctgaatttaatttgagtggcttagatccagatcaatatcctttattacctcaagtttctagagatgacgcaattcaattgtcggtaaaagtacttaaaaacgtgattgcacaaacgaattttgcagtgtccacctcagaaacacgcccagtactaactggtgtgaactggcttatacaagaaaatgaattaatatgcacagcgactgattcacaccgcttggctgtaagaaagttgcagttagaagatgtttctgaaaacaaaaatgtcatcattccaggtaaggctttagctgaattaaataaaattatgtctgacaatgaagaagacattgatatcttctttgcttcaaaccaagttttatttaaagttggaaatgtgaactttatttctcgattattagaaggacattatcctgatacaacacgtttattccctgaaaactatgaaattaaattaagtatagacaatggggagttttatcatgcgattgatcgtgcctctttattagcacgtgaaggtggtaataacgttattaaattaagtacaggtgatgacgttgttgaattatcttctacatcaccagaaattggtactgtaaaagaagaagttgatgcaaacgatgttgaaggtggtagcctgaaaatttcattcaactctaaatatatgatggatgctttaaaagcaatcgataatgatgaggttgaagttgaattcttcggtacaatgaaaccatttattctaaaaccaaaaggtgacgactcggtaacgcaattaattttaccaatcagaacttactaa

SEQ ID NO: 10 - protein, 377 aa, Staphylococcus aureus
MMEFTIKRDYFITQLNDTLKAISPRTTLPILTGIKIDAKEHEVILTGSDSEISIEITIPKTVDGEDIVNISETGSVVLPGRFFVDIIKKLPGKDVKLSTNEQFQTLITSGHSEFNLSGLDPDQYPLLPQVSRDDAIQLSVKVLKNVIAQTNFAVSTSETRPVLTGVNWLIQENELICTATDSHRLAVRKLQLEDVSENKNVIIPGKALAELNKIMSDNEEDIDIFFASNQVLFKVGNVNFISRLLEGHYPDTTRLFPENYEIKLSIDNGEFYHAIDRASLLAREGGNNVIKLSTGDDVVELSSTSPEIGTVKEEVDANDVEGGSLKISFNSKYMMDALKAIDNDEVEVEFFGTMKPFILKPKGDDSVTQLILPIRTY

SEQ ID NO: 11 - DNA, 930 bp, Staphylococcus aureus
atggatgaacagcaacaattgacgaatgcatatcattcaaataaattatcgcatgcctatttatttgaaggtgatgatgcacaaacgatgaaacaagttgcgattaattttgcaaagcttattttatgtcaaacagatagtcaatgtgaaacaaaggttagtacatataatcatccagactttatgtatatatcaacaactgagaatgcaattaagaaagaacaagttgaacaacttgtgcgtcatatgaatcaacttcctatagaaagcacaaataaagtgtacatcatcgaagactttgaagactttgaaaagttaactgttcaaggggaaaacagtatcttgaaatttcttgaagaaccaccggacaatacgattgctattttattgtctacaaaacctgagcaaattttagacacaatccattcaaggtgtcagcatgtatatttcaagcctattgataaagaaaagtttataaatagattagttgaacaaaacatgtctaagccagtagctgaaatgattagtacttatactacgcaaatagataatgcaatggctttaaatgaagaatttgatttattagcattaaggaaatcagttatacgttgggaattgttgcttactaataagccaatggcacttataggtattattgatttattgaaacaggctaaaaataaaaaactgcaatctttaactattgcagctgtgaatggtttcttcgaagatatcatacatacaaaggtaaatgtagaggataaacaaatatatagtgatttaaaaaatgatattgatcaatatgcgcaaaagttgtcgtttaatcaattaattttgatgtttgatcaactgacggaagcacataagaaattgaatcaaaatgtaaatccaacgcttgtatttgaacaaatcgtaattaagggtgtgagt

SEQ ID NO: 12 - protein, 310 aa, Staphylococcus aureus
MDEQQQLTNAYHSNKLSHAYLFEGDDAQTMKQVAINFAKLILCQTDSQCETKVSTYNHPDFMYISTTENAIKKEQVEQLVRHMNQLPIESTNKVYIIEDFEDFEKLTVQGENSILKFLEEPPDNTIAILLSTKPEQILDTIHSRCQHVYFKPIDKEKFINRLVEQNMSKPVAEMISTYTTQIDNAMALNEEFDLLALRKSVIRWELLLTNKPMALIGIIDLLKQAKNKKLQSLTIAAVNGFFEDIIHTKVNVEDKQIYSDLKNDIDQYAQKLSFNQLILMFDQLTEAHKKLNQNVNPTLVFEQIVIKGVS

SEQ ID NO: 13 - DNA, 744 bp, Staphylococcus aureus
atgagcgacaatattgtagctatttatggagatgtgcctgaattggttgaaaaacaaagtgcagaaatcatatcacaatttttgaaaagtgatagagatgactttaactttgtgaaatataatttatacgaaacagagattgcaccaattgttgaagaaacattaacattgcctttcttttcagataaaaaagcaattttggttaaaaatgcatatatatttacaggtgaaaaagcgccaaaagatatggctcataatgtagaccaattaatagaatttattgaaaaatatgatggcgaaaatttgattgtctttgagatatatcaaaataaacttgatgaaagaaaaaagttaactaaaactctaaaaaagcatgcaaggcttaaaaaaatagagcagatgtcggaggagatcaagtggattcaaaaaaaagaacaagcgattgattttgtaaaagatcttataacaatgaaagaagaaccaattaaacttcttgcacttacatcaaattatagacttttttatcaatgtaaaattctttcacaaaaaggttatagtggtcaacaaattgcaaaaacaataggtgttcatccatatagagtgaaacttgcacttggtcaagtgagacattatcaacttgatgaacttcttaatattattgatgcatgtgcagaaacagattataaacttaaatcatcatatatggataaacaacttattcttgaactttttattctttcactt

SEQ ID NO: 14 - protein, 248 aa, Staphylococcus aureus
MSDNIVAIYGDVPELVEKQSAEIISQFLKSDRDDFNFVKYNLYETEIAPIVEETLTLPFFSDKKAILVKNAYIFTGEKAPKDMAHNVDQLIEFIEKYDGENLIVFEIYQNKLDERKKLTKTLKKHARLKKIEQMSEEIKWIQKKEQAIDFVKDLITMKEEPIKLLALTSNYRLFYQCKILSQKGYSGQQIAKTIGVHPYRVKLALGQVRHYQLDELLNIIDACAETDYKLKSSYMDKQLILELFILSL

SEQ ID NO: 15 - DNA, 1719 bp, Staphylococcus aureus
atgataggtttgtgtccttttcatgatgaaaagacaccttcatttacagtttctgaagataaacaaatctgtcattgttttggttgtaaaaaaggtggcaatgtttttcaatttactcaagaaattaaagacatatcatttgttgaagcggttaaagaattaggtgatagagttaatgttgctgtagatattgaggcaacacaatctaactcaaatgttcaaattgcttctgatgatttacaaatgattgaaatgcatgagttaatacaagaattttattattacgctttaacaaagacagtcgaaggcgaacaagcattaacatacttacaagaacgtggttttacagatgcgcttattaaagagcgaggcattggctttgcacccgatagctcacatttttgtcatgattttcttcaaaaaaagggttacgatattgaattagcatatgaagccggattattatcacgtaacgaagaaaatttcagttattacgatagatttcgaaatcgtattatgtttcctttgaaaaatgcgcaaggaagaattgttggatattcaggtcgaacatataccggtcaagaaccaaaatacctaaatagtcctgaaacgcctatctttcaaaaaagaaagttgttatataacttagataaagcacgtaaatcaattagaaaattagatgaaattgtattactagaaggttttatggatgttataaaatctgatactgctggcttgaaaaacgttgttgcaacaatgggtacacagttgtcagatgaacatattacctttatacgaaagttaacatcaaatataacattaatgtttgatggggattttgcgggtagtgaagcaacacttaaaacaggtcaacatttgttacagcaagggctaaatgtatttgttatacaattgccatctggcatggatccggatgaatacattggtaagtatggcaacgacgcatttactacttttgtaaaaaatgacaaaaagtcatttgcacattataaagtaagtatattaaaagatgaaattgcacataatgacctttcatatgaacgttatttgaaagaactgagtcatgacatttcacttatgaagtcatcaattctgcaacaaaaggctataaatgatgttgcgccatttttcaatgttagtcctgagcagttagctaacgaaatacaattcaatcaagcaccagccaattattatccagaagatgagtatggcggttatgatgagtatggcggttatattgaacctgagccaattggtatggcacaatttgacaatttgagccgtcgagaaaaagcggagcgagcatttttaaaacatttaatgagagataaagatacatttttaaattattatgaaagtgttgataaggataacttcacaaatcagcattttaaatatgtattcgaagtcttacatgatttttatgcggaaaatgatcaatataatatcagtgatgctgtgcagtatgttaattcaaatgagttgagagaaacactaattagcttagaacaatataatttgaatggcgaaccatatgaaaatgaaattgatgattatgtcaatgttattaatgaaaaaggacaagaaacaattgagtcattgaatcataaattaagggaagctacaaggattggcgatgtagaattacaaaaatactatttacagcaaattgttgctaagaataaagaacgcatgtag

SEQ ID NO: 16 - protein, 572 aa, Staphylococcus aureus
MIGLCPFHDEKTPSFTVSEDKQICHCFGCKKGGNVFQFTQEIKDISFVEAVKELGDRVNVAVDIEATQSNSNVQIASDDLQMIEMHELIQEFYYYALTKTVEGEQALTYLQERGFTDALIKERGIGFAPDSSHFCHDFLQKKGYDIELAYEAGLLSRNEENFSYYDRFRNRIMFPLKNAQGRIVGYSGRTYTGQEPKYLNSPETPIFQKRKLLYNLDKARKSIRKLDEIVLLEGFMDVIKSDTAGLKNVVATMGTQLSDEHITFIRKLTSNITLMFDGDFAGSEATLKTGQHLLQQGLNVFVIQLPSGMDPDEYIGKYGNDAFTTFVKNDKKSFAHYKVSILKDEIAHNDLSYERYLKELSHDISLMKSSILQQKAINDVAPFFNVSPEQLANEIQFNQAPANYYPEDEYGGYDEYGGYIEPEPIGMAQFDNLSRREKAERAFLKHLMRDKDTFLNYYESVDKDNFTNQHFKYVFEVLHDFYAENDQYNISDAVQYVNSNELRETLISLEQYNLNGEPYENEIDDYVNVINEKGQETIESLNHKLREATRIGDVELQKYYLQQIVAKNKERM

SEQ ID NO: 17 - DNA, 4395 bp, Streptococcus pyogenes
atgtcagatttattcgctaaattgatggaccagatagaaatgccacttgacatgagacgttcaagtgccttttcatctgctgatattatcgaggtaaaggtacattcggtgtcacgcttgtgggaatttcattttgcctttgcagcggttttaccgattgcaacttatcgtgaattgcatgatcgtttgataagaacttttgaggcggctgacattaaggtaacctttgacatccaagctgctcaggtggattattcagatgatctgcttcaagcttattaccaagaagcttttgagcatgcaccgtgtaatagtgctagttttaaatcttctttctcaaagctcaaagtgacttatgaggatgacaaactcattattgcagcgccaggttttgtgaataacgatcattttagaaacaatcatctgcctaatctggtcaagcaattagaagcctttggctttggcatcttgaccatagatatggtgtcagatcaggaaatgactgagcatttgaccaagaattttgtttccagtcgtcaggctcttgtgaaaaaggctgtgcaggataatttggaagcccaaaaatctcttgaagccatgatgccaccagttgaggaagccacacctgctcctaagtttgactacaaggaacgagcagctaagcgtcaggcagggtttgaaaaagcaaccatcacaccaatgattgagattgagaccgaagaaaaccggattgtctttgagggtatggtttttgacgtggagcgtaaaacgactaggacaggtcgccatatcatcaactttaaaatgacagactatacctcctcgtttgctctccaaaaatgggctaaagacgatgaggagctccgtaaatttgatatgattgctaagggagcttggttacgggtacaagggaatattgagaccaatccttttacgaagagtctcaccatgaatgtccagcaggtcaaagaaattgtccgtcatgagcgcaaagacctgatgccagaagggcaaaagcgggtcgaacttcatgcccacaccaatatgtctaccatggatgccttaccgacagtagaaagcttgattgatacggcagccaagtggggacacaaggcgattgctatcaccgaccatgctaatgtgcaaagttttcctcatggctaccatagggctcgcaaagctgggattaaggctatttttggcctagaagccaatattgttgaggacaaggtgcctatttcttatgaacctgttgatatggatttgcacgaagccacctatgtggtctttgacgtggaaaccacaggtctatctgctatgaataatgacctgattcagattgcggcttccaaaatgtttaaaggaaatattgtagagcagtttgatgaattcattgatcctgggcatcctctttcagcctttaccaccgaattgacaggaattaccgataagcatttgcagggcgccaagccattggttactgtcctaaaagcttttcaggacttttgcaaagatagtatcttggttgcccacaacgccagttttgacgtgggctttatgaacgccaattatgaacgccacgacttgcccaaaatcacacagcctgtgattgataccttagaatttgctagaaacttgtatcctgagtacaagcgtcacggtttgggaccgctcaccaagcgtttccaagtgagtctagaccaccatcatatggccaattacgacgcggaagccacaggacgtcttttgtttatttttctaaaagatgccagagaaaagcatggcatcaaaaatcttttgcaactcaatacagatttggtggctgaggattcttacaaaaaagcgcggattaagcatgcgactatctatgtgcaaaatcaggttggtcttaaaaatatgtttaagttggtcagcctttccaatatcaaatattttgaaggggtgccgcgtattccaagaaccgtcttagatgctcacagagagggtttgttacttggtacagcttgttctgacggcgaggtttttgatgccgttctgactaaaggaattgatgcagcggttgatttggctaggtattatgattttatcgaaatcatgccaccagccatttaccagccattggttgtccgtgaattaatcaaagatcaagcaggtattgagcaggtgattcgtgacctcattgaagtagggaaacgagctaagaaacctgtgcttgccactgggaatgtgcattatctagagcctgaagaagagatttaccgtgaaattattgtgcgtagtcttggtcagggtgccatgattaatagaacaatcggccgtggggaaggggcacagcctgctcctctacctaaagcgcactttagaacaaccaatgaaatgctggatgagtttgcctttcttggaaaagacctcgcttatcaagtagttgtgcaaaatactcaggattttgcggaccgtattgaggaagtggaagtggttaagggcgatctttacaccccgtatattgataaggccgaagagacggttgccgaattaacctatcaaaaagcctttgaaatttatggtaatcctctcccagatattattgatttacgcattgaaaaagagttaacctctatcttggggaacggttttgctgtgatttatctcgcttcccaaatgcttgttaaccggtcaaatgagcgaggctacctagttggttctaggggatctgtagggtctagctttgtcgccaccatgattgggattactgaggttaatcctatgccgcctcactacgtttgcccgtcctgccaacattctgaatttatcacagatgggtcagttggatctggctatgatttgcctaataaaccctgtccgaaatgtggcaccccttatcaaaaagatgggcaagacattccctttgagacctttcttgggtttgatggggataaggtgcccgatattgatttgaacttctctggtgatgaccagcccagtgcccatttggatgtccgagatatttttggtgacgaatacgcctttcgtgctggaacagttggtaccgtagcagaaaaaacagcttatggatttgtcaaaggctatgaacgcgactatggcaagttctatcgtgatgctgaggtggatcgtctagcagcaggtgctgctggtgtgaaacgaacgactgggcagcaccctggggggattgttgttattcctaattacatggatgtttatgattttacccccgtgcaatatccagccgatgatgtaacggcttcttggcagacaactcactttaacttccatgatattgatgaaaacgtcttgaaacttgatatcctagggcatgatgatccgaccatgattcgtaaacttcaggatttatcgggcattgatcctattactattcctgctgatgatccgggagttatggctctcttttctgggacagaggttttgggcgttaccccggaacaaattgggacaccgactggtatgctaggcattccagaatttggaaccaactttgttcgcggcatggttaatgagacgcatccgaccacttttgcggagcttttgcagttgtctggactatctcatggaaccgatgtttggcttggtaatgcacaagatttgattaaagaaggcattgcaaccctaaaaaccgttatcggttgtcgtgacgacatcatggtttacctcatgcacgcaggcttagaaccaaaaatggcctttaccattatggagcgtgtgcgtaagggattatggctaaaaatttctgaggaagaacgtaatggctatattgatgccatgcgagaaaacaatgtgcccgactggtacattgaatcgtgtggaaaaatcaagtacatgttccctaaagcccatgcggcagcttatgttttgatggcccttcgggtggcttatttcaaggtgcaccaccccattatgtattattgtgcttatttctctattcgtgcgaaggcttttgaattaaaaaccatgagtggtggtttagatgctgttaaagcaagaatggaagatattactataaaacgtaaaaataatgaagccaccaatgtggaaaatgacctctttacaaccttggagattgtcaacgaaatgttagaacgcggctttaagtttggcaaattagacctttacaaaagtgatgctatagaattccaaatcaaaggagatacccttatccctccatttatagcgctagaaggtctgggtgaaaacgtggccaagcaaatcgttaaagctcgtcaagaaggcgaattcctctctaaaatggaattgcgtaaacgaggcggggcatcgtcaacgctcgttgagaaaatggatgagatgggtattttaggaaatatgccagaagataatcaattaagtctttttgatgactttttc

SEQ ID NO: 18 - protein, 1465 aa, Streptococcus pyogenes
MSDLFAKLMDQIEMPLDMRRSSAFSSADIIEVKVHSVSRLWEFHFAFAAVLPIATYRELHDRLIRTFEAADIKVTFDIQAAQVDYSDDLLQAYYQEAFEHAPCNSASFKSSFSKLKVTYEDDKLIIAAPGFVNNDHFRNNHLPNLVKQLEAFGFGILTIDMVSDQEMTEHLTKNFVSSRQALVKKAVQDNLEAQKSLEAMMPPVEEATPAPKFDYKERAAKRQAGFEKATITPMIEIETEENRIVFEGMVFDVERKTTRTGRHIINFKMTDYTSSFALQKWAKDDEELRKFDMIAKGAWLRVQGNIETNPFTKSLTMNVQQVKEIVRHERKDLMPEGQKRVELHAHTNMSTMDALPTVESLIDTAAKWGHKAIAITDHANVQSFPHGYHRARKAGIKAIFGLEANIVEDKVPISYEPVDMDLHEATYVVFDVETTGLSAMNNDLIQIAASKMFKGNIVEQFDEFIDPGHPLSAFTTELTGITDKHLQGAKPLVTVLKAFQDFCKDSILVAHNASFDVGFMNANYERHDLPKITQPVIDTLEFARNLYPEYKRHGLGPLTKRFQVSLDHHHMANYDAEATGRLLFIFLKDAREKHGIKNLLQLNTDLVAEDSYKKARIKHATIYVQNQVGLKNMFKLVSLSNIKYFEGVPRIPRTVLDAHREGLLLGTACSDGEVFDAVLTKGIDAAVDLARYYDFIEIMPPAIYQPLVVRELIKDQAGIEQVIRDLIEVGKRAKKPVLATGNVHYLEPEEEIYREIIVRSLGQGAMINRTIGRGEGAQPAPLPKAHFRTTNEMLDEFAFLGKDLAYQVVVQNTQDFADRIEEVEVVKGDLYTPYIDKAEETVAELTYQKAFEIYGNPLPDIIDLRIEKELTSILGNGFAVIYLASQMLVNRSNERGYLVGSRGSVGSSFVATMIGITEVNPMPPHYVCPSCQHSEFITDGSVGSGYDLPNKPCPKCGTPYQKDGQDIPFETFLGFDGDKVPDIDLNFSGDDQPSAHLDVRDIFGDEYAFRAGTVGTVAEKTAYGFVKGYERDYGKFYRDAEVDRLAAGAAGVKRTTGQHPGGIVVIPNYMDVYDFTPVQYPADDVTASWQTTHFNFHDIDENVLKLDILGHDDPTMIRKLQDLSGIDPITIPADDPGVMALFSGTEVLGVTPEQIGTPTGMLGIPEFGTNFVRGMVNETHPTTFAELLQLSGLSHGTDVWLGNAQDLIKEGIATLKTVIGCRDDIMVYLMHAGLEPKMAFTIMERVRKGLWLKISEEERNGYIDAMRENNVPDWYIESCGKIKYMFPKAHAAAYVLMALRVAYFKVHHPIMYYCAYFSIRAKAFELKTMSGGLDAVKARMEDITIKRKNNEATNVENDLFTTLEIVNEMLERGFKFGKLDLYKSDAIEFQIKGDTLIPPFIALEGLGENVAKQIVKARQEGEFLSKMELRKRGGASSTLVEKMDEMGILGNMPEDNQLSLFDDFF

SEQ ID NO: 19 - DNA, 3102 bp, Streptococcus pyogenes
atgtttgctcaacttgatactaaaactgtatactcatttatggatagtttaattgacttaaatcattattttgaacgagcaaagcaatttggttaccacaccataggaatcatggataaggataatctttatggtgcttaccattttattaaaggttgtcaaaaaaatggactgcagccagttttaggtttggaaatagagattctctatcaagagcggcaggtgctccttaacttaatcgcccagaatacacaaggctatcatcagcttttaaaaatttccacggcaaaaatgtctggcaagcttcatatggattacttctgccaacatttggaagggatagcggttattattcctagtaagggttggagcgatacattagtggtcccttttgactactatatgggtgttgatcagtatactgatttatctcatatggattctaagaggcagcttatacccctaaggacagttcgttattttgcgcaagatgatatggaaaccctgcacatgttgcatgccattcgagataacctcagtctggcagagacccctgtggtagaaagtgatcaagagttagcagattgtcaacaactaaccgccttctatcaaacacactgccctcaagctctacagaatttagaagacttagtgtcaggaatctattatgatttcgatacaaatttaaaattgcctcattttaatagagataagtctgccaagcaagaattgcaagacttgactgaggctggtttgaaggaaaaaggattgtggaaagagccttatcaatcgcgcttactacatgaattggtcattatttctgacatgggctttgatgattattttttgattgtgtgggatttacttcgctttggacgcagtaaaggctattatatgggaatgggacgtggctcggcggcaggtagtctagtggcttatgctctgaacattacagggattgatccagttcaacatgatttgctatttgagcgctttttaaacaaagaacgttatagcatgcctgatattgatatcgatcttccagatatttaccgttcagaatttctacggtatgtccgaaatcgttatggtagcgaccattcggcgcaaattgtgaccttttcaacctttggccaggctattcgtgatgttttcaaacggttcggggttccagaatacgaactgactaatctcactaaaaaaattggttttaaagatagcttggctactgtctatgaaaagtcaatctcttttaggcaggttattaatagtagaactgaatttcaaaaggcttttgccattgccaagcgtatcgaaggaaatccaagacaaacgtccattcacgcagctggtattgtgatgagtgatgatgccttgaccaatcatattcctctaaaatcgggcgatgacatgatgatcacccagtatgatgctcatgcggtcgaagctaatggcctgttaaaaatggattttttggggttaagaaatttgacctttgttcaaaaaatgcaagagaaggttgctaaagactacgggtgtcagattgatattacagccattgatttagaagacccgcaaacgttggcactttttgctaaaggggataccaagggaattttccaatttgaacaaaatggtgctattaatcttttaaaacggattaagccacaacgttttgaagaaattgttgccactaccagtctaaatagaccaggggcaagtgactataccactaatttcattaaacgaagagaaggacaagaaaaaattgatttgattgatcctgtgattgctcccattttagagccaacttacggtattatgctttatcaagaacaagttatgcagattgcacaggtttatgctggttttacgttaggcaaggccgacttgttaaggcgtgccatgtctaaaaaaaatctacaagaaatgcaaaaaatggaagaagactttattgcttctgctaagcacctagggagagctgaagaaacagctagaggactttttaaacggatggaaaaatttgcaggttatggttttaaccgcagccatgcctttgcctattcagctttagcttttcaattggcttatttcaaagcccattacccggctgttttttacgatatcatgatgaattattctagcagtgactatatcacagatgctctagaatcagattttcaagtagcgcaagttaccattaatagtattccttacactgataaaattgaagctagcaagatttacatggggctgaaaaatattaaggggttgccaagggattttgcttattggattatcgagcaaagaccatttaatagcgtagaggattttctcactagaactccagaaaaatatcaaaaaaaggttttccttgagcctctgataaaaataggtctgtttgattgctttgagcctaaccgtaaaaaaattctggacaatttggatggtttactggtatttgttaatgagcttggttctcttttttcagattcttcctttagttgggtagatacgaaagattactcagtaactgaaaaatattctttggaacaggagatcgttggagttggcatgagcaagcatcctttaattgatattgctgagaaaagtacccaaacttttactcctatttcacagttagtcaaagaaagcgaagcagtcgtactgattcaaatagatagcattaggatcattagaaccaaaacaagtgggcagcaaatggcttttttaagtgtgaatgacactaagaaaaagctcgatgtcacactttttccacaagagtatgccatttataaagaccaattaaaagaaggagaattctattacttaaaaggtagaataaaagaaagagaccatcgactgcagatggtgtgtcagcaagtgcaaatggctattagtcaaaaatattggttattagttgaaaaccatcagtttgattcccaaatttctgagattttaggtgcctttccaggaacgactccagttgttattcactatcaaaaaaataaggaaacaattgcattaactaagattcaggttcatgtaacagagaatttaaaggaaaaacttcgtccttttgttctgaaaacggtttttcga

SEQ ID NO: 20 - protein, 1034 aa, Streptococcus pyogenes
MFAQLDTKTVYSFMDSLIDLNHYFERAKQFGYHTIGIMDKDNLYGAYHFIKGCQKNGLQPVLGLEIEILYQERQVLLNLIAQNTQGYHQLLKISTAKMSGKLHMDYFCQHLEGIAVIIPSKGWSDTLVVPFDYYMGVDQYTDLSHMDSKRQLIPLRTVRYFAQDDMETLHMLHAIRDNLSLAETPVVESDQELADCQQLTAFYQTHCPQALQNLEDLVSGIYYDFDTNLKLPHFNRDKSAKQELQDLTEAGLKEKGLWKEPYQSRLLHELVIISDMGFDDYFLIVWDLLRFGRSKGYYMGMGRGSAAGSLVAYALNITGIDPVQHDLLFERFLNKERYSMPDIDIDLPDIYRSEFLRYVRNRYGSDHSAQIVTFSTFGPKQAIRDVFKRFGVPEYELTNLTKKIGFKDSLATVYEKSISFRQVINSRTEFQKAFAIAKRIEGNPRQTSIHAAGIVMSDDALTNHIPLKSGDDMMITQYDAHAVEANGLLKMDFLGLRNLTFVQKMQEKVAKDYGCQIDITAIDLEDPQTLALFAKGDTKGIFQFEQNGAINLLKRIKPQRFEEIVATTSLNRPGASDYTTNFIKRREGQEKIDLIDPVIAPILEPTYGIMLYQEQVMQIAQVYAGFTLGKADLLRRAMSKKNLQEMQKMEEDFIASAKHLGRAEETARGLFKRMEKFAGYGFNRSHAFAYSALAFQLAYFKAHYPAVFYDIMMNYSSSDYITDALESDFQVAQVTINSIPYTDKIEASKIYMGLKNIKGLPRDFAYWIIEQRPFNSVEDFLTRTPEKYQKKVFLEPLIKIGLFDCFEPNRKKILDNLDGLLVFVNELGSLFSDSSFSWVDTKDYSVTEKYSLEQEIVGVGMSKHPLIDIAEKSTQTFTPISQLVKESEAVVLIQIDSIRIIRTKTSGQQMAFLSVNDTKKKLDVTLFPQEYAIYKDQLKEGEFYYLKGRIKERDHRLQMVCQQVQMAISQKYWLLVENHQFDSQISEILGAFPGTTPVVIHYQKNKETIALTKIQVTENLKEKLRPFVLKTVFR

SEQ ID NO: 21 - DNA, 1038 bp, Streptococcus pyogenes
atgattgcgatagaaaagattgaaaaactgagtaaagaaaatttgggtcttataacccttgtcacaggagatgacattggtcagtatagccagttgaaatcccgcttaatggagcagattgcttttgataaggatgatttggcctattcttactttgatatgtctgaggccgcttatcaggatgcagaaatggatctagtgagcctacccttctttgctgagcagaaggtggttatttttgaccatttgttagatatcacgaccaataaaaaaagtttcttaaaagaaaaagacctaaaggcctttgaagcctatttagaaaatcccttagagactactcgactaattatctttgctccaggtaaattggatagtaagagacggcttgttaagcttttgaaacgtgatgcccttgttttagaagccaaccctctgaaagaagcagagctaagaacttattttcaaaaatacagtcatcaactgggtttaggtttcgagagtggtgcctttgaccaattacttttgaaatcaaacgatgattttagtcaaatcatgaaaaacatggcctttttaaaagcctataaaaaaacgggaaatattagcctaactgatattgagcaagccattcctaaaagtttacaagataatattttcgatctgactagacttgtcctaggaggtaaaattgatgcggctagagatttgattcatgatttacggttatctggagaagatgacattaaattaatcgctatcatgctaggccaatttcgcttatttttgcagctgactattcttgctagagatgtaaaaaacgagcaacaactagtgattagtttatcagatattcttgggcggcgggttaatccttaccaggtcaagtatgcgttaaaggattctaggaccttatctcttgcctttctaacaggagcggtgaaaaccttgattgagacagattaccagataaaaacaggactttatgagaagagttatctagttgatattgctctcttaaaaatcatgactcactctcaaaaa

SEQ ID NO: 22 - protein, 346 aa, Streptococcus pyogenes
MIAIEKIEKLSKENLGLITLVTGDDIGQYSQLKSRLMEQIAFDKDDLAYSYFDMSEAAYQDAEMDLVSLPFFAEQKVVIFDHLLDITTNKKSFLKEKDLKAFEAYLENPLETTRLIIFAPGKLDSKRRLVKLLKRDALVLEANPLKEAELRTYFQKYSHQLGLGFESGAFDQLLLKSNDDFSQIMKNMAFLKAYKKTGNISLTDIEQAIPKSLQDNIFDLTRLVLGGKIDAARDLIHDLRLSGEDDIKLIAIMLGQFRLFLQLTILARDVKNEQQLVISLSDILGRRVNPYQVKYALKDSRTLSLAFLTGAVKTLIETDYQIKTGLYEKSYLVDIALLKIMTHSQK

SEQ ID NO: 23 - DNA, 873 bp, Streptococcus pyogenes
atggatttagcgcaaaaagctcctaacgtttatcaagcttttcagacaattttaaagaaagaccgtctgaatcatgcttatcttttttcgggtgattttgctaatgaagaaatggctctttttttagctaaggtcatcttttgtgaacagaaaaaggatcagacgccctgcgggcattgtcgctcttgtcaattgattgaacaaggagattttgccgatgtgacggtattggaaccaacagggcaagtgattaaaacggatgtggtcaaagaaatgatggctaacttttctcagacaggatatgaaaacaaacgacaagtttttattatcaaagattgtgacaaaatgcatatcaatgccgctaatagcttgctaaaatacattgaggagcctcagggagaagcttacatatttttattgaccaatgatgataacaaagtgcttccgaccattaaaagtcggacacaggtttttcagtttcctaaaaacgaagcctatctttaccaattggcacaagaaaagggattattaaaccatcaggctaagctagtagccaaacttgccacaaacaccagtcatctagaacgtctgttgcaaacgagcaagcttttagaactgataactcaagcagagcgttttgtatctatttggctgaaagatcagttgcaggcatatttagcgttgaaccgtctggtacagttagcaactgaaaaagaagaacaagatttagttttgacccttttgaccttgctcttggcaagagagcgtgcgcaaacgcctttgacacaattggaggctgtctatcaggctaggctcatgtggcagagcaatgttaattttcaaaacacattagaatatatggtgatgtcagaa

SEQ ID NO: 24 - protein, 291 aa, Streptococcus pyogenes
MDLAQKAPNVYQAFQTILKKDRLNHAYLFSGDFANEEMALFLAKVIFCEQKKDQTPCGHCRSCQLIEQGDFADVTVLEPTGQVIKTDVVKEMMANFSQTGYENKRQVFIIKDCDKMHINAANSLLKYIEEPQGEAYIFLLTNDDNKVLPTIKSRTQVFQFPKNEAYLYQLAQEKGLLNHQAKLVAKLATNTSHLERLLQTSKLLELITQAERFVSIWLKDQLQAYLALNRLVQLATEKEEQDLVLTLLTLLLARERAQTPLTQLEAVYQARLMWQSNVNFQNTLEYMVMSE

SEQ ID NO: 25 - DNA, 1665 bp, Streptococcus pyogenes
atgtatcaagctctttatcggaaataccggagccaaacgtttgacgaaatggtgggacaatcggttatttccacaactttaaagcaggcagttgaatctggcaagattagccatgcttatcttttttcaggtcctagagggactgggaaaacaagtgcggcaaagatttttgcaaaggccatgaattgtcctaaccaagtcgatggtgaaccctgtaatcaatgcgatatttgccgagatatcacgaatggaagcttggaagatgtgattgaaattgatgctgcctcgaataatggtgttgatgaaattcgtgacattcgagacaaatcaacctatgcgccaagtcgtgcgacttacaaggtttatattattgatgaggttcacatgttatcaacaggggcttttaatgcgcttttgaaaactttggaagaaccgacagaatgttgtctttatcttggcaacaacggaatgcataaaattccagccactattttatctcgtgtgcaacgctttgaattcaaagctattaagcaaaaagctattcgagagcatttagcctgggttttggacaaagaaggtattgcctatgaggtggatgctttaaatctcattgcaaggcgagcagaaggaggcatgcgtgatgctttatctattttagatcaggctttgagcttgtcaccagataatcaggtcgccattgcaattgccgaagaaattacaggttctatttccatacttgctctgggtgactatgttcgatatgtctcccaagaacaggctacgcaagctctggcagccttagagaccatttatgatagtgggaagagcatgagccgctttgcgacagatttattgacctatctgcgtgatttattggtggttaaagctggcggcgacaatcaacgtcagtcagctgtttttgataccaatttgtctctctcgatagatcgtatattccaaatgataacagttgttactagtcatctccctgaaatcaaaaagggaacccatcctcggatttatgccgaaatgatgactatccaattagctcagaaagagcagattttgtcccaagtaaacttgtcaggagagttaatctcagagattgaaacgctcaaaaatgagttggcacaacttaaacaacaattgtcgcagctccaatcgcgtcctgattcactggcaagatctgataaaacgaaacctaaaaccacaagctacagggttgatcgggttaccattttgaaaatcatggaagaaacggttcgaaatagccaacaatctcgacaatatctagatgctctaaaaaatgcttggaatgaaattctagataacatttctgcccaagacagagccttattgatgggctctgagcctgtcttagcaaatagtgagaatgcgattttggctttcgaggctgcctttaatgcagaacaagtcatgagccgaaataatcttaatgatatgtttggtaacattatgagtaaagctgctggtttttctcccaatattctggcagtaccaaggacagattttcagcatattcgtaaggaatttgctcagcaaatgaaatcgcaaaaagacagtgttcaagaagaacaagaagtagcgcttgatattccagaagggtttgattttttgctcgataaaataaatactattgacgac

SEQ ID NO: 26 - protein, 555 aa, Streptococcus pyogenes
MYQALYRKYRSQTFDEMVGQSVISTTLKQAVESGKISHAYLFSGPRGTGKTSAAKIFAKAMNCPNQVDGEPCNQCDICRDITNGSLEDVIEIDAASNNGVDEIRDIRDKSTYAPSRATYKVYIIDEVHMLSTGAFNALLKTLEEPTENVFILATTELHKIPATILSRVQRFEFKAIKQKAIREHLAWVLDKEGIAYEVDALNLIARRAEGGMRDALSILDQALSLSPDNQVAIAIAEEITGSISILALGDYVRYVSQEQATQALAALETIYDSGKSMSRFATDLLTYLRDLLVVKAGGDNQRQSAVFDTNLSLSIDRIFQMITVVTSHLPEIKKGTHPRIYAEMMTIQLAQKEQILSQVNLSGELISEIETLKNELAQLKQQLSQLQSRPDSLARSDKTKPKTTSYRVDRVTILKIMEETVRNSQQSRQYLDALKNAWNEILDNISAQDRALLMGSEPVLANSENAILAFEAAFNAEQVMSRNNLNDMFGNIMSKAAGFSPNILAVPRTDFQHIRKEFAQQMKSQKDSVQEEQEVALDIPEGFDFLLDKINTIDD

SEQ ID NO: 27 - DNA, 1134 bp, Streptococcus pyogenes
atgattcaattttcaattaatcgcacattatttattcatgctttaaatacaactaaacgtgctattagcactaaaaatgccattcctattctttcatcaataaaaattgaagtcacttctacaggagtaactttaacagggtctaacggtcaaatatcaattgaaaacactattcctgtaagtaatgaaaatgctggtttgctaattacctctccaggagctattttattagaagctagtttttttattaatattatttcaagtttgccagatattagtataaatgttaaagaaattgaacaacaccaagttgttttaaccagtggtaaatcagagattaccttaaaaggaaaagatgttgaccagtatcctcgtctacaagaagtatcaacagaaaatcctttgattttaaaaacaaaattattgaagtctattattgctgaaacagcttttgcagccagtttacaagaaagtcgtcctattttaacaggagttcatattgtattaagtaatcataaagattttaaagcagtagcgactgactctcatcgtatgagccaacgtttaatcactttggacaatacttcagcagatttgatggtagttcttccaagtaaatctttgagagaattttcagcagtatttacagatgatattgagaccgttgaggtatttttctcaccaagccaaatcttgttcagaagtgaacacatttctttttatacacgcctcttagaaggaaattatcccgatacagaccgtttattaatgacagaatttgagacggaggttgttttcaatacccaatcccttcgccacgctatggaacgtgccttcttgatttctaatgctactcaaaatggtactgttaagcttgagattactcaaaatcatatttcagctcatgttaactcacctgaggttggtaaggtaaacgaggatttagatattgttagtcagtctggtagtgatttaactatcagcttcaatccaacttaccttattgagtctttaaaagctattaaaagtgaaacagtaaaaattcatttcttatcaccagttcgaccattcaccctaacaccaggcgatgaggaagaaagttttatccaattaattacaccagtacgaacaaac

SEQ ID NO: 28 - protein, 378 aa, Streptococcus pyogenes
MIQFSINRTLFIHALNTTKRAISTKNAIPILSSIKIEVTSTGVTLTGSNGQISIENTIPVSNENAGLLITSPGAILLEASFFINIISSLPDISINVKEIEQHQVVLTSGKSEITLKGKDVDQYPRLQEVSTENPLILKTKLLKSIIAETAFAASLQESRPILTGVHIVLSNHKDFKAVATDSHRMSQRLITLDNTSADLMVVLPSKSLREFSAVFTDDIETVEVFFSPSQILFRSEHISFYTRLLEGNYPDTDRLLMTEFETEVVFNTQSLRHAMERAFLISNATQNGTVKLEITQNHISAHVNSPEVGKVNEDLDIVSQSGSDLTISFNPTYLIESLKAIKSETVKIHFLSPVRPFTLTPGDEEESFIQLITPVRTN

SEQ ID NO: 29 - DNA, 492 bp, Streptococcus pyogenes
atgattaataatgtagtactagttggtcgcatgaccaaggatgcagaacttcgttacacaccaagtcaagtagctgtggctaccttcacacttgctgttaaccgtacctttaaaagccaaaatggtgaacgcgaggcagatttcattaactgtgtgatctggcgtcaaccggctgaaaatttagcgaactgggctaaaaaaggtgctttgatcggagttacgggtcgtattcatacacgtaactacgaaaaccaacaaggacaacgtgtctatgtaacagaagttgttgcagataatttccaaatgttggaaagtcgtgctacacgtgaaggtggctcaactggctcatttaatggtggttttaacaataacacttcatcatcaaacagttactcagcgcctgcacaacaaacgcctaactttggaagagatgatagcccatttgggaactcaaacccgatggatatctcagatgacgatcttccattctag

SEQ ID NO: 30 - protein, 163 aa, Streptococcus pyogenes
MINNVVLVGRMTKDAELRYTPSQVAVATFTLAVNRTFKSQNGEREADFINCVIWRQPAENLANWAKKGALIGVTGRIQTRNYENQQGQRVYVTEVVADNFQMLESRATREGGSTGSFNGGFNNNTSSSNSYSAPAQQTPNFGRDDSPFGNSNPMDISDDDLPF

SEQ ID NO: 31 - DNA, 1815 bp, Streptococcus pyogenes
atgggatttttatggggaggtgacgatttggcaattgacaaagaaatgatttcccaagtaaaaaatagcgttaatattgtcgatgtcattggagaagtggtcaaactttcccgatcagggcggcattacctcgggctttgcccatttcataaggaaaagacaccctcttttaatgttgttgaagacagacaattttttcactgctttggctgtggaaaatcaggggatgtttttaaatttattgaggaataccgccaagtccccttcttagaaagtgttcagattattgccgataagactggtatgtcgcttaatataccgccaagtcaggcagtacttgctagccaacacaagcaccctaatcacgctttgatgacacttcatgaggatgctgctaaattttaccatgcagttttgatgaccactaccattggtcaagaagctaggaagtacctttaccagagaggcttggatgaccaattaattgagcatttcaatattggtttagccccagatgagtcagattatctttatcaagctctttctaaaaaatacgaggaaggtcaattggttgcttcaggattgtttcacttgtccgatcaatccaataccatttacgacgcctttcgaaatcgtatcatgtttcccttatcagatgaccgagggcatattattgccttttcaggacgtatctggacggcagctgatatggaaaagagacaggcaaagtataaaaattcaagaggaacagttctttttaacaaatcttatgaattgtatcatctggacaaggcaaggcctgttattgccaaaacccatgaagtgtttctaatggaagggtttatggacgtgattgccgcttaccgttccggctatgaaaatgctgttgcttcaatggggacggcattgactcaagaacatgtcaatcaccttaagcaagtcactaaaaaagttgttttgatttatgatggtgacgatgctggacaacatgctattgcaaaatcactagaattgcttaaagattttgttgtcgaaattgtcagaatccccaataaaatggatcctgacgaatttgtacaacggcattccccagaagcatttgcagatttgcttaagcagtcacggatcagtagtgttgaattttttattgattacctaaaacctactaatgtagacaatttgcaatcacaaattgtttatgtggagaaaatggcaccattgattgctcaatcaccatccatcacagctcaacattcgtatattaacaagattgctgatttgttgccaaactttgactattttcaagtagaacaatcagtaaatgcattaaggattcaagataggcaaaaacatcaaggtcaaatagctcaagccgtcagcaatcttgtgaccttaccaatgccaaaaagtttgacagctattgctaagacagaaagtcatctcatgcatcggctcttacatcatgactatttattaaatgaatttcgacatcgtgatgatttttattttgatacctctaccttagaattactttatcaacggctgaagcaacaaggacacattacatcttatgatttgtcagagatgtcagaggaagttaaccgtgcttattacaatgttttagaagaaaaccttcccaaagaagtagctcttggtgagattgatgatattttatccaaacgtgccaaacttttagcagagcgcgatcttcacaaacaagggaaaaaagttagagaatctagtaacaaaggcgatcatcaagcggctctagaagtactagaacattttattgcgcagaaacgaaaaatggaatag

SEQ ID NO: 32 - protein, 600 aa, Streptococcus pyogenes
MGFLWGGDDLAIDKEMISQVKNSVNIVDVIGEVVKLSRSGRHYLGLCPFHKEKTPSFNVVEDRQFFHCFGCGKSGDVFKFIEEYRQVPFLESVQIIADKTGMSLNIPPSQAVLASQHKHPNHALMTLHEDAAKFYHAVLMTTTIGQEARKYLYQRGLDDQLIEHFNIGLAPDESDYLYQALSKKYEEGQLVASGLFHLSDQSNTIYDAFRNRIMFPLSDDRGHIIAFSGRIWTAADMEKRQAKYKNSRGTVLFNKSYELYHLDKARPVIAKTHEVFLMEGFMDVIAAYRSGYENAVASMGTALTQEHVNHLKQVTKKVVLIYDGDDAGQHAIAKSLELLKDFVVEIVRIPNKMDPDEFVQRHSPEAFADLLKQSRISSVEFFIDYLKPTNVDNLQSQIVYVEKMAPLIAQSPSITAQHSYINKIADLLPNFDYFQVEQSVNALRIQDRQKHQGQIAQAVSNLVTLPMPKSLTAIAKTESHLMHRLLHHDYLLNEFRHRDDFYFDTSTLELLYQRLKQQGHITSYDLSEMSEEVNRAYYNVLEENLPKEVALGEIDDILSKRAKLLAERDLHKQGKKVRESSNKGDHQAALEVLEHFIAQK

SEQ ID NO: 33 - DNA, 1368 bp, Streptococcus pyogenes
atgaggttgcctgaagtagctgaattacgagttcaaccccaagatttactagcagagcaatctgttcttgggtcaatctttatctcacctgataagctgattgcagtgagagaatttatcagtccagacgatttttataagtacgctcataaaattatctttcgggcaatgattaccctcagcgatcgtaatgatgccattgatgcaaccactataagaacaatcctagatgatcaagatgatctgcaaagtattggtggcttatcctatattgttgaactagttaatagtgtcccaactagtgctaatgcagaatattatgctaaaattgtagctgagaaagctatgttgcgtgatattattgctaggttgacagaatctgtcaacctagcttatgatgaaattttaaaaccagaagaggttatcgctggagttgagagagctttaattgaactcaatgaacatagtaatcgtagtgggtttcgcaaaatttcagatgtgctaaaagttaattacgaggctttagaagcacgttctaagcagacttcaaatgttacaggtttaccaactggttttagagaccttgacaagattacaacaggtttacacccagatcaattagttattttagctgctcggccagcagtggggaagactgcctttgttcttaatattgcgcaaaatgtggggactaagcaaaaaaagactgttgctattttttctttggaaatgggtgctgaaagtttagtagatcgtatgcttgcagcagaaggaatggttgattcgcacagtttaagaacagggcaactcacagatcaggattggaataatgtaacaattgctcagggagctttggcagaagcaccgatttatattgacgatacgcccgggattaaaattactgaaatccgcgcaagatcacggaaattgtctcaagaagtggatggtggtttaggtctcattgtaattgactacttacagttgattacaggaactaaacccgaaaatcgtcagcaagaggtttcagatatttcaagacagcttaaaatcctagctaaagaattgaaagtaccagttattgccctaagtcagctttctcgtggcgttgagcaaaggcaagataaacgaccagttttatcagatattcgtgaatcaggatctattgagcaggatgccgatattgtagccttcttataccgggacgattattaccgtaaagaatgtgatgatgctgaagaagctgttgaagataacacaattgaagttatcctcgagaaaaatagagctggggcgcgtggaacagtcaaactgatgttccaaaaagaatacaacaaattctcaagtatagcccagtttgaagaaagataa

SEQ ID NO: 34 - protein, 455 aa, Streptococcus pyogenes
MRLPEVAELRVQPQDLLAEQSVLGSIFISPDKLIAVREFISPDDFYKYAHKIIFRAMITLSDRNDAIDATTIRTILDDQDDLQSIGGLSYIVELVNSVPTSANAEYYAKIVAEKAMLRDIIARLTESVNLAYDEILKPEEVIAGVERAQGALAEAPIYIDDTPGIKIALIELNEHSNRSGFRKISDVLKVNYEALEARSKQTSNVTGLPTGFRDLDKITTGLHPDQLVILAARPAVGKTAFVLNIAQNVGTKQKKTVAIFSLEMGAESLVDRMLAAEGMVDSHSLRTGQLTDQDWNNVTITEIRARSRKLSQEVDGGLGLIVIDYLQLITGTKPENRQQEVSDISRQLKILAKELKVPVIALSQLSRGVEQRQDKRPVLSDIRESGSIEQDADIVAFLYRDDYYRKECDDAEEAVEDNTIEVILEKNRAGARGTVKLMFQKEYNKFSSIAQFEER

SEQ ID NO: 35 - DNA, 29 bp, Artificial Sequence
  Description of Artificial Sequence  primer
ggtggtaattgtcttgcatatgacagagc

SEQ ID NO: 36 - DNA, 31 bp, Artificial Sequence
  Description of Artificial Sequence  primer
agcgattaagtggattgccgggttgtgatgc

SEQ ID NO: 37 - DNA, 31 bp, Artificial Sequence
  Description of Artificial Sequence  primer
agcatcacaacccggcaatccacttaatcgc

SEQ ID NO: 38 - DNA, 29 bp, Artificial Sequence
  Description of Artificial Sequence  primer
gactacgccatgggcattaaataaatacc

SEQ ID NO: 39 - DNA, 30 bp, Artificial Sequence
  Description of Artificial Sequence  primer
gaagatgcatataaacgtgcaagacctagt

SEQ ID NO: 40 - DNA, 34 bp, Artificial Sequence
  Description of Artificial Sequence  primer
gtctgacgcacgaattgtaaagtaagatgcatag

SEQ ID NO: 41 - DNA, 36 bp, Artificial Sequence
  Description of Artificial Sequence  primer
cgactggaaggagttttaacatatgatggaattcac

SEQ ID NO: 42 - DNA, 30 bp, Artificial Sequence
  Description of Artificial Sequence  primer
ttatatggatccttagtaagttctgattgg

SEQ ID NO: 43 - protein, 15 aa, Escherichia coli
LLFERFLNPERVSMP

SEQ ID NO: 44 - protein, 15 aa, Escherichia coli
KFAGYGFNKSHSAAY

SEQ ID NO: 45 - DNA, 44 bp, Artificial Sequence
  Description of Artificial Sequence  primer
cttctttttgaaagatttctaaataaagaacgttattcaatgcc

SEQ ID NO: 46 - DNA, 45 bp, Artificial Sequence
  Description of Artificial Sequence  primer
ataagctgcagcatgacttttattaaaaccataacctgcaaattt

SEQ ID NO: 47 - DNA, 39 bp, Artificial Sequence
  Description of Artificial Sequence  primer
agttaaaaatgccatattttgacgtgttttagttctaat

SEQ ID NO: 48 - DNA, 42 bp, Artificial Sequence
  Description of Artificial Sequence  primer
cttgcaaaagcggttgctaaagatgttggacgaattatgggg

SEQ ID NO: 49 - protein, 10 aa, Escherichia coli
HAYLFSGPRG

SEQ ID NO: 50 - protein, 10 aa, Escherichia coli
HAYLFSGPRG

SEQ ID NO: 51 - DNA, 38 bp, Artificial Sequence
  Description of Artificial Sequence  primer
cgcggatcccatgcatatttattttcaggtccaagagg

SEQ ID NO: 52 - DNA, 39 bp, Artificial Sequence
  Description of Artificial Sequence  primer
ccggaattctggtggttcttctaatgtttttaataatgc

SEQ ID NO: 53 - DNA, 38 bp, Artificial Sequence
  Description of Artificial Sequence  primer
tttgtaaaggcattacgcaggggactaattcagatgtg

SEQ ID NO: 54 - DNA, 33 bp, Artificial Sequence
  Description of Artificial Sequence  primer
tatgacattcattacaaggttctccatcagtgc

SEQ ID NO: 55 - DNA, 29 bp, Artificial Sequence
  Description of Artificial Sequence  primer
gagcactgatgaacttagaattagatatg

SEQ ID NO: 56 - DNA, 32 bp, Artificial Sequence
  Description of Artificial Sequence  primer
gatactcagtatctttctcagatgttttattc

SEQ ID NO: 57 - protein, 14 aa, Escherichia coli
DLIIVAARPSMGKT

SEQ ID NO: 58 - protein, 15 aa, Escherichia coli
EIIIGKQRNGPIGTV

SEQ ID NO: 59 - DNA, 41 bp, Artificial Sequence
  Description of Artificial Sequence  primer
gaccttataattgtagctgcacgtccttctatgggaaaaac

SEQ ID NO: 60 - DNA, 48 bp, Artificial Sequence
  Description of Artificial Sequence  primer
aacattattaagtcagcatcttgttctattgatccagattcaacgaag

SEQ ID NO: 61 - DNA, 45 bp, Artificial Sequence
  Description of Artificial Sequence  primer
gatttgtagttctggtaatgttgactcaaaccgcttaagaaccgg

SEQ ID NO: 62 - DNA, 48 bp, Artificial Sequence
  Description of Artificial Sequence  primer
atacgtgtggttaactgatcagcaacccatctctagtgagaaaatacc

SEQ ID NO: 63 - DNA, 31 bp, Artificial Sequence
  Description of Artificial Sequence  primer
cgttttaatgcatgcttagaaacgatatcag

SEQ ID NO: 64 - DNA, 31 bp, Artificial Sequence
  Description of Artificial Sequence  primer
cattgctaagcaacgttacggtccaacaggc

SEQ ID NO: 65 - DNA, 69 bp, Artificial Sequence
  Description of Artificial Sequence  primer
ggataacaattccccgctagcaataattttgtttaactttaagaaggagatatacccatggatgaacag

SEQ ID NO: 66 - DNA, 39 bp, Artificial Sequence
  Description of Artificial Sequence  primer
aattttaaaggatccgtgtataatattctaattttcccg

SEQ ID NO: 67 - DNA, 28 bp, Artificial Sequence
  Description of Artificial Sequence  primer
gggagtttgtaatccatggatgaacagc

SEQ ID NO: 68 - DNA, 37 bp, Artificial Sequence
  Description of Artificial Sequence  primer
ctgaacacctattaccctaggcatctaactcacaccc

SEQ ID NO: 69 - DNA, 38 bp, Artificial Sequence
  Description of Artificial Sequence  primer
ggagcagattgcttttgatacatatgattggcctattc

SEQ ID NO: 70 - DNA, 46 bp, Artificial Sequence
  Description of Artificial Sequence  primer
ttgtctccgcatcaaactgggatccaagagcatcatacgcgtatgg

SEQ ID NO: 71 - DNA, 36 bp, Artificial Sequence
  Description of Artificial Sequence  primer
gcctaggataagggagggtacatatggatttagcgc

SEQ ID NO: 72 - DNA, 44 bp, Artificial Sequence
  Description of Artificial Sequence  primer
cgggcaagtcttttgacaagcttcggatccccataacgaattcc

SEQ ID NO: 73 - DNA, 33 bp, Artificial Sequence
  Description of Artificial Sequence  primer
ggagttaaaaacatatgtatcaagctctttatc

SEQ ID NO: 74 - DNA, 38 bp, Artificial Sequence
  Description of Artificial Sequence  primer
cgtgggtaagggcaaaacggatcccttatgtatttcag

SEQ ID NO: 75 - DNA, 34 bp, Artificial Sequence
  Description of Artificial Sequence  primer
ggagttcatatgattcaattttcaaattaatcgc

SEQ ID NO: 76 - DNA, 38 bp, Artificial Sequence
  Description of Artificial Sequence  primer
tatcagctcctggatccagtaccttccattgattagcc

SEQ ID NO: 77 - DNA, 74 bp, Artificial Sequence
  Description of Artificial Sequence  primer
ggataacaattccccgctagcaataattttgtttaactttaagaaggagatatacccatgtcagatttattcgc

SEQ ID NO: 78 - DNA, 57 bp, Artificial Sequence
  Description of Artificial Sequence  primer
cggtgtctctatctaaatgactcatttgggatcctcgctttatacggtatgtcacag

SEQ ID NO: 79 - DNA, 43 bp, Artificial Sequence
  Description of Artificial Sequence  primer
gggaacaagataaccaaggaggaacccatggttgctcaacttg

SEQ ID NO: 80 - DNA, 40 bp, Artificial Sequence
  Description of Artificial Sequence  primer
cgaatagcagcgttcataccaggatcctcgccgccactgg

SEQ ID NO: 81 - DNA, 49 bp, Artificial Sequence
  Description of Artificial Sequence  primer
accattttggcttttaaaggtacggttaacagcaagtgtgaaggtagcc

SEQ ID NO: 82 - DNA, 38 bp, Artificial Sequence
  Description of Artificial Sequence  primer
gaacgcgaggcagatttcattaactgtgtgatctggcg

SEQ ID NO: 83 - DNA, 48 bp, Artificial Sequence
  Description of Artificial Sequence  primer
tttaaaagagggtagcatatgattaataatgtagtactagttggtcgc

SEQ ID NO: 84 - DNA, 51 bp, Artificial Sequence
  Description of Artificial Sequence  primer
tttaaatttaaacctaggttcaatccattctgactagaatggaagatcgtc

(19) United States
(12) Patent Application Publication
(10) Pub. No.: US 2009/0240035 A1
(43) Pub. Date: Sep. 24, 2009

(54) DNA REPLICATION PROTEINS OF GRAM POSITIVE BACTERIA AND THEIR USE TO SCREEN FOR CHEMICAL INHIBITORS

(76) Inventors: Michael E. O'Donnell, Hasting-on-Hudson, NY (US); Irina Bruck, New York, NY (US); Dan Zhang, Forest Hills, NY (US); Richard Whipple, Rareton, NJ (US)

Correspondence Address: NIXON PEABODY LLP - PATENT GROUP, 1100 CLINTON SQUARE, ROCHESTER, NY, 14604, US

(21) Appl. No.: 12/146,353

(22) Filed: Jun. 25, 2008

Related U.S. Application Data
(62) Division of application No. 10/048,071, filed on Oct. 23, 2002, now Pat. No. 7,432,365.
(60) Provisional application No. 60/146,178, filed on Jul. 29, 1999.

(51) Int. Cl. C07K 14/00

(52) U.S. Cl. 530/350

(57) ABSTRACT

The present invention relates to alpha-large, alpha-small, delta, delta prime, tau, beta, SSB, DnaG, and DnaB encoding genes from Gram positive bacterium, preferably Streptococcus and Staphylococcus bacterium. The formation of functional polymerase as well as the use of such a polymerase in sequencing and amplification is also disclosed. The individual genes and proteins or polypeptides are useful in identification of compounds with antibiotic activity.

The present application is a division of U.S. patent application Ser. No. 10/048,071 filed Oct. 23, 2002, which is a national stage application under 35 U.S.C. § 371 of International Application No. PCT/US00/20666, filed Jul. 28, 2000, which claims benefit of U.S. Provisional Patent Application Ser. No. 60/146,178 filed Jul. 29, 1999, which are hereby incorporated by reference.

The present invention was made with funding from National Institutes of Health Grant No. GM38839. The United States Government may have certain rights in this invention.

### FIELD OF THE INVENTION

This invention relates to genes and proteins that replicate the chromosome of Gram positive bacteria. These proteins can be used in sequencing, amplification of DNA, and in drug discovery to screen large libraries of chemicals for identification of compounds with antibiotic activity.

### BACKGROUND OF THE INVENTION

All forms of life must duplicate the genetic material to propagate the species. The process by which the DNA in a chromosome is duplicated is called replication. The replication process is performed by numerous proteins that coordinate their actions to duplicate the DNA smoothly. The main protein actors are as follows (reviewed in Kornberg et al., DNA Replication, Second Edition, New York: W.H. Freeman and Company, pp. 165-194 (1992)). A helicase uses the energy of ATP hydrolysis to unwind the two DNA strands of the double helix. Two copies of the DNA polymerase use each “daughter” strand as a template to convert them into two new duplexes. The DNA polymerase acts by polymerizing the four monomer unit building blocks of DNA (the 4 dNTPs, or deoxynucleoside triphosphates are: dATP, dCTP, dGTP, dTTP). The polymerase rides along one strand of DNA using it as a template that dictates the sequence in which the monomer blocks are to be polymerized. Sometimes the DNA polymerase makes a mistake and includes an incorrect nucleotide (e.g., A instead of G). A proofreading exonuclease examines the polymer as it is made and excises building blocks that have been improperly inserted in the polymer.

Duplex DNA is composed of two strands that are oriented antiparallel to one another, one being oriented 3′-5′ and the other 5′ to 3′. As the helicase unwinds the duplex, the DNA polymerase moves continuously forward with the helicase on one strand (called the leading strand). However, due to the fact that DNA polymerases can only extend the DNA forward from a 3′ terminus, the polymerase on the other strand extends DNA in the opposite direction of DNA unwinding (called the lagging strand). This necessitates a discontinuous ratcheting motion on the lagging strand in which the DNA is made as a series of Okazaki fragments. DNA polymerases cannot initiate DNA synthesis de novo, but require a primed site (i.e., a short duplex region). This job is fulfilled by primase, a specialized RNA polymerase, that synthesizes short RNA primers on the lagging strand. The primed sites are extended by DNA polymerase. A single-stranded DNA binding protein (“SSB”) is also needed; it operates on the lagging strand. The function of SSB is to coat single stranded DNA (“ssDNA”), thereby melting short hairpin duplexes that would otherwise impede DNA synthesis by DNA polymerase.

The replication process is best understood for the Gram negative bacterium Escherichia coli and its bacteriophages T4 and T7 (reviewed in Kelman et al., “DNA Polymerase III Holoenzyme: Structure and Function of Chromosomal Replicating Machine,” Annu. Rev. Biochem., 64:171-200 (1995); Marians, K. J., “Prokaryotic DNA Replication,” Annu. Rev. Biochem., 61:673-719 (1992); McHenry, C. S., “DNA Polymerase III Holoenzyme: Components, Structure, and Mechanism of a True Replicative Complex,” J. Bio. Chem., 266:19127-19130 (1991); Young et al., “Structure and Function of the Bacteriophage T4 DNA Polymerase Holoenzyme,” Am. Chem. Soc., 31:8675-8690 (1992)). The eukaryotic systems of yeast (Saccharomyces cerevisiae) (Morrison et al., “A Third Essential DNA Polymerase in S. cerevisiae,” Cell, 62:1143-51 (1990) and humans (Bambara et al., “Reconstitution of Mammalian DNA Replication,” Prog. Nuc. Acid Res.,” 51:93-123 (1995)) have also been characterized in some detail as has herpes virus (Boehmer et al., “Herpes Simplex Virus DNA Replication,” Annu. Rev. Biochem., 66:347-384 (1997)) and vaccinia virus (McDonald et al., “Characterization of a Processive Form of the Vaccinia Virus DNA Polymerase,” Virology, 234:168-175 (1997)). The helicase of E. coli is encoded by the dnaB gene and is called the DnaB-helicase. In phage T4, the helicase is the product of the gene 41, and, in T7, it is the product of gene 4. Generally, the helicase contacts the DNA polymerase in E. coli. This contact is necessary for the helicase to achieve the catalytic efficiency needed to replicate a chromosome (Kim et al., “Coupling of a Replicative Polymerase and Helicase: A tau-DnaB Interaction Mediates Rapid Replication Fork Movement,” Cell, 84:643-650 (1996)). The identity of the helicase that acts at the replication fork in a eukaryotic cellular system is still not firm.

The primase of E. coli (product of the dnaG gene), phage T4 (product of gene 61), and T7 (gene 4) require the presence of their cognate helicase for activity. The primase of eukaryotes, called DNA polymerase alpha, looks and behaves differently. DNA polymerase alpha is composed of 4 subunits. The primase activity is associated with the two smaller subunits, and the largest subunit is the DNA polymerase which extends the product of the priming subunits. DNA polymerase alpha does not need a helicase for priming activity on single strand DNA that is not coated with binding protein.

The chromosomal replicating DNA polymerase of all these systems, prokaryotic and eukaryotic, share the feature that they are processive, meaning they remain continuously associated with the DNA template as they link monomer units (dNTPs) together. This catalytic efficiency can be manifest in vitro by their ability to extend a single primer around a circular ssDNA of over 5,000 nucleotide units in length. Chromosomal DNA polymerases will be referred to here as replicases to distinguish them from DNA polymerases that function in other DNA metabolic processes and are far less processive.

There are three types of replicases known thus far that differ in how they achieve processivity and how their subunits are organized. These will be referred to here as Types I-III. The Type I is exemplified by the phage T5 replicase, which is composed of only one subunit yet is highly processive (Das et al., “Mechanism of Primer-template Dependent Conversion of dNTP-dNMP by T7 DNA Polymerase,” J. Biol. Chem., 255:7149-7154 (1980)). It is possible that the T5 enzyme achieves processivity by having a cavity within it for binding DNA, with a domain of the protein acting as a lid that opens to accept the DNA and closes to trap the DNA inside, thereby keeping the polymerase on DNA during polymerization of dNTPs. Type II is exemplified by the replicases of phage T7, herpes simplex virus, and vaccinia virus. In these systems, the replicase is composed of two subunits, the DNA polymerase and an “accessory protein” which is needed for the polymerase to become highly efficient. It is presumed that the DNA polymerase binds the DNA in a groove and that the accessory protein forms a cap over the groove, trapping the DNA inside for processive action. Type III is exemplified by the replicases of E. coli, phage T4, yeast, and humans in which there are three separate components, a sliding clamp protein, a clamp loader protein complex, and the DNA polymerase. In these systems, the sliding clamp protein is an oligomer in the shape of a ring. The clamp loader is a multiprotein complex which uses ATP to assemble the clamp around DNA. The DNA polymerase then binds the clamp which tethers the polymerase to DNA for high processivity. The replicase of the E. coli system contains a fourth component called tau that acts as a glue to hold two polymerases and one clamp loader together into one structure called Pol III*. In this application, any replicase that uses a minimum of three components (i.e., clamp, clamp loader, and DNA polymerase) will be referred to as either a three component polymerase, a type III enzyme, or a DNA polymerase III-type replicase.

The E. coli replicase is also called DNA polymerase III holoenzyme. The holoenzyme is a single multiprotein particle that contains all the components; it is comprised of ten different proteins. This holoenzyme is suborganized into four functional components called: 1) Pol III core (DNA polymerase); 2) gamma complex or tau/gamma complex (clamp loader); 3) beta subunit (sliding clamp); and 4) tau (glue protein). The DNA polymerase III “core” is a tightly associated complex containing one each of the following three subunits: 1) the alpha subunit is the actual DNA polymerase (129 kDa); 2) the epsilon subunit (28 kDa) contains the proofreading 3′-5′ exonuclease activity; and 3) the theta subunit has an unknown function. The gamma complex is the clamp loader and contains the following subunits: gamma, delta, delta prime, chi and psi (U.S. Pat. No. 5,583,026 to O'Donnell). Tau can substitute for gamma, as can a tau/gamma heterooligomer. The beta subunit is a homodimer and forms the ring shaped sliding clamp. These components associate to form the holoenzyme and the entire holoenzyme can be assembled in vitro from 10 isolated pure subunits (U.S. Pat. No. 5,583,026 to O'Donnell; U.S. Pat. No. 5,668,004 to O'Donnell). The E. coli dnaX gene encodes both tau and gamma. Tau is the product of the full gene. Gamma is the product of the first ⅔ of the gene; it is truncated by an efficient translational frameshift that results in incorporation of one unique residue followed by a stop codon.

The tau subunit, encoded by the same gene that encodes gamma (dnaX), also acts as a glue to hold two cores together with one gamma complex. This subassembly is called DNA polymerase III star (Pol III*). One beta ring interacts with each core in Pol III* to form DNA polymerase III holoenzyme.

During replication, the two cores in the holoenzyme act coordinately to synthesize both strands of DNA in a duplex chromosome. At the replication fork, DNA polymerase III holoenzyme physically interacts with the DnaB helicase through the tau subunit to form a yet larger protein complex termed the “replisome” (Kim et al., “Coupling of a Replicative Polymerase and Helicase: A tau-DnaB Interaction Mediates Rapid Replication Fork Movement,” Cell, 84:643-650 (1996); Yuzhakov et al., “Replisome Assembly Reveals the Basis for Asymmetric Function in Leading and Lagging Strand Replication,” Cell, 86:877-886 (1996)). The primase repeatedly contacts the helicase during replication fork movement to synthesize RNA primers on the lagging strand (Marians, K. J., “Prokaryotic DNA Replication,” Annu. Rev. Biochem., 61:673-719 (1992)).

Intensive subtyping of prokaryotic cells has now lead to a taxonomic classification of prokaryotic cells as eubacteria (true bacteria) to distinguish them from archaebacteria. Within eubacteria are many different subcategories of cells, although they can broadly be subdivided into Gram positive- and Gram negative-like cells. Numerous complete and partial genome sequences of prokaryotes have appeared in the public databases.

In the present invention, new genes from the Gram positive bacteria, Streptococcus pyogenes (e.g., S. pyogenes) and Staphylococcus aureus (e.g., S. aureus) are identified. They are assigned names based on their nearest homology to subunits in the E. coli system. The genes encoding E. coli replication proteins are as follows: alpha (dnaE); epsilon (dnaQ); theta (holE); tau (full length dnaX); gamma (frameshift product of dnaX); delta (holA); delta prime (holB); chi (holC); psi (holD); beta (dnaN); DnaB helicase (dnaB); and primase (dnaG).

Study of the organisms for which a complete genome sequence is available reveals that no organism has identifiable homologues to all the subunits of the E. coli three component polymerase, Pol III holoenzyme (see Table 1 below). All other organisms lack the θ subunit (holE), and all except one lack genes encoding the χ and ψ subunits (holC and holD, respectively) as judged by sequence comparison searches. Further, the α and ε subunits are fused into one large α subunit in some organisms (e.g., Gram positive cells) as detailed in (Sanjanwala et al., “DNA Polymerase III Gene of Bacillus subtilis,” Proc. Natl. Acad. Sci., USA, 86:4421-4424 (1989)). Although all organisms have homologues to τ, δ, δ′ and SSB, the δ subunit has diverged significantly (either not recognized or nearly not recognized by gene searching programs), perhaps even to the point where it is no longer involved in DNA replication. The DnaX product also would appear to lack frameshift signals in most organisms. This predicts only one protein (tau) will be produced from this gene, instead of two as in E. coli. Indeed, this has been shown to be true for the Staphylococcus are s DnaX (U.S. patent application Ser. No. 09/235,245, which is hereby incorporated by reference). Finally, genetic study of Bacillus subtilis identified two genes that do not have counterparts in E. coli (dnaB, not the helicase, and dnaH) as well as one other gene, dnaI, that is only very distantly related to E. coli dnaC (Karamata et al., “Isolation and Genetic Analysis of Temperature-Sensitive Mutants of B. subtilis Defense in DNA Synthesis,” Molec. Gen. Genet., 108:277-287 (1970); Braund et al., “Nucleotide Sequence of the Bacillus subtilis dnaD Gene,” Microb., 141:321-322 (1995); Hoshino et al., “Nucleotide Sequence of Bacillus subtilis dnaB: A Gene Essential for DNA Replication Initiation and Membrane Attachment,” Proc. Natl. Acad. Sci. USA,” 84:653-657 (1987)). Keeping in mind the apparently random, or at least unpredictable process of evolution, it is possible that these apparently new genes perform novel functions that may result in a new type of polymerase for chromosomal replication. Thus, it seems possible that new proteins may have evolved to take the place of χ, ψ, θ, the frameshift product of DnaX, and possibly 6 in other eubacteria. These considerations indicate that the three component polymerase of different eubacteria may have different structures. That this may be so would not be surprising as different bacteria are often less related evolutionarily than plants are to humans. For example, the split between Gram positive and Gram negative bacteria occurred about 1.2 billion years ago. This distant split makes Gram positive cells an attractive source to examine how different other eubacterial three component polymerases are from the E. coli Pol III holoenzyme.

TABLE 1

| Organism (Order) | χ | φ | θ | ε | α | β | dnaX | δ′ | δ | |
|---|---|---|---|---|---|---|---|---|---|---|
| Escherichia coli | + | + | + | + | + | + | + | + | + | |
| Proteobacteria | | | | | | | | | | |
| Haemophilus influenzae | + | + | − | + | + | + | + | + | + | |
| Proteobacteria | | | | | | | | | | |
| Mycoplasma genitalium | − | − | − | − | + | + | + | + | + | (weak) |
| Firmicutes | | | | | | | | | | |
| Synichisystis sp. | − | − | − | − | + | + | + | + | + | (weak) |
| Cyanobacteria | | | | | | | | | | |
| Bacillus subtilis | − | − | − | − | + | + | + | + | + | (weak) |
| Firmicutes | | | | | | | | | | |
| Borrelia burgdorferi | − | − | − | − | + | + | + | + | + | (weak) |
| Spirochaetales | | | | | | | | | | |
| Aquifex aeolicus | − | − | − | + | + | + | + | + | + | (weak) |
| Aquificales | | | | | | | | | | |
| Mycobacterium tuberculosis | − | − | − | + | + | + | + | + | + | (weak) |
| Firmicutes & Actinobacteria | | | | | | | | | | |
| Treponema pallidum | − | − | − | + | + | + | + | + | + | (weak) |
| Spirochaetales | | | | | | | | | | |
| Chlamydia trachomatis | − | − | − | + | + | + | + | + | + | (weak) |
| Chiamydiales | | | | | | | | | | |
| Rickettsia prowazekii | − | − | − | + | + | + | + | + | + | (weak) |
| Proteobacteria | | | | | | | | | | |
| Helicobacter pylori | − | − | − | + | + | + | + | + | + | (weak) |
| Proteobacteria | | | | | | | | | | |
| Thermatoga maritima | − | − | − | − | + | + | + | + | + | (weak) |
| Thermotogales | | | | | | | | | | |

The goal of this invention is to learn how to form a functional three component polymerase from an organism that is highly divergent from E. coli and whether it is as rapid and processive as the E. coli Pol III holoenzyme. Namely, from bacteria lacking χ, ψ, or θ, or having a widely divergent δ subunit, or having only one DnaX product, or an α subunit that encompasses both α and ε activities. All eubacteria for which the entire genome has been sequenced have at least one of these differences from E. coli. Many Gram negative bacteria have one or more of these differences (e.g., Haemophilus influenzae and Aquifex aeolicus). Bacteria of the Gram positive class have all of these different features. Because of the distant evolutionary split between Gram positive and Gram negative bacteria, their mechanisms of replication may have diverged significantly as well. Indeed, purification of the replication polymerase from B. subtilis, a Gram positive cell, gives only a single subunit polymerase (Barnes et al., “Purification of DNA Polymerase III of Gram-Positive Bacteria,” Methods Enzy. 262:35-42 (1995); Barnes et al., “Antibody to B. subtilis DNA Polymerase III: Use in Enzyme Purification and Examination of Homology Among Replication-specific DNA Polymerases,” Nucl. Acids Res., 6:1203-209 (1979); Barnes et al., “DNA Polymerase III of Mycoplasma pulmonis: Isolation and Characterization of the Enzyme and its Structural Gene, polC,” Mol. Microb., 13:843-854, (1994); Low et al., “Purification and Characterization of DNA Polymerase III from Bacillus subtilis,” J. Biol. Chem., 251:1311-1325 (1976)) instead of a 10 subunit assembly containing the three components of a rapidly processive machine as discussed above for Pol III holoenzyme from E. coli. This finding suggests a different structural organization of the replicase and possibly different functional characteristics as well.

Although there are many studies of replication mechanisms in eukaryotes and, specifically, the Gram negative bacterium E. coli and its bacteriophages, there is very little information about how Gram positive organisms replicate. The Gram positive class of bacteria includes some of the worst human pathogens such as Staphylococcus aureus, Streptococcus pneumoniae, Streptococcus pyogenes, Enterococcus faecalis, and Mycobacterium tuberculosis (Youmans et al., The Biological and Clinical Basis of Infectious Disease (1985)). Until this invention, the best characterized Gram positive organism for chromosomal DNA synthesis was Bacillus subtilis. Fractionation of B. subtilis has identified three DNA polymerases. (Gass et al., “Further Genetic and Enzymological Characterization of the Three Bacillus subtilis Deoxyribonucleic Acid Polymerases,” J. Biol. Chem., 248:7688-7700 (1973); Ganesan et al., “DNA Replication in a Polymerase I Deficient Mutant and the Identification of DNA Polymerases II and III in Bacillus subtilis,” Biochem. Biophys. Res. Commun., 50:155-163 (1973)). These polymerases are thought to be analogous to the three DNA polymerases of E. coli (DNA polymerases I, II, and III). Studies in B. subtilis have identified a polymerase that appears to be involved in chromosome replication and is termed Pol III (Ott et al., “Cloning and Characterization of the polC Region of Bacillus subtilis,” J. Bacteriol., 165:951-957 (1986); Barnes et al., “Localization of the Exonuclease and Polymerase Domains of Bacillus subtilis DNA Polymerase III,” Gene, 111:43-49 (1992); Barnes et al., “The 3′-5′ Exonuclease Site of DNA Polymerase III From Gram-positive Bacteria: Definition of a Novel Motif Structure,” Gene” 165:45-50 (1995) or Barnes et al., “Purification of DNA Polymerase III of Gram-positive Bacteria,” Methods in Enzy., 262:35-42 (1995)). The B. subtilis Pol III (encoded by polC) is larger (about 165 kDa) than the E. coli alpha subunit (about 129 kDa) and exhibits 3′-5′ exonuclease activity. The polC gene encoding this Pol III shows weak homology to the genes encoding E. coli alpha and the E. coli epsilon subunit. Hence, this long form of the B. subtilis Pol III (herein referred to as α-large or Pol III-L) essentially comprises both the alpha and epsilon subunits of the E. coli core polymerase. The S. aureus α-large has also been sequenced, expressed in E. coli, and purified; it contains DNA polymerase and 3′-5′ exonuclease activity (Pacitti et al., “Characterization and Overexpression of the Gene Encoding Staphylococcus aureus DNA Polymerase III,” Gene, 165:51-56 (1995)). Although α-large is essential to cell growth (Clements et al., “Inhibition of Bacillus subtilis Deoxyribonucleic Acid Polymerase III by Phenylhydrazinopyrimidines: Demonstration of a Drug-induced Deoxyribonucleic Acid-Enzyme Complex,” J. Biol. Chem., 250:522-526 (1975); Cozzarelli et al., “Mutational Alteraction of Bacillus subtilis DNA Polymerase III to Hydroxyphenylazopyrimidine Resistance Polymerase III is Necessary for DNA Replication,” Biochem. And Biophy. Res. Commun., 51:151-157 (1973); Low et al., “Mechanism of Inhibition of Bacillus subtilis DNA Polymerase III by the Arylhydrazinopyrimidine Antimicrobial Agents,” Proc. Natl. Acad. Sci. USA, 71:2973-2977 (1974)), there could still be another DNA polymerase(s) that is essential to the cell, such as occurs in yeast (Morrison et al., “A Third Essential DNA Polymerase in S. cerevisiae,” Cell, 62:1143-1151 (1990)).

Purification of α-large from B. subtilis results in only this single protein without associated proteins (Barnes et al., “Localization of the Exonuclease and Polymerase Domains of Bacillus subtilis DNA Polymerase III,” Gene, 111:43-49 (1992); Barnes et al., “The 3′-5′ Exonuclease Site of DNA Polymerase III From Gram-positive Bacteria: Definition of a Novel Motif Structure,” Gene” 165:45-50 (1995) or Barnes et al., “Purification of DNA Polymerase III of Gram-positive Bacteria,” Methods in Enzymol., 262:35-42 (1995)). Hence, it is possible that α-large is a member of the Type I replicase (like T5) in which it is processive on its own without accessory proteins. B. subtilis and S. aureus also have a gene encoding a protein that has approximately 30% homology to the beta subunit of E. coli; however, the protein product has not been purified or characterized (Alonso et al., “Nucleotide Sequence of the recF Gene Cluster From Staphylococcus aureus and Complementation Analysis in Bacillus subtilis recF Mutants,” Mol. Gen. Genet., 246:680-686 (1995); Alonso et al., “Nucleotide Sequence of the recF Gene Cluster From Staphylococcus aureus and Complementation Analysis in Bacillus subtilis recF Mutants,” Mol. Gen. Genet., 248:635-636 (1995)). Whether this beta subunit has a function in replication, a ring shape, or functions as a sliding clamp was not known until recently. It was also not known whether it is functional with α-large. Recently, it was shown that S. aureus β is functional as a ring, and that it also functions with α-large (U.S. patent application Ser. No. 09/235,245, which is hereby incorporated by reference). Further, a fourth DNA polymerase was identified with greater homology to E. coli α than α-large. This polymerase, called herein α-small, is shorter than α-large and lacks the domain homologous to epsilon. This polymerase also functions with the β ring, indicating that it may participate in chromosome replication. Indeed, a recent report indicates that α-small is essential for replication in Streptomyces coelicolor A3(2) (Flett et al., “A Gram-negative type” DNA Polymerase III is Essential for Replication of the Linear Chromosome of Streptomyces Coelicolor A3(2),” Mol. Micro., 31:949-958, (1999)).

As described earlier, purification of the replicase from the Gram positive B. subtilis gives only a single subunit Pol III, instead of a multicomponent complex. Also, S. aureus dnaX has been shown to encode only one subunit (U.S. patent application Ser. No. 09/235,245, which is hereby incorporated by reference). Moreover, S. aureus and B. subtilis lack homologues to χ, ψ, θ, and the δ subunit is only weakly homologous to δ of E. coli (only 28%). Further, they lack a homologue to dnaQ encoding ε. Instead, they contain this activity (3′-5′ exonuclease) in the polC gene product which provides the α-large form of α. The ε subunit is needed for high speed and processivity of the E. coli Pol III holoenzyme; the α subunit alone is much less rapid and processive with the β ring compared to the presence of both α and ε (Studwell et al., “Processive Replication is Contingent on the Exonuclease Subunit of DNA Polymerase III Holoenzyme,” J. Biol Chem, 265: 1171-1178 (1990)).

Studies using the E. coli β ring (and γ complex) show they confer onto S. aureus a quite efficient synthesis (U.S. patent application Ser. No. 09/235,245, which is hereby incorporated by reference), but the efficiency is not equal to that of E. coli αε with β (and γ complex). This may be due to use of the heterologous combination of an α subunit from one organism (S. aureus) with the β clamp from another (E. coli). However, it is also possible that S. aureus α simply does not function with a β clamp to produce speed and processivity comparable to the E. coli polymerase. Also, as described earlier, the α-large subunit of B. subtilis purifies as a single subunit, rather than associated with accessory subunits assembled into the three components of a rapid, processive machine (i.e., like E. coli Pol III holoenzyme). The lack of two DnaX products, lack of a multicomponent structure, and lack of gene homologues encoding several subunits of the three component, Pol III, of E. coli brings into question whether other types of bacteria, such as Gram positive cells, even have an enzyme with similar structure or comparable speed and processivity to that found in the Gram negative E. coli.

The lack of gene homologues encoding several subunits of the E. coli three component polymerase creates uncertainties with respect to reconstructing a rapid and processive polymerase from a Gram positive cell that has characteristics like the Pol III system of E. coli.

The γ and δ′ proteins are homologous to one another, encoding C-shape proteins (Dong et al., “DNA Polymerase III Accessory Proteins,” J. Biol. Chem, 268:11758-11765, (1993); Guenther et al., “Crystal Structure of the δ′ Subunit of the Clamp-loader Complex of E. coli DNA Polymerase III,” Cell, 91:335-345 (1997)). The clamp loaders of yeast and humans are composed of five proteins, all of which are homologous to one another and to γ and δ′ (Cullman et al., “Characterization of the Five Replication Factor C Genes of Saccharomyces Cerevisiae,” Mol. Cell. Biol., 15:4661-4671 (1995)). This provides evidence that a clamp loader can be composed entirely of C-shape proteins. Perhaps the Gram positive DnaX-protein (hereafter referred to as τ) and δ′ are sufficient to provide function as a clamp loader. Indeed, the clamp loader of T4 phage is composed of only two different proteins, gp44/62 complex (Young et al., “Structure and Function of the Bacteriophage T4 DNA Polymerase Holoenzyme,” Biochem., 31:8675-8690 (1992)). This idea is also supported by the presence of only two RFC genes in archaebacteria, suggesting that they may utilize two C-shaped proteins for clamp loading, in contrast to yeast and humans that use five. With this consideration in mind, genes were identified and isolated and the τ protein (encoded by dnaX) and δ′ (encoded by holB) of another Gram positive organism, Streptococcus pyogenes, were expressed and purified. As was observed in S. aureus, S. pyogenes dnaX produces only a single polypeptide. The β, encoded by dnaN of S. pyogenes, was also identified, expressed, and purified, as were the α-large subunit encoded by polC and the SSB encoded by the ssb gene. These proteins were studied for interactions and characterized for their effect on α-large. However, the hypothesis was incorrect as τ and δ′ did not form a τδ′ complex, nor did they assemble β onto DNA or provide stimulation of a when using β on primed and SSB coated M13mp18 ssDNA.

In light of the inability of S. pyogenes τ protein and δ′ to function as a clamp loader, it seemed reasonable to expect that one or more other proteins are needed. The fact that E. coli has some replicase subunits that other bacteria do not, suggests that other bacteria may have some replicase subunits that E. coli does not. Indeed, genetic studies of Bacillus subtilis demonstrates that it has three genes needed for replication that E. coli does not have. Two of these novel genes, called dnaB (not the same as E. coli dnaB encoding the helicase) and dnaH, have no significant homology to genes in the E. coli genome database (Bruand et al., “Nucleotide Sequence of the Bacillus subtilis dnaD gene,” Microbiol., 141:321-322 (1995); Hoshino et al., “Nucleotide Sequence of Bacillus subtilis dnaB: A gene Essential for DNA replication Initiation and Membrane Attachment,” Proc. Natl. Acad. Sci. USA, 84:653-657 (1987)). Further, dnaI of B. subtilis is important for replication and has, at best, a very limited homology to E. coli dnaC (Karamata et al., “Isolation and Genetic Analysis of Temperature-Sensitive Mutants of B. subtilis Defective in DNA synthesis,” Molec. Gen. Genetics, 108:277-287 (1970)). Perhaps one or more of these genes encode the proteins(s) necessary to provide clamp loading activity when combined with τ and δ′, or to couple with a to provide it with speed and/or processivity as the E. coli epsilon does. The S. pyogenes homologues of B. subtilis dnaI, dnaH, and dnaB were identified, cloned, and the encoded proteins were expressed and purified. However, these proteins failed to provide activity alone or in combinations with S. pyogenes τ and δ′ in loading S. pyogenes β onto DNA, or in stimulating S. pyogenes α-large in combination with β, τ, and δ′ on SSB coated primed M13mp18 ssDNA.

Weak homology exists for the holA gene among prokaryotes. This weak homologue of holA was identified in S. pyogenes and, then, it was cloned, expressed, and the putative δ was purified. The putative δ formed an isolatable complex with τ and δ′. In fact, the τδδ′ complex loaded S. pyogenes β onto DNA, and it stimulated S. pyogenes α-large in a β dependent reaction on primed SSB coated M13mp18 ssDNA. Hence, this protein was the only missing component necessary to provide clamp loading activity. Further, a mixture of a with τδδ′, followed by ion exchange chromatography on MonoQ, indicated formation of an ατδδ′ complex. Consistent with this, τ appeared to bind α in gel filtration analysis.

Whether the S. pyogenes three component polymerase can synthesize DNA in as rapid and processive of a fashion as the E. coli Pol III holoenzyme three component polymerase is very difficult to predict, because no other DNA polymerase known to date catalyzes synthesis at the rate or processivity of the E. coli three component polymerase. For example, the three component T4 phage polymerase travels about 400 nucleotides/s, the yeast DNA polymerase delta three component polymerase travels about 120 nucleotides/s, and the human DNA polymerase delta three component enzyme appears slower and less processive than the yeast enzyme.

The standard test for these speed and processivity characteristics is examination of a time course in extension of a primer on a very long template, such as around the 7.2 kb M13mp18 ssDNA genome coated with SSB and primed with a synthetic DNA oligonucleotide. The results of experiments of this type demonstrate that the three component S. pyogenes polymerase is indeed extremely rapid in synthesis. Surprisingly, it is just as fast as the E. coli enzyme. Extension proceeds at about 700-800 nucleotides per second, completing the entire template in about 9 seconds. The enzyme was fully processive throughout replication of the M13mp18 genome, as could be determined from the fact that some templates were not extended at all, while others were extended to completion. If the enzyme had not been processive during the entire replication reaction, then when it comes off one partially extended DNA genome it would have reassociated with the unextended DNA that remained and partially replicated it as well (and so on until the entire population of DNA became fully replicated). This did not happen. Instead, the reaction showed a mixture of completely replicated templates and templates that were still untouched starting material. This indicates that the enzyme stays with a template until it completes it before it cycles over to replicate another one (i.e., it is highly processive). Each of the five proteins, α, τ, δ, δ′ and β, are needed to obtain this rapid and processive DNA synthesis.

This invention has provided an intellectual template by which the clamp loader component of these three component polymerases can be obtained from any eubacterial prokaryotic cell and how to use it with the other components to produce a rapid and processive polymerase. All prokaryotes in the eubacterial kingdom that have been sequenced to date contain homologues of these genes. As the process of lateral gene transfer appears to be a major force in evolution, it would appear that relatedness of enzymes and enzyme machines is best judged by comparisons of their genes and proteins rather than by phylogeny of which bacteria they are in (Doolittle et al., “Archaeal Genomics: Do Archaea have a Mixed Heritage?,” Curr. Biol., 8:R209-R211 (1998)). As pointed out earlier in this application, most bacteria have genetic characteristics of replication genes/proteins of S. pyogenes rather than that of E. coli (i.e., no genes encoding χ, ψ, or θ, only a weak homolog to δ, or a dnaX gene encoding only a single protein).

The dnaX gene encoding τ and γ in E. coli encodes only one protein in some organisms, but, as this application shows, it is still functional in forming a protein complex capable of rapid and processive DNA synthesis. In addition, this application shows that the delta subunit, which is only weakly homologous among different prokaryotic organisms, is an essential functional subunit of the three component polymerase (instead of having diverged so as to fulfill an entirely different function in some other intracellular process). As mentioned earlier, several genes encoding subunits of the E. coli clamp loader (γ complex; γ, δ, δ′, χ, ψ) are not obviously present in other prokaryotes (holC and holD encoding χ and ψ). Hence, one may anticipate that other genes may have evolved to encode new subunits that replace these, and that these new subunits may have been essential to the activity of the clamp loader. For example, they may have either taken over some of the functionality of another subunit, or structurally (e.g., the physical presence of a subunit could be needed for one subunit to assume its proper and active conformation, or for one or more of the subunits to form a complex together to yield the multisubunit clamp loader assembly). In addition, this application shows that the α subunit (polC gene product) is sufficient for rapid and processive synthesis with the other two components (i.e., E. coli requires ε submit to bind to α for rapid and processive synthesis of α with the β clamp). Finally, this application shows that the S. pyogenes three component polymerase synthesizes DNA as fast as the E. coli Pol III three component polymerase. Up to this point, the E. coli Pol III three component polymerase was over twice the speed of the T4 enzyme and over 5 times the speed of others. Hence, it was possible that E. coli may have been unique among prokaryotes in having a polymerase that achieves such speed. This invention shows that this is not the case. Instead, this speed in polymerization generalizes to the Gram positive prokaryotic three component DNA polymerases. It may be presumed, now that two examples of three component polymerases in widely divergent bacteria share the characteristics of rapid, processive synthesis, that the three component polymerase of other eubacteria will also be rapid and processive.

These rapid and processive three component DNA polymerases can be applied to several important uses. DNA polymerases currently in use for DNA sequencing and DNA amplification use enzymes that are much slower and thus could be improved upon. This is especially true of amplification as the three component polymerase is capable of speed and high processivity making possible amplification of very long (tens of Kb to Mb) lengths of DNA in a time efficient manner. These three component polymerases also function in conjunction with a replicative helicase (DnaB) and, thus, are capable of amplification at ambient temperature using the helicase to melt the DNA duplex. This property could be useful in amplification reaction procedures such as in polymerase chain reaction (PCR) methodology. Finally, these three component polymerases and their associated helicase (DnaB) and primase (DnaG) are attractive targets for antibiotics due to their essential and central role in cell viability.

This application provides a three component polymerase from two human pathogens in the Gram positive class. It makes possible the production of this three component polymerase from other bacteria of the Gram positive type (e.g., Streptococci, Staphylococci, Mycoplasma) and other types of bacteria lacking χ, ψ, or θ, those having only one protein produced by their dnaX gene such as obligate intracellular parasites, Mycoplasmas (possibly evolved from Gram positives), Cyanobacteria (Synechocystis), Spirochaetes such as Borrelia and Treponemia and Chlamydia, and distant relatives of E. coli in the Gram negative class (e.g., Rickettsia and Helicobacter). These three component polymerases are useful in manipulation of nucleic acids for research and diagnostic purposes (e.g., sequencing and amplification methods) and for screening chemicals for antibiotic activity (useful in human or animal therapy and agriculture such as animal feed supplements). There are several assays described previously in U.S. patent application Ser. No. 09/235,245 to O'Donnell et al., which is hereby incorporated by reference, that use these three component polymerases (or subassemblies), as well as the DnaB and DnaG homologues, either alone or in various combinations, for the purpose of screening chemicals, such as chemical libraries, for inhibitor activity. Such inhibitors can be developed further (usually by chemical manipulation and alteration) into lead compounds and then into full fledged pharmaceuticals.

There remains a need to understand the molecular details of the process of DNA replication in other cells that are quite different from E. coli, such as in Gram positive cells. It is possible that a more detailed understanding of replication proteins will lead to discovery of new antibiotics. Therefore, a deeper understanding of replication proteins of Gram positive bacteria is especially important given the emergence of drug resistant strains of these organisms. For example, Staphylococcus aureus has successfully mutated to become resistant to all common antibiotics.

The “target” protein(s) of an antibiotic drug is generally involved in a critical cell function, such that blocking its action with a drug causes the pathogenic cell to die or no longer proliferate. Current antibiotics are directed to very few targets. These include membrane synthesis proteins (e.g., vancomycin, penicillin, and its derivatives such as ampicillin, amoxicillin, and cephalosporin), the ribosome machinery (e.g., tetracycline, chloramphenicol, azithromycin, and the aminoglycosides such as kanamycin, neomycin, gentamicin, streptomycin), RNA polymerase (e.g., rifampimycin), and DNA topoisomerases (e.g., novobiocin, quinolones, and fluoroquinolones). The DNA replication apparatus is a crucial life process and, thus, the proteins involved in this process are good targets for antibiotics.

A powerful approach to discovery of a new drug is to obtain a target protein, characterize it, and develop in vitro assays of its cellular function. Large chemical libraries can then be screened in the functional assays to identify compounds that inhibit the target protein. These candidate pharmaceuticals can then be chemically modified to optimize their potency, breadth of antibiotic spectrum non-toxicity, performance in animal models and, finally, clinical trials. The screening of large chemical libraries requires a plentiful source of the target protein. An abundant supply of protein generally requires overproduction techniques using the gene encoding the protein. This is especially true for replication proteins as they are present in low abundance in the cell.

Selective and robust assays are needed to screen reliably a large chemical library. The assay should be insensitive to most chemicals in the concentration range normally used in the drug discovery process. These assays should also be selective and not show inhibition by antibiotics known to target proteins in processes outside of replication.

The present invention is directed to overcoming these deficiencies in the art.

### SUMMARY OF THE INVENTION

The present invention relates to various isolated DNA molecules from Staphylococcus aureus and Streptococcus pyogenes, both of which are Gram positive bacteria. These include DNA molecules which include a coding region from the dnaE gene (encoding α-small), dnaX gene (encoding tau), polC gene (encoding Pol III L or α-large), dnaN gene (encoding beta), holA gene (encoding delta), holB gene (encoding delta prime), ssb gene (encoding SSB), dnaB gene (encoding DnaB), and dnaG gene (encoding DnaG) of S. aureus and/or S. pyogenes. These DNA molecules can be inserted into an expression system and used to transform host cells. The isolated proteins or polypeptides encoded by these DNA molecules, and their ability to function when used in combination is also disclosed. The resulting actions provide assembling a ring onto DNA via a clamp loader, and polymerase activity dependent on this ring that is rapid and processive.

A further aspect of the present invention relates to a method of identifying compounds which inhibit activity of a polymerase product of polC or dnaE. This method is carried out by forming a reaction mixture comprising a primed DNA molecule, a polymerase product of polC or dnaE, a candidate compound, a dNTP, and optionally either a beta subunit, a tau complex, or both the beta subunit and the tau complex, wherein at least one of the polymerase product of polC or dnaE, the beta subunit, the tau complex, or a subunit or combination of subunits thereof is derived from a Eubacteria other than Escherichia coli; subjecting the reaction mixture to conditions effective to achieve nucleic acid polymerization in the absence of the candidate compound; analyzing the reaction mixture for the presence or absence of nucleic acid polymerization extension products; and identifying the candidate compound in the reaction mixture where there is an absence of nucleic acid polymerization extension products.

The present invention deciphers the structure and mechanism of the chromosomal replicase of Gram positive bacteria and other bacteria lacking holC, holD, holE or dnaQ genes, or having a dnaX gene that encodes only one protein. Rather than use a DNA polymerase that attains high efficiency on its own, or with one other subunit, the Gram positive bacteria replicase is a three component type of replicase (class III) that uses a sliding clamp protein. The Gram positive bacteria replicase also uses a clamp loader component that assembles the sliding clamp onto DNA. This knowledge, and the enzymes involved in the replication process, can be used for the purpose of screening for potential antibiotic drugs. Further, information about chromosomal replicases may be useful in DNA sequencing, DNA amplification, polymerase chain reaction, and other DNA polymerase related techniques.

The present invention identifies two DNA polymerases (both of Pol III type) in Gram positive bacteria that utilize the sliding clamp and clamp loader. The present invention also identifies a gene with homology to the alpha subunit of E. coli DNA polymerase III holoenzyme, the chromosomal replicase of E. coli. These DNA polymerases can extend a primer around a large circular natural template when the beta clamp has been assembled onto the primed ssDNA by the clamp loader or a primer on a linear DNA where the beta clamp may assemble by itself by sliding over an end.

The present invention shows that the clamp and clamp loader components of Gram negative cells can be exchanged for those of Gram positive cells in that the clamp, once assembled onto DNA, will function with Pol III obtained from either Gram positive and Gram negative sources. This result implies that important contacts between the polymerase and clamp have been conserved during evolution. Therefore, these “mixed systems” may provide assays for an inhibitor of this conserved interaction. Such an inhibitor may be expected to shut down replication, and since the interaction is apparently conserved across the evolutionary spectrum from Gram positive and Gram negative cells, the inhibitor may exhibit a broad spectrum of antibiotic activity.

The present invention demonstrates that Gram positive bacteria contain a beta subunit that behaves as a sliding clamp that encircles DNA. A dnaX gene sequence encoding a protein homolog of the gamma/tau subunit of the clamp loader (gamma/tau complex) E. coli DNA polymerase III holoenzyme is also identified. The presence of this gene confirms the presence of a clamp loading apparatus in Gram positive bacteria that will assemble beta clamps onto DNA for the DNA polymerases.

This application also outlines methods and assays for use of these replication proteins in drug screening processes.

### BRIEF DESCRIPTION OF THE DRAWINGS

FIG. 1 shows the construction of the S. aureus Pol III-L expression vector. The gene encoding Pol III-L was cloned into a pET11 expression vector in a three step cloning scheme as illustrated.

FIGS. 2A-D describe the expression and purification of S. aureus Pol III-L (alpha-large). FIG. 2A compares E. coli cells that contain the pET11PolC expression vector that are either induced or uninduced for protein expression. The gel is stained with Coomassie Blue. The induced band corresponds to the expected molecular weight of the S. aureus Pol III-L, and is indicated to the right of the gel. FIGS. 2B-C show the results of the MonoQ chromatography of a lysate of E. coli (pET11PolC-L) induced for Pol III-L. The fractions were analyzed in a Coomassie Blue stained gel (FIG. 2B) and for DNA synthesis (FIG. 2C). Fractions containing Pol III-L are indicated. In FIG. 2D, fractions containing Pol III-L from the MonoQ column were pooled and chromatographed on a phosphocellulose column. This shows an analysis of the column fractions from the phosphocellulose column in a Coomassie Blue stained polyacrylamide gel. The position of Pol III-L is indicated to the right.

FIG. 3 shows the S. aureus beta expression vector. The dnaN gene was amplified from S. aureus genomic DNA and cloned into the pET16 expression vector.

FIGS. 4A-C illustrate the expression and purification of S. aureus beta. FIG. 4A compares E. coli cells that contain the pET16beta expression vector that are either induced or uninduced for protein expression. The gel is stained with Coomassie Blue. The induced band corresponds to the expected molecular weight of the S. aureus beta, and is indicated to the right of the gel. The migration position of size standards are indicated to the left. FIG. 4B shows the results of MonoQ chromatography of an E. coli (pET16beta) lysate induced for beta. The fractions were analyzed in a Coomassie Blue stained gel, and fractions containing beta are indicated. In FIG. 4C, fractions containing beta from the MonoQ column were pooled and chromatographed on a phosphocellulose column. This shows an analysis of the column fractions from the phosphocellulose column in a Coomassie Blue stained polyacrylamide gel. The position of beta is indicated to the right.

FIGS. 5A-B demonstrate that the S. aureus beta stimulates S. aureus Pol III-L and E. coli Pol III core on linear DNA, but not circular DNA. In FIG. 5A, the indicated proteins were added to replication reactions containing polydA-oligodT as described in the Examples infra. Amounts of proteins added, when present, were: lanes 1,2: S. aureus Pol III-L, 7.5 ng; S. aureus beta, 6.2 μg; Lanes 3,4: E. coli Pol III core, 45 ng; S. aureus beta, 9.3 μg; Lanes 5,6: E. coli Pol III core, 45 ng; E. coli beta, 5 μg. Total DNA synthesis was: Lanes 1-6: 4.4, 30.3, 5.1, 35.5, 0.97, 28.1 pmol, respectively. In FIG. 5B, Lanes 1-3, the indicated proteins were added to replication reactions containing circular singly primed M13mp18 ssDNA as described in the Examples infra. S. aureus beta, 0.8 μg; S. aureus Pol III-L, 300 ng (purified through MonoQ); E. coli clamp loader complex, 1.7 μg. Results in the E. coli system are shown in Lanes 4-6. Total DNA synthesis was: Lanes 1-6: 0.6, 0.36, 0.99, 2.7, 3.5, 280 pmol, respectively.

FIG. 6 shows that S. aureus Pol III-L functions with E. coli beta and clamp loader complex on circular primed DNA. It also shows that S. aureus beta does not convert Pol III-L with sufficient processivity to extend the primer all the way around a circular DNA. Replication reactions were performed on the circular singly primed M13mp18 ssDNA. Proteins added to the assay are as indicated in this figure. The amount of each protein, when present, is: S. aureus beta, 800 ng; S. aureus Pol III-L, 1500 ng (MonoQ fraction 64); E. coli Pol III core, 450 ng; E. coli beta, 100 ng; E. coli gamma complex, 1720 ng. Total DNA synthesis in each assay is indicated at the bottom of the figure.

FIGS. 7A-B show that S. aureus contains four distinct DNA polymerases. Four different DNA polymerases were partially purified from S. aureus cells. S. aureus cell lysate was separated from DNA and, then, chromatographed on a MonoQ column. Fractions were analyzed for DNA polymerase activity. Three peaks of activity were observed. The second peak was the largest and was expected to be a mixture of two DNA polymerases based on early studies in B. subtilis. Chromatography of the second peak on phosphocellulose (FIG. 7B) resolved two DNA polymerases from one another.

FIGS. 8A-B show that S. aureus has two DNA Pol III's. The four DNA polymerases partially purified from S. aureus extract, designated peaks I-IV in FIG. 7, were assayed on circular singly primed M13mp18 ssDNA coated with E. coli SSB either in the presence or absence of E. coli beta (50 ng) and clamp loader complex (50 ng). Each reaction contained 2 μl of the partially pure polymerase (Peak 1 was Mono Q fraction 24 (1.4 μg), Peak 2 was phosphocellulose fraction 26 (0.016 mg/ml), Peak 3 was phosphocellulose fraction 46 (0.18 mg/ml), and Peak 4 was MonoQ fraction 50 (1 μg). FIG. 8A shows the product analysis in an agarose gel. FIG. 8B shows the extent of DNA synthesis in each assay.

FIG. 9 compares the homology between the polypeptide encoded by dnaE of S. aureus and other organisms. An alignment is shown for the amino acid sequence of the S. aureus dnaE product with the dnaE products (alpha subunits) of E. coli and Salmonella typhimurium.

FIG. 10 compares the homology between the N-terminal regions of the gamma/tau polypeptides of S. aureus, B. subtilis, and E. coli. The conserved ATP site and the cystines forming the zinc finger are indicated above the sequence. The organisms used in the alignment were: E. coli (GenBank); and B. subtilis.

FIG. 11 compares the homology between the DnaB polypeptide of S. aureus and other organisms. The organisms used in the alignment were: E. coli (GenBank); B. subtilis; Sal. Typ., (Salmonella typhimurium).

FIGS. 12A-B show the alignment of the delta subunit encoded by holA for E. coli and B. subtilis (FIG. 12A) and for the delta subunit of B. subtilis and S. pyogenes (FIG. 12B). FIG. 12A shows ClustalW generated alignment of S. pyogenes (Gram positive) delta to E. coli (Gram negative) delta. FIG. 12B shows ClustalW generated alignment of B. subtilis (Gram positive) delta to S. pyogenes (Gram positive) delta.

FIG. 13 is an image of an autoradiograph of an agarose gel analysis of replication products from singly primed, SSB coated M13 mp18 ssDNA using the reconstituted S. aureus Pol III holozyme. Only in the presence of the τδδ′ complex does α-large (PolC) function with β to replicate a full circular duplex DNA (RFII).

FIG. 14 shows a Comassie Blue stained SDS polyacrylamide gel of the pure S. pyogenes subunits corresponding to alpha-large, alpha-small, dnaX gene product (called tau), beta, delta, delta prime, and SSB. The first lane shows the position of molecular weight markers. Purified proteins were separated on a 15% SDS-PAGE and stained with Coomassie Brilliant Blue R-250. Each lane contains 5 microgram of each protein. Lane 1, markers; lane 2, alpha-large; lane 3, alpha-small, lane 4, tau subunit; lane 5, beta subunit; lane 6, delta subunit; lane 7, delta prime subunit; lane 8, single strand DNA binding protein.

FIGS. 15A-C document the ability to reconstitute the τδδ′ complex of S. pyogenes. Proteins were mixed and gel filtered on Superose 6, followed by analysis of the column fractions in a SDS polyacrylamide gel. FIG. 15A shows a mixture of τδδ′. FIG. 15B shows a mixture of τδ. FIG. 15C shows a mixture of τδ′.

FIGS. 16A-E show that the S. pyogenes τδδ′ complex can load the S. pyogenes beta clamp onto (circular) DNA. Loading reactions contained 500 fm nicked pBSK plasmid, 500 fm either τδδ′ complex, tau, delta, or delta prime, 1 pm ³²P labelled beta dimer, 8 mM MgCl₂, 1 mM ATP. Reaction components were preincubated for 10 min at 37° C. prior to loading onto 5 ml Biogel A15M column equilibrated with buffer A containing 100 mM NaCl. FIG. 16A demonstrates the ability of τδδ′ complex to load the beta dimer onto a nicked pBSK circular plasmid. FIGS. 16B-E show the results of using either: beta alone (FIG. 16B); δδ′ plus β (FIG. 16C); τ, δ and β (FIG. 16D); τ, δ′ and β (FIG. 16E).

FIGS. 17A-C show that τ and alpha interact. FIG. 17A shows the result of gel filtration analysis of a mixture of τ with alpha-large. Gel filtration fractions are analyzed in a SDS polyacrylamide gel. FIGS. 17B and 17C show the results using only τ or only alpha-large, respectively. Comparison of the elution positions of proteins shows that the positions of alpha and tau are shifted toward a higher molecular weight complex when they are present together. The fact they do not exactly comigrate may indicate that they initially are together in a complex, but that the complex dissociates during the time of the gel filtration experiment (over one half hour).

FIG. 18 documents the ability to reconstitute α_{L}τδδ′ (pol III*) complex of S. pyogenes. Proteins were mixed, preincubated for 20 min at 15° C., gel filtered on Superose 6, followed by analysis of the column fractions in a SDS polyacrylamide gel (FIG. 18). Proteins were loaded on a MonoQ column, then eluted with a linear gradient of 50-500 mM NaCl, followed by analysis of the column fractions in a SDS polyacrylamide gel. The α_{L}τδδ′ complex migrates early.

FIG. 19 illustrates the speed and processivity of the S. pyogenes α_{C}τδδ′ (pol III*) complex. The α_{L}τδδ′ (pol III*) complex was incubated with primed M13mp18 ssDNA (coated with S. pyogenes SSB) and only two dNTPs, then replication was initiated upon adding the remaining two dNTPs. Reactions contained 25 fmol singly primed M13mp18 ssDNA template, 300 fmol β₂, and either 75 fmol or 250 fmol α_{L}τδδ′. Time points were quenched with SDS/EDTA then analyzed in a neutral agarose gel followed by autoradiography. Each time point is a separate reaction. The time course of polymerization was performed at two different ratios of polymerase/primed template to assess speed and processivity of nucleotide incorporation.

FIGS. 20A-I show the extent of homology between S. pyogenes replication genes and other organisms. Due to the low homology of delta (FIG. 20D), one must “walk” from one organism to the next in order to recognize the homologue with high probability. Percent identity over regions of the indicated number of amino acid residues is shown for each match (i.e., the two organisms at the opposite ends of each line). Amino acid sequences were retrieved from either GenBank or individual unfinished genome databases.

FIG. 21A-F are images illustrating that the S. pyogenes DnaE (alpha-small) polymerase functions with β. FIGS. 21A-B illustrate the relationship between DnaE and β for association with ssDNA. Different amounts of DnaE polymerase were added to a SSB coated M13mp18 ssDNA circle primed with a single DNA oligonucleotide, and products were analyzed in a native agarose gel. Reactions were performed in the presence of τδδ′ and either the absence (FIG. 21C, panels 1-4) or presence (FIG. 21D, panels 1-4) of β. Positions of completed duplex (RFII) and initial primed template (ssDNA) are indicated. FIG. 21E shows an analysis of exonuclease activity by PolC and DnaE on a 5′-32P-DNA 30-mer. Aliquots were removed at the indicated times and analyzed in a sequencing gel. FIG. 21F shows the effect of TMAU on PolC and DnaE in the presence of τδδ′ and β. DNA products were analyzed in a native agarose gel. Positions of initial primed M13mp18 (ssDNA) and completed circular duplex (RFII) are indicated.

### DETAILED DESCRIPTION OF THE INVENTION

The present invention relates to various isolated nucleic acid molecules from Gram positive bacteria and other bacteria lacking holC, holD, or holE genes or having a dnaX gene encoding only one subunit. These include DNA molecules which correspond to the coding regions of the dnaE, dnaX, holA, holB, polC, dnaN, SSB, dnaB, and dnaG genes. These DNA molecules can be inserted into an expression system or used to transform host cells. The isolated proteins or polypeptides encoded by these DNA molecules and their use to form a three component polymerase are also disclosed. Also encompassed by the present invention are corresponding RNA molecules transcribed from the DNA molecules.

These DNA molecules and proteins can be derived from numerous bacteria, including Staphylococcus, Streptococcus, Enterococcus, Mycoplasma, Mycobacterium, Borrelia, Treponema, Rickettsia, Chlamydia, Helicobacter, and Thermatoga. It is particularly directed to such DNA molecules and proteins derived from Streptococcus and Staphylococcus bacteria, particularly Streptococcus pyogenes and Staphylococcus aureus (see U.S. patent application Ser. No. 09/235,245, which is hereby incorporated by reference).

The gene sequences used to obtain DNA molecules of the present invention were obtained by sequence comparisons with the E. coli counterparts, followed by detailed analysis of the raw sequence data in the contigs from the S. pyogenes database (http://dnal.chem.ou.edu/strep.html) or the S. aureus database (http://www.genome.ou.edu/staph.html) to identify the open reading frames. In many instances, nucleotide errors were observed causing frameshifts in the open reading frame thus truncating it. Therefore, upon cloning the genes via PCR, the genes were sequenced to obtain correct information. Also, the full nucleotide sequence of the ssb gene was not present in the data base. This was cloned by circular PCR and the full sequence is reported below.

The S. aureus dnaX and dnaE genes were identified by aligning genes of several organisms and designing primers for use in PCR to obtain a gene fragment, followed by steps to identify the entire gene.

One aspect of the present invention relates to a newly discovered Pol III gene (herein identified as dnaE) of S. aureus whose encoded protein is homologous to E. coli alpha (product of dnaE gene). The partial nucleotide sequence of the S. aureus dnaE gene corresponds to SEQ. ID. No. 1 as follows:

| | |
|---|---|
| atggtggcat atttaaatat tcatacggct tatgatttgt taaattcaag cttaaaaata | 60 |
| gaagatgccg taagacttgc tgtgtctgaa aatgttgatg cacttgccat aactgacacc | 120 |
| aatgtattgt atggttttcc taaattttat gatgcatgta tagcaaataa cattaaaccg | 180 |
| atttttggta tgacaatata tgtgacaaat ggattaaata cagtcgaaac agttgttcta | 240 |
| gctaaaaata atgatggatt aaaagatttg tatcaactat catcggaaat aaaaatgaat | 300 |
| gcattagaac atgtgtcgtt tgaattatta aaacgatttt ctaacaatat gattatcatt | 360 |
| tttaaaaaag tcggtgatca acatcgtgat attgtacaag tgtttgaaac ccataatgac | 420 |
| acatatatgg accaccttag tatttcgatt caaggtagaa aacatgtttg gattcaaaat | 480 |
| gtttgttacc aaacacgtca agatgccgat acgatttctg cattagcagc tattagagac | 540 |
| aatacaaaat tagacttaat tcatgatcaa gaagattttg gtgcacattt tttaactgaa | 600 |
| aaggaaatta atcaattaga tattaaccaa gaatatttaa cgcaggttga tgttatagct | 660 |
| caaaagtgtg atgcagaatt aaaatatcat caatctctac ttcctcaata tgagacacct | 720 |
| aatgatgaat cagctaaaaa atatttgtgg cgtgtcttag ttacacaatt gaaaaaatta | 780 |
| gaacttaatt atgacgtcta tttagagcga ttgaaatatg agtataaagt tattactaat | 840 |
| atgggttttg aagattattt cttaatagta agtgatttaa tccattatgc gaaaacgaat | 900 |
| gatgtgatgg taggtcctgg tcgtggttct tcagctggct cactggtcag ttatttattg | 960 |
| ggaattacaa cgattgatcc tattaaattc aatctattat ttgaacgttt tttaaaccca | 1020 |
| gaacgtgtaa caatgcctga tattgatatt gactttgaag atacacgccg agaaagggtc | 1080 |
| attcagtacg tccaagaaaa atatggcgag ctacatgtat ctggaattgt gactttcggt | 1140 |
| catctgcttg caagagcagt tgctagagat gttggaagaa ttatggggtt tgatgaagtt | 1200 |
| acattaaatg aaatttcaag tttaatccca cataaattag gaattacact tgatgaagca | 1260 |
| tatcaaattg acgattttaa agagtttgta catcgaaacc atcgacatga acgctggttc | 1320 |
| agtatttgta aaaagttaga aggtttacca agacatacat ctacacatgc ggcaggaatt | 1380 |
| attattaatg accatccatt atatgaatat gcccctttaa cgaaagggga tacaggatta | 1440 |
| ttaacgcaat ggacaatgac tgaagccgaa cgtattgggt tattaaaaat agattttcta | 1500 |
| gggttgagaa acttatcgat tattcatcaa atcttaacac aagtcaaaaa agatttaggt | 1560 |
| attaatattg atatcgaaaa gattccgttt gatgatcaaa aagtgtttga attgttgtcg | 1620 |
| caaggagata cgactggcat attccaatta gagtctgacg gtgtaagaag tgtattaaaa | 1680 |
| aaattaaagc cggaacactt tgaagatatt gttgctgtaa cttctttgta tagaccaggt | 1740 |
| ccaatggaag aaattccaac ttacattaca agaagacatg atccaagcaa agttcaatat | 1800 |
| ttacatccgc atttagaacc tatattaaaa aatacttacg gtgttattat ttatcaagag | 1860 |
| caaattatgc aaatagcgag cacatttgca aacttcagtt atggtgaagc ggatatttta | 1920 |
| agaagagcaa tgagtaaaaa aaatagagct gttcttgaaa gtgagcgtca acattttata | 1980 |
| gaaggtgcaa agcaaaatgg ttatcacgaa gacattagta agcaaatatt tgatttgatt | 2040 |
| ctgaaatttg ctgattatgg ttttcctaga gcacatgctg tcagctattc taaaattgca | 2100 |
| tacattatga gctttttaaa agtccattat ccaaattatt tttacgcaaa tattttaagt | 2160 |
| aatgttattg gaagtgagaa gaaaactgct caaatgatag aagaagcaaa aaaacaaggt | 2220 |
| atcactatat tgccaccgaa cattaacgaa agtcattggt tttataaacc ttcccaagaa | 2280 |
| ggcatttatt tatcaattgg tacaattaaa ggtgttggtt atcaaagtgt gaaagtgatt | 2340 |
| gttgatgaac gttatcagaa cggcaaattt aaagatttct ttgattttgc tagacgtata | 2400 |
| ccgaagagag tcaaaacgag aaagttactt gaagcactga ttttagtggg agcgtttgat | 2460 |
| gcttttggta aaacacgttc aacgttgttg caagctattg atcaagtgtt ggatggcgat | 2520 |
| ttaaacattg aacaagatgg ttttttattt gatattttaa cgccaaaaca gatgtatgaa | 2580 |
| gataaagaag aattgcctga tgcacttatt agtcagtacg aaaaagaata tttaggattt | 2640 |
| tatgtttcgc aacacccagt agataaaaag tttgttgcca aacaatattt aacgatattt | 2700 |
| aaattgagta acgcgcagaa ttataaacct atattagtac agtttgataa agttaaacaa | 2760 |
| attcgaacta aaaatggtca aaatatggca ttcgtcacat taaatgatgg cattgaaact | 2820 |
| ttagatggtg tgattttccc taatcagttt aaaaagtacg aagagttgtt atcacataat | 2880 |
| gacttgttta tagttagcgg gaaatttgac catagaaagc aacaacgtca actaattata | 2940 |
| aatgagattc agacattagc cacttttgaa gaacaaaaat tagcatttgc caaacaaatt | 3000 |
| ataattagaa ataaatcaca aatagatatg tttgaagaga tgattaaagc tacgaaagag | 3060 |
| aatgctaatg atgttgtgtt atccttttat gatgaaacga ttaaacaaat gactacttta | 3120 |
| ggctatatta atcaaaaaga tagtatgttt aataatttta tacaatcctt taaccctagt | 3180 |
| gatattaggc ttata | 3195 |

The S. aureus dnaE encoded protein, called α-small, has an amino acid sequence corresponding to SEQ. ID. No. 2 as follows:

```
Met Val Ala Tyr Leu Asn Ile His Thr Ala Tyr Asp Leu Leu Asn Ser
    1 5 10 15
Ser Leu Lys Ile Glu Asp Ala Val Arg Leu Ala Val Ser Glu Asn Val
                          20 25 30
Asp Ala Leu Ala Ile Thr Asp Thr Asn Val Leu Tyr Gly Phe Pro Lys
                  35 40 45
Phe Tyr Asp Ala Cys Ile Ala Asn Asn Ile Lys Pro Ile Phe Gly Met
          50 55 60
Thr Ile Tyr Val Thr Asn Gly Leu Asn Thr Val Glu Thr Val Val Leu
  65 70 75 80
Ala Lys Asn Asn Asp Gly Leu Lys Asp Leu Tyr Gln Leu Ser Ser Glu
                                  85 90 95
Ile Lys Met Asn Ala Leu Glu His Val Ser Phe Glu Leu Leu Lys Arg
                        100 105 110
Phe Ser Asn Asn Met Ile Ile Ile Phe Lys Lys Val Gly Asp Gln His
                115 120 125
Arg Asp Ile Val Gln Val Phe Glu Thr His Asn Asp Thr Tyr Met Asp
        130 135 140
His Leu Ser Ile Ser Ile Gln Gly Arg Lys His Val Trp Ile Gln Asn
145 150 155 160
Val Cys Tyr Gln Thr Arg Gln Asp Ala Asp Thr Ile Ser Ala Leu Ala
                                165 170 175
Ala Ile Arg Asp Asn Thr Lys Leu Asp Leu Ile His Asp Gln Glu Asp
                        180 185 190
Phe Gly Ala His Phe Leu Thr Glu Lys Glu Ile Asn Gln Leu Asp Ile
                195 200 205
Asn Gln Glu Tyr Leu Thr Gln Val Asp Val Ile Ala Gln Lys Cys Asp
        210 215 220
Ala Glu Leu Lys Tyr His Gln Ser Leu Leu Pro Gln Tyr Glu Thr Pro
225 230 235 240
Asn Asp Glu Ser Ala Lys Lys Tyr Leu Trp Arg Val Leu Val Thr Gln
                                245 250 255
Leu Lys Lys Leu Gln Leu Asn Tyr Asp Val Tyr Leu Glu Arg Leu Lys
                        260 265 270
Tyr Glu Tyr Lys Val Ile Thr Asn Met Gly Phe Glu Asp Tyr Phe Leu
                275 280 285
Ile Val Ser Asp Leu Ile His Tyr Ala Lys Thr Asn Asp Val Met Val
        290 295 300
Gly Pro Gly Arg Gly Ser Ser Ala Gly Ser Leu Val Ser Tyr Leu Leu
305 310 315 320
Gly Ile Thr Thr Ile Asp Pro Ile Lys Phe Asn Leu Leu Phe Glu Arg
                                325 330 335
Phe Leu Asn Pro Glu Arg Val Thr Met Pro Asp Ile Asp Ile Asp Phe
                        340 345 350
Glu Asp Thr Arg Arg Glu Arg Val Ile Gln Tyr Val Gln Glu Lys Tyr
                355 360 365
Gly Glu Leu His Val Ser Gly Ile Val Thr Phe Gly His Leu Leu Ala
        370 375 380
Arg Ala Val Ala Arg Asp Val Gly Arg Ile Met Gly Phe Asp Glu Val
385 390 395 400
Thr Leu Asn Glu Ile Ser Ser Leu Ile Pro His Lys Leu Gly Ile Thr
                                405 410 415
Leu Asp Glu Ala Tyr Gln Ile Asp Asp Phe Lys Glu Phe Val His Arg
                        420 425 430
Asn His Arg His Glu Arg Trp Phe Ser Ile Cys Lys Lys Leu Glu Gly
                435 440 445
Leu Pro Arg His Thr Ser Thr His Ala Ala Gly Ile Ile Ile Asn Asp
        450 455 460
His Pro Leu Tyr Glu Tyr Ala Pro Leu Thr Lys Gly Asp Thr Gly Leu
465 470 475 480
Leu Thr Gln Trp Thr Met Thr Glu Ala Glu Arg Ile Gly Leu Leu Lys
                                485 490 495
Ile Asp Phe Leu Gly Leu Arg Asn Leu Ser Ile Ile His Gln Ile Leu
                        500 505 510
Thr Gln Val Lys Lys Asp Leu Gly Ile Asn Ile Asp Ile Glu Lys Ile
                515 520 525
Pro Phe Asp Asp Gln Lys Val Phe Glu Leu Leu Ser Gln Gly Asp Thr
        530 535 540
Thr Gly Ile Phe Gln Leu Glu Ser Asp Gly Val Arg Ser Val Leu Lys
545 550 555 560
Lys Leu Lys Pro Glu His Phe Glu Asp Ile Val Ala Val Thr Ser Leu
                                565 570 575
Tyr Arg Pro Gly Pro Met Glu Glu Ile Pro Thr Tyr Ile Thr Arg Arg
                        580 585 590
His Asp Pro Ser Lys Val Gln Tyr Leu His Pro His Leu Glu Pro Ile
                595 600 605
Leu Lys Asn Thr Tyr Gly Val Ile Ile Tyr Gln Glu Gln Ile Met Gln
        610 615 620
Ile Ala Ser Thr Phe Ala Asn Phe Ser Tyr Gly Glu Ala Asp Ile Leu
625 630 635 640
Arg Arg Ala Met Ser Lys Lys Asn Arg Ala Val Leu Glu Ser Glu Arg
                                645 650 655
Gln His Phe Ile Glu Gly Ala Lys Gln Asn Gly Tyr His Glu Asp Ile
                        660 665 670
Ser Lys Gln Ile Phe Asp Leu Ile Leu Lys Phe Ala Asp Tyr Gly Phe
                675 680 685
Pro Arg Ala His Ala Val Ser Tyr Ser Lys Ile Ala Tyr Ile Met Ser
        690 695 700
Phe Leu Lys Val His Tyr Pro Asn Tyr Phe Tyr Ala Asn Ile Leu Ser
705 710 715 720
Asn Val Ile Gly Ser Glu Lys Lys Thr Ala Gln Met Ile Glu Glu Ala
                                725 730 735
Lys Lys Gln Gly Ile Thr Ile Leu Pro Pro Asn Ile Asn Glu Ser His
                        740 745 750
Trp Phe Tyr Lys Pro Ser Gln Glu Gly Ile Tyr Leu Ser Ile Gly Thr
                755 760 765
Ile Lys Gly Val Gly Tyr Gln Ser Val Lys Val Ile Val Asp Glu Arg
        770 775 780
Tyr Gln Asn Gly Lys Phe Lys Asp Phe Phe Asp Phe Ala Arg Arg Ile
785 790 795 800
Pro Lys Arg Val Lys Thr Arg Lys Leu Leu Glu Ala Leu Ile Leu Val
                                805 810 815
Gly Ala Phe Asp Ala Phe Gly Lys Thr Arg Ser Thr Leu Leu Gln Ala
                        820 825 830
Ile Asp Gln Val Leu Asp Gly Asp Leu Asn Ile Glu Gln Asp Gly Phe
                835 840 845
Leu Phe Asp Ile Leu Thr Pro Lys Gln Met Tyr Glu Asp Lys Glu Glu
        850 855 860
Leu Pro Asp Ala Leu Ile Ser Gln Tyr Glu Lys Glu Tyr Leu Gly Phe
865 870 875 880
Tyr Val Ser Gln His Pro Val Asp Lys Lys Phe Val Ala Lys Gln Tyr
                                885 890 895
Leu Thr Ile Phe Lys Leu Ser Asn Ala Gln Asn Tyr Lys Pro Ile Leu
                        900 905 910
Val Gln Phe Asp Lys Val Lys Gln Ile Arg Thr Lys Asn Gly Gln Asn
                915 920 925
Met Ala Phe Val Thr Leu Asn Asp Gly Ile Glu Thr Leu Asp Gly Val
        930 935 940
Ile Phe Pro Asn Gln Phe Lys Lys Tyr Glu Glu Leu Leu Ser His Asn
945 950 955 960
Asp Leu Phe Ile Val Ser Gly Lys Phe Asp His Arg Lys Gln Gln Arg
                                965 970 975
Gln Leu Ile Ile Asn Glu Ile Gln Thr Leu Ala Thr Phe Glu Glu Gln
                        980 985 990
Lys Leu Ala Phe Ala Lys Gln Ile Ile Ile Arg Asn Lys Ser Gln Ile
                995 1000 1005
Asp Met Phe Glu Glu Met Ile Lys Ala Thr Lys Glu Asn Ala Asn Asp
      1010 1015 1020
Val Val Leu Ser Phe Tyr Asp Glu Thr Ile Lys Gln Met Thr Thr Leu
1025 1030 1035 1040
Gly Tyr Ile Asn Gln Lys Asp Ser Met Phe Asn Asn Phe Ile Gln Ser
                              1045 1050 1055
Phe Asn Pro Ser Asp Ile Arg Leu Ile
                      1060 1065
```

The present invention also relates to the S. aureus dnaX gene. This S. aureus dnaX gene has a partial nucleotide sequence corresponding to SEQ. ID. No. 3 as follows:

| | |
|---|---|
| ttgaattatc aagccttata tcgtatgtac agaccccaaa gtttcgagga tgtcgtcgga | 60 |
| caagaacatg tcacgaagac attgcgcaat gcgatttcga aagaaaaaca gtcgcatgca | 120 |
| tatattttta gtggtccgag aggtacgggg aaaacgagta ttgccaaagt gtttgctaaa | 180 |
| gcaatcaact gtttaaatag cactgatgga gaaccttgta atgaatgtca tatttgtaaa | 240 |
| ggcattacgc aggggactaa ttcagatgtg atagaaattg atgctgctag taataatggc | 300 |
| gttgatgaaa taagaaatat tagagacaaa gttaaatatg caccaagtga atcgaaatat | 360 |
| aaagtttata ttatagatga ggtgcacatg ctaacaacag gtgcttttaa tgccctttta | 420 |
| aagacgttag aagaacctcc agcacacgct atttttatat tggcaacgac agaaccacat | 480 |
| aaaatccctc caacaatcat ttctagggca caacgttttg attttaaagc aattagccta | 540 |
| gatcaaattg ttgaacgttt aaaatttgta gcagatgcac aacaaattga atgtgaagat | 600 |
| gaagccttgg catttatcgc taaagcgtct gaagggggta tgcgtgatgc attaagtatt | 660 |
| atggatcagg ctattgcttt cggcgatggc acattgacat tacaagatgc cctaaatgtt | 720 |
| acgggtagcg ttcatgatga agcgttggat cacttgtttg atgatattgt acaaggtgac | 780 |
| gtacaagcat cttttaaaaa ataccatcag tttataacag aaggtaaaga agtgaatcgc | 840 |
| ctaataaatg atatgattta ttttgtcaga gatacgatta tgaataaaac atctgagaaa | 900 |
| gatactgagt atcgagcact gatgaactta gaattagata tgttatatca aatgattgat | 960 |
| cttattaatg atacattagt gtcgattcgt tttagtgtga atcaaaacgt tcattttgaa | 1020 |
| gtattgttag taaaattagc tgagcagatt aagggtcaac cacaagtgat tgcgaatgta | 1080 |
| gctgaaccag cacaaattgc ttcatcgcca aacacagatg tattgttgca acgtatggaa | 1140 |
| cagttagagc aagaactaaa aacactaaaa gcacaaggag tgagtgttgc tcctactcaa | 1200 |
| aaatcttcga aaaagcctgc gagaggtata caaaaatcta aaaatgcatt ttcaatgcaa | 1260 |
| caaattgcaa aagtgctaga taaagcgaat aaggcagata tcaaattgtt gaaagatcat | 1320 |
| tggcaagaag tgattgacca tgcccaaaac aatgataaaa aatcactcgt tagtttattg | 1380 |
| caaaattcgg aacctgtggc ggcaagtgaa gatcacgtcc ttgtgaaatt tgaggaagag | 1440 |
| atccattgtg aaatcgtcaa taaagacgac gagaaacgta gtagtataga aagtgttgta | 1500 |
| tgtaatatcg ttaataaaaa cgttaaagtt gttggtgtac catcagatca atggcaaaga | 1560 |
| gttcgaacgg agtatttaca aaatcgtaaa aacgaaggcg atgatatgcc aaagcaacaa | 1620 |
| gcacaacaaa cagatattgc tcaaaaagca aaagatcttt tcggtgaaga aactgtacat | 1680 |
| ggtgatagatg aagagtga | 1698 |

The S. aureus dnaX encoded protein (i.e., the tau subunit) has a partial amino acid sequence corresponding to SEQ. ID. No. 4 as follows:

```
Leu Asn Tyr Gln Ala Leu Tyr Arg Met Tyr Arg Pro Gln Ser Phe Glu
1 5 10 15
Asp Val Val Gly Gln Glu His Val Thr Lys Thr Leu Arg Asn Ala Ile
                        20 25 30
Ser Lys Glu Lys Gln Ser His Ala Tyr Ile Phe Ser Gly Pro Arg Gly
                35 40 45
Thr Gly Lys Thr Ser Ile Ala Lys Val Phe Ala Lys Ala Ile Asn Cys
        50 55 60
Leu Asn Ser Thr Asp Gly Glu Pro Cys Asn Glu Cys His Ile Cys Lys
65 70 75 80
Gly Ile Thr Gln Gly Thr Asn Ser Asp Val Ile Glu Ile Asp Ala Ala
                                85 90 95
Ser Asn Asn Gly Val Asp Glu Ile Arg Asn Ile Arg Asp Lys Val Lys
                        100 105 110
Tyr Ala Pro Ser Glu Ser Lys Tyr Lys Val Tyr Ile Ile Asp Glu Val
                115 120 125
His Met Leu Thr Thr Gly Ala Phe Asn Ala Leu Leu Lys Thr Leu Glu
        130 135 140
Glu Pro Pro Ala His Ala Ile Phe Ile Leu Ala Thr Thr Glu Pro His
145 150 155 160
Lys Ile Pro Pro Thr Ile Ile Ser Arg Ala Gln Arg Phe Asp Phe Lys
                                165 170 175
Ala Ile Ser Leu Asp Gln Ile Val Glu Arg Leu Lys Phe Val Ala Asp
                        180 185 190
Ala Gln Gln Ile Glu Cys Glu Asp Glu Ala Leu Ala Phe Ile Ala Lys
                195 200 205
Ala Ser Glu Gly Gly Met Arg Asp Ala Leu Ser Ile Met Asp Gln Ala
        210 215 220
Ile Ala Phe Gly Asp Gly Thr Leu Thr Leu Gln Asp Ala Leu Asn Val
225 230 235 240
Thr Gly Ser Val His Asp Glu Ala Leu Asp His Leu Phe Asp Asp Ile
                                245 250 255
Val Gln Gly Asp Val Gln Ala Ser Phe Lys Lys Tyr His Gln Phe Ile
                        260 265 270
Thr Gln Gly Lys Glu Val Asn Arg Leu Ile Asn Asp Met Ile Tyr Phe
                275 280 285
Val Arg Asp Thr Ile Met Asn Lys Thr Ser Glu Lys Asp Thr Glu Tyr
        290 295 300
Arg Ala Leu Met Asn Leu Glu Leu Asp Met Leu Tyr Gln Met Ile Asp
305 310 315 320
Leu Ile Asn Asp Thr Leu Val Ser Ile Arg Phe Ser Val Asn Gln Asn
                                325 330 335
Val His Phe Glu Val Leu Leu Val Lys Leu Ala Glu Gln Ile Lys Gly
                        340 345 350
Gln Pro Gln Val Ile Ala Asn Val Ala Glu Pro Ala Gln Ile Ala Ser
                355 360 365
Ser Pro Asn Thr Asp Val Leu Leu Gln Arg Met Glu Gln Leu Glu Gln
        370 375 380
Glu Leu Lys Thr Leu Lys Ala Gln Gly Val Ser Val Ala Pro Thr Gln
385 390 395 400
Lys Ser Ser Lys Lys Pro Ala Arg Gly Ile Gln Lys Ser Lys Asn Ala
                                405 410 415
Phe Ser Met Gln Gln Ile Ala Lys Val Leu Asp Lys Ala Asn Lys Ala
                        420 425 430
Asp Ile Lys Leu Leu Lys Asp His Trp Gln Glu Val Ile Asp His Ala
                435 440 445
Gln Asn Asn Asp Lys Lys Ser Leu Val Ser Leu Leu Gln Asn Ser Glu
        450 455 460
Pro Val Ala Ala Ser Glu Asp His Val Leu Val Lys Phe Glu Glu Glu
465 470 475 480
Ile His Cys Glu Ile Val Asn Lys Asp Asp Glu Lys Arg Ser Ser Ile
                                485 490 495
Glu Ser Val Val Cys Asn Ile Val Asn Lys Asn Val Lys Val Val Gly
                        500 505 510
Val Pro Ser Asp Gln Trp Gln Arg Val Arg Thr Glu Tyr Leu Gln Asn
                515 520 525
Arg Lys Asn Glu Gly Asp Asp Met Pro Lys Gln Gln Ala Gln Gln Thr
        530 535 540
Asp Ile Ala Gln Lys Ala Lys Asp Leu Phe Gly Glu Glu Thr Val His
545 550 555 560
Val Ile Asp Glu Glu Glx
                                565
```

The tau subunit of S. aureus functions as does both the tau subunit and the gamma subunit of E. coli.

This invention also relates to the partial nucleotide sequence of the S. aureus dnaB gene. The partial nucleotide sequence of this dnaB gene corresponds to SEQ. ID. No. 5 as follows:

| | |
|---|---|
| atggatagaa tgtatgagca aaatcaaatg ccgcataaca atgaagctga acagtctgtc | 60 |
| ttaggttcaa ttattataga tccagaattg attaatacta ctcaggaagt tttgcttcct | 120 |
| gagtcgtttt ataggggtgc ccatcaacat attttccgtg caatgatgca cttaaatgaa | 180 |
| gataataaag aaattgatgt tgtaacattg atggatcaat tatcgacgga aggtacgttg | 240 |
| aatgaagcgg gtggcccgca atatcttgca gagttatcta caaatgtacc aacgacgcga | 300 |
| aatgttcagt attatactga tatcgtttct aagcatgcat taaaacgtag attgattcaa | 360 |
| actgcagata gtattgccaa tgatggatat aatgatgaac ttgaactaga tgcgatttta | 420 |
| agtgatgcag aacgtcgaat tttagagcta tcatcttctc gtgaaagcga tggctttaaa | 480 |
| gacattcgag acgtcttagg acaagtgtat gaaacagctg aagagcttga tcaaaatagt | 540 |
| ggtcaaacac caggtatacc tacaggatat cgagatttag accaaatgac agcagggttc | 600 |
| aaccgaaatg atttaattat ccttgcagcg cgtccatctg taggtaagac tgcgttcgca | 660 |
| cttaatattg cacaaaaagt tgcaacgcat gaagatatgt atacagttgg tattttctcg | 720 |
| ctagagatgg gtgctgatca gttagccaca cgtatgattt gtagttctgg aaatgttgac | 780 |
| tcaaaccgct taagaacggg tactatgact gaggaagatt ggagtcgttt tactatagcg | 840 |
| gtaggtaaat tatcacgtac gaagattttt attgatgata caccgggtat tcgaattaat | 900 |
| gatttacgtt ctaaatgtcg tcgattaaag caagaacatg gcttagacat gattgtgatt | 960 |
| gactacttac agttgattca aggtagtggt tcacgtgcgt ccgataacag acaacaggaa | 1020 |
| gtttctgaaa tctctcgtac attaaaagca ttagcccgtg aattaaaatg tccagttatc | 1080 |
| gcattaagtc agttatctcg tggtgttgaa caacgacaag ataaacgtcc aatgatgagt | 1140 |
| gatattcgtg aatctggttc gattgagcaa gatgccgata tcgttgcatt cttataccgt | 1200 |
| gatgattact ataaccgtgg cggcgatgaa gatgatgacg atgatggtgg tttcgagcca | 1260 |
| caaacgaatg atgaaaacgg tgaaattgaa attatcattg ctaagcaacg taacggtcca | 1320 |
| acaggcacag ttaagttaca ttttatgaaa caatataata aatttaccga tatcgattat | 1380 |
| gcacatgcag atatgatg | 1398 |

The amino acid sequence of S. aureus DnaB encoded by the dnaB gene corresponds to SEQ. ID. No. 6 as follows:

```
Met Asp Arg Met Tyr Glu Gln Asn Gln Met Pro His Asn Asn Glu Ala
    1 5 10 15
Glu Gln Ser Val Leu Gly Ser Ile Ile Ile Asp Pro Glu Leu Ile Asn
                          20 25 30
Thr Thr Gln Glu Val Leu Leu Pro Glu Ser Phe Tyr Arg Gly Ala His
                  35 40 45
Gln His Ile Phe Arg Ala Met Met His Leu Asn Glu Asp Asn Lys Glu
          50 55 60
Ile Asp Val Val Thr Leu Met Asp Gln Leu Ser Thr Glu Gly Thr Leu
  65 70 75 80
Asn Glu Ala Gly Gly Pro Gln Tyr Leu Ala Glu Leu Ser Thr Asn Val
                                  85 90 95
Pro Thr Thr Arg Asn Val Gln Tyr Tyr Thr Asp Ile Val Ser Lys His
                        100 105 110
Ala Leu Lys Arg Arg Leu Ile Gln Thr Ala Asp Ser Ile Ala Asn Asp
                115 120 125
Gly Tyr Asn Asp Glu Leu Glu Leu Asp Ala Ile Leu Ser Asp Ala Glu
        130 135 140
Arg Arg Ile Leu Glu Leu Ser Ser Ser Arg Glu Ser Asp Gly Phe Lys
145 150 155 160
Asp Ile Arg Asp Val Leu Gly Gln Val Tyr Glu Thr Ala Glu Glu Leu
                                165 170 175
Asp Gln Asn Ser Gly Gln Thr Pro Gly Ile Pro Thr Gly Tyr Arg Asp
                        180 185 190
Leu Asp Gln Met Thr Ala Gly Phe Asn Arg Asn Asp Leu Ile Ile Leu
                195 200 205
Ala Ala Arg Pro Ser Val Gly Lys Thr Ala Phe Ala Leu Asn Ile Ala
        210 215 220
Gln Lys Val Ala Thr His Glu Asp Met Tyr Thr Val Gly Ile Phe Ser
225 230 235 240
Leu Glu Met Gly Ala Asp Gln Leu Ala Thr Arg Met Ile Cys Ser Ser
                                245 250 255
Gly Asn Val Asp Ser Asn Arg Leu Arg Thr Gly Thr Met Thr Glu Glu
                        260 265 270
Asp Trp Ser Arg Phe Thr Ile Ala Val Gly Lys Leu Ser Arg Thr Lys
                275 280 285
Ile Phe Ile Asp Asp Thr Pro Gly Ile Arg Ile Asn Asp Leu Arg Ser
        290 295 300
Lys Cys Arg Arg Leu Lys Gln Glu His Gly Leu Asp Met Ile Val Ile
305 310 315 320
Asp Tyr Leu Gln Leu Ile Gln Gly Ser Gly Ser Arg Ala Ser Asp Asn
                                325 330 335
Arg Gln Gln Glu Val Ser Glu Ile Ser Arg Thr Leu Lys Ala Leu Ala
                        340 345 350
Arg Glu Leu Lys Cys Pro Val Ile Ala Leu Ser Gln Leu Ser Arg Gly
                355 360 365
Val Glu Gln Arg Gln Asp Lys Arg Pro Met Met Ser Asp Ile Arg Glu
        370 375 380
Ser Gly Ser Ile Glu Gln Asp Ala Asp Ile Val Ala Phe Leu Tyr Arg
385 390 395 400
Asp Asp Tyr Tyr Asn Arg Gly Gly Asp Glu Asp Asp Asp Asp Asp Gly
                                405 410 415
Gly Phe Glu Pro Gln Thr Asn Asp Glu Asn Gly Glu Ile Glu Ile Ile
                        420 425 430
Ile Ala Lys Gln Arg Asn Gly Pro Thr Gly Thr Val Lys Leu His Phe
                435 440 445
Met Lys Gln Tyr Asn Lys Phe Thr Asp Ile Asp Tyr Ala His Ala Asp
        450 455 460
Met Met
465
```

The present invention also relates to the S. aureus polC gene (encoding Pol III-L or α-large). The partial nucleotide sequence of this polC gene corresponds to SEQ. ID. No. 7 as follows:

| | |
|---|---|
| atgacagagc aacaaaaatt taaagtgctt gctgatcaaa ttaaaatttc aaatcaatta | 60 |
| gatgctgaaa ttttaaattc aggtgaactg acacgtatag atgtttctaa caaaaacaga | 120 |
| acatgggaat ttcatattac attaccacaa ttcttagctc atgaagatta tttattattt | 180 |
| ataaatgcaa tagagcaaga gtttaaagat atcgccaacg ttacatgtcg ttttacggta | 240 |
| acaaatggca cgaatcaaga tgaacatgca attaaatact ttgggcactg tattgaccaa | 300 |
| acagctttat ctccaaaagt taaaggtcaa ttgaaacaga aaaagcttat tatgtctgga | 360 |
| aaagtattaa aagtaatggt atcaaatgac attgaacgta atcattttga taaggcatgt | 420 |
| aatggaagtc ttatcaaagc gtttagaaat tgtggttttg atatcgataa aatcatattc | 480 |
| gaaacaaatg ataatgatca agaacaaaac ttagcttctt tagaagcaca tattcaagaa | 540 |
| gaagacgaac aaagtgcacg attggcaaca gagaaacttg aaaaaatgaa agctgaaaaa | 600 |
| gcgaaacaac aagataacaa cgaaagtgct gtcgataagt gtcaaattgg taagccgatt | 660 |
| caaattgaaa atattaaacc aattgaatct attattgagg aagagtttaa agttgcaata | 720 |
| gagggtgtca tttttgatat aaacttaaaa gaacttaaaa gtggtcgcca tatcgtagaa | 780 |
| attaaagtga ctgactatac ggactcttta gttttaaaaa tgtttactcg taaaaacaaa | 840 |
| gatgatttag aacattttaa agcgctaagt gttggtaaat gggttagggc tcaaggtcgt | 900 |
| attgaagaag atacatttat tagagattta gttatgatga tgtctgatat tgaagagatt | 960 |
| aaaaaagcga caaaaaaaga taaggctgaa gaaaagcgtg tagaattcca cttgcatact | 1020 |
| gcaatgagcc aaatggatgg tatacccaat attggtgcgt atgttaaaca ggcagcagac | 1080 |
| tggggacatc cagccattgc ggttacagac cataatgttg tgcaagcatt tccagatgct | 1140 |
| cacgcagcag cggaaaaaca tggcattaaa atgatatacg gtatggaagg tatgttagtt | 1200 |
| gatgatggtg ttccgattgc atacaaacca caagatgtcg tattaaaaga tgctacttat | 1260 |
| gttgtgttcg acgttgagac aactggttta tcaaatcagt atgataaaat catcgagctt | 1320 |
| gcagctgtga aagttcataa cggtgaaatc atcgataagt ttgaaaggtt tagtaatccg | 1380 |
| catgaacgat tatcggaaac gattatcaat ttgacgcata ttactgatga tatgttagta | 1440 |
| gatgcccctg agattgaaga agtacttaca gagtttaaag aatgggttgg cgatgcgata | 1500 |
| ttcgtagcgc ataatgcttc gtttgatatg ggcttcatcg atacgggata tgaacgtctt | 1560 |
| gggtttggac catcaacgaa tggtgttatc gatactttag aattatctcg tacgattaat | 1620 |
| actgaatatg gtaaacatgg tttgaatttc ttggctaaaa aatatggcgt agaattaacg | 1680 |
| caacatcacc gtgccattta tgatacagaa gcaacagctt acattttcat aaaaatggtt | 1740 |
| caacaaatga aagaattagg cgtattaaat cataacgaaa tcaacaaaaa actcagtaat | 1800 |
| gaagatgcat ataaacgtgc aagacctagt catgtcacat taattgtaca aaaccaacaa | 1860 |
| ggtcttaaaa atctatttaa aattgtaagt gcatcattgg tgaagtattt ctaccgtaca | 1920 |
| cctcgaattc cacgttcatt gttagatgaa tatcgtgagg gattattggt aggtacagcg | 1980 |
| tgtgatgaag gtgaattatt tacggcagtt atgcagaagg accagagtca agttgaaaaa | 2040 |
| attgccaaat attatgattt tattgaaatt caaccaccgg cactttatca agatttaatt | 2100 |
| gatagagage ttattagaga tactgaaaca ttacatgaaa tttatcaacg tttaatacat | 2160 |
| gcaggtgaca cagcgggtat acctgttatt gcgacaggaa atgcacacta tttgtttgaa | 2220 |
| catgatggta tcgcacgtaa aattttaata gcatcacaac ccggcaatcc acttaatcgc | 2280 |
| tcaactttac cggaagcaca ttttagaact acagatgaaa tgttaaacga gtttcatttt | 2340 |
| ttaggtgaag aaaaagcgca tgaaattgtt gtgaaaaata caaacgaatt agcagatcga | 2400 |
| attgaacgtg ttgttcctat taaagatgaa ttatacacac cgcgtatgga aggtgctaac | 2460 |
| gaagaaatta gagaactaag ttatgcaaat gcgcgtaaac tgtatggtga agacctgcct | 2520 |
| caaatcgtaa ttgatcgatt agaaaaagaa ttaaaaagta ttatcggtaa tggatttgcg | 2580 |
| gtaatttact taatttcgca acgtttagtt aaaaaatcat tagatgatgg atacttagtt | 2640 |
| ggttcccgtg gttcagtagg ttctagtttt gtagcgacaa tgactgagat tactgaagta | 2700 |
| aacccgttac cgccacacta tatttgtccg aactgtaaaa cgagtgaatt tttcaatgat | 2760 |
| ggttcagtag gatcaggatt tgatttacct gataagacgt gtgaaacttg tggagcgcca | 2820 |
| cttattaaag aaggacaaga tattccgttt gaaacatttt taggatttaa gggagataaa | 2880 |
| gttcctgata tcgacttaaa ctttagtggt gaatatcaac cgaatgccca taactacaca | 2940 |
| aaagtattat ttggtgagga taaagtattc cgtgcaggta caattggtac tgttgctgaa | 3000 |
| aagactgctt ttggttatgt taaaggttat ttgaatgatc aaggtatcca caaaagaggt | 3060 |
| gctgaaatag atcgactcgt taaaggatgt acaggtgtta aacgtacaac tggacagcat | 3120 |
| ccagggggta ttattgtagt acctgattac atggatattt atgattttac gccgatacaa | 3180 |
| tatcctgccg atgatcaaaa ttcagcatgg atgacgacac attttgattt ccattctatt | 3240 |
| catgataatg tattaaaact tgatatactt ggacacgatg atccaacaat gattcgtatg | 3300 |
| cttcaagatt tatcaggaat tgatccaaaa acaatacctg tagatgataa agaagttatg | 3360 |
| cagatattta gtacacctga aagtttgggt gttactgaag atgaaatttt atgtaaaaca | 3420 |
| ggtacatttg gggtaccaga attcggtaca ggattcgtgc gtcaaatgtt agaagataca | 3480 |
| aagccaacaa cattttctga attagttcaa atctcaggat tatctcatgg tacagatgtg | 3540 |
| tggttaggca atgctcaaga attaattaaa accggtatat gtgatttatc aagtgtaatt | 3600 |
| ggttgtcgtg atgatatcat ggtttattta atgtatgctg gtttagaacc atcaatggct | 3660 |
| tttaaaataa tggagtcagt acgtaaaggt aaaggtttaa ctgaagaaat gattgaaacg | 3720 |
| atgaaagaaa atgaagtgcc agattggtat ttagattcat gtcttaaaat taagtacatg | 3780 |
| ttccctaaag cccatgcagc agcatacgtt ttaatggcag tacgtatcgc atatttcaaa | 3840 |
| gtacatcatc cactttatta ctatgcatct tactttacaa ttcgtgcgtc agactttgat | 3900 |
| ttaatcacga tgattaaaga taaaacaagc attcgaaata ctgtaaaaga catgtattct | 3960 |
| cgctatatgg atctaggtaa aaaagaaaaa gacgtattaa cagtcttgga aattatgaat | 4020 |
| gaaatggcgc atcgaggtta tcgaatgcaa ccgattagtt tagaaaagag tcaggcgttc | 4080 |
| gaatttatca ttgaaggcga tacacttatt ccgccgttca tatcagtgcc tgggcttggc | 4140 |
| gaaaacgttg cgaaacgaat tgttgaagct cgtgacgatg gcccattttt atcaaaagaa | 4200 |
| gatttaaaca aaaaagctgg attatctcag aaaattattg agtatttaga tgagttaggc | 4260 |
| tcattaccga atttaccaga taaagctcaa ctttcgatat ttgatatg | 4308 |

The amino acid sequence of the S. aureus polC gene product, α-large, corresponds to SEQ. ID. No. 8 as follows:

```
Met Thr Glu Gln Gln Lys Phe Lys Val Leu Ala Asp Gln Ile Lys Ile
    1 5 10 15
Ser Asn Gln Leu Asp Ala Glu Ile Leu Asn Ser Gly Glu Leu Thr Arg
                          20 25 30
Ile Asp Val Ser Asn Lys Asn Arg Thr Trp Glu Phe His Ile Thr Leu
                  35 40 45
Pro Gln Phe Leu Ala His Glu Asp Tyr Leu Leu Phe Ile Asn Ala Ile
          50 55 60
Glu Gln Glu Phe Lys Asp Ile Ala Asn Val Thr Cys Arg Phe Thr Val
  65 70 75 80
Thr Asn Gly Thr Asn Gln Asp Glu His Ala Ile Lys Tyr Phe Gly His
                                  85 90 95
Cys Ile Asp Gln Thr Ala Leu Ser Pro Lys Val Lys Gly Gln Leu Lys
                        100 105 110
Gln Lys Lys Leu Ile Met Ser Gly Lys Val Leu Lys Val Met Val Ser
                115 120 125
Asn Asp Ile Glu Arg Asn His Phe Asp Lys Ala Cys Asn Gly Ser Leu
        130 135 140
Ile Lys Ala Phe Arg Asn Cys Gly Phe Asp Ile Asp Lys Ile Ile Phe
145 150 155 160
Glu Thr Asn Asp Asn Asp Gln Glu Gln Asn Leu Ala Ser Leu Glu Ala
                                165 170 175
His Ile Gln Glu Glu Asp Glu Gln Ser Ala Arg Leu Ala Thr Glu Lys
                        180 185 190
Leu Glu Lys Met Lys Ala Glu Lys Ala Lys Gln Gln Asp Asn Lys Gln
                195 200 205
Ser Ala Val Asp Lys Cys Gln Ile Gly Lys Pro Ile Gln Ile Glu Asn
        210 215 220
Ile Lys Pro Ile Glu Ser Ile Ile Glu Glu Glu Phe Lye Val Ala Ile
225 230 235 240
Glu Gly Val Ile Phe Asp Ile Asn Leu Lys Glu Leu Lys Ser Gly Arg
                                245 250 255
His Ile Val Glu Ile Lys Val Thr Asp Tyr Thr Asp Ser Leu Val Leu
                        260 265 270
Lys Met Phe Thr Arg Lys Asn Lys Asp Asp Leu Glu His Phe Lys Ala
                275 280 285
Leu Ser Val Gly Lys Trp Val Arg Ala Gln Gly Arg Ile Glu Glu Asp
        290 295 300
Thr Phe Ile Arg Asp Leu Val Met Met Met Ser Asp Ile Glu Glu Ile
305 310 315 320
Lys Lys Ala Thr Lys Lys Asp Lys Ala Glu Glu Lys Arg Val Glu Phe
                                325 330 335
His Leu His Thr Ala Met Ser Gln Met Asp Gly Ile Pro Asn Ile Gly
                        340 345 350
Ala Tyr Val Lys Gln Ala Ala Asp Trp Gly His Pro Ala Ile Ala Val
                355 360 365
Thr Asp His Asn Val Val Gln Ala Phe Pro Asp Ala His Ala Ala Ala
        370 375 380
Glu Lys His Gly Ile Lys Met Ile Tyr Gly Met Glu Gly Met Leu Val
385 390 395 400
Asp Asp Gly Val Pro Ile Ala Tyr Lys Pro Gln Asp Val Val Leu Lys
                                405 410 415
Asp Ala Thr Tyr Val Val Phe Asp Val Glu Thr Thr Gly Leu Ser Asn
                        420 425 430
Gln Tyr Asp Lys Ile Ile Glu Leu Ala Ala Val Lys Val His Asn Gly
                435 440 445
Glu Ile Ile Asp Lys Phe Glu Arg Phe Ser Asn Pro His Glu Arg Leu
        450 455 460
Ser Glu Thr Ile Ile Asn Leu Thr His Ile Thr Asp Asp Met Leu Val
465 470 475 480
Asp Ala Pro Glu Ile Glu Glu Val Leu Thr Glu Phe Lys Glu Trp Val
                                485 490 495
Gly Asp Ala Ile Phe Val Ala His Asn Ala Ser Phe Asp Met Gly Phe
                        500 505 510
Ile Asp Thr Gly Tyr Glu Arg Leu Gly Phe Gly Pro Ser Thr Asn Gly
                515 520 525
Val Ile Asp Thr Leu Glu Leu Ser Arg Thr Ile Asn Thr Glu Tyr Gly
        530 535 540
Lys His Gly Leu Asn Phe Leu Ala Lys Lys Tyr Gly Val Glu Leu Thr
545 550 555 560
Gln His His Arg Ala Ile Tyr Asp Thr Glu Ala Thr Ala Tyr Ile Phe
                                565 570 575
Ile Lys Met Val Gln Gln Met Lys Glu Leu Gly Val Leu Asn His Asn
                        580 585 590
Glu Ile Asn Lys Lys Leu Ser Asn Glu Asp Ala Tyr Lys Arg Ala Arg
                595 600 605
Pro Ser His Val Thr Leu Ile Val Gln Asn Gln Gln Gly Leu Lys Asn
        610 615 620
Leu Phe Lys Ile Val Ser Ala Ser Leu Val Lys Tyr Phe Tyr Arg Thr
625 630 635 640
Pro Arg Ile Pro Arg Ser Leu Leu Asp Glu Tyr Arg Glu Gly Leu Leu
                                645 650 655
Val Gly Thr Ala Cys Asp Glu Gly Glu Leu Phe Thr Ala Val Met Gln
                        660 665 670
Lys Asp Gln Ser Gln Val Glu Lys Ile Ala Lys Tyr Tyr Asp Phe Ile
                675 680 685
Glu Ile Gln Pro Pro Ala Leu Tyr Gln Asp Leu Ile Asp Arg Glu Leu
        690 695 700
Ile Arg Asp Thr Glu Thr Leu His Glu Ile Tyr Gln Arg Leu Ile His
705 710 715 720
Ala Gly Asp Thr Ala Gly Ile Pro Val Ile Ala Thr Gly Asn Ala His
                                725 730 735
Tyr Leu Phe Glu His Asp Gly Ile Ala Arg Lys Ile Leu Ile Ala Ser
                        740 745 750
Gln Pro Gly Asn Pro Leu Asn Arg Ser Thr Leu Pro Glu Ala His Phe
                755 760 765
Arg Thr Thr Asp Glu Met Leu Asn Glu Phe His Phe Leu Gly Glu Glu
        770 775 780
Lys Ala His Glu Ile Val Val Lys Asn Thr Asn Glu Leu Ala Asp Arg
785 790 795 800
Ile Glu Arg Val Val Pro Ile Lys Asp Glu Leu Tyr Thr Pro Arg Met
                                805 810 815
Glu Gly Ala Asn Glu Glu Ile Arg Glu Leu Ser Tyr Ala Asn Ala Arg
                        820 825 830
Lys Leu Tyr Gly Glu Asp Leu Pro Gln Ile Val Ile Asp Arg Leu Glu
                835 840 845
Lys Glu Leu Lys Ser Ile Ile Gly Asn Gly Phe Ala Val Ile Tyr Leu
        850 855 860
Ile Ser Gln Arg Leu Val Lys Lys Ser Leu Asp Asp Gly Tyr Leu Val
865 870 875 880
Gly Ser Arg Gly Ser Val Gly Ser Ser Phe Val Ala Thr Met Thr Glu
                                885 890 895
Ile Thr Glu Val Asn Pro Leu Pro Pro His Tyr Ile Cys Pro Asn Cys
                        900 905 910
Lys Thr Ser Glu Phe Phe Asn Asp Gly Ser Val Gly Ser Gly Phe Asp
                915 920 925
Leu Pro Asp Lys Thr Cys Glu Thr Cys Gly Ala Pro Leu Ile Lys Glu
        930 935 940
Gly Gln Asp Ile Pro Phe Glu Lys Phe Leu Gly Phe Lys Gly Asp Lys
945 950 955 960
Val Pro Asp Ile Asp Leu Asn Phe Ser Gly Glu Tyr Gln Pro Asn Ala
                                965 970 975
His Asn Tyr Thr Lys Val Leu Phe Gly Glu Asp Lys Val Phe Arg Ala
                        980 985 990
Gly Thr Ile Gly Thr Val Ala Glu Lys Thr Ala Phe Gly Tyr Val Lys
                995 1000 1005
Gly Tyr Leu Asn Asp Gln Gly Ile His Lys Arg Gly Ala Glu Ile Asp
      1010 1015 1020
Arg Leu Val Lys Gly Cys Thr Gly Val Lys Ala Thr Thr Gly Gln His
1025 1030 1035 1040
Pro Gly Gly Ile Ile Val Val Pro Asp Tyr Met Asp Ile Tyr Asp Phe
                              1045 1050 1055
Thr Pro Ile Gln Tyr Pro Ala Asp Asp Gln Asn Ser Ala Trp Met Thr
                      1060 1065 1070
Thr His Phe Asp Phe His Ser Ile His Asp Asn Val Leu Lys Leu Asp
              1075 1080 1085
Ile Leu Gly His Asp Asp Pro Thr Met Ile Arg Met Leu Gln Asp Leu
      1090 1095 1100
Ser Gly Ile Asp Pro Lys Thr Ile Pro Val Asp Asp Lys Glu Val Met
1105 1110 1115 1120
Gln Ile Phe Ser Thr Pro Glu Ser Leu Gly Val Thr Glu Asp Glu Ile
                              1125 1130 1135
Leu Cys Lys Thr Gly Thr Phe Gly Val Pro Asn Ser Asp Arg Ile Arg
                      1140 1145 1150
Arg Gln Met Leu Glu Asp Thr Lys Pro Thr Thr Phe Ser Glu Leu Val
              1155 1160 1165
Gln Ile Ser Gly Leu Ser His Gly Thr Asp Val Trp Leu Gly Asn Ala
      1170 1175 1180
Gln Glu Leu Ile Lys Thr Gly Ile Cys Asp Leu Ser Ser Val Ile Gly
1185 1190 1195 1200
Cys Arg Asp Asp Ile Met Val Tyr Leu Met Tyr Ala Gly Leu Gln Pro
                              1205 1210 1215
Ser Met Ala Phe Lys Ile Met Glu Ser Val Arg Lys Gly Lys Gly Leu
                      1220 1225 1230
Thr Glu Glu Met Ile Glu Thr Met Lys Glu Asn Glu Val Pro Asp Trp
              1235 1240 1245
Tyr Leu Asp Ser Cys Leu Lys Ile Lys Tyr Ile Phe Pro Lys Ala His
      1250 1255 1260
Ala Ala Ala Tyr Val Leu Met Ala Val Arg Ile Ala Tyr Phe Lys Val
1265 1270 1275 1280
His His Pro Leu Tyr Tyr Tyr Ala Ser Tyr Phe Thr Ile Arg Ala Ser
                              1285 1290 1295
Asp Phe Asp Leu Ile Thr Met Ile Lys Asp Lys Thr Ser Ile Arg Asn
                      1300 1305 1310
Thr Val Lys Asp Met Tyr Ser Arg Tyr Met Asp Leu Gly Lys Lys Glu
              1315 1320 1325
Lys Asp Val Leu Thr Val Leu Glu Ile Met Asn Glu Met Ala His Arg
      1330 1335 1340
Gly Tyr Arg Met Gln Pro Ile Ser Leu Glu Lys Ser Gln Ala Phe Glu
1345 1350 1355 1360
Phe Ile Ile Glu Gly Asp Thr Leu Ile Pro Pro Phe Ile Ser Val Pro
                              1365 1370 1375
Gly Leu Gly Glu Asn Val Ala Lys Arg Ile Val Glu Ala Arg Asp Asp
                      1380 1385 1390
Gly Pro Phe Leu Ser Lys Glu Asp Leu Asn Lys Lys Ala Gly Leu Tyr
              1395 1400 1405
Gln Lys Ile Ile Glu Tyr Leu Asp Glu Leu Gly Ser Leu Pro Asn Leu
      1410 1415 1420
Pro Asp Lys Ala Gln Leu Ser Ile Phe Asp Met
1425 1430 1435
```

This invention also relates to the S. aureus dnaN gene encoding the beta subunit. The partial nucleotide sequence of this dnaN gene corresponds to SEQ. ID. No. 9 as follows:

| | |
|---|---|
| atgatggaat tcactattaa aagagattat tttattacac aattaaatga cacattaaaa | 60 |
| gctatttcac caagaacaac attacctata ttaactggta tcaaaatcga tgcgaaagaa | 120 |
| catgaagtta tattaactgg ttcagactct gaaatttcaa tagaaatcac tattcctaaa | 180 |
| actgtagatg gcgaagatat tgtcaatatt tcagaaacag gctcagtagt acttcctgga | 240 |
| cgattctttg ttgatattat aaaaaaatta cctggtaaag atgttaaatt atctacaaat | 300 |
| gaacaattcc agacattaat tacatcaggt cattctgaat ttaatttgag tggcttagat | 360 |
| ccagatcaat atcctttatt acctcaagtt tctagagatg acgcaattca attgtcggta | 420 |
| aaagtactta aaaacgtgat tgcacaaacg aattttgcag tgtccacctc agaaacacgc | 480 |
| ccagtactaa ctggtgtgaa ctggcttata caagaaaatg aattaatatg cacagcgact | 540 |
| gattcacacc gcttggctgt aagaaagttg cagttagaag atgtttctga aaacaaaaat | 600 |
| gtcatcattc caggtaaggc tttagctgaa ttaaataaaa ttatgtctga caatgaagaa | 660 |
| gacattgata tcttctttgc ttcaaaccaa gttttattta aagttggaaa tgtgaacttt | 720 |
| atttctcgat tattagaagg acattatcct gatacaacac gtttattccc tgaaaactat | 780 |
| gaaattaaat taagtataga caatggggag ttttatcatg cgattgatcg tgcctcttta | 840 |
| ttagcacgtg aaggtggtaa taacgttatt aaattaagta caggtgatga cgttgttgaa | 900 |
| ttatcttcta catcaccaga aattggtact gtaaaagaag aagttgatgc aaacgatgtt | 960 |
| gaaggtggta gcctgaaaat ttcattcaac tctaaatata tgatggatgc tttaaaagca | 1020 |
| atcgataatg atgaggttga agttgaattc ttcggtacaa tgaaaccatt tattctaaaa | 1080 |
| ccaaaaggtg acgactcggt aacgcaatta attttaccaa tcagaactta ctaa | 1134 |

This amino acid sequence of S. aureus beta subunit is as follows (SEQ. ID. No. 10):

```
Met Met Glu Phe Thr Ile Lys Arg Asp Tyr Phe Ile Thr Gln Leu Asn
1 5 10 15
Asp Thr Leu Lys Ala Ile Ser Pro Arg Thr Thr Leu Pro Ile Leu Thr
                        20 25 30
Gly Ile Lys Ile Asp Ala Lys Glu His Glu Val Ile Leu Thr Gly Ser
                35 40 45
Asp Ser Glu Ile Ser Ile Glu Ile Thr Ile Pro Lys Thr Val Asp Gly
        50 55 60
Glu Asp Ile Val Asn Ile Ser Glu Thr Gly Ser Val Val Leu Pro Gly
65 70 75 80
Arg Phe Phe Val Asp Ile Ile Lys Lys Leu Pro Gly Lys Asp Val Lys
                                85 90 95
Leu Ser Thr Asn Glu Gln Phe Gln Thr Leu Ile Thr Ser Gly His Ser
                        100 105 110
Glu Phe Asn Leu Ser Gly Leu Asp Pro Asp Gln Tyr Pro Leu Leu Pro
                115 120 125
Gln Val Ser Arg Asp Asp Ala Ile Gln Leu Ser Val Lys Val Leu Lys
        130 135 140
Asn Val Ile Ala Gln Thr Asn Phe Ala Val Ser Thr Ser Glu Thr Arg
145 150 155 160
Pro Val Leu Thr Gly Val Asn Trp Leu Ile Gln Glu Asn Glu Leu Ile
                                165 170 175
Cys Thr Ala Thr Asp Ser His Arg Leu Ala Val Arg Lys Leu Gln Leu
                        180 185 190
Glu Asp Val Ser Glu Asn Lys Asn Val Ile Ile Pro Gly Lys Ala Leu
                195 200 205
Ala Glu Leu Asn Lys Ile Met Ser Asp Asn Glu Glu Asp Ile Asp Ile
        210 215 220
Phe Phe Ala Ser Asn Gln Val Leu Phe Lys Val Gly Asn Val Asn Phe
225 230 235 240
Ile Ser Arg Leu Leu Glu Gly His Tyr Pro Asp Thr Thr Arg Leu Phe
                                245 250 255
Pro Glu Asn Tyr Glu Ile Lys Leu Ser Ile Asp Asn Gly Glu Phe Tyr
                        260 265 270
His Ala Ile Asp Arg Ala Ser Leu Leu Ala Arg Glu Gly Gly Asn Asn
                275 280 285
Val Ile Lys Leu Ser Thr Gly Asp Asp Val Val Glu Leu Ser Ser Thr
        290 295 300
Ser Pro Glu Ile Gly Thr Val Lys Glu Glu Val Asp Ala Asn Asp Val
305 310 315 320
Glu Gly Gly Ser Leu Lys Ile Ser Phe Asn Ser Lys Tyr Met Met Asp
                                325 330 335
Ala Leu Lys Ala Ile Asp Asn Asp Glu Val Glu Val Glu Phe Phe Gly
                        340 345 350
Thr Met Lys Pro Phe Ile Leu Lys Pro Lys Gly Asp Asp Ser Val Thr
                355 360 365
Gln Leu Ile Leu Pro Ile Arg Thr Tyr
        370 375
```

This invention also relates to the S. aureus holA gene encoding the delta subunit. The partial nucleotide sequence of this holA gene corresponds to SEQ. ID. No. 1 as follows:

| | |
|---|---|
| atggatgaac agcaacaatt gacgaatgca tatcattcaa ataaattatc gcatgcctat | 60 |
| ttatttgaag gtgatgatgc acaaacgatg aaacaagttg cgattaattt tgcaaagctt | 120 |
| attttatgtc aaacagatag tcaatgtgaa acaaaggtta gtacatataa tcatccagac | 180 |
| tttatgtata tatcaacaac tgagaatgca attaagaaag aacaagttga acaacttgtg | 240 |
| cgtcatatga atcaacttcc tatagaaagc acaaataaag tgtacatcat cgaagacttt | 300 |
| gaagactttg aaaagttaac tgttcaaggg gaaaacagta tcttgaaatt tcttgaagaa | 360 |
| ccaccggaca atacgattgc tattttattg tctacaaaac ctgagcaaat tttagacaca | 420 |
| atccattcaa ggtgtcagca tgtatatttc aagcctattg ataaagaaaa gtttataaat | 480 |
| agattagttg aacaaaacat gtctaagcca gtagctgaaa tgattagtac ttatactacg | 540 |
| caaatagata atgcaatggc tttaaatgaa gaatttgatt tattagcatt aaggaaatca | 600 |
| gttatacgtt gggaattgtt gcttactaat aagccaatgg cacttatagg tattattgat | 660 |
| ttattgaaac aggctaaaaa taaaaaactg caatctttaa ctattgcagc tgtgaatggt | 720 |
| ttcttcgaag atatcataca tacaaaggta aatgtagagg ataaacaaat atatagtgat | 780 |
| ttaaaaaatg atattgatca atatgcgcaa aagttgtcgt ttaatcaatt aattttgatg | 840 |
| tttgatcaac tgacggaagc acataagaaa ttgaatcaaa atgtaaatcc aacgcttgta | 900 |
| tttgaacaaa tcgtaattaa gggtgtgagt | 930 |

The amino acid sequence of the delta subunit encoded by S. aureus holA corresponds to SEQ. ID. No. 12 as follows:

```
Met Asp Glu Gln Gln Gln Leu Thr Asn Ala Tyr His Ser Asn Lys Leu
    1 5 10 15
Ser His Ala Tyr Leu Phe Glu Gly Asp Asp Ala Gln Thr Met Lys Gln
                          20 25 30
Val Ala Ile Asn Phe Ala Lys Leu Ile Leu Cys Gln Thr Asp Ser Gln
                  35 40 45
Cys Glu Thr Lys Val Ser Thr Tyr Asn His Pro Asp Phe Met Tyr Ile
          50 55 60
Ser Thr Thr Glu Asn Ala Ile Lys Lys Glu Gln Val Glu Gln Leu Val
  65 70 75 80
Arg His Met Asn Gln Leu Pro Ile Glu Ser Thr Asn Lys Val Tyr Ile
                                  85 90 95
Ile Glu Asp Phe Glu Asp Phe Glu Lys Leu Thr Val Gln Gly Glu Asn
                        100 105 110
Ser Ile Leu Lys Phe Leu Glu Glu Pro Pro Asp Asn Thr Ile Ala Ile
                115 120 125
Leu Leu Ser Thr Lys Pro Glu Gln Ile Leu Asp Thr Ile His Ser Arg
        130 135 140
Cys Gln His Val Tyr Phe Lys Pro Ile Asp Lys Glu Lys Phe Ile Asn
145 150 155 160
Arg Leu Val Glu Gln Asn Met Ser Lys Pro Val Ala Glu Met Ile Ser
                                165 170 175
Thr Tyr Thr Thr Gln Ile Asp Asn Ala Met Ala Leu Asn Glu Glu Phe
                        180 185 190
Asp Leu Leu Ala Leu Arg Lys Ser Val Ile Arg Trp Glu Leu Leu Leu
                195 200 205
Thr Asn Lys Pro Met Ala Leu Ile Gly Ile Ile Asp Leu Leu Lys Gln
        210 215 220
Ala Lys Asn Lys Lys Leu Gln Ser Leu Thr Ile Ala Ala Val Asn Gly
225 230 235 240
Phe Phe Glu Asp Ile Ile His Thr Lys Val Asn Val Glu Asp Lys Gln
                                245 250 255
Ile Tyr Ser Asp Leu Lys Asn Asp Ile Asp Gln Tyr Ala Gln Lys Leu
                        260 265 270
Ser Phe Asn Gln Leu Ile Leu Met Phe Asp Gln Leu Thr Glu Ala His
                275 280 285
Lys Lys Leu Asn Gln Asn Val Asn Pro Thr Leu Val Phe Glu Gln Ile
        290 295 300
Val Ile Lys Gly Val Ser
305 310
```

This invention also relates to the S. aureus holB gene encoding the delta prime subunit. The partial nucleotide sequence of this holB gene corresponds to SEQ. ID. No. 13 as follows:

| | |
|---|---|
| atgagcgaca atattgtagc tatttatgga gatgtgcctg aattggttga aaaacaaagt | 60 |
| gcagaaatca tatcacaatt tttgaaaagt gatagagatg actttaactt tgtgaaatat | 120 |
| aatttatacg aaacagagat tgcaccaatt gttgaagaaa cattaacatt gcctttcttt | 180 |
| tcagataaaa aagcaatttt ggttaaaaat gcatatatat ttacaggtga aaaagcgcca | 240 |
| aaagatatgg ctcataatgt agaccaatta atagaattta ttgaaaaata tgatggcgaa | 300 |
| aatttgattg tctttgagat atatcaaaat aaacttgatg aaagaaaaaa gttaactaaa | 360 |
| actctaaaaa agcatgcaag gcttaaaaaa atagagcaga tgtcggagga gatcaagtgg | 420 |
| attcaaaaaa aagaacaagc gattgatttt gtaaaagatc ttataacaat gaaagaagaa | 480 |
| ccaattaaac ttcttgcact tacatcaaat tatagacttt tttatcaatg taaaattctt | 540 |
| tcacaaaaag gttatagtgg tcaacaaatt gcaaaaacaa taggtgttca tccatataga | 600 |
| gtgaaacttg cacttggtca agtgagacat tatcaacttg atgaacttct taatattatt | 660 |
| gatgcatgtg cagaaacaga ttataaactt aaatcatcat atatggataa acaacttatt | 720 |
| cttgaacttt ttattctttc actt | 744 |

The amino acid sequence of the delta prime subunit encoded by S. aureus holB corresponds to SEQ. ID. No. 14 as follows:

```
Met Ser Asp Asn Ile Val Ala Ile Tyr Gly Asp Val Pro Glu Leu Val
    1 5 10 15
Glu Lys Gln Ser Ala Glu Ile Ile Ser Gln Phe Leu Lys Ser Asp Arg
                          20 25 30
Asp Asp Phe Asn Phe Val Lys Tyr Asn Leu Tyr Glu Thr Glu Ile Ala
                  35 40 45
Pro Ile Val Glu Glu Thr Leu Thr Leu Pro Phe Phe Ser Asp Lys Lys
          50 55 60
Ala Ile Leu Val Lys Asn Ala Tyr Ile Phe Thr Gly Glu Lys Ala Pro
  65 70 75 80
Lys Asp Met Ala His Asn Val Asp Gln Leu Ile Glu Phe Ile Glu Lys
                                  85 90 95
Tyr Asp Gly Glu Asn Leu Ile Val Phe Glu Ile Tyr Gln Asn Lys Leu
                        100 105 110
Asp Glu Arg Lys Lys Leu Thr Lys Thr Leu Lys Lys His Ala Arg Leu
                115 120 125
Lys Lys Ile Glu Gln Met Ser Glu Glu Ile Lys Trp Ile Gln Lys Lys
        130 135 140
Glu Gln Ala Ile Asp Phe Val Lys Asp Leu Ile Thr Met Lys Glu Glu
145 150 155 160
Pro Ile Lys Leu Leu Ala Leu Thr Ser Asn Tyr Arg Leu Phe Tyr Gln
                                165 170 175
Cys Lys Ile Leu Ser Gln Lys Gly Tyr Ser Gly Gln Gln Ile Ala Lys
                        180 185 190
Thr Ile Gly Val His Pro Tyr Arg Val Lys Leu Ala Leu Gly Gln Val
                195 200 205
Arg His Tyr Gln Leu Asp Glu Leu Leu Asn Ile Ile Asp Ala Cys Ala
        210 215 220
Glu Thr Asp Tyr Lys Leu Lys Ser Ser Tyr Met Asp Lys Gln Leu Ile
225 230 235 240
Leu Glu Leu Phe Ile Leu Ser Leu
                                245
```

This invention also relates to the S. aureus dnaG gene encoding a primase. The partial nucleotide sequence of this dnaG gene corresponds to SEQ. ID. No. 15 as follows:

| | |
|---|---|
| atgataggtt tgtgtccttt tcatgatgaa aagacacctt catttacagt ttctgaagat | 60 |
| aaacaaatct gtcattgttt tggttgtaaa aaaggtggca atgtttttca atttactcaa | 120 |
| gaaattaaag acatatcatt tgttgaagcg gttaaagaat taggtgatag agttaatgtt | 180 |
| gctgtagata ttgaggcaac acaatctaac tcaaatgttc aaattgcttc tgatgattta | 240 |
| caaatgattg aaatgcatga gttaatacaa gaattttatt attacgcttt aacaaagaca | 300 |
| gtcgaaggcg aacaagcatt aacatactta caagaacgtg gttttacaga tgcgcttatt | 360 |
| aaagagcgag gcattggctt tgcacccgat agctcacatt tttgtcatga ttttcttcaa | 420 |
| aaaaagggtt acgatattga attagcatat gaagccggat tattatcacg taacgaagaa | 480 |
| aatttcagtt attacgatag atttcgaaat cgtattatgt ttcctttgaa aaatgcgcaa | 540 |
| ggaagaattg ttggatattc aggtcgaaca tataccggtc aagaaccaaa atacctaaat | 600 |
| agtcctgaaa cgcctatctt tcaaaaaaga aagttgttat ataacttaga taaagcacgt | 660 |
| aaatcaatta gaaaattaga tgaaattgta ttactagaag gttttatgga tgttataaaa | 720 |
| tctgatactg ctggcttgaa aaacgttgtt gcaacaatgg gtacacagtt gtcagatgaa | 780 |
| catattacct ttatacgaaa gttaacatca aatataacat taatgtttga tggggatttt | 840 |
| gcgggtagtg aagcaacact taaaacaggt caacatttgt tacagcaagg gctaaatgta | 900 |
| tttgttatac aattgccatc tggcatggat ccggatgaat acattggtaa gtatggcaac | 960 |
| gacgcattta ctacttttgt aaaaaatgac aaaaagtcat ttgcacatta taaagtaagt | 1020 |
| atattaaaag atgaaattgc acataatgac ctttcatatg aacgttattt gaaagaactg | 1080 |
| agtcatgaca tttcacttat gaagtcatca attctgcaac aaaaggctat aaatgatgtt | 1140 |
| gcgccatttt tcaatgttag tcctgagcag ttagctaacg aaatacaatt caatcaagca | 1200 |
| ccagccaatt attatccaga agatgagtat ggcggttatg atgagtatgg cggttatatt | 1260 |
| gaacctgagc caattggtat ggcacaattt gacaatttga gccgtcgaga aaaagcggag | 1320 |
| cgagcatttt taaaacattt aatgagagat aaagatacat ttttaaatta ttatgaaagt | 1380 |
| gttgataagg ataacttcac aaatcagcat tttaaatatg tattcgaagt cttacatgat | 1440 |
| ttttatgcgg aaaatgatca atataatatc agtgatgctg tgcagtatgt taattcaaat | 1500 |
| gagttgagag aaacactaat tagcttagaa caatataatt tgaatggcga accatatgaa | 1560 |
| aatgaaattg atgattatgt caatgttatt aatgaaaaag gacaagaaac aattgagtca | 1620 |
| ttgaatcata aattaaggga agctacaagg attggcgatg tagaattaca aaaatactat | 1680 |
| ttacagcaaa ttgttgctaa gaataaagaa cgcatgtag | 1719 |

The amino acid sequence of primase encoded by S. aureus dnaG corresponds to SEQ. ID. No. 16 as follows:

```
Met Ile Gly Leu Cys Pro Phe His Asp Glu Lys Thr Pro Ser Phe Thr
1 5 10 15
Val Ser Glu Asp Lys Gln Ile Cys His Cys Phe Gly Cys Lys Lys Gly
                        20 25 30
Gly Asn Val Phe Gln Phe Thr Gln Glu Ile Lys Asp Ile Ser Phe Val
                35 40 45
Glu Ala Val Lys Glu Leu Gly Asp Arg Val Asn Val Ala Val Asp Ile
        50 55 60
Glu Ala Thr Gln Ser Asn Ser Asn Val Gln Ile Ala Ser Asp Asp Leu
65 70 75 80
Gln Met Ile Glu Met His Glu Leu Ile Gln Glu Phe Tyr Tyr Tyr Ala
                                85 90 95
Leu Thr Lys Thr Val Glu Gly Glu Gln Ala Leu Thr Tyr Leu Gln Glu
                        100 105 110
Arg Gly Phe Thr Asp Ala Leu Ile Lys Glu Arg Gly Ile Gly Phe Ala
                115 120 125
Pro Asp Ser Ser His Phe Cys His Asp Phe Leu Gln Lys Lys Gly Tyr
        130 135 140
Asp Ile Glu Leu Ala Tyr Glu Ala Gly Leu Leu Ser Arg Asn Glu Glu
145 150 155 160
Asn Phe Ser Tyr Tyr Asp Arg Phe Arg Asn Arg Ile Met Phe Pro Leu
                                165 170 175
Lys Asn Ala Gln Gly Arg Ile Val Gly Tyr Ser Gly Arg Thr Tyr Thr
                        180 185 190
Gly Gln Glu Pro Lys Tyr Leu Asn Ser Pro Glu Thr Pro Ile Phe Gln
                195 200 205
Lys Arg Lys Leu Leu Tyr Asn Leu Asp Lys Ala Arg Lys Ser Ile Arg
        210 215 220
Lys Leu Asp Glu Ile Val Leu Leu Glu Gly Phe Met Asp Val Ile Lys
225 230 235 240
Ser Asp Thr Ala Gly Leu Lys Asn Val Val Ala Thr Met Gly Thr Gln
                                245 250 255
Leu Ser Asp Glu His Ile Thr Phe Ile Arg Lys Leu Thr Ser Asn Ile
                        260 265 270
Thr Leu Met Phe Asp Gly Asp Phe Ala Gly Ser Glu Ala Thr Leu Lys
                275 280 285
Thr Gly Gln His Leu Leu Gln Gln Gly Leu Asn Val Phe Val Ile Gln
        290 295 300
Leu Pro Ser Gly Met Asp Pro Asp Glu Tyr Ile Gly Lys Tyr Gly Asn
305 310 315 320
Asp Ala Phe Thr Thr Phe Val Lys Asn Asp Lys Lys Ser Phe Ala His
                                325 330 335
Tyr Lys Val Ser Ile Leu Lys Asp Glu Ile Ala His Asn Asp Leu Ser
                        340 345 350
Tyr Glu Arg Tyr Leu Lys Glu Leu Ser His Asp Ile Ser Leu Met Lys
                355 360 365
Ser Ser Ile Leu Gln Gln Lys Ala Ile Asn Asp Val Ala Pro Phe Phe
        370 375 380
Asn Val Ser Pro Glu Gln Leu Ala Asn Glu Ile Gln Phe Asn Gln Ala
385 390 395 400
Pro Ala Asn Tyr Tyr Pro Glu Asp Glu Tyr Gly Gly Tyr Asp Glu Tyr
                                405 410 415
Gly Gly Tyr Ile Glu Pro Glu Pro Ile Gly Met Ala Gln Phe Asp Asn
                        420 425 430
Leu Ser Arg Arg Glu Lys Ala Glu Arg Ala Phe Leu Lys His Leu Met
                435 440 445
Arg Asp Lys Asp Thr Phe Leu Asn Tyr Tyr Glu Ser Val Asp Lys Asp
        450 455 460
Asn Phe Thr Asn Gln His Phe Lys Tyr Val Phe Glu Val Leu His Asp
465 470 475 480
Phe Tyr Ala Glu Asn Asp Gln Tyr Asn Ile Ser Asp Ala Val Gln Tyr
                                485 490 495
Val Asn Ser Asn Glu Leu Arg Glu Thr Leu Ile Ser Leu Glu Gln Tyr
                        500 505 510
Asn Leu Asn Gly Glu Pro Tyr Glu Asn Glu Ile Asp Asp Tyr Val Asn
                515 520 525
Val Ile Asn Glu Lys Gly Gln Glu Thr Ile Glu Ser Leu Asn His Lys
        530 535 540
Leu Arg Glu Ala Thr Arg Ile Gly Asp Val Glu Leu Gln Lys Tyr Tyr
545 550 555 560
Leu Gln Gln Ile Val Ala Lys Asn Lys Glu Arg Met
                                565 570
```

This invention also relates to the polC gene of Streptococcus pyogenes encoding the α-large subunit. The partial nucleotide sequence of polC (α-large) corresponds to SEQ. ID. No. 17 as follows:

| | |
|---|---|
| atgtcagatt tattcgctaa attgatggac cagatagaaa tgccacttga catgagacgt | 60 |
| tcaagtgcct tttcatctgc tgatattatc gaggtaaagg tacattcggt gtcacgcttg | 120 |
| tgggaatttc attttgcctt tgcagcggtt ttaccgattg caacttatcg tgaattgcat | 180 |
| gatcgtttga taagaacttt tgaggcggct gacattaagg taacctttga catccaagct | 240 |
| gctcaggtgg attattcaga tgatctgctt caagcttatt accaagaagc ttttgagcat | 300 |
| gcaccgtgta atagtgctag ttttaaatct tctttctcaa agctcaaagt gacttatgag | 360 |
| gatgacaaac tcattattgc agcgccaggt tttgtgaata acgatcattt tagaaacaat | 420 |
| catctgccta atctggtcaa gcaattagaa gcctttggct ttggcatctt gaccatagat | 480 |
| atggtgtcag atcaggaaat gactgagcat ttgaccaaga attttgtttc cagtcgtcag | 540 |
| gctcttgtga aaaaggctgt gcaggataat ttggaagccc aaaaatctct tgaagccatg | 600 |
| atgccaccag ttgaggaagc cacacctgct cctaagtttg actacaagga acgagcagct | 660 |
| aagcgtcagg cagggtttga aaaagcaacc atcacaccaa tgattgagat tgagaccgaa | 720 |
| gaaaaccgga ttgtctttga gggtatggtt tttgacgtgg agcgtaaaac gactaggaca | 780 |
| ggtcgccata tcatcaactt taaaatgaca gactatacet cctcgtttgc tctccaaaaa | 840 |
| tgggctaaag acgatgagga gctccgtaaa tttgatatga ttgctaaggg agcttggtta | 900 |
| cgggtacaag ggaatattga gaccaatcct tttacgaaga gtctcaccat gaatgtccag | 960 |
| caggtcaaag aaattgtccg tcatgagcgc aaagacctga tgccagaagg gcaaaagcgg | 1020 |
| gtcgaacttc atgcccacac caatatgtct accatggatg ccttaccgac agtagaaagc | 1080 |
| ttgattgata cggcagccaa gtggggacac aaggcgattg ctatcaccga ccatgctaat | 1140 |
| gtgcaaagtt ttcctcatgg ctaccatagg gctcgcaaag ctgggattaa ggctattttt | 1200 |
| ggcctagaag ccaatattgt tgaggacaag gtgcctattt cttatgaacc tgttgatatg | 1260 |
| gatttgcacg aagccaccta tgtggtcttt gacgtggaaa ccacaggtct atctgctatg | 1320 |
| aataatgacc tgattcagat tgcggcttcc aaaatgttta aaggaaatat tgtagagcag | 1380 |
| tttgatgaat tcattgatcc tgggcatcct ctttcagcct ttaccaccga attgacagga | 1440 |
| attaccgata agcatttgca gggcgccaag ccattggtta ctgtcctaaa agcttttcag | 1500 |
| gacttttgca aagatagtat cttggttgcc cacaacgcca gttttgacgt gggctttatg | 1560 |
| aacgccaatt atgaacgcca cgacttgccc aaaatcacac agcctgtgat tgatacctta | 1620 |
| gaatttgcta gaaacttgta tcctgagtac aagcgtcacg gtttgggacc gctcaccaag | 1680 |
| cgtttccaag tgagtctaga ccaccatcat atggccaatt acgacgcgga agccacagga | 1740 |
| cgtcttttgt ttatttttct aaaagatgcc agagaaaagc atggcatcaa aaatcttttg | 1800 |
| caactcaata cagatttggt ggctgaggat tcttacaaaa aagcgcggat taagcatgcg | 1860 |
| actatctatg tgcaaaatca ggttggtctt aaaaatatgt ttaagttggt cagcctttcc | 1920 |
| aatatcaaat attttgaagg ggtgccgcgt attccaagaa ccgtcttaga tgctcacaga | 1980 |
| gagggtttgt tacttggtac agcttgttct gacggcgagg tttttgatgc cgttctgact | 2040 |
| aaaggaattg atgcagcggt tgatttggct aggtattatg attttatcga aatcatgcca | 2100 |
| ccagccattt accagccatt ggttgtccgt gaattaatca aagatcaagc aggtattgag | 2160 |
| caggtgattc gtgacctcat tgaagtaggg aaacgagcta agaaacctgt gcttgccact | 2220 |
| gggaatgtgc attatctaga gcctgaagaa gagatttacc gtgaaattat tgtgcgtagt | 2280 |
| cttggtcagg gtgccatgat taatagaaca atcggccgtg gggaaggggc acagcctgct | 2340 |
| cctctaccta aagcgcactt tagaacaacc aatgaaatgc tggatgagtt tgcctttctt | 2400 |
| ggaaaagacc tcgcttatca agtagttgtg caaaatactc aggattttgc ggaccgtatt | 2460 |
| gaggaagtgg aagtggttaa gggcgatctt tacaccccgt atattgataa ggccgaagag | 2520 |
| acggttgccg aattaaccta tcaaaaagcc tttgaaattt atggtaatcc tctcccagat | 2580 |
| attattgatt tacgcattga aaaagagtta acctctatct tggggaacgg ttttgctgtg | 2640 |
| atttatctcg cttcccaaat gcttgttaac cggtcaaatg agcgaggcta cctagttggt | 2700 |
| tctaggggat ctgtagggtc tagctttgtc gccaccatga ttgggattac tgaggttaat | 2760 |
| cctatgccgc ctcactacgt ttgcccgtcc tgccaacatt ctgaatttat cacagatggg | 2820 |
| tcagttggat ctggctatga tttgcctaat aaaccctgtc cgaaatgtgg caccccttat | 2880 |
| caaaaagatg ggcaagacat tccctttgag acctttcttg ggtttgatgg ggataaggtg | 2940 |
| cccgatattg atttgaactt ctctggtgat gaccagccca gtgcccattt ggatgtccga | 3000 |
| gatatttttg gtgacgaata cgcctttcgt gctggaacag ttggtaccgt agcagaaaaa | 3060 |
| acagcttatg gatttgtcaa aggctatgaa cgcgactatg gcaagttcta tcgtgatgct | 3120 |
| gaggtggatc gtctagcagc aggtgctgct ggtgtgaaac gaacgactgg gcagcaccct | 3180 |
| ggggggattg ttgttattcc taattacatg gatgtttatg attttacccc cgtgcaatat | 3240 |
| ccagccgatg atgtaacggc ttcttggcag acaactcact ttaacttcca tgatattgat | 3300 |
| gaaaacgtct tgaaacttga tatcctaggg catgatgatc cgaccatgat tcgtaaactt | 3360 |
| caggatttat cgggcattga tcctattact attcctgctg atgatccggg agttatggct | 3420 |
| ctcttttctg ggacagaggt tttgggcgtt accccggaac aaattgggac accgactggt | 3480 |
| atgctaggca ttccagaatt tggaaccaac tttgttcgcg gcatggttaa tgagacgcat | 3540 |
| ccgaccactt ttgcggagct tttgcagttg tctggactat ctcatggaac cgatgtttgg | 3600 |
| cttggtaatg cacaagattt gattaaagaa ggcattgcaa ccctaaaaac cgttatcggt | 3660 |
| tgtcgtgacg acatcatggt ttacctcatg cacgcaggct tagaaccaaa aatggccttt | 3720 |
| accattatgg agcgtgtgcg taagggatta tggctaaaaa tttctgagga agaacgtaat | 3780 |
| ggctatattg atgccatgcg agaaaacaat gtgcccgact ggtacattga atcgtgtgga | 3840 |
| aaaatcaagt acatgttccc taaagcccat gcggcagctt atgttttgat ggcccttcgg | 3900 |
| gtggcttatt tcaaggtgca ccaccccatt atgtattatt gtgcttattt ctctattcgt | 3960 |
| gcgaaggctt ttgaattaaa aaccatgagt ggtggtttag atgctgttaa agcaagaatg | 4020 |
| gaagatatta ctataaaacg taaaaataat gaagccacca atgtggaaaa tgacctcttt | 4080 |
| acaaccttgg agattgtcaa cgaaatgtta gaacgcggct ttaagtttgg caaattagac | 4140 |
| ctttacaaaa gtgatgctat agaattccaa atcaaaggag atacccttat ccctccattt | 4200 |
| atagcgctag aaggtctggg tgaaaacgtg gccaagcaaa tcgttaaagc tcgtcaagaa | 4260 |
| ggcgaattcc tctctaaaat ggaattgcgt aaacgaggcg gggcatcgtc aacgctcgtt | 4320 |
| gagaaaatgg atgagatggg tattttagga aatatgccag aagataatca attaagtctt | 4380 |
| tttgatgact ttttc | 4395 |

The encoded α-large subunit has an amino acid sequence corresponding to SEQ. ID. No. 18 as follows:

```
Met Ser Asp Leu Phe Ala Lys Leu Met Asp Gln Ile Glu Met Pro Leu
    1 5 10 15
Asp Met Arg Arg Ser Ser Ala Phe Ser Ser Ala Asp Ile Ile Glu Val
                          20 25 30
Lys Val His Ser Val Ser Arg Leu Trp Glu Phe His Phe Ala Phe Ala
                  35 40 45
Ala Val Leu Pro Ile Ala Thr Tyr Arg Glu Leu His Asp Arg Leu Ile
          50 55 60
Arg Thr Phe Glu Ala Ala Asp Ile Lys Val Thr Phe Asp Ile Gln Ala
  65 70 75 80
Ala Gln Val Asp Tyr Ser Asp Asp Leu Leu Gln Ala Tyr Tyr Gln Glu
                                  85 90 95
Ala Phe Glu His Ala Pro Cys Asn Ser Ala Ser Phe Lys Ser Ser Phe
                        100 105 110
Ser Lys Leu Lys Val Thr Tyr Glu Asp Asp Lys Leu Ile Ile Ala Ala
                115 120 125
Pro Gly Phe Val Asn Asn Asp His Phe Arg Asn Asn His Leu Pro Asn
        130 135 140
Leu Val Lys Gln Leu Glu Ala Phe Gly Phe Gly Ile Leu Thr Ile Asp
145 150 155 160
Met Val Ser Asp Gln Glu Met Thr Glu His Leu Thr Lys Asn Phe Val
                                165 170 175
Ser Ser Arg Gln Ala Leu Val Lys Lys Ala Val Gln Asp Asn Leu Glu
                        180 185 190
Ala Gln Lys Ser Leu Glu Ala Met Met Pro Pro Val Glu Glu Ala Thr
                195 200 205
Pro Ala Pro Lys Phe Asp Tyr Lys Glu Arg Ala Ala Lys Arg Gln Ala
        210 215 220
Gly Phe Glu Lys Ala Thr Ile Thr Pro Met Ile Glu Ile Glu Thr Glu
225 230 235 240
Glu Asn Arg Ile Val Phe Glu Gly Met Val Phe Asp Val Glu Arg Lys
                                245 250 255
Thr Thr Arg Thr Gly Arg His Ile Ile Asn Phe Lys Met Thr Asp Tyr
                        260 265 270
Thr Ser Ser Phe Ala Leu Gln Lys Trp Ala Lys Asp Asp Glu Glu Leu
                275 280 285
Arg Lys Phe Asp Met Ile Ala Lys Gly Ala Trp Leu Arg Val Gln Gly
        290 295 300
Asn Ile Glu Thr Asn Pro Phe Thr Lys Ser Leu Thr Met Asn Val Gln
305 310 315 320
Gln Val Lys Glu Ile Val Arg His Glu Arg Lys Asp Leu Met Pro Glu
                                325 330 335
Gly Gln Lys Arg Val Glu Leu His Ala His Thr Asn Met Ser Thr Met
                        340 345 350
Asp Ala Leu Pro Thr Val Glu Ser Leu Ile Asp Thr Ala Ala Lys Trp
                355 360 365
Gly His Lys Ala Ile Ala Ile Thr Asp His Ala Asn Val Gln Ser Phe
        370 375 380
Pro His Gly Tyr His Arg Ala Arg Lys Ala Gly Ile Lys Ala Ile Phe
385 390 395 400
Gly Leu Glu Ala Asn Ile Val Glu Asp Lys Val Pro Ile Ser Tyr Glu
                                405 410 415
Pro Val Asp Met Asp Leu His Glu Ala Thr Tyr Val Val Phe Asp Val
                        420 425 430
Glu Thr Thr Gly Leu Ser Ala Met Asn Asn Asp Leu Ile Gln Ile Ala
                435 440 445
Ala Ser Lys Met Phe Lys Gly Asn Ile Val Glu Gln Phe Asp Glu Phe
        450 455 460
Ile Asp Pro Gly His Pro Leu Ser Ala Phe Thr Thr Glu Leu Thr Gly
465 470 475 480
Ile Thr Asp Lys His Leu Gln Gly Ala Lys Pro Leu Val Thr Val Leu
                                485 490 495
Lys Ala Phe Gln Asp Phe Cys Lys Asp Ser Ile Leu Val Ala His Asn
                        500 505 510
Ala Ser Phe Asp Val Gly Phe Met Asn Ala Asn Tyr Glu Arg His Asp
                515 520 525
Leu Pro Lys Ile Thr Gln Pro Val Ile Asp Thr Leu Glu Phe Ala Arg
        530 535 540
Asn Leu Tyr Pro Glu Tyr Lys Arg His Gly Leu Gly Pro Leu Thr Lys
545 550 555 560
Arg Phe Gln Val Ser Leu Asp His His His Met Ala Asn Tyr Asp Ala
                                565 570 575
Glu Ala Thr Gly Arg Leu Leu Phe Ile Phe Leu Lys Asp Ala Arg Glu
                        580 585 590
Lys His Gly Ile Lys Asn Leu Leu Gln Leu Asn Thr Asp Leu Val Ala
                595 600 605
Glu Asp Ser Tyr Lys Lys Ala Arg Ile Lys His Ala Thr Ile Tyr Val
        610 615 620
Gln Asn Gln Val Gly Leu Lys Asn Met Phe Lys Leu Val Ser Leu Ser
625 630 635 640
Asn Ile Lys Tyr Phe Glu Gly Val Pro Arg Ile Pro Arg Thr Val Leu
                                645 650 655
Asp Ala His Arg Glu Gly Leu Leu Leu Gly Thr Ala Cys Ser Asp Gly
                        660 665 670
Glu Val Phe Asp Ala Val Leu Thr Lys Gly Ile Asp Ala Ala Val Asp
                675 680 685
Leu Ala Arg Tyr Tyr Asp Phe Ile Glu Ile Met Pro Pro Ala Ile Tyr
        690 695 700
Gln Pro Leu Val Val Arg Glu Leu Ile Lys Asp Gln Ala Gly Ile Glu
705 710 715 720
Gln Val Ile Arg Asp Leu Ile Glu Val Gly Lys Arg Ala Lys Lys Pro
                                725 730 735
Val Leu Ala Thr Gly Asn Val His Tyr Leu Glu Pro Glu Glu Glu Ile
                        740 745 750
Tyr Arg Gln Ile Ile Val Arg Ser Leu Gly Gln Gly Ala Met Ile Asn
                755 760 765
Arg Thr Ile Gly Arg Gly Glu Gly Ala Gln Pro Ala Pro Leu Pro Lys
        770 775 780
Ala His Phe Arg Thr Thr Asn Glu Met Leu Asp Glu Phe Ala Phe Leu
785 790 795 800
Gly Lys Asp Leu Ala Tyr Gln Val Val Val Gln Asn Thr Gln Asp Phe
                                805 810 815
Ala Asp Arg Ile Glu Glu Val Glu Val Val Lys Gly Asp Leu Tyr Thr
                        820 825 830
Pro Tyr Ile Asp Lys Ala Glu Glu Thr Val Ala Glu Leu Thr Tyr Gln
                835 840 845
Lys Ala Phe Glu Ile Tyr Gly Asn Pro Leu Pro Asp Ile Ile Asp Leu
        850 855 860
Arg Ile Glu Lys Glu Leu Thr Ser Ile Leu Gly Asn Gly Phe Ala Val
865 870 875 880
Ile Tyr Leu Ala Ser Gln Met Leu Val Asn Arg Ser Asn Glu Arg Gly
                                885 890 895
Tyr Leu Val Gly Ser Arg Gly Ser Val Gly Ser Ser Phe Val Ala Thr
                        900 905 910
Met Ile Gly Ile Thr Glu Val Asn Pro Met Pro Pro His Tyr Val Cys
                915 920 925
Pro Ser Cys Gln His Ser Glu Phe Ile Thr Asp Gly Ser Val Gly Ser
        930 935 940
Gly Tyr Asp Leu Pro Asn Lys Pro Cys Pro Lys Cys Gly Thr Pro Tyr
945 950 955 960
Gln Lys Asp Gly Gln Asp Ile Pro Phe Glu Thr Phe Leu Gly Phe Asp
                                965 970 975
Gly Asp Lys Val Pro Asp Ile Asp Leu Asn Phe Ser Gly Asp Asp Gln
                        980 985 990
Pro Ser Ala His Leu Asp Val Arg Asp Ile Phe Gly Asp Glu Tyr Ala
                995 1000 1005
Phe Arg Ala Gly Thr Val Gly Thr Val Ala Glu Lys Thr Ala Tyr Gly
      1010 1015 1020
Phe Val Lys Gly Tyr Glu Arg Asp Tyr Gly Lys Phe Tyr Arg Asp Ala
1025 1030 1035 1040
Glu Val Asp Arg Leu Ala Ala Gly Ala Ala Gly Val Lys Arg Thr Thr
                              1045 1050 1055
Gly Gln His Pro Gly Gly Ile Val Val Ile Pro Asn Tyr Met Asp Val
                      1060 1065 1070
Tyr Asp Phe Thr Pro Val Gln Tyr Pro Ala Asp Asp Val Thr Ala Ser
              1075 1080 1085
Trp Gln Thr Thr His Phe Asn Phe His Asp Ile Asp Glu Asn Val Leu
      1090 1095 1100
Lys Leu Asp Ile Leu Gly His Asp Asp Pro Thr Met Ile Arg Lys Leu
1105 1110 1115 1120
Gln Asp Leu Ser Gly Ile Asp Pro Ile Thr Ile Pro Ala Asp Asp Pro
                              1125 1130 1135
Gly Val Met Ala Leu Phe Ser Gly Thr Glu Val Leu Gly Val Thr Pro
                      1140 1145 1150
Glu Gln Ile Gly Thr Pro Thr Gly Met Leu Gly Ile Pro Glu Phe Gly
              1155 1160 1165
Thr Asn Phe Val Arg Gly Met Val Asn Glu Thr His Pro Thr Thr Phe
      1170 1175 1180
Ala Glu Leu Leu Gln Leu Ser Gly Leu Ser His Gly Thr Asp Val Trp
1185 1190 1195 1200
Leu Gly Asn Ala Gln Asp Leu Ile Lys Glu Gly Ile Ala Thr Leu Lys
                              1205 1210 1215
Thr Val Ile Gly Cys Arg Asp Asp Ile Met Val Tyr Leu Met His Ala
                      1220 1225 1230
Gly Leu Glu Pro Lys Met Ala Phe Thr Ile Met Glu Arg Val Arg Lys
              1235 1240 1245
Gly Leu Trp Leu Lys Ile Ser Glu Glu Glu Arg Asn Gly Tyr Ile Asp
      1250 1255 1260
Ala Met Arg Glu Asn Asn Val Pro Asp Trp Tyr Ile Glu Ser Cys Gly
1265 1270 1275 1280
Lys Ile Lys Tyr Met Phe Pro Lys Ala His Ala Ala Ala Tyr Val Leu
                              1285 1290 1295
Met Ala Leu Arg Val Ala Tyr Phe Lys Val His His Pro Ile Met Tyr
                      1300 1305 1310
Tyr Cys Ala Tyr Phe Ser Ile Arg Ala Lys Ala Phe Glu Leu Lys Thr
              1315 1320 1325
Met Ser Gly Gly Leu Asp Ala Val Lys Ala Arg Met Glu Asp Ile Thr
      1330 1335 1340
Ile Lys Arg Lys Asn Asn Glu Ala Thr Asn Val Glu Asn Asp Leu Phe
1345 1350 1355 1360
Thr Thr Leu Glu Ile Val Asn Glu Met Leu Glu Arg Gly Phe Lys Phe
                              1365 1370 1375
Gly Lys Leu Asp Leu Tyr Lys Ser Asp Ala Ile Glu Phe Gln Ile Lys
                      1380 1385 1390
Gly Asp Thr Leu Ile Pro Pro Phe Ile Ala Leu Glu Gly Leu Gly Glu
              1395 1400 1405
Asn Val Ala Lys Gln Ile Val Lys Ala Arg Gln Glu Gly Glu Phe Leu
      1410 1415 1420
Ser Lys Met Glu Leu Arg Lys Arg Gly Gly Ala Ser Ser Thr Leu Val
1425 1430 1435 1440
Glu Lys Met Asp Glu Met Gly Ile Leu Gly Asn Met Pro Glu Asp Asn
                              1445 1450 1455
Gln Leu Ser Leu Phe Asp Asp Phe Phe
                      1460 1465
```

The present invention also relates to the dnaE gene of Streptococcus pyogenes encoding the α-small subunit. The partial nucleotide sequence of the dnaE gene corresponds to SEQ. ID. No. 19 as follows:

| | |
|---|---|
| atgtttgctc aacttgatac taaaactgta tactcattta tggatagttt aattgactta | 60 |
| aatcattatt ttgaacgagc aaagcaattt ggttaccaca ccataggaat catggataag | 120 |
| gataatcttt atggtgctta ccattttatt aaaggttgtc aaaaaaatgg actgcagcca | 180 |
| gttttaggtt tggaaataga gattctctat caagagcggc aggtgctcct taacttaatc | 240 |
| gcccagaata cacaaggcta tcatcagctt ttaaaaattt ccacggcaaa aatgtctggc | 300 |
| aagcttcata tggattactt ctgccaacat ttggaaggga tagcggttat tattcctagt | 360 |
| aagggttgga gcgatacatt agtggtccct tttgactact atatgggtgt tgatcagtat | 420 |
| actgatttat ctcatatgga ttctaagagg cagcttatac ccctaaggac agttcgttat | 480 |
| tttgcgcaag atgatatgga aaccctgcac atgttgcatg ccattcgaga taacctcagt | 540 |
| ctggcagaga cccctgtggt agaaagtgat caagagttag cagattgtca acaactaacc | 600 |
| gccttctatc aaacacactg ccctcaagct ctacagaatt tagaagactt agtgtcagga | 660 |
| atctattatg atttcgatac aaatttaaaa ttgcctcatt ttaatagaga taagtctgcc | 720 |
| aagcaagaat tgcaagactt gactgaggct ggtttgaagg aaaaaggatt gtggaaagag | 780 |
| ccttatcaat cgcgcttact acatgaattg gtcattattt ctgacatggg ctttgatgat | 840 |
| tattttttga ttgtgtggga tttacttcgc tttggacgca gtaaaggcta ttatatggga | 900 |
| atgggacgtg gctcggcggc aggtagtcta gtggcttatg ctctgaacat tacagggatt | 960 |
| gatccagttc aacatgattt gctatttgag cgctttttaa acaaagaacg ttatagcatg | 1020 |
| cctgatattg atatcgatct tccagatatt taccgttcag aatttctacg gtatgtccga | 1080 |
| aatcgttatg gtagcgacca ttcggcgcaa attgtgacct tttcaacctt tggccaggct | 1140 |
| attcgtgatg ttttcaaacg gttcggggtt ccagaatacg aactgactaa tctcactaaa | 1200 |
| aaaattggtt ttaaagatag cttggctact gtctatgaaa agtcaatctc ttttaggcag | 1260 |
| gttattaata gtagaactga atttcaaaag gcttttgcca ttgccaagcg tatcgaagga | 1320 |
| aatccaagac aaacgtccat tcacgcagct ggtattgtga tgagtgatga tgccttgacc | 1380 |
| aatcatattc ctctaaaatc gggcgatgac atgatgatca cccagtatga tgctcatgcg | 1440 |
| gtcgaagcta atggcctgtt aaaaatggat tttttggggt taagaaattt gacctttgtt | 1500 |
| caaaaaatgc aagagaaggt tgctaaagac tacgggtgtc agattgatat tacagccatt | 1560 |
| gatttagaag acccgcaaac gttggcactt tttgctaaag gggataccaa gggaattttc | 1620 |
| caatttgaac aaaatggtgc tattaatctt ttaaaacgga ttaagccaca acgttttgaa | 1680 |
| gaaattgttg ceactaccag tctaaataga ccaggggcaa gtgactatac cactaatttc | 1740 |
| attaaacgaa gagaaggaca agaaaaaatt gatttgattg atcctgtgat tgctcccatt | 1800 |
| ttagagccaa cttacggtat tatgctttat caagaacaag ttatgcagat tgcacaggtt | 1860 |
| tatgctggtt ttacgttagg caaggccgac ttgttaaggc gtgccatgtc taaaaaaaat | 1920 |
| ctacaagaaa tgcaaaaaat ggaagaagac tttattgctt ctgctaagca cctagggaga | 1980 |
| gctgaagaaa cagctagagg actttttaaa cggatggaaa aatttgcagg ttatggtttt | 2040 |
| aaccgcagcc atgcctttgc ctattcagct ttagcttttc aattggctta tttcaaagcc | 2100 |
| cattacccgg ctgtttttta cgatatcatg atgaattatt ctagcagtga ctatatcaca | 2160 |
| gatgctctag aatcagattt tcaagtagcg caagttacca ttaatagtat tccttacact | 2220 |
| gataaaattg aagctagcaa gatttacatg gggctgaaaa atattaaggg gttgccaagg | 2280 |
| gattttgctt attggattat cgagcaaaga ccatttaata gcgtagagga ttttctcact | 2340 |
| agaactccag aaaaatatca aaaaaaggtt ttccttgagc ctctgataaa aataggtctg | 2400 |
| tttgattgct ttgagcctaa ccgtaaaaaa attctggaca atttggatgg tttactggta | 2460 |
| tttgttaatg agcttggttc tcttttttca gattcttcct ttagttgggt agatacgaaa | 2520 |
| gattacteag taactgaaaa atattctttg gaacaggaga tcgttggagt tggcatgagc | 2580 |
| aagcatcctt taattgatat tgctgagaaa agtacccaaa cttttactcc tatttcacag | 2640 |
| ttagtcaaag aaagcgaagc agtcgtactg attcaaatag atagcattag gateattaga | 2700 |
| accaaaacaa gtgggcagca aatggctttt ttaagtgtga atgacactaa gaaaaagctc | 2760 |
| gatgtcacac tttttccaca agagtatgcc atttataaag accaattaaa agaaggagaa | 2820 |
| ttctattact taaaaggtag aataaaagaa agagaccatc gactgcagat ggtgtgtcag | 2880 |
| caagtgcaaa tggctattag tcaaaaatat tggttattag ttgaaaacca tcagtttgat | 2940 |
| tcccaaattt ctgagatttt aggtgccttt ccaggaacga ctccagttgt tattcactat | 3000 |
| caaaaaaata aggaaacaat tgcattaact aagattcagg ttcatgtaac agagaattta | 3060 |
| aaggaaaaac ttcgtccttt tgttctgaaa acggtttttc ga | 3102 |

The encoded α-small subunit has an amino acid sequence corresponding to SEQ. ID. No. 20 as follows:

```
Met Phe Ala Gln Leu Asp Thr Lys Thr Val Tyr Ser Phe Met Asp Ser
    1 5 10 15
Leu Ile Asp Leu Asn His Tyr Phe Glu Arg Ala Lys Gln Phe Gly Tyr
                          20 25 30
His Thr Ile Gly Ile Met Asp Lys Asp Asn Leu Tyr Gly Ala Tyr His
                  35 40 45
Phe Ile Lys Gly Cys Gln Lys Asn Gly Leu Gln Pro Val Leu Gly Leu
          50 55 60
Glu Ile Glu Ile Leu Tyr Gln Glu Arg Gln Val Leu Leu Asn Leu Ile
  65 70 75 80
Ala Gln Asn Thr Gln Gly Tyr His Gln Leu Leu Lys Ile Ser Thr Ala
                                  85 90 95
Lys Met Ser Gly Lys Leu His Met Asp Tyr Phe Cys Gln His Leu Glu
                        100 105 110
Gly Ile Ala Val Ile Ile Pro Ser Lys Gly Trp Ser Asp Thr Leu Val
                115 120 125
Val Pro Phe Asp Tyr Tyr Met Gly Val Asp Gln Tyr Thr Asp Leu Ser
        130 135 140
His Met Asp Ser Lys Arg Gln Leu Ile Pro Leu Arg Thr Val Arg Tyr
145 150 155 160
Phe Ala Gln Asp Asp Met Glu Thr Leu His Met Leu His Ala Ile Arg
                                165 170 175
Asp Asn Leu Ser Leu Ala Glu Thr Pro Val Val Glu Ser Asp Gln Glu
                        180 185 190
Leu Ala Asp Cys Gln Gln Leu Thr Ala Phe Tyr Gln Thr His Cys Pro
                195 200 205
Gln Ala Leu Gln Asn Leu Glu Asp Leu Val Ser Gly Ile Tyr Tyr Asp
        210 215 220
Phe Asp Thr Asn Leu Lys Leu Pro His Phe Asn Arg Asp Lys Ser Ala
225 230 235 240
Lys Gln Glu Leu Gln Asp Leu Thr Glu Ala Gly Leu Lys Glu Lys Gly
                                245 250 255
Leu Trp Lys Glu Pro Tyr Gln Ser Arg Leu Leu His Glu Leu Val Ile
                        260 265 270
Ile Ser Asp Met Gly Phe Asp Asp Tyr Phe Leu Ile Val Trp Asp Leu
                275 280 285
Leu Arg Phe Gly Arg Ser Lys Gly Tyr Tyr Met Gly Met Gly Arg Gly
        290 295 300
Ser Ala Ala Gly Ser Leu Val Ala Tyr Ala Leu Asn Ile Thr Gly Ile
305 310 315 320
Asp Pro Val Gln His Asp Leu Leu Phe Glu Arg Phe Leu Asn Lys Glu
                                325 330 335
Arg Tyr Ser Met Pro Asp Ile Asp Ile Asp Leu Pro Asp Ile Tyr Arg
                        340 345 350
Ser Glu Phe Leu Arg Tyr Val Arg Asn Arg Tyr Gly Ser Asp His Ser
                355 360 365
Ala Gln Ile Val Thr Phe Ser Thr Phe Gly Pro Lys Gln Ala Ile Arg
        370 375 380
Asp Val Phe Lys Arg Phe Gly Val Pro Glu Tyr Glu Leu Thr Asn Leu
385 390 395 400
Thr Lys Lys Ile Gly Phe Lys Asp Ser Leu Ala Thr Val Tyr Glu Lys
                                405 410 415
Ser Ile Ser Phe Arg Gln Val Ile Asn Ser Arg Thr Glu Phe Gln Lys
                        420 425 430
Ala Phe Ala Ile Ala Lys Arg Ile Glu Gly Asn Pro Arg Gln Thr Ser
                435 440 445
Ile His Ala Ala Gly Ile Val Met Ser Asp Asp Ala Leu Thr Asn His
        450 455 460
Ile Pro Leu Lys Ser Gly Asp Asp Met Met Ile Thr Gln Tyr Asp Ala
465 470 475 480
His Ala Val Glu Ala Asn Gly Leu Leu Lys Met Asp Phe Leu Gly Leu
                                485 490 495
Arg Asn Leu Thr Phe Val Gln Lys Met Gln Glu Lys Val Ala Lys Asp
                        500 505 510
Tyr Gly Cys Gln Ile Asp Ile Thr Ala Ile Asp Leu Glu Asp Pro Gln
                515 520 525
Thr Leu Ala Leu Phe Ala Lys Gly Asp Thr Lys Gly Ile Phe Gln Phe
        530 535 540
Glu Gln Asn Gly Ala Ile Asn Leu Leu Lys Arg Ile Lys Pro Gln Arg
545 550 555 560
Phe Glu Glu Ile Val Ala Thr Thr Ser Leu Asn Arg Pro Gly Ala Ser
                                565 570 575
Asp Tyr Thr Thr Asn Phe Ile Lys Arg Arg Glu Gly Gln Glu Lys Ile
                        580 585 590
Asp Leu Ile Asp Pro Val Ile Ala Pro Ile Leu Glu Pro Thr Tyr Gly
                595 600 605
Ile Met Leu Tyr Gln Glu Gln Val Met Gln Ile Ala Gln Val Tyr Ala
        610 615 620
Gly Phe Thr Leu Gly Lys Ala Asp Leu Leu Arg Arg Ala Met Ser Lys
625 630 635 640
Lys Asn Leu Gln Glu Met Gln Lys Met Glu Glu Asp Phe Ile Ala Ser
                                645 650 655
Ala Lys His Leu Gly Arg Ala Glu Glu Thr Ala Arg Gly Leu Phe Lys
                        660 665 670
Arg Met Glu Lys Phe Ala Gly Tyr Gly Phe Asn Arg Ser His Ala Phe
                675 680 685
Ala Tyr Ser Ala Leu Ala Phe Gln Leu Ala Tyr Phe Lys Ala His Tyr
        690 695 700
Pro Ala Val Phe Tyr Asp Ile Met Met Asn Tyr Ser Ser Ser Asp Tyr
705 710 715 720
Ile Thr Asp Ala Leu Glu Ser Asp Phe Gln Val Ala Gln Val Thr Ile
                                725 730 735
Asn Ser Ile Pro Tyr Thr Asp Lys Ile Glu Ala Ser Lys Ile Tyr Met
                        740 745 750
Gly Leu Lys Asn Ile Lys Gly Leu Pro Arg Asp Phe Ala Tyr Trp Ile
                755 760 765
Ile Glu Gln Arg Pro Phe Asn Ser Val Glu Asp Phe Leu Thr Arg Thr
        770 775 780
Pro Glu Lys Tyr Gln Lys Lys Val Phe Leu Glu Pro Leu Ile Lys Ile
785 790 795 800
Gly Leu Phe Asp Cys Phe Glu Pro Asn Arg Lys Lys Ile Leu Asp Asn
                                805 810 815
Leu Asp Gly Leu Leu Val Phe Val Asn Glu Leu Gly Ser Leu Phe Ser
                        820 825 830
Asp Ser Ser Phe Ser Trp Val Asp Thr Lys Asp Tyr Ser Val Thr Glu
                835 840 845
Lys Tyr Ser Leu Glu Gln Glu Ile Val Gly Val Gly Met Ser Lys His
        850 855 860
Pro Leu Ile Asp Ile Ala Glu Lys Ser Thr Gln Thr Phe Thr Pro Ile
865 870 875 880
Ser Gln Leu Val Lys Glu Ser Glu Ala Val Val Leu Ile Gln Ile Asp
                                885 890 895
Ser Ile Arg Ile Ile Arg Thr Lys Thr Ser Gly Gln Gln Met Ala Phe
                        900 905 910
Leu Ser Val Asn Asp Thr Lys Lys Lys Leu Asp Val Thr Leu Phe Pro
                915 920 925
Gln Glu Tyr Ala Ile Tyr Lys Asp Gln Leu Lys Glu Gly Glu Phe Tyr
        930 935 940
Tyr Leu Lys Gly Arg Ile Lys Glu Arg Asp His Arg Leu Gln Met Val
945 950 955 960
Cys Gln Gln Val Gln Met Ala Ile Ser Gln Lys Tyr Trp Leu Leu Val
                                965 970 975
Glu Asn His Gln Phe Asp Ser Gln Ile Ser Glu Ile Leu Gly Ala Phe
                        980 985 990
Pro Gly Thr Thr Pro Val Val Ile His Tyr Gln Lys Asn Lys Glu Thr
                995 1000 1005
Ile Ala Leu Thr Lys Ile Gln Val Thr Glu Asn Leu Lys Glu Lys Leu
      1010 1015 1020
Arg Pro Phe Val Leu Lys Thr Val Phe Arg
1025 1030
```

The present invention also relates to the holA gene of Streptococcus pyogenes encoding the 6 subunit. The holA gene has a nucleotide sequence which corresponds to SEQ. ID. No. 21 as follows:

| | |
|---|---|
| atgattgcga tagaaaagat tgaaaaactg agtaaagaaa atttgggtct tataaccctt | 60 |
| gtcacaggag atgacattgg tcagtatagc cagttgaaat cccgcttaat ggagcagatt | 120 |
| gcttttgata aggatgattt ggcctattct tactttgata tgtctgaggc cgcttatcag | 180 |
| gatgcagaaa tggatctagt gagcctaccc ttctttgctg agcagaaggt ggttattttt | 240 |
| gaccatttgt tagatateac gaccaataaa aaaagtttct taaaagaaaa agacctaaag | 300 |
| gcctttgaag cctatttaga aaatccctta gagactactc gactaattat ctttgctcca | 360 |
| ggtaaattgg atagtaagag acggcttgtt aagcttttga aacgtgatgc ccttgtttta | 420 |
| gaagccaacc ctctgaaaga agcagagcta agaacttatt ttcaaaaata cagtcatcaa | 480 |
| ctgggtttag gtttcgagag tggtgccttt gaccaattac ttttgaaatc aaacgatgat | 540 |
| tttagtcaaa tcatgaaaaa catggccttt ttaaaagcct ataaaaaaac gggaaatatt | 600 |
| agcctaactg atattgagca agccattcct aaaagtttac aagataatat tttcgatctg | 660 |
| actagacttg tcctaggagg taaaattgat gcggctagag atttgattca tgatttacgg | 720 |
| ttatctggag aagatgacat taaattaatc gctatcatgc taggccaatt tcgcttattt | 780 |
| ttgcagctga ctattcttgc tagagatgta aaaaacgagc aacaactagt gattagttta | 840 |
| tcagatattc ttgggcggcg ggttaatcct taccaggtca agtatgcgtt aaaggattct | 900 |
| aggaccttat ctcttgcctt tctaacagga gcggtgaaaa ccttgattga gacagattac | 960 |
| cagataaaaa caggacttta tgagaagagt tatctagttg atattgctct cttaaaaatc | 1020 |
| atgactcact ctcaaaaa | 1038 |

The encoded δ subunit has an amino acid sequence corresponding to SEQ. ID. No. 22 as follows:

```
Met Ile Ala Ile Glu Lys Ile Glu Lys Leu Ser Lys Glu Asn Leu Gly
    1 5 10 15
Leu Ile Thr Leu Val Thr Gly Asp Asp Ile Gly Gln Tyr Ser Gln Leu
                          20 25 30
Lys Ser Arg Leu Met Glu Gln Ile Ala Phe Asp Lys Asp Asp Leu Ala
                  35 40 45
Tyr Ser Tyr Phe Asp Met Ser Glu Ala Ala Tyr Gln Asp Ala Glu Met
          50 55 60
Asp Leu Val Ser Leu Pro Phe Phe Ala Glu Gln Lys Val Val Ile Phe
  65 70 75 80
Asp His Leu Leu Asp Ile Thr Thr Asn Lys Lys Ser Phe Leu Lys Glu
                                  85 90 95
Lys Asp Leu Lys Ala Phe Glu Ala Tyr Leu Glu Asn Pro Leu Glu Thr
                        100 105 110
Thr Arg Leu Ile Ile Phe Ala Pro Gly Lys Leu Asp Ser Lys Arg Arg
                115 120 125
Leu Val Lys Leu Leu Lys Arg Asp Ala Leu Val Leu Glu Ala Asn Pro
        130 135 140
Leu Lys Glu Ala Glu Leu Arg Thr Tyr Phe Gln Lys Tyr Ser His Gln
145 150 155 160
Leu Gly Leu Gly Phe Glu Ser Gly Ala Phe Asp Gln Leu Leu Leu Lys
                                165 170 175
Ser Asn Asp Asp Phe Ser Gln Ile Met Lys Asn Met Ala Phe Leu Lys
                        180 185 190
Ala Tyr Lys Lys Thr Gly Asn Ile Ser Leu Thr Asp Ile Glu Gln Ala
                195 200 205
Ile Pro Lys Ser Leu Gln Asp Asn Ile Phe Asp Leu Thr Arg Leu Val
        210 215 220
Leu Gly Gly Lys Ile Asp Ala Ala Arg Asp Leu Ile His Asp Leu Arg
225 230 235 240
Leu Ser Gly Glu Asp Asp Ile Lys Leu Ile Ala Ile Met Leu Gly Gln
                                245 250 255
Phe Arg Leu Phe Leu Gln Leu Thr Ile Leu Ala Arg Asp Val Lys Asn
                        260 265 270
Glu Gln Gln Leu Val Ile Ser Leu Ser Asp Ile Leu Gly Arg Arg Val
                275 280 285
Asn Pro Tyr Gln Val Lys Tyr Ala Leu Lys Asp Ser Arg Thr Leu Ser
        290 295 300
Leu Ala Phe Leu Thr Gly Ala Val Lys Thr Leu Ile Glu Thr Asp Tyr
305 310 315 320
Gln Ile Lys Thr Gly Leu Tyr Glu Lys Ser Tyr Leu Val Asp Ile Ala
                                325 330 335
Leu Leu Lys Ile Met Thr His Ser Gln Lys
                        340 345
```

The present invention also relates to the holB gene of Streptococcus pyogenes encoding the δ′ subunit. The holB gene has a nucleotide sequence which corresponds to SEQ. ID. No. 23 as follows:

| | |
|---|---|
| atggatttag cgcaaaaagc tcctaacgtt tatcaagctt ttcagacaat tttaaagaaa | 60 |
| gaccgtctga atcatgctta tcttttttcg ggtgattttg ctaatgaaga aatggctctt | 120 |
| tttttagcta aggtcatctt ttgtgaacag aaaaaggatc agacgccctg cgggcattgt | 180 |
| cgctcttgtc aattgattga acaaggagat tttgccgatg tgacggtatt ggaaccaaca | 240 |
| gggcaagtga ttaaaacgga tgtggtcaaa gaaatgatgg ctaacttttc tcagacagga | 300 |
| tatgaaaaca aacgacaagt ttttattatc aaagattgtg acaaaatgca tatcaatgcc | 360 |
| gctaatagct tgctaaaata cattgaggag cctcagggag aagcttacat atttttattg | 420 |
| accaatgatg ataacaaagt gcttccgacc attaaaagtc ggacacaggt ttttcagttt | 480 |
| cctaaaaacg aagcctatct ttaccaattg gcacaagaaa agggattatt aaaccatcag | 540 |
| gctaagctag tagccaaact tgccacaaac accagtcatc tagaacgtct gttgcaaacg | 600 |
| agcaagcttt tagaactgat aactcaagca gagcgttttg tatctatttg gctgaaagat | 660 |
| cagttgcagg catatttagc gttgaaccgt ctggtacagt tagcaactga aaaagaagaa | 720 |
| caagatttag ttttgaccct tttgaccttg ctcttggcaa gagagcgtgc gcaaacgcct | 780 |
| ttgacacaat tggaggctgt ctatcaggct aggctcatgt ggcagagcaa tgttaatttt | 840 |
| caaaacacat tagaatatat ggtgatgtca gaa | 873 |

The encoded δ′ subunit has an amino acid sequence corresponding to SEQ. ID. No. 24 as follows:

```
Met Asp Leu Ala Gln Lys Ala Pro Asn Val Tyr Gln Ala Phe Gln Thr
    1 5 10 15
Ile Leu Lys Lys Asp Arg Leu Asn His Ala Tyr Leu Phe Ser Gly Asp
                          20 25 30
Phe Ala Asn Glu Glu Met Ala Leu Phe Leu Ala Lys Val Ile Phe Cys
                  35 40 45
Glu Gln Lys Lys Asp Gln Thr Pro Cys Gly His Cys Arg Ser Cys Gln
          50 55 60
Leu Ile Glu Gln Gly Asp Phe Ala Asp Val Thr Val Leu Glu Pro Thr
  65 70 75 80
Gly Gln Val Ile Lys Thr Asp Val Val Lys Glu Met Met Ala Asn Phe
                                  85 90 95
Ser Gln Thr Gly Tyr Glu Asn Lys Arg Gln Val Phe Ile Ile Lys Asp
                        100 105 110
Cys Asp Lys Met His Ile Asn Ala Ala Asn Ser Leu Leu Lys Tyr Ile
                115 120 125
Glu Glu Pro Gln Gly Glu Ala Tyr Ile Phe Leu Leu Thr Asn Asp Asp
        130 135 140
Asn Lys Val Leu Pro Thr Ile Lys Ser Arg Thr Gln Val Phe Gln Phe
145 150 155 160
Pro Lys Asn Glu Ala Tyr Leu Tyr Gln Leu Ala Gln Glu Lys Gly Leu
                                165 170 175
Leu Asn His Gln Ala Lys Leu Val Ala Lys Leu Ala Thr Asn Thr Ser
                        180 185 190
His Leu Glu Arg Leu Leu Gln Thr Ser Lys Leu Leu Glu Leu Ile Thr
                195 200 205
Gln Ala Glu Arg Phe Val Ser Ile Trp Leu Lys Asp Gln Leu Gln Ala
        210 215 220
Tyr Leu Ala Leu Asn Arg Leu Val Gln Leu Ala Thr Glu Lys Glu Glu
225 230 235 240
Gln Asp Leu Val Leu Thr Leu Leu Thr Leu Leu Leu Ala Arg Glu Arg
                                245 250 255
Ala Gln Thr Pro Leu Thr Gln Leu Glu Ala Val Tyr Gln Ala Arg Leu
                        260 265 270
Met Trp Gln Ser Asn Val Asn Phe Gln Asn Thr Leu Glu Tyr Met Val
                275 280 285
Met Ser Glu
        290
```

The present invention also relates to the dnaX gene of Streptococcus pyogenes encoding the τ subunit. The dnaX gene has a nucleotide sequence which corresponds to SEQ. ID. No. 25 as follows:

| | |
|---|---|
| atgtatcaag ctctttatcg gaaataccgg agccaaacgt ttgacgaaat ggtgggacaa | 60 |
| tcggttattt ccacaacttt aaagcaggca gttgaatctg gcaagattag ccatgcttat | 120 |
| cttttttcag gtcctagagg gactgggaaa acaagtgcgg caaagatttt tgcaaaggcc | 180 |
| atgaattgtc ctaaccaagt cgatggtgaa ccctgtaatc aatgcgatat ttgccgagat | 240 |
| atcacgaatg gaagcttgga agatgtgatt gaaattgatg ctgcctcgaa taatggtgtt | 300 |
| gatgaaattc gtgacattcg agacaaatca acctatgcgc caagtcgtgc gacttacaag | 360 |
| gtttatatta ttgatgaggt tcacatgtta tcaacagggg cttttaatgc gcttttgaaa | 420 |
| actttggaag aaccgacaga atgttgtctt tatcttggca acaacggaat gcataaaatt | 480 |
| ccagccacta ttttatctcg tgtgcaacgc tttgaattca aagctattaa gcaaaaagct | 540 |
| attcgagagc atttagcctg ggttttggac aaagaaggta ttgcctatga ggtggatgct | 600 |
| ttaaatctca ttgcaaggcg agcagaagga ggcatgcgtg atgctttatc tattttagat | 660 |
| caggctttga gcttgtcacc agataatcag gtcgccattg caattgccga agaaattaca | 720 |
| ggttctattt ccatacttgc tctgggtgac tatgttcgat atgtctccca agaacaggct | 780 |
| acgcaagctc tggcagcctt agagaccatt tatgatagtg ggaagagcat gagccgcttt | 840 |
| gcgacagatt tattgaccta tctgcgtgat ttattggtgg ttaaagctgg cggcgacaat | 900 |
| caacgtcagt cagctgtttt tgataccaat ttgtctctct cgatagatcg tatattccaa | 960 |
| atgataacag ttgttactag tcatctccct gaaatcaaaa agggaaccca tcctcggatt | 1020 |
| tatgccgaaa tgatgactat ccaattagct cagaaagagc agattttgtc ccaagtaaac | 1080 |
| ttgtcaggag agttaatctc agagattgaa acgctcaaaa atgagttggc acaacttaaa | 1140 |
| caacaattgt cgcagctcca atcgcgtcct gattcactgg caagatctga taaaacgaaa | 1200 |
| cctaaaacca caagctacag ggttgatcgg gttaccattt tgaaaatcat ggaagaaacg | 1260 |
| gttcgaaata gccaacaatc tcgacaatat ctagatgctc taaaaaatgc ttggaatgaa | 1320 |
| attctagata acatttctgc ccaagacaga gccttattga tgggctctga gcctgtctta | 1380 |
| gcaaatagtg agaatgcgat tttggctttc gaggctgcct ttaatgcaga acaagtcatg | 1440 |
| agccgaaata atcttaatga tatgtttggt aacattatga gtaaagctgc tggtttttct | 1500 |
| cccaatattc tggcagtacc aaggacagat tttcagcata ttcgtaagga atttgctcag | 1560 |
| caaatgaaat cgcaaaaaga cagtgttcaa gaagaacaag aagtagcgct tgatattcca | 1620 |
| gaagggtttg attttttgct cgataaaata aatactattg acgac | 1665 |

The encoded τ subunit has an amino acid sequence corresponding to SEQ. ID. No. 26 as follows:

```
Met Tyr Gln Ala Leu Tyr Arg Lys Tyr Arg Ser Gln Thr Phe Asp Glu
    1 5 10 15
Met Val Gly Gln Ser Val Ile Ser Thr Thr Leu Lys Gln Ala Val Glu
                          20 25 30
Ser Gly Lys Ile Ser His Ala Tyr Leu Phe Ser Gly Pro Arg Gly Thr
                  35 40 45
Gly Lys Thr Ser Ala Ala Lys Ile Phe Ala Lys Ala Met Asn Cys Pro
          50 55 60
Asn Gln Val Asp Gly Glu Pro Cys Asn Gln Cys Asp Ile Cys Arg Asp
  65 70 75 80
Ile Thr Asn Gly Ser Leu Glu Asp Val Ile Glu Ile Asp Ala Ala Ser
                                  85 90 95
Asn Asn Gly Val Asp Glu Ile Arg Asp Ile Arg Asp Lys Ser Thr Tyr
                        100 105 110
Ala Pro Ser Arg Ala Thr Tyr Lys Val Tyr Ile Ile Asp Glu Val His
                115 120 125
Met Leu Ser Thr Gly Ala Phe Asn Ala Leu Leu Lys Thr Leu Glu Glu
        130 135 140
Pro Thr Glu Asn Val Phe Ile Leu Ala Thr Thr Glu Leu His Lys Ile
145 150 155 160
Pro Ala Thr Ile Leu Ser Arg Val Gln Arg Phe Glu Phe Lys Ala Ile
                                165 170 175
Lys Gln Lys Ala Ile Arg Glu His Leu Ala Trp Val Leu Asp Lys Glu
                        180 185 190
Gly Ile Ala Tyr Glu Val Asp Ala Leu Asn Leu Ile Ala Arg Arg Ala
                195 200 205
Glu Gly Gly Met Arg Asp Ala Leu Ser Ile Leu Asp Gln Ala Leu Ser
        210 215 220
Leu Ser Pro Asp Asn Gln Val Ala Ile Ala Ile Ala Glu Glu Ile Thr
225 230 235 240
Gly Ser Ile Ser Ile Leu Ala Leu Gly Asp Tyr Val Arg Tyr Val Ser
                                245 250 255
Gln Glu Gln Ala Thr Gln Ala Leu Ala Ala Leu Glu Thr Ile Tyr Asp
                        260 265 270
Ser Gly Lys Ser Met Ser Arg Phe Ala Thr Asp Leu Leu Thr Tyr Leu
                275 280 285
Arg Asp Leu Leu Val Val Lys Ala Gly Gly Asp Asn Gln Arg Gln Ser
        290 295 300
Ala Val Phe Asp Thr Asn Leu Ser Leu Ser Ile Asp Arg Ile Phe Gln
305 310 315 320
Met Ile Thr Val Val Thr Ser His Leu Pro Glu Ile Lys Lys Gly Thr
                                325 330 335
His Pro Arg Ile Tyr Ala Glu Met Met Thr Ile Gln Leu Ala Gln Lys
                        340 345 350
Glu Gln Ile Leu Ser Gln Val Asn Leu Ser Gly Glu Leu Ile Ser Glu
                355 360 365
Ile Glu Thr Leu Lys Asn Glu Leu Ala Gln Leu Lys Gln Gln Leu Ser
        370 375 380
Gln Leu Gln Ser Arg Pro Asp Ser Leu Ala Arg Ser Asp Lys Thr Lys
385 390 395 400
Pro Lys Thr Thr Ser Tyr Arg Val Asp Arg Val Thr Ile Leu Lys Ile
                                405 410 415
Met Glu Glu Thr Val Arg Asn Ser Gln Gln Ser Arg Gln Tyr Leu Asp
                        420 425 430
Ala Leu Lys Asn Ala Trp Asn Glu Ile Leu Asp Asn Ile Ser Ala Gln
                435 440 445
Asp Arg Ala Leu Leu Met Gly Ser Glu Pro Val Leu Ala Asn Ser Glu
        450 455 460
Asn Ala Ile Leu Ala Phe Glu Ala Ala Phe Asn Ala Glu Gln Val Met
465 470 475 480
Ser Arg Asn Asn Leu Asn Asp Met Phe Gly Asn Ile Met Ser Lys Ala
                                485 490 495
Ala Gly Phe Ser Pro Asn Ile Leu Ala Val Pro Arg Thr Asp Phe Gln
                        500 505 510
His Ile Arg Lys Glu Phe Ala Gln Gln Met Lys Ser Gln Lys Asp Ser
                515 520 525
Val Gln Glu Glu Gln Glu Val Ala Leu Asp Ile Pro Glu Gly Phe Asp
        530 535 540
Phe Leu Leu Asp Lys Ile Asn Thr Ile Asp Asp
545 550 555
```

The present invention also relates to the dnaN gene of Streptococcus pyogenes encoding the β subunit. The dnaN gene has a nucleotide sequence which corresponds to SEQ. ID. No. 27 as follows:

| | |
|---|---|
| atgattcaat tttcaattaa tcgcacatta tttattcatg ctttaaatac aactaaacgt | 60 |
| gctattagca ctaaaaatgc cattcctatt ctttcatcaa taaaaattga agtcacttct | 120 |
| acaggagtaa ctttaacagg gtctaacggt caaatatcaa ttgaaaacac tattcctgta | 180 |
| agtaatgaaa atgctggttt gctaattacc tctccaggag ctattttatt agaagctagt | 240 |
| ttttttatta atattatttc aagtttgcca gatattagta taaatgttaa agaaattgaa | 300 |
| caacaccaag ttgttttaac cagtggtaaa teagagatta ccttaaaagg aaaagatgtt | 360 |
| gaccagtatc ctcgtctaca agaagtatca acagaaaatc ctttgatttt aaaaacaaaa | 420 |
| ttattgaagt ctattattgc tgaaacagct tttgcagcca gtttacaaga aagtcgtcct | 480 |
| attttaacag gagttcatat tgtattaagt aatcataaag attttaaagc agtagcgact | 540 |
| gactctcatc gtatgagcca acgtttaatc actttggaca atacttcagc agatttgatg | 600 |
| gtagttcttc caagtaaatc tttgagagaa ttttcagcag tatttacaga tgatattgag | 660 |
| accgttgagg tatttttctc accaagccaa atcttgttca gaagtgaaca catttctttt | 720 |
| tatacacgcc tcttagaagg aaattatccc gatacagacc gtttattaat gacagaattt | 780 |
| gagacggagg ttgttttcaa tacccaatcc cttcgccacg ctatggaacg tgccttcttg | 840 |
| atttctaatg ctactcaaaa tggtactgtt aagcttgaga ttactcaaaa tcatatttca | 900 |
| gctcatgtta actcacctga ggttggtaag gtaaacgagg atttagatat tgttagtcag | 960 |
| tctggtagtg atttaactat cagcttcaat ccaacttacc ttattgagtc tttaaaagct | 1020 |
| attaaaagtg aaacagtaaa aattcatttc ttatcaccag ttcgaccatt caccctaaca | 1080 |
| ccaggcgatg aggaagaaag ttttatccaa ttaattacac cagtacgaac aaac | 1134 |

The encoded β subunit has an amino acid sequence corresponding to SEQ. ID. No. 28 as follows:

```
Met Ile Gln Phe Ser Ile Asn Arg Thr Leu Phe Ile His Ala Leu Asn
    1 5 10 15
Thr Thr Lys Arg Ala Ile Ser Thr Lys Asn Ala Ile Pro Ile Leu Ser
                          20 25 30
Ser Ile Lys Ile Glu Val Thr Ser Thr Gly Val Thr Leu Thr Gly Ser
                  35 40 45
Asn Gly Gln Ile Ser Ile Glu Asn Thr Ile Pro Val Ser Asn Glu Asn
          50 55 60
Ala Gly Leu Leu Ile Thr Ser Pro Gly Ala Ile Leu Leu Glu Ala Ser
  65 70 75 80
Phe Phe Ile Asn Ile Ile Ser Ser Leu Pro Asp Ile Ser Ile Asn Val
                                  85 90 95
Lys Glu Ile Glu Gln His Gln Val Val Leu Thr Ser Gly Lys Ser Glu
                        100 105 110
Ile Thr Leu Lys Gly Lys Asp Val Asp Gln Tyr Pro Arg Leu Gln Glu
                115 120 125
Val Ser Thr Glu Asn Pro Leu Ile Leu Lys Thr Lys Leu Leu Lys Ser
        130 135 140
Ile Ile Ala Glu Thr Ala Phe Ala Ala Ser Leu Gln Glu Ser Arg Pro
145 150 155 160
Ile Leu Thr Gly Val His Ile Val Leu Ser Asn His Lys Asp Phe Lys
                                165 170 175
Ala Val Ala Thr Asp Ser His Arg Met Ser Gln Arg Leu Ile Thr Leu
                        180 185 190
Asp Asn Thr Ser Ala Asp Leu Met Val Val Leu Pro Ser Lys Ser Leu
                195 200 205
Arg Glu Phe Ser Ala Val Phe Thr Asp Asp Ile Glu Thr Val Glu Val
        210 215 220
Phe Phe Ser Pro Ser Gln Ile Leu Phe Arg Ser Glu His Ile Ser Phe
225 230 235 240
Tyr Thr Arg Leu Leu Glu Gly Asn Tyr Pro Asp Thr Asp Arg Leu Leu
                                245 250 255
Met Thr Glu Phe Glu Thr Glu Val Val Phe Asn Thr Gln Ser Leu Arg
                        260 265 270
His Ala Met Glu Arg Ala Phe Leu Ile Ser Asn Ala Thr Gln Asn Gly
                275 280 285
Thr Val Lys Leu Glu Ile Thr Gln Asn His Ile Ser Ala His Val Asn
        290 295 300
Ser Pro Glu Val Gly Lys Val Asn Glu Asp Leu Asp Ile Val Ser Gln
305 310 315 320
Ser Gly Ser Asp Leu Thr Ile Ser Phe Asn Pro Thr Tyr Leu Ile Glu
                                325 330 335
Ser Leu Lys Ala Ile Lys Ser Glu Thr Val Lys Ile His Phe Leu Ser
                        340 345 350
Pro Val Arg Pro Phe Thr Leu Thr Pro Gly Asp Glu Glu Glu Ser Phe
                355 360 365
Ile Gln Leu Ile Thr Pro Val Arg Thr Asn
        370 375
```

The present invention also relates to the ssb gene of Streptococcus pyogenes encoding the single strand-binding protein (SSB). The ssb gene has a nucleotide sequence which corresponds to SEQ. ID. No. 29 as follows:

| | |
|---|---|
| atgattaata atgtagtact agttggtcgc atgaccaagg atgcagaact tcgttacaca | 60 |
| ccaagtcaag tagctgtggc taccttcaca cttgctgtta accgtacctt taaaagccaa | 120 |
| aatggtgaac gcgaggcaga tttcattaac tgtgtgatct ggcgtcaacc ggctgaaaat | 180 |
| ttagcgaact gggctaaaaa aggtgctttg atcggagtta cgggtcgtat tcatacacgt | 240 |
| aactacgaaa accaacaagg acaacgtgtc tatgtaacag aagttgttgc agataatttc | 300 |
| caaatgttgg aaagtcgtgc tacacgtgaa ggtggctcaa ctggctcatt taatggtggt | 360 |
| tttaacaata acacttcatc atcaaacagt tactcagcgc ctgcacaaca aacgcctaac | 420 |
| tttggaagag atgatagccc atttgggaac tcaaacccga tggatatctc agatgacgat | 480 |
| cttccattct ag | 492 |

The encoded SSB protein has an amino acid sequence corresponding to SEQ. ID. No. 30 as follows:

```
Met Ile Asn Asn Val Val Leu Val Gly Arg Met Thr Lys Asp Ala Glu
    1 5 10 15
Leu Arg Tyr Thr Pro Ser Gln Val Ala Val Ala Thr Phe Thr Leu Ala
                          20 25 30
Val Asn Arg Thr Phe Lys Ser Gln Asn Gly Glu Arg Glu Ala Asp Phe
                  35 40 45
Ile Asn Cys Val Ile Trp Arg Gln Pro Ala Glu Asn Leu Ala Asn Trp
          50 55 60
Ala Lys Lys Gly Ala Leu Ile Gly Val Thr Gly Arg Ile Gln Thr Arg
  65 70 75 80
Asn Tyr Glu Asn Gln Gln Gly Gln Arg Val Tyr Val Thr Glu Val Val
                                  85 90 95
Ala Asp Asn Phe Gln Met Leu Glu Ser Arg Ala Thr Arg Glu Gly Gly
                        100 105 110
Ser Thr Gly Ser Phe Asn Gly Gly Phe Asn Asn Asn Thr Ser Ser Ser
                115 120 125
Asn Ser Tyr Ser Ala Pro Ala Gln Gln Thr Pro Asn Phe Gly Arg Asp
        130 135 140
Asp Ser Pro Phe Gly Asn Ser Asn Pro Met Asp Ile Ser Asp Asp Asp
145 150 155 160
Leu Pro Phe
```

The present invention also relates to the dnaG gene of Streptococcus pyogenes encoding the primase. The dnaG gene has a nucleotide sequence which corresponds to SEQ. ID. No. 31 as follows:

| | |
|---|---|
| atgggatttt tatggggagg tgacgatttg gcaattgaca aagaaatgat ttcccaagta | 60 |
| aaaaatagcg ttaatattgt cgatgtcatt ggagaagtgg tcaaactttc ccgatcaggg | 120 |
| cggcattacc tcgggctttg cccatttcat aaggaaaaga caccctcttt taatgttgtt | 180 |
| gaagacagac aattttttca ctgctttggc tgtggaaaat caggggatgt ttttaaattt | 240 |
| attgaggaat accgccaagt ccccttctta gaaagtgttc agattattgc cgataagact | 300 |
| ggtatgtcgc ttaatatacc gccaagtcag gcagtacttg ctagccaaca caagcaccct | 360 |
| aatcacgctt tgatgacact tcatgaggat gctgctaaat tttaccatgc agttttgatg | 420 |
| accactacca ttggtcaaga agctaggaag tacctttacc agagaggctt ggatgaccaa | 480 |
| ttaattgagc atttcaatat tggtttagcc ccagatgagt cagattatct ttatcaagct | 540 |
| ctttctaaaa aatacgagga aggtcaattg gttgcttcag gattgtttca cttgtccgat | 600 |
| caatccaata ccatttacga cgcctttcga aatcgtatca tgtttccctt atcagatgac | 660 |
| cgagggcata ttattgcctt ttcaggacgt atctggacgg cagctgatat ggaaaagaga | 720 |
| caggcaaagt ataaaaattc aagaggaaca gttcttttta acaaatctta tgaattgtat | 780 |
| catctggaca aggcaaggcc tgttattgcc aaaacccatg aagtgtttct aatggaaggg | 840 |
| tttatggacg tgattgccgc ttaccgttcc ggctatgaaa atgctgttgc ttcaatgggg | 900 |
| acggcattga ctcaagaaca tgtcaatcac cttaagcaag tcactaaaaa agttgttttg | 960 |
| atttatgatg gtgacgatgc tggacaacat gctattgcaa aatcactaga attgcttaaa | 1020 |
| gattttgttg tcgaaattgt cagaatcccc aataaaatgg atcctgacga atttgtacaa | 1080 |
| cggcattccc cagaagcatt tgcagatttg cttaagcagt cacggatcag tagtgttgaa | 1140 |
| ttttttattg attacctaaa acctactaat gtagacaatt tgcaatcaca aattgtttat | 1200 |
| gtggagaaaa tggcaccatt gattgctcaa tcaccatcca tcacagctca acattcgtat | 1260 |
| attaacaaga ttgctgattt gttgccaaac tttgactatt ttcaagtaga acaatcagta | 1320 |
| aatgcattaa ggattcaaga taggcaaaaa catcaaggtc aaatagctca agccgtcagc | 1380 |
| aatcttgtga ccttaccaat gccaaaaagt ttgacagcta ttgctaagac agaaagtcat | 1440 |
| ctcatgcatc ggctcttaca tcatgactat ttattaaatg aatttcgaca tcgtgatgat | 1500 |
| ttttattttg atacctctac cttagaatta ctttatcaac ggctgaagca acaaggacac | 1560 |
| attacatctt atgatttgtc agagatgtca gaggaagtta accgtgctta ttacaatgtt | 1620 |
| ttagaagaaa accttcccaa agaagtagct cttggtgaga ttgatgatat tttatccaaa | 1680 |
| cgtgccaaac ttttagcaga gcgcgatctt cacaaacaag ggaaaaaagt tagagaatct | 1740 |
| agtaacaaag gcgatcatca agcggctcta gaagtactag aacattttat tgcgcagaaa | 1800 |
| cgaaaaatgg aatag | 1815 |

The encoded primase has an amino acid sequence corresponding to SEQ. ID. No. 32 as follows:

```
Met Gly Phe Leu Trp Gly Gly Asp Asp Leu Ala Ile Asp Lys Glu Met
    1 5 10 15
Ile Ser Gln Val Lys Asn Ser Val Asn Ile Val Asp Val Ile Gly Glu
                          20 25 30
Val Val Lys Leu Ser Arg Ser Gly Arg His Tyr Leu Gly Leu Cys Pro
                  35 40 45
Phe His Lys Glu Lys Thr Pro Ser Phe Asn Val Val Glu Asp Arg Gln
          50 55 60
Phe Phe His Cys Phe Gly Cys Gly Lys Ser Gly Asp Val Phe Lys Phe
  65 70 75 80
Ile Glu Glu Tyr Arg Gln Val Pro Phe Leu Glu Ser Val Gln Ile Ile
                                  85 90 95
Ala Asp Lys Thr Gly Met Ser Leu Asn Ile Pro Pro Ser Gln Ala Val
                        100 105 110
Leu Ala Ser Gln His Lys His Pro Asn His Ala Leu Met Thr Leu His
                115 120 125
Glu Asp Ala Ala Lys Phe Tyr His Ala Val Leu Met Thr Thr Thr Ile
        130 135 140
Gly Gln Glu Ala Arg Lys Tyr Leu Tyr Gln Arg Gly Leu Asp Asp Gln
145 150 155 160
Leu Ile Gln His Phe Asn Ile Gly Leu Ala Pro Asp Glu Ser Asp Tyr
                                165 170 175
Leu Tyr Gln Ala Leu Ser Lys Lys Tyr Glu Glu Gly Gln Leu Val Ala
                        180 185 190
Ser Gly Leu Phe His Leu Ser Asp Gln Ser Asn Thr Ile Tyr Asp Ala
                195 200 205
Phe Arg Asn Arg Ile Met Phe Pro Leu Ser Asp Asp Arg Gly His Ile
        210 215 220
Ile Ala Phe Ser Gly Arg Ile Trp Thr Ala Ala Asp Met Glu Lys Arg
225 230 235 240
Gln Ala Lys Tyr Lys Asn Ser Arg Gly Thr Val Leu Phe Asn Lys Ser
                                245 250 255
Tyr Glu Leu Tyr His Leu Asp Lys Ala Arg Pro Val Ile Ala Lys Thr
                        260 265 270
His Glu Val Phe Leu Met Glu Gly Phe Met Asp Val Ile Ala Ala Tyr
                275 280 285
Arg Ser Gly Tyr Glu Asn Ala Val Ala Ser Met Gly Thr Ala Leu Thr
        290 295 300
Gln Glu His Val Asn His Leu Lys Gln Val Thr Lys Lys Val Val Leu
305 310 315 320
Ile Tyr Asp Gly Asp Asp Ala Gly Gln His Ala Ile Ala Lys Ser Leu
                                325 330 335
Glu Leu Leu Lys Asp Phe Val Val Glu Ile Val Arg Ile Pro Asn Lys
                        340 345 350
Met Asp Pro Asp Glu Phe Val Gln Arg His Ser Pro Glu Ala Phe Ala
                355 360 365
Asp Leu Leu Lys Gln Ser Arg Ile Ser Ser Val Glu Phe Phe Ile Asp
        370 375 380
Tyr Leu Lys Pro Thr Asn Val Asp Asn Leu Gln Ser Gln Ile Val Tyr
385 390 395 400
Val Glu Lys Met Ala Pro Leu Ile Ala Gln Ser Pro Ser Ile Thr Ala
                                405 410 415
Gln His Ser Tyr Ile Asn Lys Ile Ala Asp Leu Leu Pro Asn Phe Asp
                        420 425 430
Tyr Phe Gln Val Glu Gln Ser Val Asn Ala Leu Arg Ile Gln Asp Arg
                435 440 445
Gln Lys His Gln Gly Gln Ile Ala Gln Ala Val Ser Asn Leu Val Thr
        450 455 460
Leu Pro Met Pro Lys Ser Leu Thr Ala Ile Ala Lys Thr Glu Ser His
465 470 475 480
Leu Met His Arg Leu Leu His His Asp Tyr Leu Leu Asn Glu Phe Arg
                                485 490 495
His Arg Asp Asp Phe Tyr Phe Asp Thr Ser Thr Leu Glu Leu Leu Tyr
                        500 505 510
Gln Arg Leu Lys Gln Gln Gly His Ile Thr Ser Tyr Asp Leu Ser Glu
                515 520 525
Met Ser Glu Glu Val Asn Arg Ala Tyr Tyr Asn Val Leu Glu Glu Asn
        530 535 540
Leu Pro Lys Glu Val Ala Leu Gly Glu Ile Asp Asp Ile Leu Ser Lys
545 550 555 560
Arg Ala Lys Leu Leu Ala Glu Arg Asp Leu His Lys Gln Gly Lys Lys
                                565 570 575
Val Arg Glu Ser Ser Asn Lys Gly Asp His Gln Ala Ala Leu Glu Val
                        580 585 590
Leu Glu His Phe Ile Ala Gln Lys
                595 600
```

The present invention also relates to the dnaB gene of Streptococcus pyogenes encoding DnaB. The dnaB gene has a nucleotide sequence which corresponds to SEQ. ID. No. 33 as follows:

| | |
|---|---|
| atgaggttgc ctgaagtagc tgaattacga gttcaacccc aagatttact agcagagcaa | 60 |
| tctgttcttg ggtcaatctt tatctcacct gataagctga ttgcagtgag agaatttatc | 120 |
| agtccagacg atttttataa gtacgctcat aaaattatct ttcgggcaat gattaccctc | 180 |
| agcgatcgta atgatgccat tgatgcaacc actataagaa caatcctaga tgatcaagat | 240 |
| gatctgcaaa gtattggtgg cttatcctat attgttgaac tagttaatag tgtcccaact | 300 |
| agtgctaatg cagaatatta tgctaaaatt gtagctgaga aagctatgtt gcgtgatatt | 360 |
| attgctaggt tgacagaatc tgtcaaccta gcttatgatg aaattttaaa accagaagag | 420 |
| gttatcgctg gagttgagag agctttaatt gaactcaatg aacatagtaa tcgtagtggg | 480 |
| tttcgcaaaa tttcagatgt gctaaaagtt aattacgagg ctttagaagc acgttctaag | 540 |
| cagacttcaa atgttacagg tttaccaact ggttttagag accttgacaa gattacaaca | 600 |
| ggtttacacc cagatcaatt agttatttta gctgctcggc cagcagtggg gaagactgcc | 660 |
| tttgttctta atattgcgca aaatgtgggg actaagcaaa aaaagactgt tgctattttt | 720 |
| tctttggaaa tgggtgctga aagtttagta gatcgtatgc ttgcagcaga aggaatggtt | 780 |
| gattcgcaca gtttaagaac agggcaactc acagatcagg attggaataa tgtaacaatt | 840 |
| gctcagggag ctttggcaga agcaccgatt tatattgacg atacgcccgg gattaaaatt | 900 |
| actgaaatcc gcgcaagatc acggaaattg tctcaagaag tggatggtgg tttaggtctc | 960 |
| attgtaattg actacttaca gttgattaca ggaactaaac ccgaaaatcg tcagcaagag | 1020 |
| gtttcagata tttcaagaca gcttaaaatc ctagctaaag aattgaaagt accagttatt | 1080 |
| gccctaagtc agctttctcg tggcgttgag caaaggcaag ataaacgacc agttttatca | 1140 |
| gatattcgtg aatcaggatc tattgagcag gatgccgata ttgtagcctt cttataccgg | 1200 |
| gacgattatt accgtaaaga atgtgatgat gctgaagaag ctgttgaaga taacacaatt | 1260 |
| gaagttatcc tcgagaaaaa tagagctggg gcgcgtggaa cagtcaaact gatgttccaa | 1320 |
| aaagaataca acaaattctc aagtatagcc cagtttgaag aaagataa | 1368 |

The encoded DnaB has an amino acid sequence corresponding to SEQ. ID. No. 34 as follows:

```
Met Arg Leu Pro Glu Val Ala Glu Leu Arg Val Gln Pro Gln Asp Leu
    1 5 10 15
Leu Ala Glu Gln Ser Val Leu Gly Ser Ile Phe Ile Ser Pro Asp Lys
                          20 25 30
Leu Ile Ala Val Arg Glu Phe Ile Ser Pro Asp Asp Phe Tyr Lys Tyr
                  35 40 45
Ala His Lys Ile Ile Phe Arg Ala Met Ile Thr Leu Ser Asp Arg Asn
          50 55 60
Asp Ala Ile Asp Ala Thr Thr Ile Arg Thr Ile Leu Asp Asp Gln Asp
  65 70 75 80
Asp Leu Gln Ser Ile Gly Gly Leu Ser Tyr Ile Val Glu Leu Val Asn
                                  85 90 95
Ser Val Pro Thr Ser Ala Asn Ala Glu Tyr Tyr Ala Lys Ile Val Ala
                        100 105 110
Gln Lys Ala Met Leu Arg Asp Ile Ile Ala Arg Leu Thr Glu Ser Val
                115 120 125
Asn Leu Ala Tyr Asp Glu Ile Leu Lys Pro Glu Glu Val Ile Ala Gly
        130 135 140
Val Glu Arg Ala Gln Gly Ala Leu Ala Glu Ala Pro Ile Tyr Ile Asp
145 150 155 160
Asp Thr Pro Gly Ile Lys Ile Ala Leu Ile Glu Leu Asn Glu His Ser
                                165 170 175
Asn Arg Ser Gly Phe Arg Lys Ile Ser Asp Val Leu Lys Val Asn Tyr
                        180 185 190
Glu Ala Leu Glu Ala Arg Ser Lys Gln Thr Ser Asn Val Thr Gly Leu
                195 200 205
Pro Thr Gly Phe Arg Asp Leu Asp Lys Ile Thr Thr Gly Leu His Pro
        210 215 220
Asp Gln Leu Val Ile Leu Ala Ala Arg Pro Ala Val Gly Lys Thr Ala
225 230 235 240
Phe Val Leu Asn Ile Ala Gln Asn Val Gly Thr Lys Gln Lys Lys Thr
                                245 250 255
Val Ala Ile Phe Ser Leu Glu Met Gly Ala Glu Ser Leu Val Asp Arg
                        260 265 270
Met Leu Ala Ala Glu Gly Met Val Asp Ser His Ser Leu Arg Thr Gly
                275 280 285
Gln Leu Thr Asp Gln Asp Trp Asn Asn Val Thr Ile Thr Glu Ile Arg
        290 295 300
Ala Arg Ser Arg Lys Leu Ser Gln Glu Val Asp Gly Gly Leu Gly Leu
305 310 315 320
Ile Val Ile Asp Tyr Leu Gln Leu Ile Thr Gly Thr Lys Pro Glu Asn
                                325 330 335
Arg Gln Gln Glu Val Ser Asp Ile Ser Arg Gln Leu Lys Ile Leu Ala
                        340 345 350
Lys Glu Leu Lys Val Pro Val Ile Ala Leu Ser Gln Leu Ser Arg Gly
                355 360 365
Val Glu Gln Arg Gln Asp Lys Arg Pro Val Leu Ser Asp Ile Arg Glu
        370 375 380
Ser Gly Ser Ile Glu Gln Asp Ala Asp Ile Val Ala Phe Leu Tyr Arg
385 390 395 400
Asp Asp Tyr Tyr Arg Lys Glu Cys Asp Asp Ala Glu Glu Ala Val Glu
                                405 410 415
Asp Asn Thr Ile Glu Val Ile Leu Glu Lys Asn Arg Ala Gly Ala Arg
                        420 425 430
Gly Thr Val Lys Leu Met Phe Gln Lys Glu Tyr Asn Lys Phe Ser Ser
                435 440 445
Ile Ala Gln Phe Glu Glu Arg
        450 455
```

Fragments of the above polypeptides or proteins are also encompassed by the present invention.

Suitable fragments can be produced by several means. In the first, subclones of the gene encoding the protein of the present invention are produced by conventional molecular genetic manipulation by subcloning gene fragments. The subclones then are expressed in vitro or in vivo in bacterial cells to yield a smaller protein or peptide that can be tested for activity according to the procedures described below.

As an alternative, fragments of replication proteins can be produced by digestion of a full-length replication protein with proteolytic enzymes like chymotrypsin or Staphylococcus proteinase A, or trypsin. Different proteolytic enzymes are likely to cleave replication proteins at different sites based on the amino acid sequence of the protein. Some of the fragments that result from proteolysis may be active and can be tested for activity as described below.

In another approach, based on knowledge of the primary structure of the protein, fragments of a replication protein gene may be synthesized by using the PCR technique together with specific sets of primers chosen to represent particular portions of the protein. These then would be cloned into an appropriate vector for increased expression of a truncated peptide or protein.

Chemical synthesis can also be used to make suitable fragments. Such a synthesis is carried out using known amino acid sequences of replication proteins being produced. Alternatively, subjecting a full length replication protein to high temperatures and pressures will produce fragments. These fragments can then be separated by conventional procedures (e.g., chromatography, SDS-PAGE).

Variants may also (or alternatively) be modified by, for example, the deletion or addition of amino acids that have minimal influence on the properties, secondary structure, and hydropathic nature of the polypeptide. For example, a polypeptide may be conjugated to a signal (or leader) sequence at the N-terminal end of the protein which cotranslationally or post-translationally directs transfer of the protein. The polypeptide may also be conjugated to a linker or other sequence for ease of synthesis, purification, or identification of the polypeptide.

Suitable DNA molecules are those that hybridize to a DNA molecule comprising a nucleotide sequence of at least about 20, more preferably at least about 30 to about 50, continuous bases of either SEQ. ID. Nos. 1, 3, 5, 7, 9, 11, 13, 15, 17, 19, 21, 23, 25, 27, 29, 31, or 33 under stringent conditions such as those characterized by a hybridization buffer comprising 0.9M sodium citrate (“SSC”) buffer at a temperature of about 37° C. and remaining bound when subject to washing the SSC buffer at a temperature of about 37° C.; and preferably in a hybridization buffer comprising 20% formamide in 0.9M SSC buffer at a temperature of about 42° C. and remaining bound when subject to washing at about 42° C. with 0.2×SSC buffer. Stringency conditions can be further varied by modifying the temperature and/or salt content of the buffer, or by modifying the length of the hybridization probe.

The proteins or polypeptides of the present invention are preferably produced in purified form (preferably at least 80%, more preferably 90%, pure) by conventional techniques. Typically, the proteins or polypeptides of the present invention is secreted into the growth medium of recombinant host cells. Alternatively, the proteins or polypeptides of the present invention are produced but not secreted into growth medium. In such cases, to isolate the protein, the host cell (e.g., E. coli) carrying a recombinant plasmid is propagated, lysed by sonication, heat, or chemical treatment, and the homogenate is centrifuged to remove bacterial debris. The supernatant is then subjected to purification procedures such as ammonium sulfate precipitation, gel filtration, ion exchange chromatography, FPLC, and HPLC.

The DNA molecule encoding replication polypeptides or proteins derived from Gram positive bacteria can be incorporated in cells using conventional recombinant DNA technology. Generally, this involved inserting the DNA molecule into an expression system to which the DNA molecule is heterologous (i.e. not normally present). The heterologous DNA molecule is inserted into the expression system or vector in proper sense orientation and correct reading frame. The vector contains the necessary elements for the transcription and translation of the inserted protein-coding sequences.

U.S. Pat. No. 4,237,224 to Cohen and Boyer, which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

Recombinant genes may also be introduced into viruses, such as vaccina virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC11, SV 40, pBluescript II SK +/− or KS +/− (see “Stratagene Cloning Systems” Catalog (1993) from Stratagene, La Jolla, Calif., which is hereby incorporated by reference), pQE, pIH821, pGEX, pET series (see F. W. Studier et al., “Use of T7 RNA Polymerase to Direct Expression of Cloned Genes,” Gene Expression Technology vol. 185 (1990), which is hereby incorporated by reference), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eucaryotic promoters differ from those of procaryotic promoters. Furthermore, eucaryotic promoters and accompanying genetic signals may not be recognized in or may not function in a procaryotic system, and, further procaryotic promoters are not recognized and do not function in eucaryotic cells.

Similarly, translation of mRNA in procaryotes depends upon the presence of the proper procaryotic signals which differ from those of eukaryotes. Efficient translation of mRNA in procaryotes requires a ribosome binding site called the Shine-Dalgarno (“SD”) sequence on the mRNA. This sequence is a short nucleotide sequence of mRNA that is located before the same codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference.

Promoters vary in their “strength” (i.e. their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promotor, trp promotor, recA promotor, ribosomal RNA promotor, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promotor or other E. coli promoters produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls. Additionally, the cell may carry the gene for a heterologous RNA polymerase such as from phage T7. Thus, a promoter specific for T7 RNA polymerase is used. The T7 RNA polymerase may be under inducible control.

Specific initiation signals are also required for efficient gene transcription and translation in procaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, an SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Once the isolated DNA molecule encoding a replication polypeptide or protein has been cloned into an expression system, it is ready to be incorporated into a host cell. Such incorporation can be carried out by the various forms of transformation noted above, depending upon the vector/host cell system. Suitable host cells include, but are not limited to, bacteria, viruses, yeast, mammalian cells, insects, plants, and the like.

The invention provides efficient methods of identifying pharmacological agents or lead compounds for agents active at the level of a replication protein function, particularly DNA replication. Generally, these screening methods involve assaying for compounds which interfere with the replication activity. The methods are amenable to automated, cost-effective high throughput screening of chemical libraries for lead compounds. Identified reagents find use in the pharmaceutical industries for animal and human trials; for example, the reagents may be derivatized and rescreened in in vitro and in vivo assays to optimize activity and minimize toxicity for pharmaceutical development. Target therapeutic indications are limited only in that the target cellular function be subject to modulation, usually inhibition, by disruption of a replication activity or the formation of a complex comprising a replication protein and one or more natural intracellular binding targets. Target indications may include arresting cell growth or causing cell death resulting in recovery from the bacterial infection in animal studies.

A wide variety of assays for activity and binding agents are provided, including DNA synthesis, ATPase, clamp loading onto DNA, protein-protein binding assays, immunoassays, cell based assays, etc. The replication protein compositions, used to identify pharmacological agents, are in isolated, partially pure or pure form and are typically recombinantly produced. The replication protein may be part of a fusion product with another peptide or polypeptide (e.g., a polypeptide that is capable of providing or enhancing protein-protein binding, stability under assay conditions (e.g., a tag for detection or anchoring), etc.). The assay mixtures comprise a natural intracellular replication protein binding target such as DNA, another protein, NTP, or dNTP. For binding assays, while native binding targets may be used, it is frequently preferred to use portions (e.g., peptides, nucleic acid fragments) thereof so long as the portion provides binding affinity and avidity to the subject replication protein conveniently measurable in the assay. The assay mixture also comprises a candidate pharmacological agent. Generally, a plurality of assay mixtures are run in parallel with different agent concentrations to obtain a differential response to the various concentrations. Typically, one of these concentrations serves as a negative control (i.e., at zero concentration or below the limits of assay detection). Additional controls are often present such as a positive control, a dose response curve, use of known inhibitors, use of control heterologous proteins, etc. Candidate agents encompass numerous chemical classes, though typically they are organic compounds; preferably they are small organic compounds and are obtained from a wide variety of sources, including libraries of synthetic or natural compounds. A variety of other reagents may also be included in the mixture. These include reagents like salts, buffers, neutral proteins (e.g., albumin, detergents, etc.), which may be used to facilitate optimal binding and/or reduce nonspecific or background interactions, etc. Also reagents that otherwise improve the efficiency of the assay (e.g., protease inhibitors, nuclease inhibitors, antimicrobial agents, etc.) may be used.

The invention provides replication protein specific assays and the binding agents including natural intracellular binding targets such as other replication proteins, etc., and methods of identifying and making such agents, and their use in a variety of diagnostic and therapeutic applications, especially where disease is associated with excessive cell growth. Novel replication protein-specific binding agents include replication protein-specific antibodies and other natural intracellular binding agents identified with assays such as one- and two-hybrid screens, non-natural intracellular binding agents identified in screens of chemical libraries, etc.

Generally, replication protein-specificity of the binding agent is shown by binding equilibrium constants. Such agents are capable of selectively binding a replication protein (i.e., with an equilibrium constant at least about 10⁷ M⁻¹, preferably, at least about 10⁸ M⁻¹, more preferably, at least about 10⁹ M⁻¹). A wide variety of cell-based and cell-free assays may be used to demonstrate replication protein-specific activity, binding, gel shift assays, immunoassays, etc.

The resultant mixture is incubated under conditions whereby, but for the presence of the candidate pharmacological agent, the replication protein specifically binds the cellular binding target, portion, or analog. The mixture of components can be added in any order that provides for the requisite bindings. Incubations may be performed at any temperature which facilitates optimal binding, typically between 4° C. and 40° C., more commonly between 15° C. and 40° C. Incubation periods are likewise selected for optimal binding but also minimized to facilitate rapid, high-throughput screening, and are typically between 0.1 and 10 hours, preferably less than 5 hours, more preferably less than 2 hours.

After incubation, the presence or absence of activity or specific binding between the replication protein and one or more binding targets is detected by any convenient way. For cell-free activity and binding type assays, a separation step may be used to separate the activity product or the bound from unbound components. Separation may be effected by precipitation (e.g., immunoprecipitation), immobilization (e.g., on a solid substrate such as a microtiter plate), etc., followed by washing. Many assays that do not require separation are also possible such as use of europium conjugation in proximity assays or a detection system that is dependent on a product or loss of substrate.

Detection may be effected in any convenient way. For cell-free activity and binding assays, one of the components usually comprises or is coupled to a label. A wide variety of labels may be employed essentially any label that provides for detection of DNA product, loss of DNA substrate, conversion of a nucleotide substrate, or bound protein is useful. The label may provide for direct detection such as radioactivity, fluorescence, luminescence, optical, or electron density, etc. or indirect detection such as an epitope tag, an enzyme, etc. The label may be appended to the protein (e.g., a phosphate group comprising a radioactive isotope of phosphorous), or incorporated into the DNA substrate or the protein structure (e.g., a methionine residue comprising a radioactive isotope of sulfur.) A variety of methods may be used to detect the label depending on the nature of the label and other assay components. For example, the label may be detected bound to the solid substrate, or a portion of the bound complex containing the label may be separated from the solid substrate, and thereafter the label detected. Labels may be directly detected through optical or electron density, radioactive emissions, nonradiative energy transfer, fluorescence emission, etc. or indirectly detected with antibody conjugates, etc. For example, in the case of radioactive labels, emissions may be detected directly (e.g., with particle counters) or indirectly (e.g., with scintillation cocktails and counters).

The present invention identifies the set of proteins that together result in a three component polymerase from bacteria that are distantly related to E. coli such as Gram positive bacteria. Specifically, these bacteria lack several genes that E. coli DNA polymerase III has, such as holD, holD or holE. Further, dnaX is believed to encode only one protein, tau. Also, holA is quite divergent in homology suggesting it may function in another process in these organisms. Gram positive cells even have replication genes that E. coli does not, implying that they may not utilize the replication strategies exemplified by E. coli.

The present invention identifies genes and proteins that form a three component polymerase in Gram positive organisms, such as S. pyogenes and S. aureus. In S. pyogenes and S. aureus, the polymerase α-large, functions with a β clamp and a clamp loader component of τδδ′. They display high speed and processivity in synthesis of ssDNA coated with SSB and primed with a DNA oligonucleotide.

This invention also expresses and purifies a protein from a Gram positive bacteria that is homologous to the E. coli beta subunit. The invention demonstrates that it behaves like a circular protein. Further, this invention shows that a beta subunit from a Gram positive bacteria is functional with both Pol III-L (α-large) from a Gram positive bacteria and with DNA polymerase III from a Gram negative bacteria. This result can be explained by an interaction between the clamp and the polymerase that has been conserved during the evolutionary divergence of Gram positive and Gram negative cells. A chemical inhibitor that would disrupt this interaction would be predicted to have a broad spectrum of antibiotic activity, shutting down replication in gram negative and gram positive cells alike. This assay, and others based on this interaction, can be devised to screen chemicals for such inhibition. Further, since all the proteins in this assay are highly overexpressed through recombinant techniques, sufficient quantities of the protein reagents can be obtained for screening hundreds of thousands of compounds.

This invention also shows that the DnaE polymerase (α-small), encoded by the dnaE gene, functions with the beta clamp and τδδ′ complex. The speed of DnaE is not significantly increased by τδδ′ and β, but the processivity of DnaE is greatly increased by τδδ′ and β. Hence, the DnaE polymerase, coupled with its β clamp on DNA (loaded by τδδ′) may also be an important target for a candidate pharmaceutical drug.

The present invention provides methods by which replication proteins from a Gram positive bacteria are used to discover new pharmaceutical agents. The function of replication proteins is quantified in the presence of different chemical compounds. A chemical compound that inhibits the function is a candidate antibiotic. Some replication proteins from a Gram positive bacteria and from a Gram negative bacteria can be interchanged for one another. Hence, they can function as mixtures. Reactions that assay for the function of enzyme mixtures consisting of proteins from Gram positive bacteria and from Gram negative bacteria can also be used to discover drugs. Suitable E. coli replication proteins are the subunits of its Pol III holoenzyme which are described in U.S. Pat. Nos. 5,583,026 and 5,668,004 to O'Donnell, which are hereby incorporated by reference.

The methods described herein to obtain genes, and the assays demonstrating activity behavior of S. pyogenes and S. aureus replication proteins are likely to generalize to all members of the Streptococcus and Staphylococcus genuses, as well as to all Gram positive bacteria. Such assays are also likely to generalize to other cells besides Gram positive bacteria which also share features in common with S. pyogenes and S. aureus that are different from E. coli (i.e., lacking holC, holD, or holE; having a dnaX gene encoding a single protein; or having a weak homology to holA encoding delta).

The present invention describes a method of identifying compounds which inhibit the activity of a polymerase product of polC or dnaE. This method is carried out by forming a reaction mixture that includes a primed DNA molecule, a polymerase product of polC or dnaE, a candidate compound, a dNTP, and optionally either a beta subunit, a tau complex, or both the beta subunit and the tau complex, wherein at least one of the polymerase product of polC or dnaE, the beta subunit, the tau complex, or a subunit or combination of subunits thereof is derived from a Eubacteria other than Escherichia coli; subjecting the reaction mixture to conditions effective to achieve nucleic acid polymerization in the absence of the candidate compound; analyzing the reaction mixture for the presence or absence of nucleic acid polymerization extension products; and identifying the candidate compound in the reaction mixture where there is an absence of nucleic acid polymerization extension products. Preferably, the polymerase product of polC or dnaE, the beta subunit, the tau complex, or the subunit or combination of subunits thereof is derived from a Gram positive bacterium, more preferably a Streptococcus bacterium such as S. pyogenes or a Staphylococcus bacterium such as S. aureus.

The present invention describes a method to identify chemicals that inhibit the activity of the three component polymerase. This method involves contacting primed DNA with the DNA polymerase in the presence of the candidate pharmaceutical, and dNTPs (or modified dNTPs) to form a reaction mixture. The reaction mixture is subjected to conditions effective to achieve nucleic acid polymerization in the absence of the candidate pharmaceutical and the presence or absence of the extension product in the reaction mixture is analyzed. The candidate pharmaceutical is detected by the absence of product.

The present invention describes a method to identify candidate pharmaceuticals that inhibit the activity of a clamp loader complex and a beta subunit in stimulating the DNA polymerase. The method includes contacting a primed DNA (which may be coated with SSB) with a DNA polymerase, a beta subunit, and a tau complex (or subunit or subassembly of the tau complex) in the presence of the candidate pharmaceutical, and dNTPs (or modified dNTPs) to form a reaction mixture. The reaction mixture is subjected to conditions which, in the absence of the candidate pharmaceutical, would effect nucleic acid polymerization and the presence or absence of the extension product in the reaction mixture is analyzed. The candidate pharmaceutical is detected by the absence of product. The DNA polymerase, the beta subunit, and/or the tau complex or subunit(s) thereof are derived from a Gram positive bacterium.

The present invention describes a method to identify chemicals that inhibit the ability of a beta subunit and a DNA polymerase to interact physically. This method involves contacting the beta subunit with the DNA polymerase in the presence of the candidate pharmaceutical to form a reaction mixture. The reaction mixture is subjected to conditions under which the DNA polymerase and the beta subunit would interact in the absence of the candidate pharmaceutical. The reaction mixture is then analyzed for interaction between the beta unit and the DNA polymerase. The candidate pharmaceutical is detected by the absence of interaction between the beta subunit and the DNA polymerase. The DNA polymerase and/or the beta subunit are derived from a Gram positive bacterium.

The present invention describes a method to identify chemicals that inhibit the ability of a beta subunit and a tau complex (or a subunit or subassembly of the tau complex) to interact. This method includes contacting the beta subunit with the tau complex (or subunit or subassembly of the tau complex) in the presence of the candidate pharmaceutical to form a reaction mixture. The reaction mixture is subjected to conditions under which the tau complex (or the subunit or subassembly of the tau complex) and the beta subunit would interact in the absence of the candidate pharmaceutical. The reaction mixture is then analyzed for interaction between the beta subunit and the tau complex (or the subunit or subassembly of the tau complex). The candidate pharmaceutical is detected by the absence of interaction between the beta subunit and the tau complex (or the subunit or subassembly of the tau complex). The beta subunit and/or the tau complex or subunit thereof is derived from a Gram positive bacterium.

The present invention describes a method to identify chemicals that inhibit the ability of a tau complex (or a subassembly of the tau complex) to assemble a beta subunit onto a DNA molecule. This method involves contacting a circular primed DNA molecule (which may be coated with SSB) with the tau complex (or the subassembly thereof) and the beta subunit in the presence of the candidate pharmaceutical, and ATP or dATP to form a reaction mixture. The reaction mixture is subjected to conditions under which the tau complex (or subassembly) assembles the beta subunit on the DNA molecule absent the candidate pharmaceutical. The presence or absence of the beta subunit on the DNA molecule in the reaction mixture is analyzed. The candidate pharmaceutical is detected by the absence of the beta subunit on the DNA molecule. The beta subunit and/or the tau complex are derived from a Gram positive bacterium.

The present invention describes a method to identify chemicals that inhibit the ability of a tau complex (or a subunit(s) of the tau complex) to disassemble a beta subunit from a DNA molecule. This method comprises contacting a DNA molecule onto which the beta subunit has been assembled in the presence of the candidate pharmaceutical, to form a reaction mixture. The reaction mixture is subjected to conditions under which the tau complex (or a subunit(s) or subassembly of the tau complex) disassembles the beta subunit from the DNA molecule absent the candidate pharmaceutical. The presence or absence of the beta subunit on the DNA molecule in the reaction mixture is analyzed. The candidate pharmaceutical is detected by the presence of the beta subunit on the DNA molecule. The beta subunit and/or the tau complex are derived from a Gram positive bacterium.

The present invention describes a method to identify chemicals that disassemble a beta subunit from a DNA molecule. This method involves contacting a circular primed DNA molecule (which may be coated with SSB) upon which the beta subunit has been assembled (e.g. by action of the tau complex) with the candidate pharmaceutical. The presence or absence of the beta subunit on the DNA molecule in the reaction mixture is analyzed. The candidate pharmaceutical is detected by the absence of the beta subunit on the DNA molecule. The beta subunit is derived from a Gram positive bacterium.

The present invention describes a method to identify chemicals that inhibit the dATP/ATP binding activity of a tau complex or a tau complex subunit (e.g. tau subunit). This method includes contacting the tau complex (or the tau complex subunit) with dATP/ATP either in the presence or absence of a DNA molecule and/or the beta subunit in the presence of the candidate pharmaceutical to form a reaction. The reaction mixture is subjected to conditions in which the tau complex (or the subunit of tau complex) interacts with dATP/ATP in the absence of the candidate pharmaceutical. The reaction is analyzed to determine if dATP/ATP is bound to the tau complex (or the subunit of tau complex) in the presence of the candidate pharmaceutical. The candidate pharmaceutical is detected by the absence of hydrolysis. The tau complex and/or the beta subunit is derived from a Gram positive bacterium.

The present invention describes a method to identify chemicals that inhibit the dATP/ATPase activity of a tau complex or a tau complex subunit (e.g., the tau subunit). This method involves contacting the tau complex (or the tau complex subunit) with dATP/ATP either in the presence or absence of a DNA molecule and/or a beta subunit in the presence of the candidate pharmaceutical to form a reaction mixture. The reaction mixture is subjected to conditions in which the tau subunit (or complex) hydrolyzes dATP/ATP in the absence of the candidate pharmaceutical. The reaction is analyzed to determine if dATP/ATP was hydrolyzed. Suitable candidate pharmaceuticals are identified by the absence of hydrolysis. The tau complex and/or the beta subunit is derived from a Gram positive bacterium.

Further methods for identifying chemicals that inhibit the activity of a DNA polymerase encoded by either the dnaE gene, polC gene, or their accessory proteins (i.e., clamp loader, clamp, etc.), are as follows:

1) Contacting a primed DNA molecule with the encoded product of the dnaE gene or polC gene in the presence of the candidate pharmaceutical, and dNTPs (or modified dNTPs) to form a reaction mixture. The reaction mixture is subjected to conditions, which in the absence of the candidate pharmaceutical, affect nucleic acid polymerization and the presence or absence of the extension product in the reaction mixture is analyzed. The candidate pharmaceutical is detected by the absence of extension product. The protein encoded by the dnaE gene and PolC gene is derived from a Gram positive bacterium.

2) Contacting a linear primed DNA molecule with a beta subunit and the encoded product of dnaE or PolC in the presence of the candidate pharmaceutical, and dNTPs (or modified dNTPs) to form a reaction mixture. The reaction mixture is subjected to conditions, which in the absence of the candidate pharmaceutical, affect nucleic acid polymerization, and the presence or absence of the extension product in the reaction mixture is analyzed. The candidate pharmaceutical is detected by the absence of extension product. The protein encoded by the dnaE gene and PolC gene is derived from a Gram positive bacterium.

3) Contacting a circular primed DNA molecule (may be coated with SSB) with a tau complex, a beta subunit and the encoded product of a dnaE gene or PolC gene in the presence of the candidate pharmaceutical, and dNTPs (or modified dNTPs) to form a reaction mixture. The reaction mixture is subjected to conditions, which in the absence of the candidate pharmaceutical, affect nucleic acid polymerization, and the presence or absence of the extension product in the reaction mixture is analyzed. The candidate pharmaceutical is detected by the absence of product. The protein encoded by the dnaE gene and PolC gene, the beta subunit, and/or the tau complex are derived from a Gram positive bacterium.

4) Contacting a beta subunit with the product encoded by a dnaE gene or PolC gene in the presence of the candidate pharmaceutical to form a reaction mixture. The reaction mixture is then analyzed for interaction between the beta subunit and the product encoded by the dnaE gene or PolC gene. The candidate pharmaceutical is detected by the absence of interaction between the beta subunit and the product encoded by the dnaE gene or PolC gene. The beta subunit and/or the protein encoded by the dnaE gene and PolC gene is derived from a Gram positive bacterium.

5) The present invention discloses a method to identify chemicals that inhibit a DnaB helicase. The method includes contacting the DnaB helicase with a DNA molecule substrate that has a duplex region in the presence of a nucleoside or deoxynucleoside triphosphate energy source and a candidate pharmaceutical to form a reaction mixture. The reaction mixture is subjected to conditions that support helicase activity in the absence of the candidate pharmaceutical. The DNA duplex molecule in the reaction mixture is analyzed for whether it is converted to ssDNA. The candidate pharmaceutical is detected by the absence of conversion of the duplex DNA molecule to the ssDNA molecule. The DnaB helicase is derived from a Gram positive bacterium.

6) The present invention describes a method to identify chemicals that inhibit the nucleoside or deoxynucleoside triphosphatase activity of a DnaB helicase. The method includes contacting the DnaB helicase with a DNA molecule substrate that has a duplex region in the presence of a nucleoside or deoxynucleoside triphosphate energy source and the candidate pharmaceutical to form a reaction mixture. The reaction mixture is subjected to conditions that support nucleoside or deoxynucleoside triphosphatase activity of the DnaB helicase in the absence of the candidate pharmaceutical. The candidate pharmaceutical is detected by the absence of conversion of nucleoside or deoxynucleoside triphosphate to nucleoside or deoxynucleoside diphosphate. The DnaB helicase is derived from a Gram positive bacterium.

7) The present invention describes a method to identify chemicals that inhibit a primase. The method includes contacting primase with a ssDNA molecule in the presence of a candidate pharmaceutical to form a reaction mixture. The reaction mixture is subjected to conditions that support primase activity (e.g., the presence of nucleoside or deoxynucleoside triphosphates, appropriate buffer, presence or absence of DnaB helicase) in the absence of the candidate pharmaceutical. Suitable candidate pharmaceuticals are identified by the absence of primer formation detected either directly or indirectly. The primase is derived from a Gram positive bacterium.

8) The present invention describes a method to identify chemicals that inhibit the ability of a primase and the protein encoded by a dnaB gene to interact. This method includes contacting the primase with the protein encoded by the dnaB gene in the presence of the candidate pharmaceutical to form a reaction mixture. The reaction mixture is subjected to conditions under which the primase and the protein encoded by the dnaB gene interact in the absence of the candidate pharmaceutical. The reaction mixture is then analyzed for interaction between the primase and the protein encoded by the dnaB gene. The candidate pharmaceutical is detected by the absence of interaction between the primase and the protein encoded by the dnaB gene. The primase and/or the dnaB gene are derived from a Gram positive bacterium.

9) The present invention describes a method to identify chemicals that inhibit the ability of a protein encoded by a dnaB gene to interact with a DNA molecule. This method includes contacting the protein encoded by the dnaB gene with the DNA molecule in the presence of the candidate pharmaceutical to form a reaction mixture. The reaction mixture is subjected to conditions under which the DNA molecule and the protein encoded by the dnaB gene interact in the absence of the candidate pharmaceutical. The reaction mixture is then analyzed for interaction between the protein encoded by the dnaB gene and the DNA molecule. The candidate pharmaceutical is detected by the absence of interaction between the DNA molecule and the protein encoded by the dnaB gene. The dnaB gene is derived from a Gram positive bacterium.

### EXAMPLES

The following examples are provided to illustrate embodiments of the present invention, but they are by no means intended to limit its scope.

### Example 1

### Materials

Labeled deoxy- and ribonucleoside triphosphates were from Dupont-New England Nuclear; unlabelled deoxy- and ribonucleoside triphosphates were from Pharmacia-LKB; E. coli replication proteins were purified as described, alpha, epsilon, gamma, and tau (Studwell et al., “Processive Replication is Contingent on the Exonuclease Subunit of DNA Polymerase III Holoenzyme,” J. Biol. Chem., 265:1171-1178 (1990), which is hereby incorporated by reference), beta (Kong et al., “Three Dimensional Structure of the Beta Subunit of Escherichia coli DNA Polymerase III Holoenzyme: A Sliding DNA Clamp,” Cell, 69:425-437 (1992), which is hereby incorporated by reference), delta and delta prime (Dong et al., “DNA Polymerase III Accessory Proteins. I. HolA and holB Encoding δ and δ′,” J. Biol. Chem., 268:11758-11765 (1993), which is hereby incorporated by reference), chi and psi (Xiao et al., “DNA Polymerase III Accessory Proteins. III. HolC and holD Encoding chi and psi,” J. Biol. Chem., 268:11773-11778 (1993), which is hereby incorporated by reference), theta (Studwell-Vaughan et al., “DNA Polymerase III Accessory Proteins. V. Theta Encoded by holE,” J. Biol. Chem., 268:11785-11791 (1993), which is hereby incorporated by reference), and SSB (Weiner et al., “The Deoxyribonucleic Acid Unwinding Protein of Escherichia coli,” J. Biol. Chem., 250:1972-1980 (1975), which is hereby incorporated by reference). E. coli Pol III core and clamp loader complex (composed of subunits gamma, delta, delta prime, chi, and psi) were reconstituted as described in Onrust et al., “Assembly of a Chromosomal Replication Machine: Two DNA Polymerases, a Clamp Loader and Sliding Clamps in One Holoenzyme Particle. I. Organization of the Clamp Loader,” J. Biol. Chem., 270:13348-13357 (1995), which is hereby incorporated by reference. Pol III* was reconstituted and purified as described in Onrust et al., “Assembly of a Chromosomal Replication Machine: Two DNA Polymerases, a Clamp Loader and Sliding Clamps in One Holoenzyme Particle. III. Interface Between Two Polymerases and the Clamp Loader,” J. Biol. Chem., 270:13366-13377 (1995), which is hereby incorporated by reference. Protein concentrations were quantitated by the Protein Assay (Bio-Rad) method using bovine serum albumin (BSA) as a standard. DNA oligonucleotides were synthesized by Oligos etc. Calf thymus DNA was from Sigma. Buffer A is 20 mM Tris-HCl (pH=7.5), 0.5 mM EDTA, 2 mM DTT, and 20% glycerol. Replication buffer is 20 mM Tris-Cl (pH 7.5), 8 mM MgCl₂, 5 mM DTT, 0.5 mM EDTA, 40 μg/ml BSA, 4% glycerol, 0.5 mM ATP, 3 mM each dCTP, dGTP, dATP, and 20 μM [α-³²P]dTTP. P-cell buffer is 50 mM potassium phosphate (pH 7.6), 5 mM DTT, 0.3 mM EDTA, 20% glycerol. T.E. buffer is 10 mM Tris-HCl (pH 7.5), 1 mM EDTA. Cell lysis buffer is 50 mM Tris-HCl (pH 8.0) 10% sucrose, 1 M NaCl, 0.3 mM spermidine.

### Example 2

### Calf Thymus DNA Replication Assays

These assays were used in the purification of DNA polymerases from S. aureus cell extracts. Assays contained 2.5 μg activated calf thymus DNA in a final volume of 25 μl replication buffer. An aliquot of the fraction to be assayed was added to the assay mixture on ice followed by incubation at 37° C. for 5 min. DNA synthesis was quantitated using DE81 paper as described in Rowen et al., “Primase, the DnaG Protein of Escherichia coli. An Enzyme Which Starts DNA Chains,” J. Biol. Chem., 253:758-764 (1979), which is hereby incorporated by reference.

### Example 3

### PolydA-oligodT Replication Assays

PolydA-oligodT was prepared as follows. PolydA of average length 4500 nucleotides was purchased from SuperTecs. OligodT35 was synthesized by Oligos etc. 145 ul of 5.2 mM (as nucleotide) polydA and 22 μl of 1.75 mM (as nucleotide) oligodT were mixed in a final volume of 2100 μl T.E. buffer (ratio as nucleotide was 21:1 polydA to oligodT). The mixture was heated to boiling in a 1 ml eppendorf tube, then removed and allowed to cool to room temperature. Assays were performed in a final volume of 25 μl 20 mM Tris-Cl (pH 7.5), 8 mM MgCl₂, 5 mM DTT, 0.5 mM EDTA, 40 μg/ml BSA, 4% glycerol, containing 20 μM [α-³²P]dTTP and 0.36 μg polydA-oligodT. Proteins were added to the reaction on ice, then shifted to 37° C. for 5 min. DNA synthesis was quantitated using DE81 paper as described in Rowen et al., “Primase, the DnaG Protein of Escherichia coli. An Enzyme Which Starts DNA Chains,” J. Biol. Chem., 253:758-764 (1979), which is hereby incorporated by reference.

### Example 4

### Singly Primed M13mp18 ssDNA Replication Assays

M13mp18 was phenol extracted from phage and purified by two successive bandings (one downward and one upward) in cesium chloride gradients. M13mp18 ssDNA was singly primed with a DNA 30mer (map position 6817-6846) as described in Studwell et al. “Processive Replication is Contingent on the Exonuclease Subunit of DNA Polymerase III Holoenzyme,” J. Biol. Chem., 265:1171-1178 (1990), which is hereby incorporated by reference. Replication assays contained 72 ng of singly primed M13mp18 ssDNA in a final volume of 25 μl of replication buffer. Other proteins added to the assay, and their amounts, are indicated in the Brief Description of the Drawings. Reactions were incubated for 5 min. at 37° C. and then were quenched upon adding an equal volume of 1% SDS and 40 mM EDTA. DNA synthesis was quantitated using DE81 paper as described in Rowen et al., “Primase, the DnaG Protein of Escherichia coli. An Enzyme Which Starts DNA Chains,” J. Biol. Chem., 253:758-764 (1979), which is hereby incorporated by reference, and product analysis was performed in a 0.8% native agarose gel followed by autoradiography.

### Example 5

### Genomic Staphylococcus aureus DNA

Two strains of S. aureus were used. For PCR of the first fragment of the dnaX gene sequence, the strain was ATCC 25923. For all other work the strain was strain 4220 (a gift of Dr. Pat Schlievert, University of Minnesota). This strain lacks a gene needed for producing toxic shock (Kreiswirth et al., “The Toxic Shock Syndrome Exotoxin Structural Gene is Not Detectably Transmitted by a Prophage,” Nature, 305:709-712 (1996) and Balan et al., “Autocrine Regulation of Toxin Synthesis by Staphylococcus aureus,” Proc. Natl. Acad. Sci. USA, 92:1619-1623 (1995), which are hereby incorporated by reference). S. aureus cells were grown overnight at 37° C. in LB containing 0.5% glucose. Cells were collected by centrifugation (24 g wet weight). Cells were resuspended in 80 ml solution I (50 mM glucose, 10 mM EDTA, 25 mM Tris-HCL (pH 8.0)). SDS and NaOH were then added to 1% and 0.2 N, respectively, followed by incubation at 65° C. for 30 min. to lyse the cells. 68.5 ml of 3 M sodium acetate (pH 5.0) was added followed by centrifugation at 12,000 rpm for 30 min. The supernatant was discarded and the pellet was washed twice with 50 ml of 6M urea, 10 mM Tris-HCL (pH 7.5), 1 mM EDTA using a dounce homogenizer. After each wash, the resuspended pellet was collected by centrifugation (12,000 rpm for 20 min.). After the second wash, the pellet was resuspended in 50 ml 10 mM T.E. buffer using a dounce homogenizer and then incubated for 30 min. at 65° C. The solution was centrifuged at 12,000 rpm for 20 min., and the viscous supernatant was collected. 43.46 g CsCl₂ was added to the 50 ml of supernatant (density between 1.395-1.398) and poured into two 35 ml quick seal ultracentrifuge tubes (tubes were completely filled using the same density of CsCl₂ in T.E.). To each tube was added 0.5 ml of a 10 mg/ml stock of ethidium bromide. Tubes were spun at 55,000 rpm for 18 h at 18° C. in a Sorvall TV860 rotor. The band of genomic DNA was extracted using a syringe and needle. Ethidium bromide was removed using two butanol extractions and then dialyzed against 4 l of T.E. at pH 8.0 overnight. The DNA was recovered by ethanol precipitation and then resuspended in T.E. buffer (1.7 mg total) and stored at −20° C.

### Example 6

### Cloning and Purification of S. aureus Pol III-L

To further characterize the mechanism of DNA replication in S. aureus, large amounts of its replication proteins were produced through use of the genes. The polC gene encoding S. aureus Pol III-L (alpha-large) subunit has been sequenced and expressed in E. coli (Pacitti et al., “Characterization and Overexpression of the Gene Encoding Staphylococcus aureus DNA Polymerase III,” Gene, 165:51-56 (1995), which is hereby incorporated by reference). The previous work utilized a pBS[KS] vector for expression in which the E. coli RNA polymerase is used for gene transcription. In the earlier study, the S. aureus polC gene was precisely cloned at the 5′ end encoding the N-terminus, but the amount of the gene that remained past the 3′ end was not disclosed and the procedure for subcloning the gene into the expression vector was only briefly summarized. Furthermore, the previous study does not show the level of expression of the S. aureus Pol III-L, nor the amount of S. aureus Pol III-L that is obtained from the induced cells. Since the previously published procedure could not be repeated and the efficiency of the expression vector could not be assessed, another strategy outlined below had to be developed.

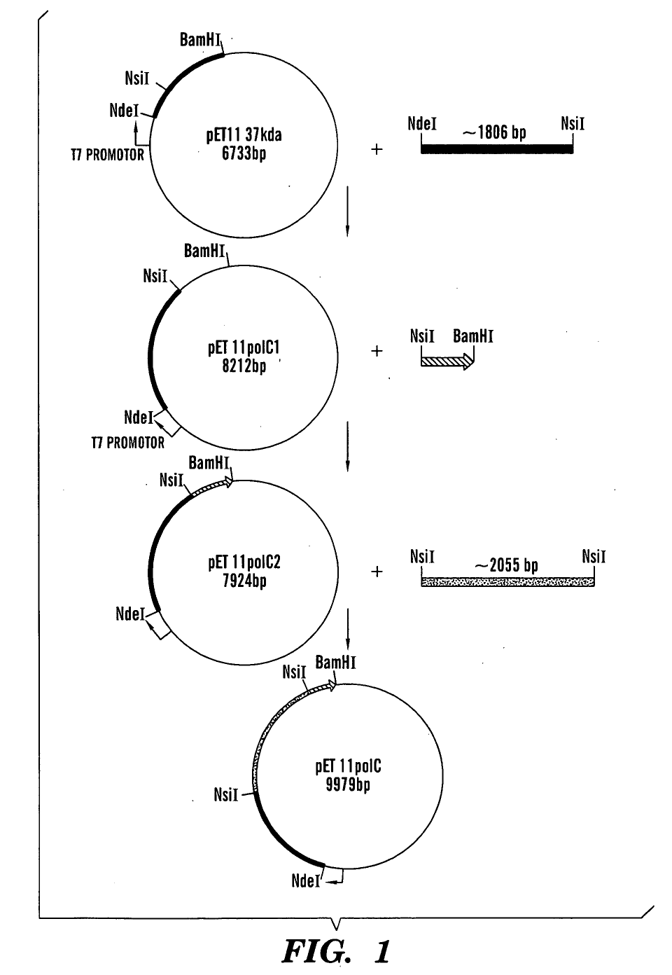

The isolated polC gene was cloned into a vector that utilizes T7 RNA polymerase for transcription as this process generally expresses a large amount of protein. Hence, the S. aureus polC gene was cloned precisely into the start codon at the NdeI site downstream of the T7 promotor in a pET vector. As the polC gene contains an internal NdeI site, the entire gene could not be amplified and placed it into the NdeI site of a pET vector. Hence, a three step cloning strategy that yielded the desired clone was devised (FIG. 1). These attempts were quite frustrating initially as no products of cloning in standard E. coli strains such as DH5α, a typical laboratory strain for preparation of DNA, could be obtained. Finally, a cell that was mutated in several genes affecting DNA stability was useful in obtaining the desired products of cloning.

In brief, the cloning strategy required use of another expression vector (called pET1137 kDa) in which the 37 kDa subunit of human RFC, the clamp loader of the human replication system, had been cloned into the pET11 vector. The gene encoding the 37 kDa subunit contains an internal NsiI site, which was needed for the precise cloning of the isolated polC gene. This three step strategy is shown in FIG. 1. In the first step, an approximately 2.3 kb section of the 5′ section of the gene (encoding the N-terminus of Pol III-L) was amplified using the polymerase chain reaction (PCR). Primers were as follows:

Upstream

(SEQ. ID. No. 35)

| | |
|---|---|
| ggtggtaatt gtcttgcata tgacagagc | 29 |
| Downstream | |

(SEQ. ID. No. 36)

| | |
|---|---|
| agcgattaag tggattgccg ggttgtgatg c | 31 |

Amplification was performed using 500 ng genomic DNA, 0.5 mM EDTA, 1 μM of each primer, 1 mM MgSO₄, 2 units vent DNA polymerase (New England Biolabs) in 100 μl of vent buffer (New England Biolabs). Forty cycles were performed using the following cycling scheme: 94° C., 1 min; 60° C., 1 min.; 72° C., 2.5 min. The product was digested with NdeI (underlined in the upstream primer) and NsiI (an internal site in the product) and the approximately 1.8 kb fragment was gel purified. A pET11 vector containing as an insert the 37 kDa subunit of human replication factor C (pET1137 kDa) was digested with NdeI and NsiI and gel purified. The PCR fragment was ligated into the digested pET1137 kDa vector and the ligation reaction was transformed into Epicurean coli supercompetent SURE 2 cells (Stratagene) and colonies were screened for the correct chimera (pET11PolC1) by examining minipreps for proper length and correct digestion products using NdeI and NsiI. In the second step, an approximately 2076 bp fragment containing the DNA encoding the C-terminus of Pol III-L subunit was amplified using the following sequences as primers:

Upstream

(SEQ. ID. No. 37)

| | |
|---|---|
| agcatcacaa cccggcaatc cacttaatcg c | 31 |
| Downstream | |

(SEQ. ID. No. 38)

| | |
|---|---|
| gactacgcca tgggcattaa ataaatacc | 29 |

The amplification cycling scheme was as described above except the elongation step at 72° C. was for 2 min. The product was digested with BamHI (underlined in the downstream primer) and NsiI (internal to the product) and the approximately 480 bp product was gel purified and ligated into the pET11PolC1 that had been digested with NsiI/BamHI and gel purified (ligated product is pET11PolC2). To complete the expression vector, an approximately 2080 bp PCR product was amplified over the two NsiI sites internal to the gene using the following primers:

Upstream

(SEQ. ID. No. 39)

| | |
|---|---|
| gaagatgcat ataaacgtgc aagacctagt | 30 |
| Downstream | |

(SEQ. ID. No. 40)

| | |
|---|---|
| gtctgacgca cgaattgtaa agtaagatgc atag | 34 |

The amplification cycling scheme was as described above except the 72° C. elongation step was 2 min. The PCR product, and the pET11PolC2 vector, were digested with NsiI and gel purified. The ligation mixture was transformed as described above and colonies were screened for the correct chimera (pET11PolC).

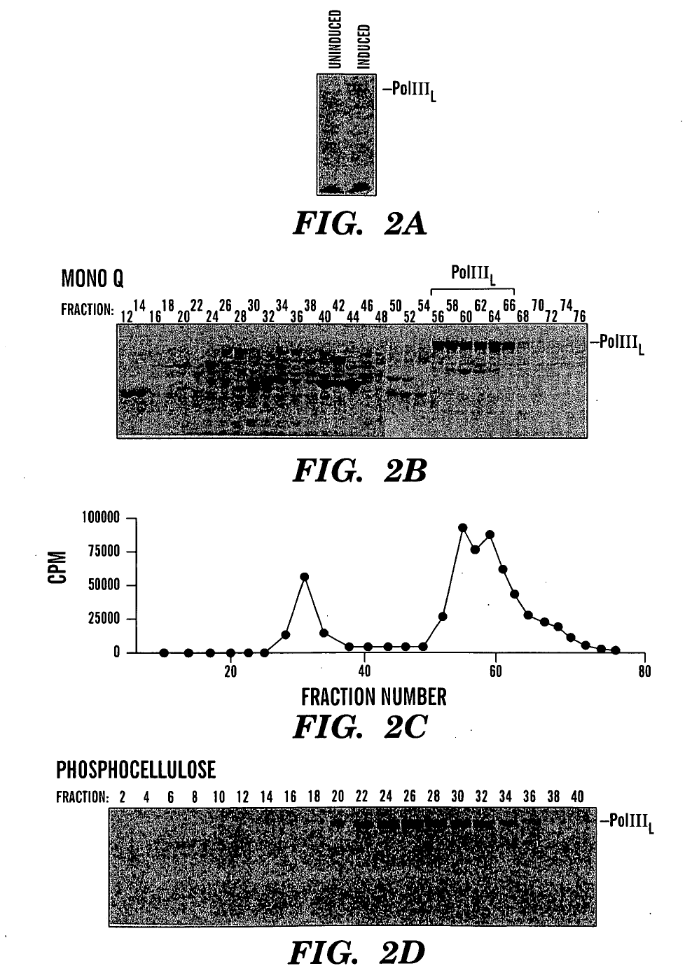

To express Pol III-L polymerase, the pET11PolC plasmid was transformed into E. coli strain BL21(DE3). 24 L of E. coli BL21(DE3)pET11PolC were grown in LB media containing 50 μg/ml ampicillin at 37° C. to an OD of 0.7 and then the temperature was lowered to 15° C. Cells were then induced for Pol III-L expression upon addition of 1 mM IPTG to produce the T7 RNA polymerase needed to transcribe polC. This step was followed by further incubation at 15° C. for 18 h. Expression of S. aureus Pol III-L polymerase was so high that it could easily be visualized by Coomassie staining of a SDS polyacrylamide gel of whole cells (FIG. 2A). The expressed protein migrated in the SDS polyacrylamide gel in a position expected for a 165 kDa polypeptide. In this procedure, it is important that cells are induced at 15° C., as induction at 37° C. produces a truncated version of Pol III-L polymerase, of approximately 130 kDa.

Cells were collected by centrifugation at 5° C. Cells (12 g wet weight) were stored at −70° C. The following steps were performed at 4° C. Cells were thawed and lysed in cell lysis buffer as described (final volume=50 ml) and were passed through a French Press (Amico) at a minimum of 20,000 psi. PMSF (2 mM) was added to the lysate as the lysate was collected from the French Press. DNA was removed and the lysate was clarified by centrifugation. The supernatent was dialyzed for 1 h against Buffer A containing 50 mM NaCl. The final conductivity was equivalent to 190 mM NaCl. Supernatent (24 ml, 208 mg) was diluted to 50 ml using Buffer A to bring the conductivity to 96 mM MgCl₂, and then was loaded onto an 8 ml MonoQ column equilibrated in Buffer A containing 50 mM NaCl. The column was eluted with a 160 ml linear gradient of Buffer A from 50 mM NaCl to 500 mM NaCl. Seventy five fractions (1.3 ml each) were collected (FIGS. 2B-C). Aliquots were analyzed for their ability to synthesize DNA, and 20 μl of each fraction was analyzed by Coomassie staining of an SDS polyacrylamide gel. Based on the DNA synthetic capability, and the correct size band in the gel, fractions 56-65 containing Pol III-L polymerase were pooled (22 ml, 31 mg). The pooled fractions were dialyzed overnight at 4° C. against 50 mM phosphate (pH 7.6), 5 mM DTT, 0.1 mM EDTA, 2 mM PMSF, and 20% glycerol (P-cell buffer). The dialyzed pool was loaded onto a 4.5 ml phosphocellulose column equilibrated in P-cell buffer, and then eluted with a 25 ml linear gradient of P-cell buffer from 0 M NaCl to 0.5 M NaCl. Fractions of 1 ml were collected and analyzed in a SDS polyacrylamide gel stained with Coomassie Blue (FIG. 2D). Fractions 20-36 contained the majority of the Pol III-large at a purity of greater than 90% (5 mg).

### Example 7

### S. aureus Pol III-L is not Processive on its Own

The Pol III-L polymerase purifies from B. subtilis as a single subunit without accessory factors (Barnes et al., “Purification of DNA Polymerase III of Gram-positive Bacteria,” Methods in Enzy., 262:35-42 (1995), which is hereby incorporated by reference). Hence, it seemed possible that it may be a Type I replicase (e.g., like T5 polymerase) and, thus, be capable of extending a single primer full length around a long singly primed template. To perform this experiment, a template M13mp18 ssDNA primed with a single DNA oligonucleotide either in the presence or absence of SSB was used. DNA products were analyzed in a neutral agarose gel which resolved products by size. The results showed that Pol III-L polymerase was incapable of extending the primer around the DNA (to form a completed duplex circle referred to as replicative form II (“RFII”)) whether SSB was present or not. This experiment has been repeated using more enzyme and longer times, but no full length RFII products are produced. Hence, Pol III-L would appear not to follow the paradigm of the T5 system (Type I replicase) in which the polymerase is efficient in synthesis in the absence of any other protein(s).

### Example 8

### Cloning and Purification of S. aureus Beta Subunit

The sequence of an S. aureus homolog of the E. coli dnaN gene (encoding the beta subunit) was obtained in a study in which the large recF region of DNA was sequenced (Alonso et al., “Nucleotide Sequence of the recF Gene Cluster From Staphylococcus aureus and Complementation Analysis in Bacillus subtilis recF Mutants,” Mol. Gen. Genet., 246:680-686 (1995), Alonso et al., “Nucleotide Sequence of the recF Gene Cluster From Staphylococcus aureus and Complementation Analysis in Bacillus subtilis recF Mutants,” Mol. Gen. Genet., 248:635-636 (1995), which are hereby incorporated by reference). Sequence alignment of the S. aureus beta and E. coli beta show approximately 30% identity. Overall this level of homology is low and makes it uncertain that S. aureus beta will have the same shape and function as the E. coli beta subunit.

To obtain S. aureus beta protein, the dnaN gene was isolated and precisely cloned into a pET vector for expression in E. coli. S. aureus genomic DNA was used as template to amplify the homolog of the dnaN gene (encoding the putative beta). The upstream and downstream primers were designed to isolate the dnaN gene by PCR amplification from genomic DNA. Primers were:

Upstream

(SEQ. ID. No. 41)

| | |
|---|---|
| cgactggaag gagttttaac atatgatgga attcac | 36 |
| Downstream | |

(SEQ. ID. No. 42)

| | |
|---|---|
| ttatatggat ccttagtaag ttctgattgg | 30 |

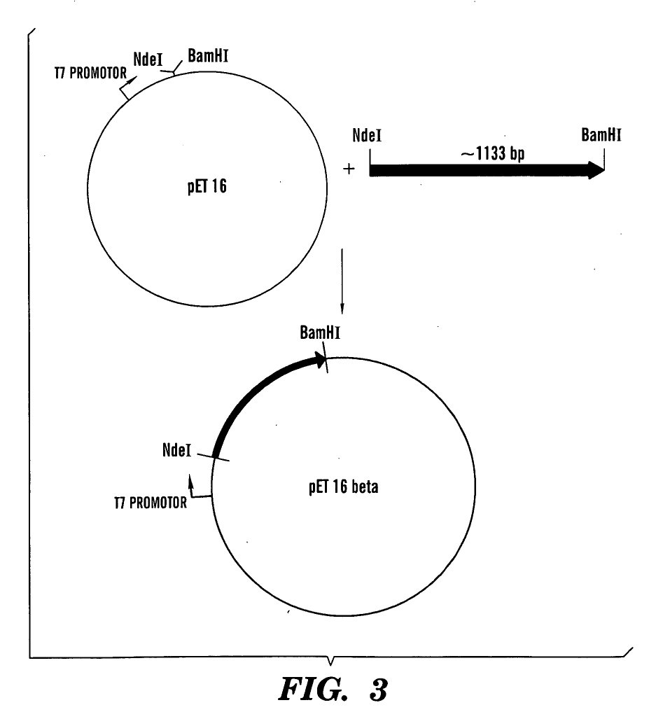

The NdeI site used for cloning into pET16b (Novagen) is underlined in the Upstream primer and the BamHI site used for cloning into pET16b is underlined in the Downstream primer. The NdeI and BamHI sites were used for directional cloning into pET16 (FIG. 3). Amplification was performed using 500 ng genomic DNA, 0.5 mM dNTPs, 1 μM of each primer, 1 mM MgSO₄, 2 units vent DNA polymerase in 100 ul of vent buffer. Forty cycles were performed using the following cycling scheme: 94° C., 1 min; 60° C., 1 min.; 72° C., 1 min. 10 s. The 1167 bp product was digested with NdeI and BamHI and purified in a 0.7% agarose gel. The pure digested fragment was ligated into the pET16b vector which had been digested with NdeI and BamHI and gel purified in a 0.7% agarose gel. Ligated products were transformed into E. coli competent SURE II cells (Stratagene) and colonies were screened for the correct chimera by examining minipreps for proper length and correct digestion products using NdeI and BamHI.

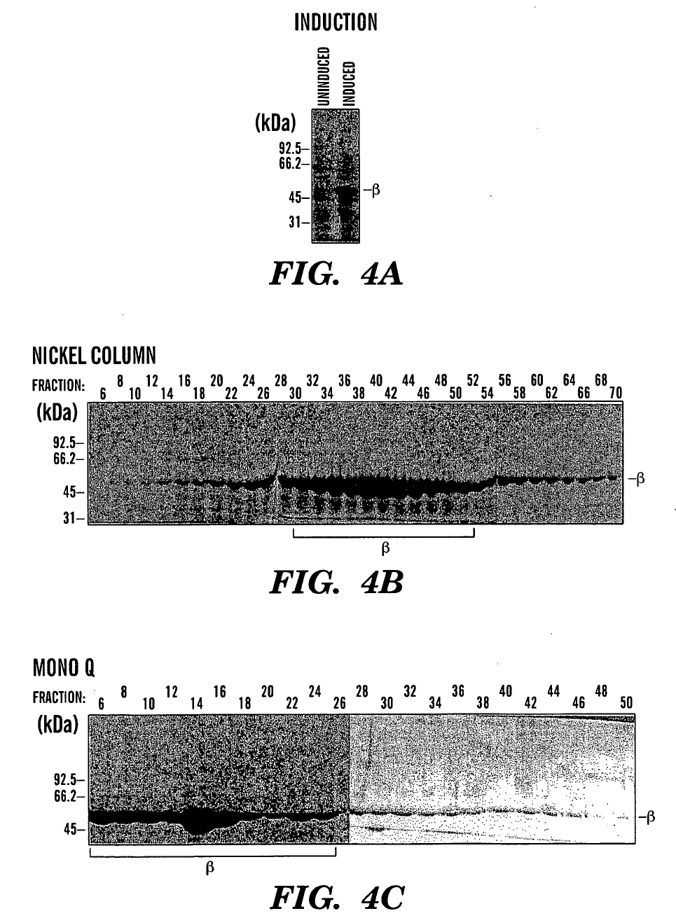

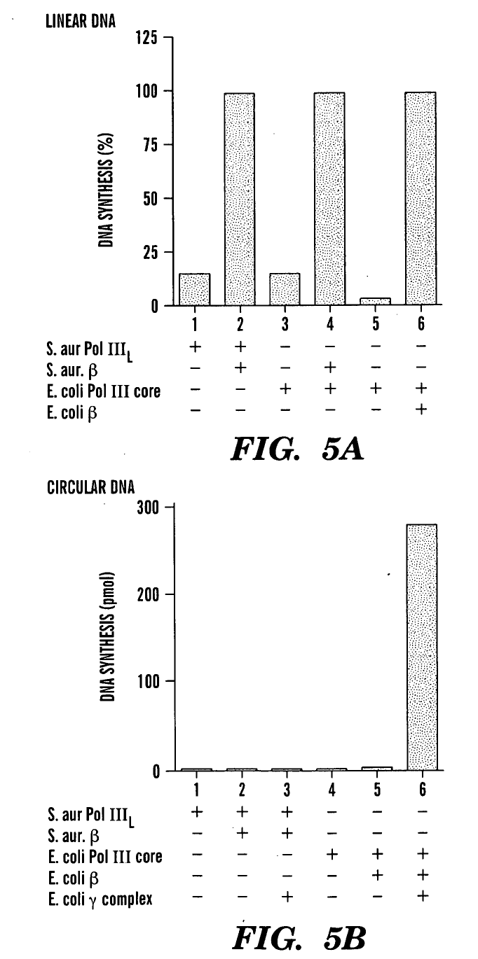

24 L of BL21(DE3)pETbeta cells were grown in LB containing 50 μg/ml ampicillin at 37° C. to an O.D. of 0.7, and, then, the temperature was lowered to 15° C. IPTG was added to a concentration of 2 mM and after a further 18 h at 15° C. to induce expression of S. aureus beta (FIG. 4A). It is interesting to note that the beta subunit, when induced at 37° C., was completely insoluble. However, induction of cells at 15° C. provided strong expression of beta and, upon cell lysis, over 50% of the beta was present in the soluble fraction.

Cells were harvested by centrifugation (44 g wet weight) and stored at −70° C. The following steps were performed at 4° C. Cells (44 g wet weight) were thawed and resuspended in 45 ml 1× binding buffer (5 mM imidazole, 0.5 M NaCl, 20 mM Tris HCl (final pH 7.5)) using a dounce homogenizer. Cells were lysed using a French Pressure cell (Aminco) at 20,000 psi, and then 4.5 ml of 10% polyamine P (Sigma) was added. Cell debris and DNA was removed by centrifugation at 13,000 rpm for 30 min. at 4° C. The pET16beta vector places a 20 residue leader containing 10 histidine residues at the N-terminus of beta. Hence, upon lysing the cells, the S. aureus beta was greatly purified by chromatography on a nickel chelate resin (FIG. 4B). The supernatant (890 mg protein) was applied to a 10 ml HiTrap Chelating Separose column (Pharmacia-LKB) equilibrated in binding buffer. The column was washed with binding buffer, then eluted with a 100 ml linear gradient of 60 mM imidazole to 1 M imidazole in binding buffer. Fractions of 1.35 ml were collected. Fractions were analyzed for the presence of beta in an SDS polyacrylamide gel stained with Coomassie Blue. Fractions 28-52, containing most of the beta subunit, were pooled (35 ml, 82 mg). Remaining contaminating protein was removed by chromatography on MonoQ. The S. aureus beta becomes insoluble as the ionic strength is lowered and, thus, the pool of beta was dialyzed overnight against Buffer A containing 400 mM NaCl. The dialyzed pool became slightly turbid indicating it was at its solubility limit at these concentrations of protein and NaCl. The insoluble material was removed by centrifugation (64 mg remaining) and, then, diluted 2-fold with Buffer A to bring the conductivity to 256. The protein was then applied to an 8 ml MonoQ column equilibrated in Buffer A plus 250 mM NaCl and then eluted with a 100 ml linear gradient of Buffer A from 0.25M NaCl to 0.75 M NaCl; fractions of 1.25 ml were collected (FIG. 4C). Under these conditions, approximately 27 mg of the beta flowed through the column and the remainder eluted in fractions 1-18 (24 mg).

### Example 9

### The S. aureus Beta Subunit Protein Stimulates S. aureus Pol III-L and E. coli Core

The experiment of FIG. 5A, tests the ability of S. aureus beta to stimulate S. aureus Pol III-L on a linear polydA-oligodT template. Reactions are also performed with E. coli beta and Pol III core. The linear template was polydA of average length of 4500 nucleotides primed with a 30mer oligonucleotide of T residues. The first two lanes show the activity of Pol III-L either without (lane 1) or with S. aureus beta (lane 2). The result shows that the S. aureus beta stimulates Pol III-L approximately 5-6 fold. Lanes 5 and 6 show the corresponding experiment using E. coli core with (lane 6) or without (lane 5) E. coli beta. The core is stimulated over 10-fold by the E. coli beta subunit under the conditions used.

Although Gram positive and Gram negative cells diverged from one another long ago and components of one polymerase machinery would not be expected to be interchangable, it was decided to test the activity of the S. aureus beta with E. coli Pol III core. Lanes 3 and 4 shows that the S. aureus beta also stimulates E. coli core about 5-fold. This result can be explained by an interaction between the clamp and the polymerase that has been conserved during the evolutionary divergence of gram positive and gram negative cells. A chemical inhibitor that would disrupt this interaction would be predicted to have a broad spectrum of antibiotic activity, shutting down replication in Gram negative and Gram positive cells alike. This assay, and others based on this interaction, can be devised to screen chemicals for such inhibition. Further, since all the proteins in this assay are highly overexpressed through recombinant techniques, sufficient quantities of the protein reagents can be obtained for screening hundreds of thousands of compounds.

In summary, the results show that S. aureus beta, produced in E. coli, is indeed an active protein (i.e., it stimulates polymerase activity). Furthermore, the results shows that Pol III-L functions with a second protein (i.e., S. aureus beta). Before this experiment, there was no assurance that Pol III-L, which is significantly different in structure from E. coli alpha, would function with another protein. For example, unlike E. coli alpha, which copurifies with several accessory proteins, Pol III-L purified from B. subtilis as a single protein with no other subunits attached (Barnes et al., “Purification of DNA Polymerase III of Gram-positive Bacteria,” Methods in Enzy., 262:35-42 (1995), which is hereby incorporated by reference). Finally, if one were to assume that S. aureus beta would function with a polymerase, the logical candidate would have been the product of the dnaE gene (alpha-small) instead of polC (Pol III-L) since the dnaE product is more homologous to E. coli alpha subunit than Pol III-L.

### Example 10

### The S. aureus Beta Subunit Behaves as a Circular Sliding Clamp

The ability of S. aureus beta to stimulate Pol III-L could be explained by formation of a 2-protein complex between Pol III-L and beta to form a processive replicase similar to the Type II class (e.g., T7 type). Alternatively, the S. aureus replicase is organized as the Type III replicase which operates with a circular sliding clamp and a clamp loader. In this case, the S. aureus beta would be a circular protein and would require a clamp loading apparatus to load it onto DNA. The ability of the beta subunit to stimulate Pol III-L in FIG. 5A could be explained by the fact that the polydA-oligodT template is a linear DNA and a circular protein could thread itself onto the DNA over an end. Such “end threading” has been observed with PCNA and explains its ability to stimulate DNA polymerase delta in the absence of the RFC clamp loader (Burgers et al., “ATP-Independent Loading of the Proliferating Cell Nuclear Antigen Requires DNA Ends,” J. Biol. Chem., 268:19923-19926 (1993), which is hereby incorporated by reference).

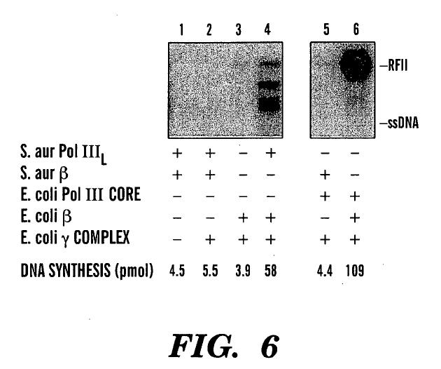

To distinguish between these possibilities, S. aureus beta was examined for ability to stimulate Pol III-L on a circular primed template. In FIG. 5B, assays were performed using circular M13mp18 ssDNA coated with E. coli SSB and primed with a single oligonucleotide to test the activity of beta on circular DNA. Lane 1 shows the extent of DNA synthesis using Pol III-L alone. In lane 2, Pol III-L was supplemented with S. aureus beta. The S. aureus beta did not stimulate the activity of Pol III-L on this circular DNA (nor in the absence of SSB). Inability of S. aureus beta to stimulate Pol III-L is supported by the results of FIG. 6, lane 1 that analyzes the product of Pol III-L action on the circular DNA in an agarose gel in the presence of S. aureus beta. In summary, these results show that S. aureus beta only stimulates Pol III-L on linear DNA, not circular DNA. Hence, the S. aureus beta subunit behaves as a circular protein.

Lane 3 shows the result of adding both S. aureus beta and E. coli gamma complex to Pol III-L. Again, no stimulation was observed (compare with lane 1). This result indicates that the functional contacts between the clamp and clamp loader were not conserved during evolution of Gram positive and Gram negative cells.

Controls for these reactions on circular DNA are shown for the E. coli system in Lanes 4-6. Addition of only beta to E. coli Pol III core did not result in stimulating the polymerase (compare lanes 4 and 5). However, when clamp loader complex was included with beta and core, a large stimulation of synthesis was observed (lane 6). In summary, stimulation of synthesis is only observed when both beta and clamp loader complex were present, consistent with inability of the circular beta ring to assemble onto circular DNA by itself.

### Example 11

### Pol III-L Functions as a Pol III-Type Replicase with Beta and a Clamp Loader Complex to Become Processive

Next, it was determined whether S. aureus Pol III-L requires two components (a beta clamp and a clamp loader) to extend a primer full length around a circular primed template. In FIG. 6, a template circular M13mp18 ssDNA primed with a single DNA oligonucleotide was used. DNA products were analyzed in a neutral agarose gel which resolves starting materials (labeled ssDNA in FIG. 6) from completed duplex circles (labelled RFII for replicative form II). The first two lanes show, as demonstrated in other examples, that Pol III-L is incapable of extending the primer around the circular DNA in the presence of only S. aureus beta. In lane 4 of FIG. 6, E. coli clamp loader complex (also known as gamma complex) and beta subunit were mixed with S. aureus Pol III-L in the assay containing singly primed M13mp18 ssDNA coated with SSB. If the beta clamp, assembled on DNA by clamp loader complex, provides processivity to S. aureus Pol III-L, the ssDNA circle should be converted into a fully duplex circle (RFII) which would be visible in an agarose gel analysis. The results of the experiment showed that the E. coli beta and clamp loader complex did indeed provide Pol III-L with ability to fully extend the primer around the circular DNA to form the RFII (lane 4). The negative control using only E. coli clamp loader complex and beta is shown in lane 3. For comparison, lane 6 shows the result of mixing the three components of the E. coli system (Pol III core, beta, and clamp loader complex). This reaction gives almost exclusively full length RFII product. The qualitatively different product profile that Pol III-L gives in the agarose gel analysis compared to E. coli Pol III core with beta and clamp loader complex shows that the products observed using Pol III-L is not due to a contaminant of E. coli Pol III core in the S. aureus Pol III-L preparation (compare lanes 4 and 6).

It is generally thought that the polymerase of one system is specific for its SSB. However, these reactions are performed on ssDNA coated with the E. coli SSB protein. Hence, the S. aureus Pol III-L appears capable of utilizing E. coli SSB and the E. coli beta. It would appear that the only component that is not interchangeable between the Gram positive and Gram negative systems is the clamp loader complex.

Thus, the S. aureus Pol III-L functions as a Pol III type replicase with the E. coli beta clamp assembled onto DNA by a clamp loader complex.

### Example 12

### Purification of Two DNA Polymerase III-Type Enzymes from S. aureus Cells

The MonoQ resin by Pharmacia has very high resolution which would resolve the three DNA polymerases of S. aureus. Hence, S. aureus cells were lysed, DNA was removed from the lysate, and the clarified lysate was applied onto a MonoQ column. The details of this procedure are: 300 L of S. aureus (strain 4220, a gift of Dr. Pat Schlievert, University of Minnesota) was grown in 2×LB media at 37° C. to an O.D. of approximately 1.5 and then were collected by centrifugation. Approximately 2 kg of wet cell paste was obtained and stored at −70° C. 122 g of cell paste was thawed and resuspended in 192 ml of cell lysis buffer followed by passage through a French Press cell (Aminco) at 40,000 psi. The resultant lysate was clarified by high speed centrifugation (1.3 g protein in 120 ml). A 20 ml aliquot of the supernatant was dialyzed 2 h against 2 L of buffer A containing 50 mM NaCl. The dialyzed material (148 mg, conductivity=101 mM NaCl) was diluted 2-fold with Buffer A containing 50 mM NaCl and then loaded onto an 8 ml MonoQ column equilibrated in Buffer A containing 50 mM NaCl. The column was washed with Buffer A containing 50 mM NaCl, and then eluted with a 160 ml linear gradient of 0.05 M NaCl to 0.5 M NaCl in Buffer A. Fractions of 2.5 ml (64 total) were collected, followed by analysis in an SDS polyacrylamide gel for their replication activity in assays using calf thymus DNA.

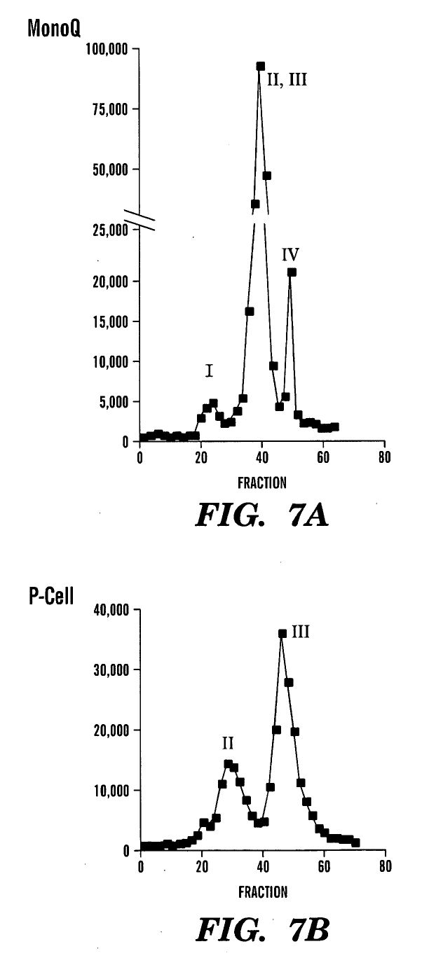

Three peaks of DNA polymerase activity were identified (FIG. 7). Previous studies of cell extracts prepared from the Gram positive organism Bacillus subtilis identified only two peaks of activity off a DEAE column (similar charged resin to MonoQ). The first peak was Pol II, and the second peak was a combination of DNA polymerases I and III. The DNA polymerases I and III were then separated on a subsequent phosphocellulose column. The middle peak in FIG. 7 is much larger than the other two peaks and, thus, it was decided to chromatograph this peak on a phosphocellulose column. The second peak of DNA synthetic activity was pooled (fractions 37-43; 28 mg in 14 ml) and dialyzed against 1.5 L P-cell buffer for 2.5 h. Then, the sample (ionic strength equal to 99 mM NaCl) was applied to a 5 ml phosphocellulose column equilibrated in P-cell buffer. After washing the column in 10 ml P-cell buffer, the column was eluted with a 60 ml gradient of 0-0.5 M NaCl in P-cell buffer. Seventy fractions were collected and then analyzed for DNA synthesis using calf thymus DNA as template. This column resolved the polymerase activity into two distinct peaks (FIG. 7B).

Hence, there appear to be four DNA polymerases in Staphylococcus aureus. They were designated here as peak 1 (first peak off MonoQ), peak 2 (first peak off phosphocellulose), peak 3 (second peak of phosphocellulose), and peak 4 (last peak off Mono Q) (see FIG. 7). Peak 4 was presumably Pol III-L, as it elutes from MonoQ in a similar position as the Pol III-L expressed in E. coli (compare FIG. 7A with FIG. 2).

### Example 13

### Demonstration that Peak 1 (Pol III-2) Functions as a Pol III-Type Replicase with E. coli Beta Assembled on DNA by E. coli Clamp Loader Complex

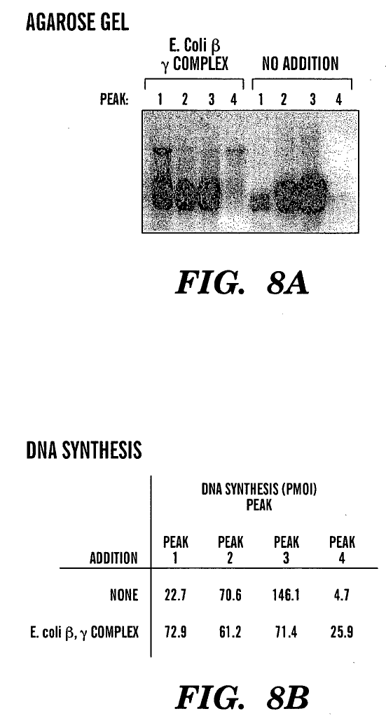

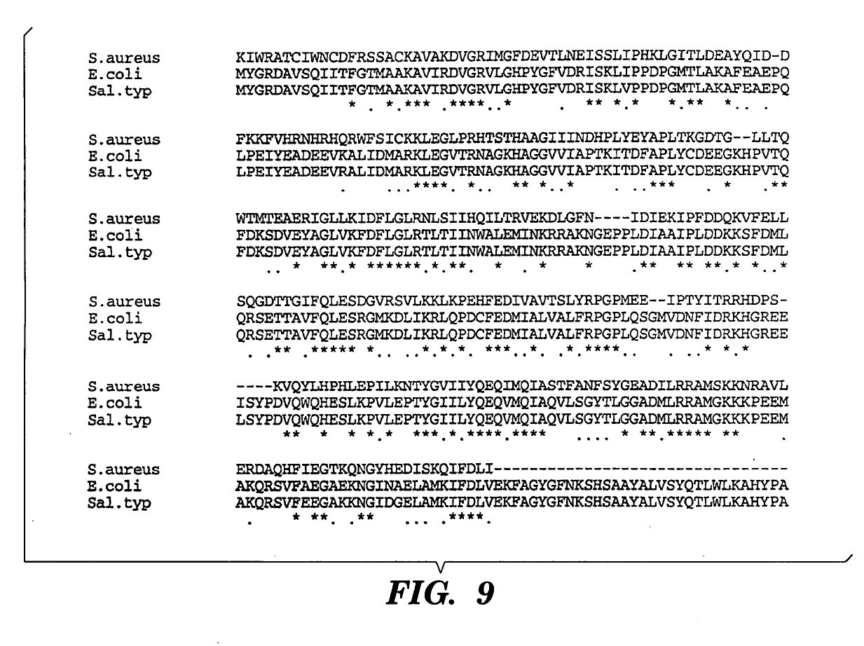

To test which peak contained a Pol III-type of polymerase, an assay was used in which the E. coli clamp loader complex and beta support formation of full length RFII product starting from E. coli SSB coated circular M13mp18 ssDNA primed with a single oligonucleotide. In FIG. 8, both Peaks 1 and 2 are stimulated by the E. coli clamp loader complex and beta subunit and, in fact, Peaks 2 and 3 are inhibited by these proteins (the quantitation is shown below the gel in the figure). Further, the product analysis in the agarose gel shows full length RFII duplex DNA circles only for peaks 1 and 4. These results, combined with the NEM, pCMB, and KCl characteristics in Tables 2 and 3 below, suggest that there are two Pol III-type DNA polymerases in S. aureus and that these are partially purified in peaks 1 and 4.

Next, it was determined which of these peaks of DNA polymerase activity correspond to DNA polymerases I, II, and III, and which peak is the unidentified DNA polymerase. In the Gram positive bacterium B. subtilis, Pol III is inhibited by pCMB, NEM, and 0.15 M NaCl, Pol II is inhibited by KCl, but not NEM or 0.15 M KCL, and Pol I is not inhibited by any of these treatments (Gass et al., “Further Genetic and Enzymological Characterization of the Three Bacillus subtilis Deoxyribonucleic Acid Polymerases,” J. Biol. Chem., 248:7688-7700 (1973), which is hereby incorporated by reference). Hence, assays were performed in the presence or absence of pCMB, NEM, and 0.15 M KCl (see Tables 2 and 3 below). Peak 3 clearly corresponded to Pol I, because it was not inhibited by NEM, pCMB, or 0.15 M NaCl Peak 2 correspond to Pol II, because it was not inhibited by NEM, but was inhibited by pCMB and 0.15 M NaCl. Peaks 1 and 4 both had characteristics that mimic Pol III; however, peak 4 elutes on MonoQ at a similar position as Pol III-L expressed in E. coli (see FIG. 2B). Hence, peak 4 is likely Pol III-L, and peak 1 is likely the unknown polymerase.

TABLE 2
Expected Characteristics of Polymerases

| Polymerase | pCMB | NEM | 0.15M KCl |
|---|---|---|---|
| Pol I | not inhibited* | not inhibited | not inhibited |
| Pol II | inhibited** | not inhibited | not inhibited |
| Pol III-L | inhibited | inhibited | not inhibited |

*Not inhibited is defined as greater than 75% remaining activity
**Inhibited is defined as less than 40% remaining activity

TABLE 3
Observed Characteristics

| Peak | pCMB | NEM | 0.15M KCL assignment |
|---|---|---|---|
| Peak1 | inhibited | inhibited | new polymerase |
| Peak2 | inhibited | not inhibited | Pol II |
| Peak3 | not inhibited | not inhibited | Pol I |
| Peak4 | inhibited | inhibited | Pol III-L |

### Example 14

### Identification and Cloning of S. aureus dnaE

This invention describes the finding of two DNA polymerases that function with a sliding clamp assembled onto DNA by a clamp loader. One of these DNA polymerases is likely Pol III-L, but the other has not been identified previously. Presumably, the chromatographic resins used in earlier studies did not have the resolving power to separate the enzyme from other polymerases. This would be compounded by the low activity of Pol III-2. To identify a gene encoding the second Pol III, the amino acid sequences of the Pol III alpha subunit of Escherichia coli, Salmonella typhimurium, Vibrio cholerae, Haemophilis influenzae, and Helicobacter pylori were aligned using Clustal W (1.5). Two regions about 400 residues apart were conserved and primers were designed for the following amino acid sequences:

Upstream, corresponding in E. coli to residues
385-399

(SEQ. ID. No. 43)

```
Leu Leu Phe Glu Arg Phe Leu Asn Pro Glu Arg Val
    1 5 10
Ser Met Pro
                  15
Downstream, corresponding in E. coli to residues
750-764
```

(SEQ. ID. No. 44)

```
Lys Phe Ala Gly Tyr Gly Phe Asn Lys Ser His Ser
    1 5 10
Ala Ala Tyr
                  15
```

The following primers were designed to these two peptide regions using codon preferences for S. aureus:

| Upstream | |
|---|---|
| (SEQ. ID. No. 45) | |
| cttctttttg aaagatttct aaataaagaa cgttattcaa tgcc | 44 |
| Downstream | |
| (SEQ. ID. No. 46) | |
| ataagctgca gcatgacttt tattaaaacc ataacctgca aattt | 45 |

Amplification was performed using 2.5 units of Taq DNA Polymerase (Gibco, BRL), 100 ng S. aureus genomic DNA, 1 mM of each of the four dNTPs, 1 μM of each primer, and 3 mM MgCl₂ in 100 μl of Taq buffer. Thirty-five cycles of the following scheme were repeated: 94° C., 1 min; 55° C., 1 min; 72° C., 90 sec. The PCR product (approximately 1.1 kb) was electrophoresed in a 0.8% agarose gel and purified using a Geneclean III kit (Bio 101). The product was then divided equally into ten separate aliquots and used as a template for PCR reactions, according to the above protocol, to reamplify the fragment for sequencing. The final PCR product was purified using a Quiagen Quiaquick PCR Purification kit, quantitated via optical density at 260 nM, and sequenced by the Protein/DNA Technology Center at Rockefeller University. The same primers used for PCR were used to prime the sequencing reactions.

Next, the following additional PCR primers were designed to obtain more sequence information 3′ to the first amplified section.

Upstream

(SEQ. ID. No. 47)

| | |
|---|---|
| agttaaaaat gccatatttt gacgtgtttt agttctaat | 39 |
| Downstream | |

(SEQ. ID. No. 48)

| | |
|---|---|
| cttgcaaaag cggttgctaa agatgttgga cgaattatgg gg | 42 |

These primers were used in a PCR reaction using 2.5 units of Taq DNA Polymerase (Gibco, BRL) with 100 ng S. aureus genomic DNA as a template, 1 mM dNTP's, 1 μM of each primer, and 3 mM MgCl₂ in 100 l of Taq buffer. Thirty-five cycles of the following scheme were repeated: 94° C., 1 min; 55° C., 1 min; 72° C., 2 min 30 seconds. The 1.6 Kb product was then divided into 5 aliquots, and used as a template in a set of 5 PCR reactions, as described above, to amplify the product for sequencing. The products of these reactions were purified using a Qiagen Qiaquick PCR Purification kit, quantitated via optical density at 260 nm, and sequenced by the Protein/DNA Technology Center at Rockefeller University. The sequence of this product yielded about 740 bp of new sequence 3′ of the first sequence.

As this gene shows better homology to the Gram negative Pol III α subunit compared to Gram positive Pol III-L, it will be designated the dnaE gene.

### Example 15

### Identification and Cloning of S. aureus dnaX

The fact that the S. aureus beta stimulates Pol III-L and has a ring shape suggests that the Gram positive replication machinery is of the three component type. This implies the presence of a clamp loader complex. This is not a simple determination to make as the B. subtilis genome shows homologs to only two of the five subunits of the E. coli clamp loader (dnaX encoding gamma, and holB encoding delta prime). On the basis of the experiments in this application, which suggests that there is a clamp loader, it was believed that these two subunit homologues are part of the clamp loader for the S. aureus beta.

As a start in obtaining the clamp loading apparatus, a strategy was devised to obtain the gene encoding the tau subunit of S. aureus. In E. coli, the tau and gamma subunits are derived from the same gene. Tau is the full length product, and gamma is about ⅔ the length of tau. Gamma is derived from the dnaX gene by what was originally believed to be an efficient translational frameshift mechanism that, after it occurs, incorporates only one unique C-terminal residue before encountering a stop codon. To identify the dnaX gene of S. aureus by PCR analysis, the dnaX genes of B. subtilis, E. coli and H. influenzae were aligned. Upon comparison of the amino acid sequence encoded by these dnaX genes, two areas of high homology were used to predict the amino acid sequence of the S. aureus dnaX gene product. PCR primers were designed to these sequences, and a PCR product of the expected size was indeed produced. DNA primers were designed to two regions of high similarity for use in PCR that were about 100 residues apart. The amino acid sequences of these regions were:

Upstream, corresponding to residues 39-48 of
E. coli

(SEQ. ID. No. 49)

His Ala Tyr Leu Phe Ser Gly Pro Arg Gly
1 5 10
Downstream, corresponding to residues 138-148 of
E. coli

(SEQ. ID. No. 50)

His Ala Tyr Leu Phe Ser Gly Pro Arg Gly
1 5 10

The DNA sequence of the PCR primers was based upon the codon usage of S. aureus. The primers are as follows:

Upstream

(SEQ. ID. No. 51)

| | |
|---|---|
| cgcggatccc atgcatattt attttcaggt ccaagagg | 38 |
| Downstream | |

(SEQ. ID. No. 52)

| | |
|---|---|
| ccggaattct ggtggttctt ctaatgtttt taataatgc | 39 |

The first 9 nucleotides of the upstream primer (SEQ. ID. No. 51) contain a BamHI site, which is underlined, and do not correspond to amino acid codons; the 3′ 29 nucleotides correspond to the amino acid sequence of SEQ. ID. No. 49. The EcoRI site of the downstream primer (SEQ. ID. No. 52) is underlined and the 3′ 33 nucleotides correspond to the amino acid sequence of SEQ. ID. No. 50.

The expected PCR product, based on the alignment, is approximately 268 bp between the primer sequences. Amplification was performed using 500 ng genomic DNA, 0.5 mM dNTPs, 1 μM of each primer, 1 mM MgSO₄, 2 units vent DNA polymerase in 100 μl of vent buffer. Forty cycles were performed using the following cycling scheme: 94° C., 1 min; 60° C., 1 min.; 72° C., 30 s. The approximately 300 bp product was digested with EcoRI and BamHI and purified in a 0.7% agarose gel. The pure digested fragment was ligated into pUC18 which had been digested with EcoRI and BamHI and gel purified in a 0.7% agarose gel. Ligated products were transformed into E. coli competent DH5α cells (Stratagene), and colonies were screened for the correct chimera by examining minipreps for proper length and correct digestion products using EcoRI and BamHI. The sequence of the insert was determined and was found to have high homology to the dnaX genes of several bacteria. This sequence was used to design circular PCR primers. Two new primers were designed for circular PCR based on this sequence.

A circular PCR product of approximately 1.6 kb was obtained from a HincII digest of chromosomal DNA that was recircularized with ligase. This first circular PCR yielded most of the remaining dnaX gene. The two primers were as follows:

Rightward

(SEQ. ID. No. 53)

| | |
|---|---|
| tttgtaaagg cattacgcag gggactaatt cagatgtg | 38 |
| Leftward | |

(SEQ. ID. No. 54)

| | |
|---|---|
| tatgacattc attacaaggt tctccatcag tgc | 33 |

Genomic DNA (3 μg) was digested with HincII, purified with phenol/chloroform extraction, ethanol precipitated and redissolved in 70 μl T.E. buffer. The genomic DNA was recircularized upon adding 4000 units T4 ligase (New England Biolabs) in a final volume of 100 μl T4 ligase buffer (New England Biolabs) at 16° C. overnight. The PCR reaction consisted of 90 ng recircularized genomic DNA, 0.5 mM each dNTP, 100 pmol of each primer, 1.4 mM magnesium sulfate, and 1 unit of elongase (GIBCO) in a final volume of 100 μl elongase buffer (GIBCO). 40 cycles were performed using the following scheme: 94° C., 1 min.; 55° C., 1 min.; and 68° C., 2 min. The resulting PCR product was approximately 1.6 kb. The PCR product was purified from a 0.7% agarose gel and sequenced directly. A stretch of approximately 750 nucleotides was obtained using the rightward primer used in the circular PCR reaction. To obtain the rest of the sequence, other sequencing primers were designed in succession based on the information of each new sequencing run.

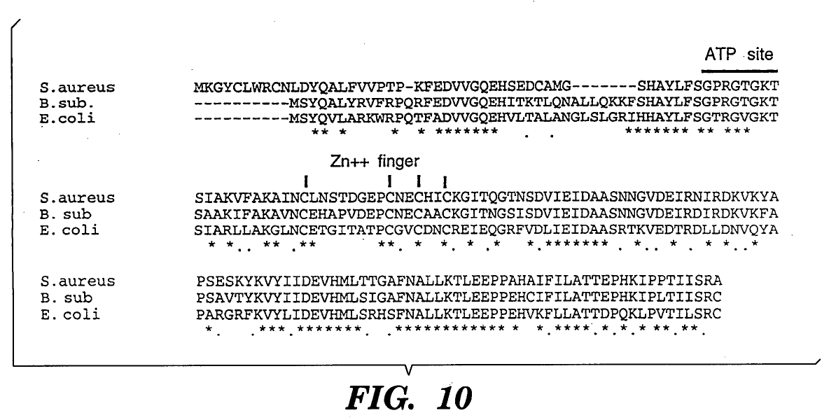

This sequence, when spliced together with the previous 300 bp PCR sequence, contained the complete N-terminus of the gene product (stop codons are present upstream) and possibly lacked only about 50 residues of the C-terminus. The amino terminal region of E. coli tau shares what appears to be the most conserved region of the gene as this area shares homology with RFC subunit of the human clamp loader and with the gene 44 protein of the phage T4 clamp loader. An alignment of the N-terminal region of the S. aureus tau protein with that of B. subtilis and E. coli is shown in FIG. 10. Among the highly conserved residues are the ATP binding site consensus sequence and the four cystine residues that form a Zn²⁺ finger.

After obtaining 1 kb of sequence in the 5′ region of dnaX, it was sought to determine the remaining 3′ end of the gene. Circular PCR products of approximately 800 bps, 600 bps, and 1600 bps were obtained from Apo I, or Nsi I or Ssp I digest of chromosomal DNA that were recircularized with ligase.

Rightward

(SEQ. ID. No. 55)

| | |
|---|---|
| gagcactgat gaacttagaa ttagatatg | 29 |
| Leftward | |

(SEQ. ID. No. 56)

| | |
|---|---|
| gatactcagt atctttctca gatgttttat tc | 32 |

Genomic DNA (3 g) was digested with, Apo I, or Nsi I or Ssp I, purified with phenol/chloroform extraction, ethanol precipitated, and redissolved in 70 l T.E. buffer. The genomic DNA was recircularized upon adding 4000 units of T4 ligase (New England Biolabs) in a final volume of 100 l T4 ligase buffer (New England Biolabs) at 16° C. overnight. The PCR reaction consisted of 90 ng recircularized genomic DNA, 0.5 mM each dNTP, 100 pmol of each primer, 1.4 mM magnesium sulfate, and 1 unit of elongase (GIBCO) in a final volume of 100 l elongase buffer (GIBCO). 40 cycles were performed using the following scheme: 94° C., 1 min.; 55° C., 1 min.; 68° C., 2 min. The PCR products were directly cloned into pCR II TOPO vector using the TOPO TA cloning kit (Invitrogen Corporation) for obtaining the rest of the C terminal sequence of S. aureus dnaX. DNA sequencing was performed by the Rockefeller University sequencing facility.

### Example 16

### Identification and Cloning of S. aureus dnaB

In E. coli, the DnaB helicase assembles with the DNA polymerase III holoenzyme to form a replisome assembly. The DnaB helicase also interacts directly with the primase to complete the machinery needed to duplicate a double helix. As a first step in studying how the S. aureus helicase acts with the replicase and primase, S. aureus was examined for presence of a dnaB gene.

The amino acid sequences of the DnaB helicase of Escherichia coli, Salmonella typhimurium, Haemophilis influenzae, and Helicobacter pylori were aligned using Clustal W (1.5). Two regions about 200 residues apart showed good homology. These peptide sequences were:

Upstream, corresponding to residues 225-238 of
E. coli DnaB

(SEQ. ID. No. 57)

Asp Leu Ile Ile Val Ala Ala Arg Pro Ser Met Gly
1 5 10
Lys Thr
Downstream, corresponding to residues 435-449 of
E. coli DnaB

(SEQ. ID. No. 58)

```
Glu Ile Ile Ile Gly Lys Gln Arg Asn Gly Pro Ile
    1 5 10
Gly Thr Val
                  15
```

The following primers were designed from regions which contained conserved sequences using codon preferences for S. aureus:

| Upstream | |
|---|---|
| (SEQ. ID. No. 59) | |
| gaccttataa ttgtagctgc acgtccttct atgggaaaaa c | 41 |
| Downstream | |
| (SEQ. ID. No. 60) | |
| aacattatta agtcagcatc ttgttctatt gatccagatt caacgaag | 48 |

A PCR reaction was carried out using 2.5 units of Taq DNA Polymerase (Gibco, BRL) with 100 ng. S. aureus genomic DNA as template, 1 mM dNTP's, 1 μM of each primer, 3 mM MgCl₂ in 100 μl of Taq buffer. Thirty-five cycles of the following scheme were repeated: 94° C., 1 min.; 55° C., 1 min.; and 72° C., 1 min. Two PCR products were produced, one was about 1.1 kb, and another was 0.6 kb. The smaller one was the size expected. The 0.6 kb product was gel purified and used as a template for a second round of PCR as follows. The 0.6 kb PCR product was purified from a 0.8% agarose gel using a Geneclean III kit (Bio 101) and then divided equally into five separate aliquots, as a template for PCR reactions. The final PCR product was purified using a Quiagen Quiaquick PCR Purification kit, quantitated via optical density at 260 nM, and sequenced by the Protein/DNA Technology Center at Rockefeller University. The same primers used for PCR were used to prime the sequencing reaction. The amino acid sequence was determined by translation of the DNA sequence in all three reading frames, and selecting the longest open reading frame. The PCR product contained an open reading frame over its entire length. The predicted amino acid sequence shares homology to the amino acid sequences encoded by dnaB gene of other organisms.

Additional sequence information was determined using the circular PCR technique. Briefly, S. aureus genomic DNA was digested with various endonucleases, then religated with T4 DNA ligase to form circular templates. To perform PCR, two primers were designed from the initial sequence.

| First primer | |
|---|---|
| (SEQ. ID. No. 61) | |
| gatttggagt tctggtaatg ttgactcaaa ccgcttaaga accgg | 45 |
| Second primer | |
| (SEQ. ID. No. 62) | |
| atacgtgtgg ttaactgatc agcaacccat ctctagtgag aaaatacc | 48 |

The first primer matches the sequence of the coding strand and the second primer matches the sequence of the complementary strand. These two primers are directed outwards from a central point, and allow determination of new sequence information up to the ligated endonuclease site. A PCR product of approximately 900 bases in length was produced using the above primers and template derived from the ligation of S. aureus genomic DNA which had been cut with the restriction endonuclease Apo I. This PCR product was electrophoresed in a 0.8% agarose gel, eluted with a Qiagen gel elution kit, divided into five separate aliquots, and used as a template for reamplification by PCR using the same primers as described above. The final product was electrophoresed in an 0.8% agarose gel, visualized via staining with ethidium bromide under ultraviolet light, and excised from the gel. The excised gel slice was frozen, and centrifuged at 12,000 rpm for 15 minutes. The supernatant was extracted with phenol/chloroform to remove ethidium bromide, and was then cleaned using a Qiagen PCR purification kit. The material was then quantitated from its optical density at 260 nm and sequenced by the Protein/DNA Technology Center at the Rockefeller University.

The nucleotide sequence contained an open reading frame over its length, up to a sequence which corresponded to the consensus sequence of a cleavage site of the enzyme Apo I. Following this point, a second open reading frame encoded a different reading frame up to the end of the product. The initial sequence information was found to match the initial sequence and to extend it yet further towards the C-terminus of the protein. The second reading frame was found to end in a sequence which matched the 5′-terminus of the previously determined sequence and, thus, represents an extension of the sequence towards the N-terminus of the protein.

Additional sequence information was obtained using the above primers and a template generated using S. aureus genomic DNA circularized via ligation with T4 ligase following digestion with Cla I. The PCR product was generated using 35 cycles of the following program: denaturation at 94° C. for 1 min.; annealing at 55° C. for 1 min.; and extension at 68° C. for 3 minutes and 30 s. The PCR products were electrophoresed in a 0.8% agarose gel, eluted with a Qiagen gel elution kit, divided into five separate aliquots, and used as a template reamplification via PCR with the same primers described above. The final product was electrophoresed in an 0.8% agarose gel, visualized via staining with ethidium bromide under ultraviolet light, and excised from the gel. The excised gel slice was frozen, and centrifuged at 12,000 rpm for 15 min. The supernatant was cleaned using a Qiagen PCR purification kit. The material was then quantitated via optical density at 260 nm and sequenced by the Protein/DNA Technology Center at Rockefeller University. The open reading frames continued past 500 bases. Therefore, the following additional sequencing primers were designed from the sequence to obtain further information:

First primer

(SEQ. ID. No. 63)

| | |
|---|---|
| cgttttaatg catgcttaga aacgatatca g | 31 |
| Second primer | |

(SEQ. ID. No. 64)

| | |
|---|---|
| cattgctaag caacgttacg gtccaacagg c | 31 |

The N-terminal and C-terminal nucleotide sequence extensions generated using this circular PCR product completed the 5′ region of the gene (encoding the N-terminus of DnaB); however, a stop codon was not reached in the 3′ region and, thus, a small amount of sequence is still needed to complete this gene.

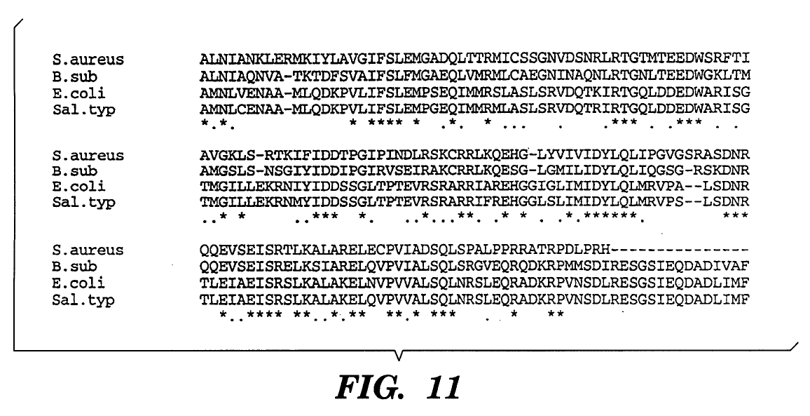

The alignment of the S. aureus dnaB with E. coli dnaB and the dnaB genes of B. subtilis and S. typhimurium is shown in FIG. 11.

### Example 17

### Identification and Cloning of S. aureus holB

The S. aureus holB was identified by searching the S. aureus database with the sequences of S. pyogenes δ′ subunit. The S. aureus holB encodes a 253 residue protein of about 28 kDa. The holB gene was amplified by PCR using an upstream 69-mer primer as follows:

| Upstream Primer (SEQ. ID. No. 65): | |
|---|---|
| ggataacaat tccccgctag caataatttt gtttaacttt aagaaggaga tata cccatg | 60 |
| gatgaacag | 69 |

which contains an NcoI site (underlined), and a downstream 39-mer primer as follows:

| Downstream Primer (SEQ. ID. No. 66): | |
|---|---|
| aattttaaag gatccgtgta taatattcta attttcccg | 39 |

which contains a BamHI site (underlined). The PCR product was digested with NcoI and BamHI, purified, and ligated into the NcoI and BamHI sites of pET11a to produce plasmid pETSaholB.

### Example 18

### Purification of S. aureus δ′

The pETSaholB plasmid of Example 17 was transformed into E. coli BL21(DE3)recA. A single colony was used to innoculate 2 L of LB media supplemented with 200 μg/ml ampicillin. Cells (2 L) were grown at 37° C. to OD₆₀₀=0.5 at which point the temperature was lowered to 15° C. and 0.5 mM IPTG was added. After 16 hr of induction, cells were collected by centrifugation, resuspended in 50 mM Tris-HCl (pH 7.5), 10% sucrose, 1 M NaCl, 30 mM spermidine, 5 mM DTT, and 2 mM EDTA. Cells were lysed by two passages through a French press (15,000 psi), followed by centrifugation at 13,000 rpm for 30 min at 4° C. Ammonium sulfate (0.3 g/ml) was added to the clarified lysate. The pellet was backwashed in 30 ml buffer A containing 0.1 M NaCl and 0.24 g/ml ammonium sulfate using a Dounce homogenizer, then the pellet was recovered by centrifugation. The resulting pellet was resuspended in 20 ml of buffer A and dialyzed against buffer A. The dialyzed protein was applied to a 20 ml FFQ Sepharose column equilibrated in buffer A and eluted with a 200 ml linear gradient of 0-500 mM NaCl in buffer A; 80 fractions were collected. Peak fractions (54-75) were combined (72 mg) and dialyzed against buffer A. The δ′ preparation was aliquoted and stored frozen at −80° C.

### Example 19

### Identification and Cloning of S. aureus holA

The S. aureus holA gene was identified by searching the S. aureus database with the sequences of E. coli and S. pyogenes δ subunits. The S. aureus holA gene encodes a 288 residue protein of about 32 kDa. The holA gene was amplified by PCR using an upstream 28-mer primer as follows:

| Upstream Primer (SEQ. ID. No. 67): | |
|---|---|
| gggagtttgt aatccatgga tgaacagc | 28 |

which contains a NcoI site (underlined), and a downstream 37-mer primer as follows:

| Downstream Primer (SEQ. ID. No. 68): | |
|---|---|
| ctgaacacct attaccctag gcatctaact cacaccc | 37 |

which contains a BamHI site (underlined). The PCR product was digested with NcoI and BamHI, purified, and ligated into the NcoI and BamHI sites of pET11a to produce plasmid pETSaholA.

### Example 20

### Purification of S. aureus 8

The pETSaholA plasmid of Example 19 was transformed into E. coli NovaBlue (recA1 lac[F′proA⁺B⁺ lac^{q}ZΔM]::Tn10(Tc^{R})) (Novagen). A single colony was used to innoculate 12 L of LB media supplemented with 200 μg/ml ampicillin. Cells (12 L) were grown at 37° C. to OD₆₀₀=0.5 at which point the temperature was lowered to 15° C. and 0.5 mM IPTG was added. After 16 hr of induction, cells were collected by centrifugation, resuspended in 50 mM Tris-HCl (pH 7.5), 10% sucrose, 1M NaCl, 30 mM spermidine, 5 mM DTT, and 2 mM EDTA. Cells were lysed by two passages through a French press (15,000 psi), followed by centrifugation at 13,000 rpm for 30 min at 4° C. Ammonium sulfate (0.3 g/ml) was added to the clarified lysate. The resulting pellet was resuspended in 250 ml of buffer A. The dialyzed protein was applied to a 100 ml FFQ Sepharose column equilibrated in buffer A and eluted with a 1000 ml linear gradient of 0-500 mM NaCl in buffer A; 80 fractions were collected. Peak fractions (40-49) were combined (65 mg) and dialyzed against buffer A. The dialyzed protein was applied to a 8 ml MonoQ Sepharose column equilibrated in buffer A and eluted with a 80 ml linear gradient of 0-500 mM NaCl in buffer A; 80 fractions were collected. Peak fractions of the δ preparation were stored frozen at −80° C.

### Example 21

### Constitution of a Processive S. aureus DNA Polymerase III Enzyme from Three Components

The PolC (alpha-large) requires the β clamp for processivity, which in turn requires the clamp loader (τδδ′) for assembly onto DNA. The S. aureus clamp loader, τδδ′ complex, was assembled by mixing the three proteins as follows: 400 μg of τ and 80 μg each of τδ and δ′ were mixed in buffer A containing no NaCl and preincubated at 15° C. for 10 min. The mixture was injected onto a 1 ml MonoQ column equilibrated in buffer A, and then eluted with a 30 ml linear gradient of 0-500 mM NaCl in buffer A; 60 fractions were collected. Fractions were analyzed in a 10% SDS-polyacrylamide gel stained with Coomassie Blue. Peak fractions (40-50) were combined and concentrated using a Centricon 30 concentrator.

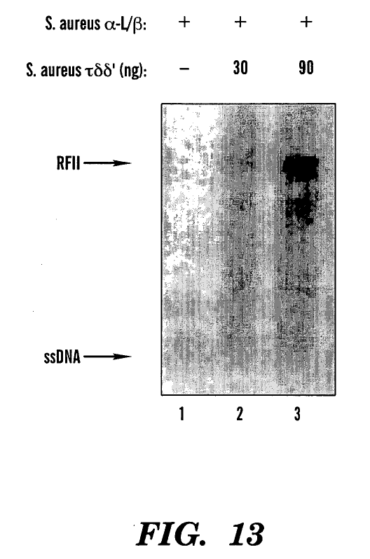

The ability of the three components to work together to form the processive Pol III was tested by determining whether τδδ′ and β clamp could confer the ability of PolC to completely extend a single primer full circle around a large 7.2 kb circular M13mp18 ssDNA genome. Replication reaction contained 70 ng (25 fmol) on singly primed M13mp18 ssDNA, 20 ng S. aureus β, 50 ng S. aureus PolC, either 30 ng or 90 ng of S. aureus τδδ′ (when indicated), and 0.82 μg of S. pyogenes SSB in 24 μl of 20 mM Tris-HCl (pH 7.5), 4% glycerol, 0.1 mM EDTA, 5 mM DTT, 2 mM ATP, 8 mM MgCl₂, 40 μg/ml BSA, and 60 mM each of dGTP and dCTP. Reactions were pre-incubated for 2 min at 37° C. to assemble protein complexes on the primer terminus. DNA synthesis was initiated upon addition of 1.5 μl dATP and ³²P-TTP (specific activity 2,000-4,000 cpm/pmol) and synthesis was allowed to proceed for 1 min before being quenched with an equal volume (25 μl) of a solution of 1% SDS and 40 mM EDTA. One-half of the quenched reaction was analyzed for total DNA synthesis using DE81 paper as described, and the other half was analyzed by agarose gel phoresis. An autoradiogram of the agarose gel analysis of the replication products is depicted in FIG. 13, which shows that the presence of PolC and β, but absence of τδδ′ (lane 1) gives no full length circular duplex (RFII). However, in the presence of τδδ′ (lanes 2 and 3), full length circular duplex DNA (RFII) is produced, as expected for the action of a processive Pol III holozyme.

### Example 22

### General Induction/Purification Conditions for S. pyogenes

The purification protocols for S. pyogenes proteins were performed using following standardized conditions. Cells were grown from a single colony, freshly transformed overnight. Cells were grown in 200 μg/ml Ampicillin to OD600=0.3-0.4, at which point cultures were chilled prior to addition of IPTG (to a final concentration of 0.5 mM) and were allowed to incubate for 16 hrs at 15° C. Following this, all procedures were performed at 4° C. Cell paste (1-2 g/liter of culture) was resuspended (10 ml/g cell paste) in 50 mM Tris-HCl (pH 7.5)/10% Sucrose/1 M NaCl/5 mM DTT/30 mM Spermidine/1× Heat lysis buffer (50 mM Tris-HCl (pH 7.5), 1% Sucrose, 100 mM NaCl, 2 mM EDTA). Cells were lysed by two passages through the French Press (15,000 psi) followed by centrifugation at 14,000 rpm at 4° C. Ammonium sulfate, when added to the cleared lysate, was added gradually. Precipitate was allowed to settle on ice for a minimum of 30 min prior to collection by centrifugation. Protein pellets were resuspended in buffer A (50 mM Tris-HCl pH 7.5, 1 mM EDTA, 5 mM DTT, 10% glycerol) and dialyzed for over 3 hours in the same buffer. Column design is based on the manufacturer's suggested capacities: Fast Flow Q (FFQ) and MonoQ are 20 mg protein/ml resin, Heparin-Affigel agarose is 1.2 mg protein/ml resin. Elution was performed using 10 column volume (c.v.) gradients, and the entire gradient elution profile was collected in 80 fractions. Unless mentioned otherwise all columns were equilibrated and eluted with buffer A.

### Example 23

### Identification of a S. pyogenes holA gene Encoding a Functional Delta Subunit and Purification of the Delta Subunit

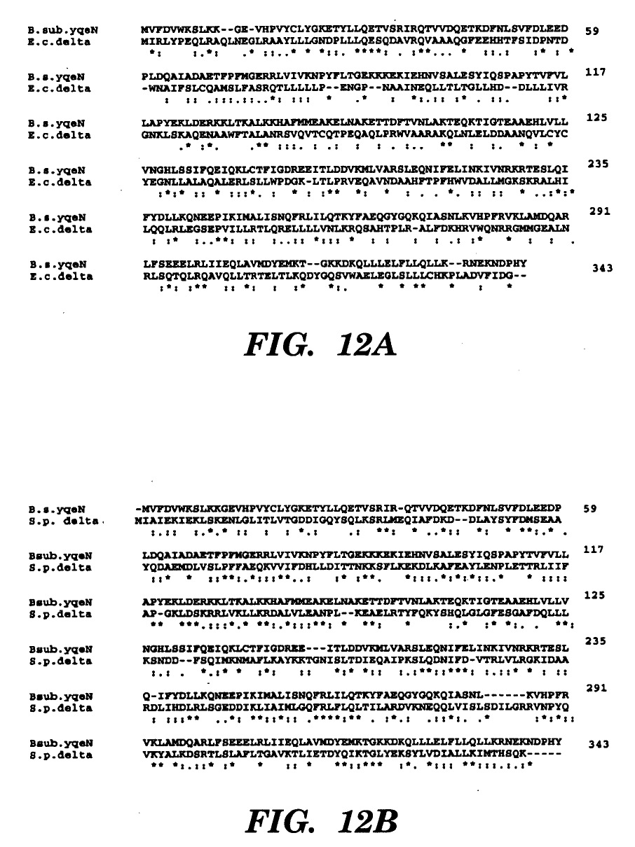

Alignment of E. coli delta subunit with 10 other putative holA products from unfinished genome databases of Gram negative bacteria indicates a region of conserved amino acid sequence. Amino acids Q140 to L230 of E. coli delta were used to search the B. subtilis genome database for a Gram positive delta homolog. This search revealed yqeN, a potential reading frame of unknown function, as the highest scoring sequence. Although the score was low, it was treated as a candidate for Gram positive delta. The alignment with E. coli delta is shown in FIG. 12A. A Streptococcus pyogenes genome database was searched with yqeN. Two contigs which represent N- (contig 206) and C- (contig 264) termini of S. pyogenes delta subunit were identified. The alignment of the putative S. pyogenes holA with B. subtilis yqeN is shown in FIG. 12B. The following primers were used to obtain PCR products for delta subunit:

| holA Upstream | |
|---|---|
| (SEQ. ID No. 69) | |
| ggagcagatt gcttttgata catatgattg gcctattc | 38 |
| holA Downstream | |
| (SEQ. ID No. 70) | |
| ttgtctccgc atcaaactgg gatccaagag catcatacgc gtatgg | 46 |

These primers were used to amplify the holA gene from S. pyogenes genomic DNA. The PCR product was digested with NdeI and BamHI, purified and ligated into the pET11a vector to produce pET11a.S.p.holA.

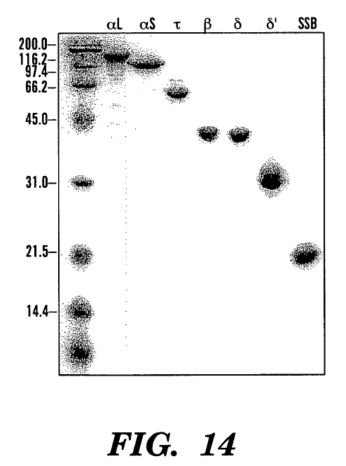

The pET11a.S.p.holA plasmid was transformed into the BL21(DE3)RecA-strain of E. coli. A single colony from an overnight transformation was used to innoculate 12 L LB broth supplemented with 200 μg/ml Ampicillin. Cells were grown at 37° C. to OD600=0.5, at which point the temperature was lowered to 15° C. and 0.5 mM IPTG was added. Induction proceeded for 16 hrs. In the morning, cells were collected by centrifugation and resuspended in 50 mM Tris-HCl (pH 7.5)/10% Sucrose/1× Heat Lysis Buffer/1M NaCl/30 mM Spermidine/5 mM DTT. Cells were lysed by two passages through the French press (15,000 psi), followed by centrifugation at 13,000 rpm for 30 min. The supernatant was decanted and ammonium sulfate was added to a final concentration of 0.226 g/ml. The resulting pellet was collected by centrifugation and resuspended in 20 ml of buffer A. The resuspended pellet was dialyzed against buffer A containing no salt. The dialyzed protein (500 mg) was loaded onto a FFQ-Sepharose (35 ml) column and eluted with a linear gradient from 0-500 mM NaCl (10 c.v.). The peak fractions (21-45) were combined and dialyzed against buffer A (0 NaCl) for 3 hrs, then diluted to a conductivity of 50 mM NaCl and loaded (160 mg) onto a 120 ml Heparin-Affigel column. Protein was eluted with a linear gradient of 0-500 mM NaCl (10 c.v.). The fractions containing the least contaminants (39-51) were precipitated with ammonium sulfate (0.226 g), collected by centrifugation, resuspended 5 ml of buffer A, and dialyzed in buffer A containing 200 mM NaCl. The delta subunit was stored at −80° C. The final delta subunit preparation is shown in the lane marked 6 of the Coomassie Blue stained SDS-polyacrylamide gel of FIG. 14. Yield=65 mg.

### Example 24

### Identification of S. pyogenes holB Encoding Delta Prime and Purification of the Delta Prime Subunit

A search of the S. pyogenes genome database with the predicted B. subtilis delta prime amino acid sequence revealed a DNA sequence in contig #209 (previously known as contig #210) that predicted a high scoring match for a gene encoding a delta prime protein. The following primers were used to obtain PCR products for holB:

| holB Upstream | |
|---|---|
| (SEQ. ID. No. 71) | |
| gcctaggata agggagggta catatggatt tagcgc | 36 |
| holB Downstream | |
| (SEQ. ID. No. 72) | |
| cgggcaagtc ttttgacaag cttcggatcc ccataacgaa ttcc | 44 |

The PCR product obtained from these primers was digested with NdeI and BamHI, purified and ligated into the pET11a vector to produce pET11a.S.p.holB.

The pET11a.S.p.holB plasmid was transformed into the BL21(DE3)RecA-strain of E. coli. A single colony from an overnight transformation was used to innoculate 12 L LB broth supplemented with 200 μg/ml Ampicillin. Cells were grown at 37° C. to O.D.600=0.4, at which point the temperature was lowered to 15° C. and 0.5 mM IPTG was added. Induction proceeded for 16 hrs. In the morning, cells were collected by centrifugation and resuspended in 100 ml 50 mM Tris-HCl (pH 7.5)/10% Sucrose/1× Heat Lysis Buffer. Lysis was initiated upon addition of 0.4 mg/ml lysozyme followed by a 1 hr incubation on ice. Lysate was clarified by centrifugation at 13,000 rpm for 30 min. Ammonium sulfate was added to the supernatant to a final concentration of 0.3 g/ml. The protein pellet was resuspended in buffer A (0.1 M NaCl)+0.24 g/ml ammonium sulfate and clarified by centrifugation. The resulting protein pellet was resuspended in 20 ml of buffer A and dialyzed against buffer A. The dialyzed protein (450 mg) was loaded onto a 30 ml FFQ-Sepharose column and eluted with a linear gradient from 0-500 mM NaCl. The peak fractions were combined (fr#20-30 containing 130 mg) and dialyzed against buffer A and loaded (70 mg) onto a 50 ml Heparin-Affigel column. Protein was eluted with a linear gradient of 0-500 mM NaCl. Delta prime binds weakly to both resins and elutes in the beginning of the gradient. This delta prime subunit was stored frozen at −80° C. The final delta prime subunit preparation is shown in lane marked δ′ of the Coomassie Blue stained SDS-polyacrylamide gel of FIG. 14. Yield=40 mg.

### Example 25

### Identification of the S. pyogenes dnaX Gene and Purification of the Tau Subunit

A search of the S. pyogenes genome database with the putative B. subtilis tau amino acid sequence revealed a DNA sequence in contig #284 (previously known as contig #289) with a high scoring match which predicted a gene encoding for a tau subunit protein. A set of PCR primers to 5′- and 3′-termini of the putative gene sequence were designed to include restriction enzyme recognition sequences for NdeI and BamHI sites, respectively. These primers are:

dnaX Upstream

(SEQ. ID. No. 73)

| | |
|---|---|
| ggagttaaaa acatatgtat caagctcttt atc | 33 |
| dnaX Downstream | |

(SEQ. ID. No. 74)

| | |
|---|---|
| cgtgggtaag ggcaaaacgg atcccttatg tatttcag | 38 |

A PCR product obtained with the above primers was digested with NdeI and BamHI, purified and ligated into pET11a vector to produce pET11a.S.p.dnaX.

The pET11a.S.p.dnaX plasmid was transformed into the BL21(DE3)RecA-strain of E. coli. A single colony from an overnight transformation was used to innoculate 24 L LB broth supplemented with 200 μg/ml Ampicillin. Cells were grown at 37° C. to O.D.600=0.5, at which point the temperature was lowered to 15° C. and 0.5 mM IPTG was added. Induction proceeded for 16 hrs. In the morning, cells were collected by centrifugation and resuspended in 200 mls of 50 mM Tris-HCl (pH 7.5)/10% Sucrose/1× Heat Lysis Buffer/1M NaCl/30 mM Spermidine/5 mM DTT/5 mM EDTA. Cells were lysed by two passages through the French press (15,000 psi), followed by centrifugation at 13,000 rpm for 30 min. The supernatant (2.4 gm) was dialyzed against buffer A containing 50 mM NaCl, loaded onto a 120 ml FFQ column (without ammonium sulfate precipitation) and eluted with a linear gradient of 100-700 mM NaCl. The peak fractions (fr#41-55) were combined, diluted with buffer A containing no salt (a dilution of 1/5) to a conductivity of 100 mM NaCl, loaded (310 mg) onto a 300 ml Heparin-Affigel column, and eluted with a linear gradient of 100-500 mM NaCl. The peak fractions (fr#21-36) were combined, dialyzed against buffer A, loaded (87 mg) onto 10 ml FFQ column, and eluted as described for the first FFQ column. The peak fractions (fr#27-41) were concentrated by centrifugation in Centriprep 30 filtration unit and frozen at −80° C. The final tau subunit preparation is shown in the lane marked τ of the Coomassie Blue stained SDS-polyacrylamide gel of FIG. 14. Yield=103 mg.

### Example 26

### Identification of the S. pyogenes dnaN Gene and Purification of the Beta Subunit

A search of the S. pyogenes genome database with the putative B. subtilis beta subunit amino acid sequence revealed a DNA sequence (contig #266) with a high scoring match which predicted a gene encoding for a beta subunit protein. A set of PCR primers to 5′- and 3′-termini of the putative gene sequence were designed to include restriction enzyme recognition sequences for NdeI and BamHI, respectively. The primers were:

dnaN Upstream

(SEQ. ID. No. 75)

| | |
|---|---|
| ggagttcata tgattcaatt ttcaaattaa tcgc | 34 |
| dnaN Downstream | |

(SEQ. ID. No. 76)

| | |
|---|---|
| tatcagctcc tggatccagt accttccatt gattagcc | 38 |

A PCR product obtained with these primers was digested with NdeI and BamHI, purified and ligated into pET16b vector to produce pET16b.S.p.dnaN.

The pET16b.S.p.dnaN plasmid was transformed into the BL21(DE3)RecA-strain of E. coli. A single colony from an overnight transformation was used to innoculate 15 L LB broth supplemented with 200 μg/ml Ampicillin. Cells were grown at 37° C. to O.D.600=0.4, at which the point temperature was lowered to 15° C. and 0.5 mM IPTG was added. Induction proceeded for 16 hrs. In the morning, cells were collected by centrifugation and resuspended in 100 ml 50 mM Tris-HCl (pH 7.5)/10% Sucrose/1× Heat Lysis Buffer/1 M NaCl/5 mM DTT/30 mM Spermidine/5 mM EDTA. Cells were lysed by two passages through the French press (15,000 psi), followed by centrifugation at 13,000 rpm for 30 min. Ammonium sulfate was added to the supernatant to a final concentration of 0.3 g/ml. The resulting protein pellet was resuspended and dialyzed against buffer A containing 50 mM NaCl. The dialyzed protein (300 mg) was loaded onto a 45 ml FFQ-Sepharose column and eluted with a linear gradient from 50-500 mM NaCl. The peak fractions (16-30) were combined, dialyzed against buffer A containing 50 mM NaCl, loaded onto a 25 ml EAH-Sepharose column, and eluted with a linear gradient of 50-500 mM NaCl. The fractions containing the least contaminants were combined into two pools (pool I 10-17, pool II 19-27). Each pool was further purified on a 8 ml MonoQ column (performed under conditions described for the FFQ column above). The final beta subunit preparation is shown in the lane marked β of the Coomassie Blue stained SDS-polyacrylamide gel of FIG. 14. Yield=48 mg.

### Example 27

### Identification of the S. pyogenes polC Gene and Purification of the Alpha-Large Polymerase Subunit

A search of the B. subtilis genome database with the E. coli alpha subunit amino acid sequence revealed two DNA sequences with a high scoring match which predicted two genes encoding alpha-like polymerase subunits. The DNA sequence with the second highest scoring match which encoded the largest of the two polymerase subunits also appeared to encode for the epsilon exonuclease domain at the N-terminus of the putative alpha subunit. A search of the B. subtilis genome database with S. pyogenes DNA sequence confirmed this nucleotide sequence to encode the Gram positive homolog of the E. coli replicative polymerase subunit (alpha). This Gram negative alpha-like subunit lacked homology to epsilon. The gene encoding the large alpha polypeptide sequence (alpha-large) will be referred to as the product of the polC gene and the gene encoding the smaller Gram-negative alpha-like polymerase (alpha-small) will be referred to as the product of the polE or dnaE gene (see Example 28).

The alpha-large polymerase polypeptide is a product of two overlapping contigs; contig #197 (renamed #193) encodes the N-terminal 630 amino acids, and contig #278 (renamed #273) encodes the C-terminal 1392 amino acids. The putative Open Reading Frame generates a 1464 amino acid polypeptide (SEQ. ID. No. 18). Since the polC nucleotide sequence contained several NdeI sites, a primer was designed to mutate two restriction endonuclease sites in the pET11a nucleotide sequence upstream of the N-terminus of the gene; an XbaI restriction site was mutated to an NheI restriction site and an NdeI restriction site at the starting ATG was removed. A 74mer primer which spans from mutated XbaI site upstream of T7 promoter includes NheI site, rbs site (ribosome binding site), mutated NdeI site and first 10 amino acid codons of polC gene sequence. The following primers were used in a PCR reaction to amplify polC gene from S. pyogenes genomic DNA:

| polC Upstream | |
|---|---|
| (SEQ. ID. No. 77) | |
| ggataacaat tccccgctag caataatttt gtttaacttt aagaaggaga tatacccatg | 60 |
| tcagatttat tcgc | 74 |
| polC Downstream | |
| (SEQ. ID. No. 78) | |
| cggtgtctct atctaaatga ctcatttggg atcctcgctt tatacggtat gtcacag | 57 |

Elongase (BRL) produced the best amplification results. PCR reaction conditions were: 5 μg genomic DNA, 20 ng of each primer, 1 ml Elongase, 60 μM each dNTP, in 100 ml Elongase reaction buffer for 1 min at 94° C., 1 min at 55° C., and 6 min at 60° C. repeated for 40 cycles. The resulting 4000 bp PCR fragment was digested with NheI and BamHI, purified and ligated into the pET11a vector (digested with XbaI and BamHI) to produce pET11a.S.p.polC.

The pET11a.S.p.polC plasmid was transformed into the BL21(DE3)RecA-strain of E. coli. A single colony from an overnight transformation was used to innoculate 24 L LB broth supplemented with 200 μg/ml Ampicillin. Cells were grown at 37° C. to OD600=0.4 at which point temperature was lowered to 15° C. and 0.5 mM IPTG was added. Induction proceeded for 16 hrs. In the morning, cells (12 g) were collected by centrifugation and resuspended in 100 ml 50 mM Tris-HCl (pH 7.5)/10% Sucrose/1× Heat Lysis Buffer/1 M NaCl/5 mM DTT/30 mM Spermidine/5 mM EDTA. Cells were lysed by two passages through the French press (15,000 psi), followed by centrifugation at 13,000 rpm for 30 min. Ammonium sulfate was added to the supernatant to a final concentration of 0.226 g/ml. The precipitate was collected by centrifugation. The protein pellet (220 mg resuspended in buffer A) was dialyzed against buffer A containing 150 mM NaCl, loaded onto an 8 ml FFQ column equilibrated with buffer A containing 150 mM NaCl, and eluted with a linear gradient of buffer A containing 150-600 mM NaCl. The fractions containing the least contaminants (fr#42-64) were combined and precipitated with ammonium sulfate (0.226 g/ml). The precipitate was collected by centrifugation and resuspended in buffer A (10 mg/ml in 5 ml). A fraction (1 ml=10 mgs) of the concentrated protein was dialyzed, loaded onto 10 ml ssDNA-agarose column, and eluted with a linear gradient of 50-500 mM NaCl. The peak fractions (fr#30-50) were combined and concentrated with ammonium sulfate (as above). The final alpha-large subunit preparation is shown in lane marked α_{L} of the Coomassie Blue stained SDS-polyacrylamide gel of FIG. 14. Yield 4 mgs.

### Example 28

### Identification of the S. pyogenes dnaE Gene and Purification of the Alpha-Small Polymerase

A search of the B. subtilis genome database using the E. coli alpha subunit amino acid sequence revealed two DNA sequences with a high scoring match which predicted two genes encoding for alpha-like polymerase subunits. The DNA sequence with the highest scoring match encodes a smaller alpha polymerase which does not contain an exonuclease domain. The putative short alpha DNA sequence is a product of the open reading frame in contig #253 of the S. pyogenes genome database. A set of PCR primers to 5′- and 3′-termini of the putative gene sequence were designed to include restriction enzyme recognition sequences for NdeI and BamHI, respectively. The primers were:

α-short Upstream

(SEQ. ID. No. 79)

| | |
|---|---|
| gggaacaaga taaccaagga ggaacccatg gttgctcaac ttg | 43 |
| α-short Downstream | |

(SEQ. ID. No. 80)

| | |
|---|---|
| cgaatagcag cgttcatacc aggatcctcg ccgccactgg | 40 |

A PCR product obtained with these primers was digested with NdeI and BamHI, purified and ligated into pET11a vector to produce pET11a.S.p.dnaE.

The pET11a.S.p.dnaE plasmid was transformed into the BL21(DE3)RecA-strain of E. coli. A single colony from an overnight transformation was used to innoculate 12 L LB broth supplemented with 200 l g/ml Ampicillin. Cells were grown at 37° C. to OD600=0.4, at which point temperature was lowered to 15° C. and 0.5 mM IPTG was added. Induction proceeded for 16 hrs. In the morning, cells were collected by centrifugation and resuspended in 100 mls 50 mM Tris-HCl (pH 7.5)/10% Sucrose/1× Heat Lysis Buffer/5 mM DTT/30 mM Spermidine/1M NaCl/5 mM EDTA. Cells were lysed by two passages through the French press (15,000 psi), followed by centrifugation at 13,000 rpm for 30 min. Ammonium sulfate was added to the supernatant to a final concentration of 0.226 g/ml. The precipitate was collected by centrifugation. The protein pellet (resuspended in buffer A) was then dialyzed against buffer A. The dialyzed protein (600 mg) was loaded onto a 30 ml FFQ and eluted with a linear gradient of buffer A containing 50-500 mM NaCl. The peak fractions (200 mg in fr #70-79) were dialyzed and loaded onto a 100 ml Heparin-Affigel column. The fractions containing the least contaminants (100 mg from fr #18-30) were pooled and dialyzed against buffer A containing 300 mM NaCl. The dialysate (50 mg) was loaded onto a 50 ml ssDNA-agarose column and eluted with a linear gradient of 300 mM-1M NaCl. The final alpha-small subunit preparation is shown in lane marked α_{S} of the Coomassie Blue stained SDS-polyacrylamide gel of FIG. 14. Yield=25 mg.

### Example 29

### Identification of the S. pyogenes ssb Gene and Purification of the Single Strand DNA-Binding Protein

Search of the S. pyogenes genome using the B. subtilis SSB amino acid sequence identified a polypeptide in contig #230(212) as having highest homology to single strand binding protein of several Gram negative bacteria. This contig lacked the first 26 amino acids at the N-terminus. Circular PCR was employed to identify the DNA encoding the N-terminus of the putative SSB protein. S. pyogenes genomic DNA was digested overnight with ApoI (5 μg chromosomal DNA in a 50 μl reaction). The DNA was extracted with phenol and precipitated with ethanol. The ApoI digested chromosomal DNA was self-ligated to generate circular template for future use in the circular PCR. A circular PCR was performed with primers designed to anneal back-to-back to amplify circularized ApoI reaction fragments. The primers were:

| ssb.circ Upstream | |
|---|---|
| (SEQ. ID. No. 81) | |
| caccattttgg cttttaaagg tacggttaac agcaagtgtg aaggtagcc | 49 |
| ssb.circ Downstream | |
| (SEQ. ID. No. 82) | |
| gaacgcgagg cagatttcat taactgtgtg atctggcg | 38 |

The PCR reaction conditions were as follows: 100 ng circularized S. pyogenes genomic DNA, 20 ng each primer, 1 ml Elongase, 60 μM each dNTP, 100 l Elongase reaction buffer. Amplification was performed for 40 cycles as follows: denature, 1 min at 94° C.; anneal, 1 min at 55° C.; and extend, 5 min at 68° C. PCR products were cloned into the Topo TA vector following instructions of the manufacturer (Promega). Several positive clones were sequenced to obtain N-terminal nucleotide sequence. This information lead to design of the following primers with which the use of a standard PCR reaction generated whole ssb gene products. The primers were:

| ssb Upstream | |
|---|---|
| (SEQ. ID. No. 83) | |
| tttaaaagag ggtagcatat gattaataat gtagtactag ttggtcgc | 48 |
| ssb Downstream | |
| (SEQ. ID. No. 84) | |
| tttaaattta aacctaggtt caatccattc tgactagaat ggaagatcgt c | 51 |

The resulting PCR product was digested with NdeI and BamHI, purified and ligated into pET11a vector to produce pET11a.S.p.ssb.

The pET11a.S.p.ssb plasmid was transformed into the BL21(DE3)RecA-strain of E. coli. A single colony from an overnight transformation was used to innoculate 12 L LB broth supplemented with 200 μg/ml Ampicillin. Cells were grown at 37° C. to OD600=0.5, at which point 0.5 mM IPTG was added. At the end of the 3 hr induction, cells were collected by centrifugation and resuspended in 100 ml of 50 mM Tris-HCl (pH 7.5)/10% Sucrose/1× Heat Lysis Buffer/5 mM DTT/5 mM EDTA. The cell lysis was initiated upon addition of 0.4 mg/ml lysozyme followed by a 1 hr incubation on ice. The lysate was clarified by centrifugation at 13,000 rpm for 30 min. The SSB protein was significantly purified by sequential fractionation with ammonium sulfate in the following manner. Solid ammonium sulfate was added to the clarified lysate to a final concentration of 0.24 g/ml and the precipitated protein was collected by centrifugation at 13,000 rpm for 30 min. The resulting pellet was homogenized in buffer A (0.1 M NaCl)+0.24 g/ml ammonium sulfate and the precipitate was collected by centrifugation. This procedure was repeated with buffer A (0.1 M NaCl)+0.2 g/ml ammonium sulfate, buffer A (0.1 M NaCl+0.15 g/ml ammonium sulfate, and buffer A (0.1 M NaCl)+0.13 g/ml ammonium sulfate. The final pellet was resuspended in buffer A+0.15 M NaCl and dialyzed against the same buffer. The resulting pellet was resuspended in buffer A and dialyzed against buffer A containing 500 mM NaCl. The dialysate (300 mg) was diluted to 0.15 M NaCl before it was loaded onto a 20 ml MonoQ column and eluted with a linear gradient of 0.15 M-0.5 M NaCl in buffer A. The SSB protein elutes in the very beginning of the gradient. The peak fractions were combined (150 mg in fractions 16-30), diluted to 0.05 M NaCl, loaded onto a 10 ml ssDNA-agarose column, and eluted with 0.5 M NaCl. The peak fractions (32-62) were combined and frozen. The SSB was further purified over a MonoQ column to remove contaminating polymerase activity. The final single strand DNA binding protein preparation is shown in lane marked ssb of the Coomassie Blue stained SDS-polyacrylamide gel of FIG. 14. Yield=120 mg.

### Example 30

### First Demonstration that S. pyogenes holA Encodes a Delta Subunit Involved in Replication: Assembly of τδδ′ Complex

Gel filtration is a standard analytical technique to demonstrate direct protein-protein interaction. Purified τ, δ, δ′ proteins were used to examine whether they form a protein complex assembly. Gel filtration of τ mixed with either δ, δ′, or both δ and δ′ was performed using an HR 10/30 Superose 6 column equilibrated with buffer A containing 100 mM NaCl. Either δ (200 μg), δ′ (200 μg), or a mixture of δ and δ′ (200 μg each) was incubated for 30 min at 15° C. in 100 μl of buffer A containing 100 mM NaCl, and the entire mixture was injected onto the column. The mixture was resolved on the column by collection of 170 μL fractions after the initial void (6.6 μl) volume was collected. Fractions were analyzed by 10% SDS-polyacrylamide gels (30 μl/lane) stained with Coomassie Blue.

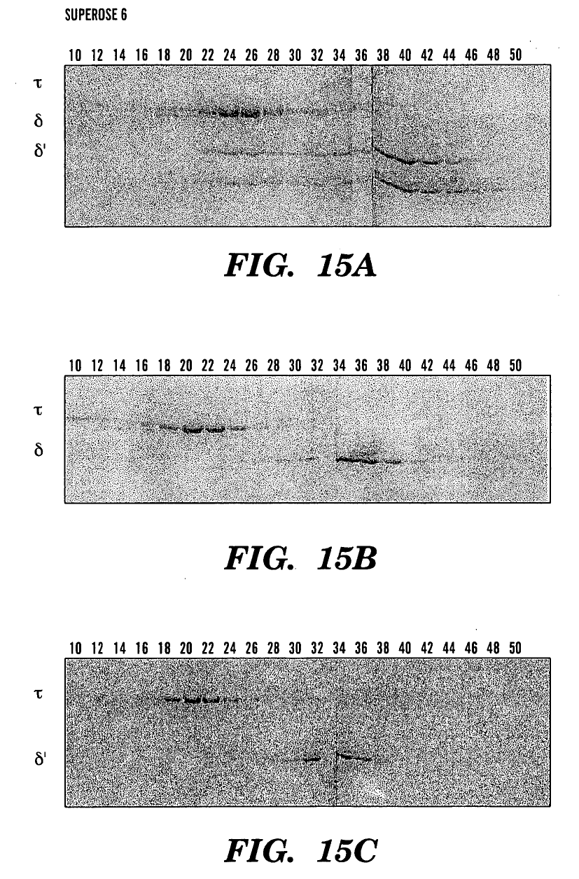

The results, in FIG. 15, demonstrate that under these conditions the τ protein exhibits no (weak) interaction with the delta (FIG. 15B) and the delta prime subunits (FIG. 15C) individually, and yet assembles readily into a complex when all the subunits are mixed in the reaction (FIG. 15A). The τ protein was mixed with a 2-fold molar excess of each δ and δ′, then gel filtered. A complex of τδδ′ was formed as demonstrated by coellution of δ and δ′ with τ (fr#22-30) whereas excess δδ′ complex elutes in later fractions (fr#38-46). To determine whether individual δ or δ′ subunits interact with τ, the τ subunit was mixed with either δ or δ′ and then gel filtered. The results demonstrate that a gel filterable complex does not form when τ is mixed with δ (FIG. 15B) or δ′ (FIG. 15C) subunits individually, as indicated by the absence of these subunits in the τ containing fractions (fr#20-26). Therefore, it appears that the presence of both δ and δ′ subunits is essential for the formation of the τδδ′ complex.

### Example 31

### Second Demonstration that S. pyogenes holA Encodes Delta: Functional Assembly of β on DNA

Gel filtration was used to demonstrate that the τ, δ, δ′ proteins form a functional clamp loading complex which is able to load the β clamp onto a circular DNA molecule. The reaction contained 0.5 pmol of gp2 nicked pBluescript plasmid (a circular double strand plasmid with a single nick produced by M13 gp2 protein), 1 pmol [³²P]β, 0.5 pmol τδδ′ complex, 0.25 pmol of either δ, δ′, τ were used in individual experiments when a subassembly of the complex was tested (τδ, τδ′, δδ′) in 75 μl buffer B (20 mM Tris-HCl (pH 7.5), 20% glycerol, 0.1 mM EDTA, 5 mM DTT, 2 mM ATP, 8 mM MgCl₂). β was incubated with nicked DNA for 10 min at 37° C. either alone, or in combination with various assemblies of the τ complex. All gel filtration experiments were performed at 4° C. The reaction mixtures were applied to a 5 ml column of Bio-Gel 15M (Bio-Rad) equilibrated in buffer B containing 100 mM NaCl. Fractions of 170 μl were collected and quantitated in the Scintillation counter.

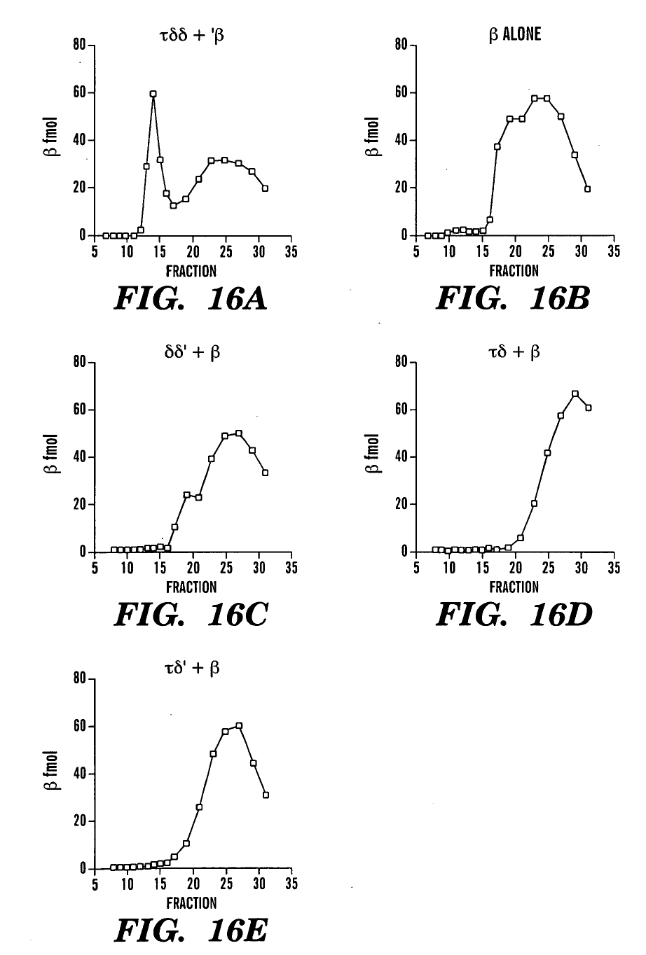

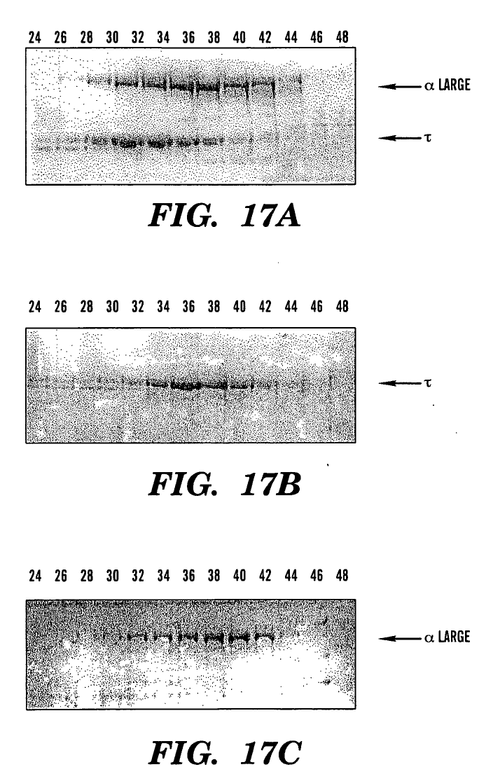

The results, in FIG. 16, demonstrate that the assembly of the ring onto a circular DNA molecule requires the presence of τ, δ, and δ′ proteins (FIG. 16A). In absence of any one of the subunits, loading onto DNA does not occur (FIG. 16B-E). The clamp loader complex (τδδ′) can be supplied as a mixture of τ, δ, δ′ subunits or as an assembled complex (purified from unassembled subunits by gel filtration, or by ion exchange chromatography on MonoQ). Proteins bound to the large DNA molecule elute in the early fractions (void fr#10-17) and resolve from free proteins that elute in later fractions (fr#18-35).

### Example 32

### The τ Subunit Product of the dnaX Gene Binds α-large

The interaction of S. pyogenes α and τ proteins was examined by analyzing a mixture of the proteins by gel filtration. Gel filtration of τ, α-large or a mixture of α-large and τ was performed using an HR 10/30 Superose 6 column equilibrated with buffer A containing 100 mM NaCl. Either α-large (400 μg) (200 μM) or a mixture of α-large and τ was incubated for 30 min at 15° C. in 100 μl of buffer A containing 100 mM NaCl, and the entire mixture was injected onto the column. The mixture was resolved on the column by collection of 170 μL fractions after the initial void (6.6 ml) volume was collected. Fractions were analyzed by 10% SDS-polyacrylamide gels (30 μl/lane) stained with Coomassie Blue.

The results show a complex of α_{L}τ was formed as demonstrated by coellution of α-large and τ (fr#30-38) proteins (FIG. 17A) compared to the elution profile of individual proteins (FIG. 17B-C). Also, the migration of the τ in the α_{L}τ complex changes significantly to a larger complex (4 fractions, from fr#37 to fr#33).

### Example 33

### Formation of α_{L}τδδ′ Complex

To determine whether a α_{L}τδδ′ complex could form, the following components were mixed: α-large (400 μg, 2.5 nmol), τ (200 μg, 1.3 nmol), δ (200 μg, 4.8 nmol), δ′ (200 μg, 5.75 pmol) in a final volume of 150 μL. The mixture was diluted to 300 ml with buffer A to lower conductivity of the sample to that equivalent of 100 mM NaCl and incubated for 30 min at 15° C. The mixture was injected onto a Superose 6 column (equilibrated with buffer A containing 100 mM NaCl) and fractions (170 μl) were collected after an initial 6.6 ml of void volume was collected. Fractions were analyzed by 10% SDS-polyacrylamide gels (30 μl/lane) stained with Coomassie Blue.

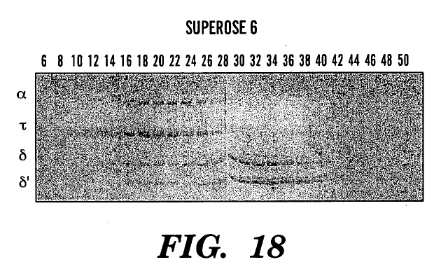

A gel filterable complex (FIG. 18) of α_{L}τδδ′ was formed as demonstrated by coellution of τ, δ and δ′ with α-large (fr#14-26), whereas excess δδ′ complex elutes in later fractions (fr#30-38). The migration of the τδδ′ protein complex in the α_{L}τδδ′ complex does not change significantly. The complex might dissociate under the nonequilibrium conditions of gel filtration due to low concentration of proteins, salt concentration and speed of resolution.

Next, ion exchange chromatography was used to analyze the protein mixture to prepare the reconstituted α_{L}τδδ′ complex of S. pyogenes. The α_{L}τδδ′ complex was reconstituted upon mixing α-large (10 mg, 62 nmol), τ (6 mg, 72 nmol), δ (3.3 mg, 80 nmol), δ′ (1.6 mg, 90 nmol). The α, τ, δ, δ′ protein mixture was dialyzed for 2 hrs against buffer A containing 50 mM NaCl. The entire mixture was loaded onto a 1 ml MonoQ column equilibrated in buffer A containing 50 mM NaCl. Proteins were eluted with a 20 column volume linear gradient of 50-500 mM NaCl in buffer A and 0.25 ml fractions were collected. Fractions were analyzed by 10% SDS-polyacrylamide gels (20 μl/lane) stained with Coomassie Blue.

Generally, the reconstitution of the α_{L}τδδ′ complex on a MonoQ column results in a tight salt resistant complex which elutes at 500 mM NaCl. The high concentration of the proteins in the eluted fractions contributes to stability of the complex.

### Example 34

### The S. pyogenes Three Component Pol III-L Polymerase is Rapid and Processive In DNA Synthesis

It was previously demonstrated (i.e., in Examples 29 and 30) that the putative delta subunit plays an integral part in the assembly of the τδδ′ complex (FIG. 15) and that this complex is sufficient to assemble β clamps onto circular primed DNA (FIG. 16). It was also shown that the strong interaction between the α-large and τ subunits (FIG. 17) results in an isolatable α_{L}τδδ′ complex (FIG. 18), similar to that of the E. coli DNA polymerase III*.

The MonoQ fractions containing α_{L}τδδ′ complex were then used to assemble β onto primed DNA and determine whether this now resulted in rapid and processive DNA synthesis. Replication reactions contained 70 ng of singly primed M1 mp18 ssDNA and 0.82 μg of S. pyogenes SSB in 25 μl buffer C (20 mM Tris-HCl (pH 7.5), 4% glycerol, 0.1 mM EDTA, 5 mM DTT, 2 mM ATP, 8 mM MgCl₂) with 60 μM each of dGTP, dCTP, and dATP, 30 μM cold TTP and 20 μM [α-³²P] TTP (specific activity of 2,000-4,000 cpm/pmol). The complex is assembled onto DNA in the following manner: 40 ng (3:1) or 140 ng (10:1) of the α_{L}τδδ′ complex and 60 ng of 3 protein were preincubated for 2 min at 30° C. in presence of SSB coated primed M13 DNA and two nucleotides (dCTP and dGTP). Reactions were initiated by addition of the two remaining nucleotides dATP and TTP and quenched with an equal volume of 1% SDS/40 mM EDTA. Each time point is a separate reaction.

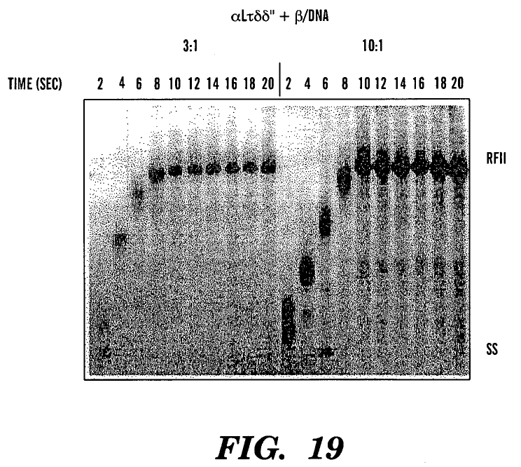

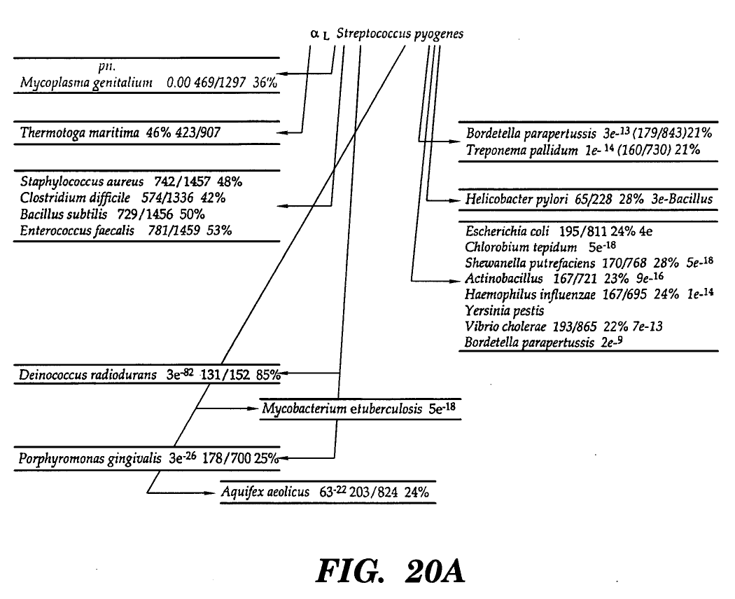

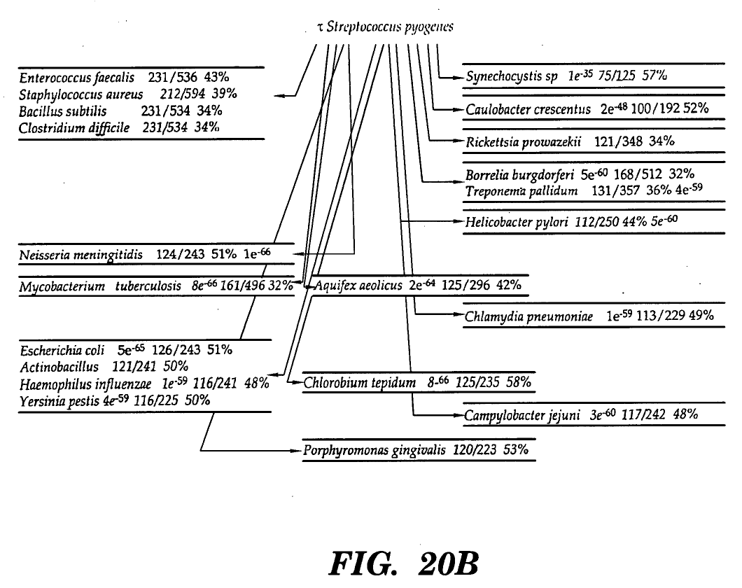

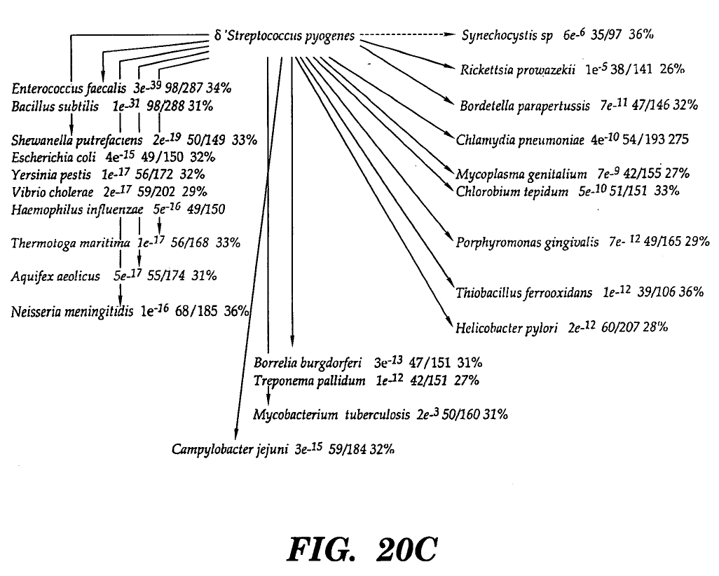

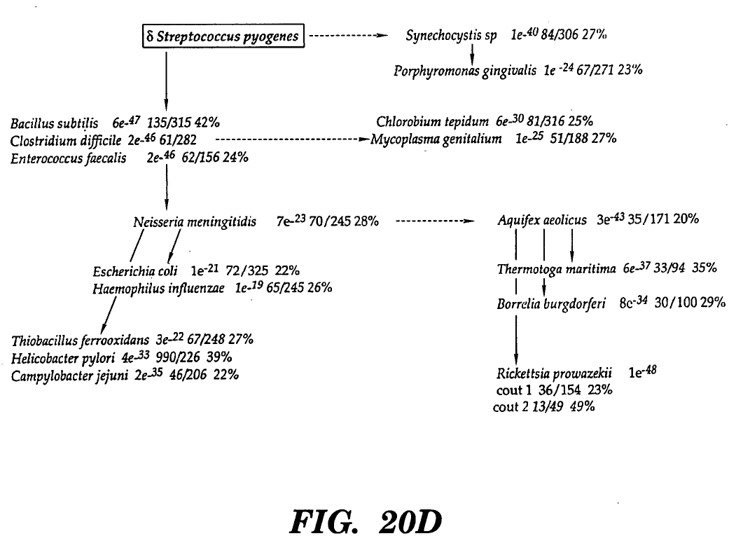

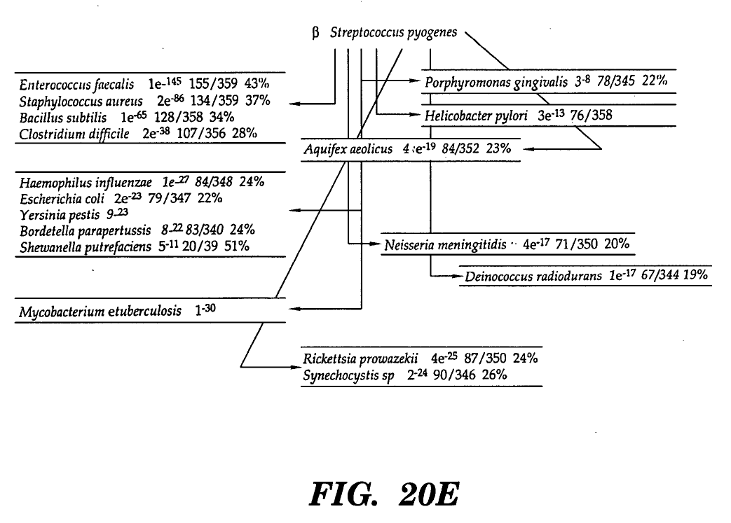

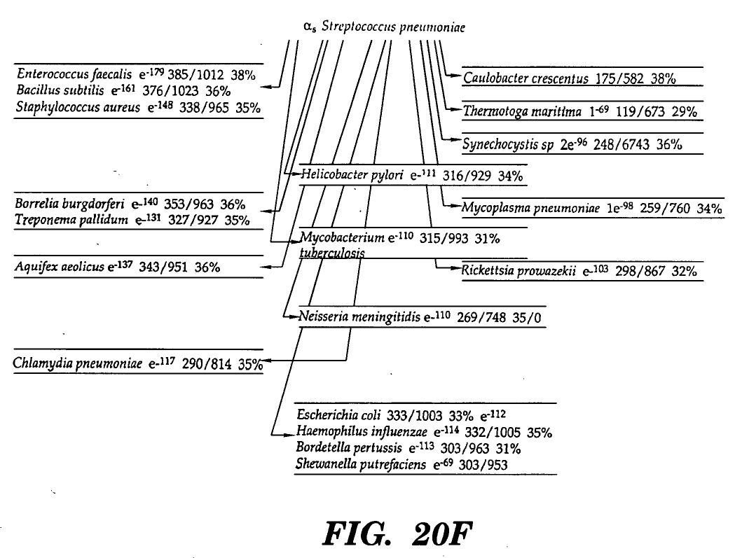

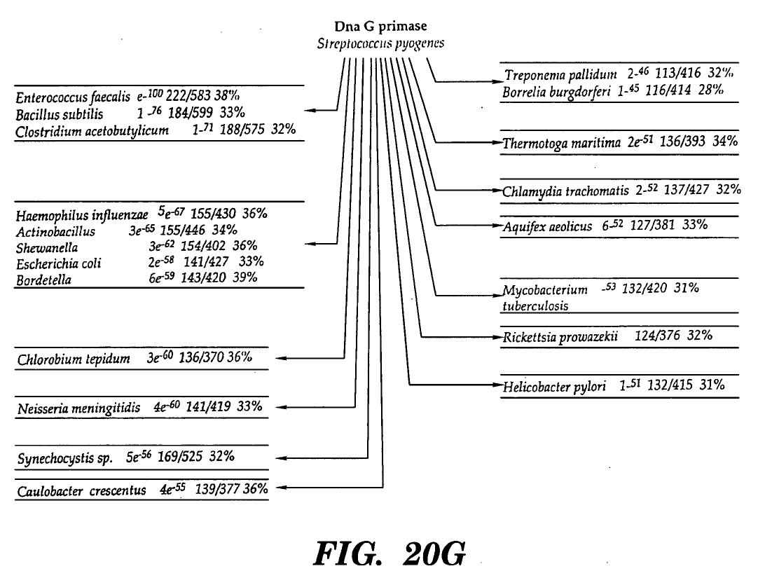

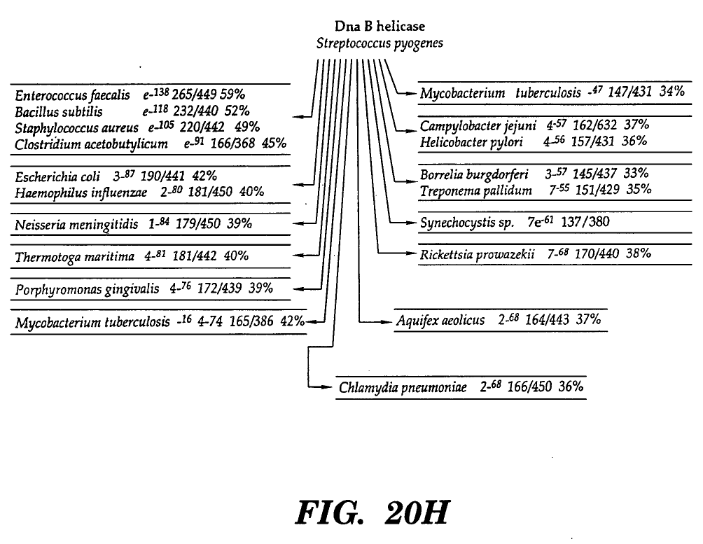

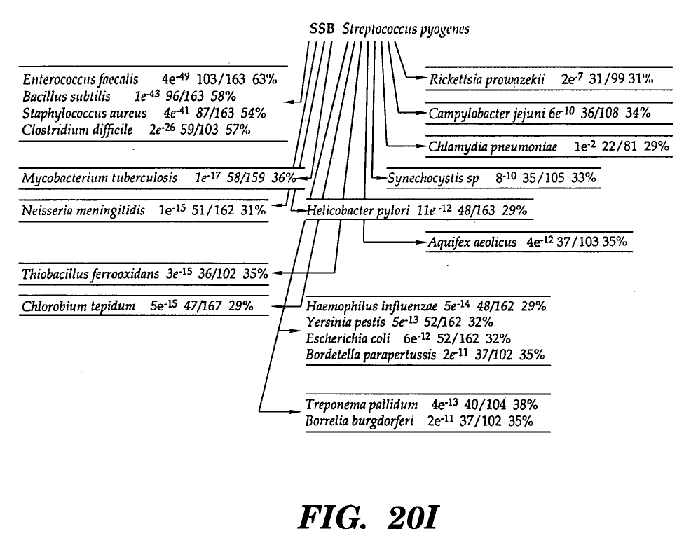

A time course of replication on singly primed circular M13mp18 ssDNA is shown in FIG. 19. The agarose gel analysis shows conversion of the oligonucleotide primed single stranded DNA to the slower migrating replicative form II. The fact that the speed of synthesis is independent of the concentration of polymerase in the reaction indicates that the α_{L}τδδ′ complex synthesizes DNA in a rapid and a highly processive manner. The S. pyogenes α_{L}τδδ′ complex in presence of the β clamp, completely replicates (is able to complete replication of) 7250 nt of M13mp18 ssDNA in 8-9 sec.

### Example 35

### The S. pyogenes DnaE (α-Small) Forms a Three-Component Polymerase with τδδ′ and β

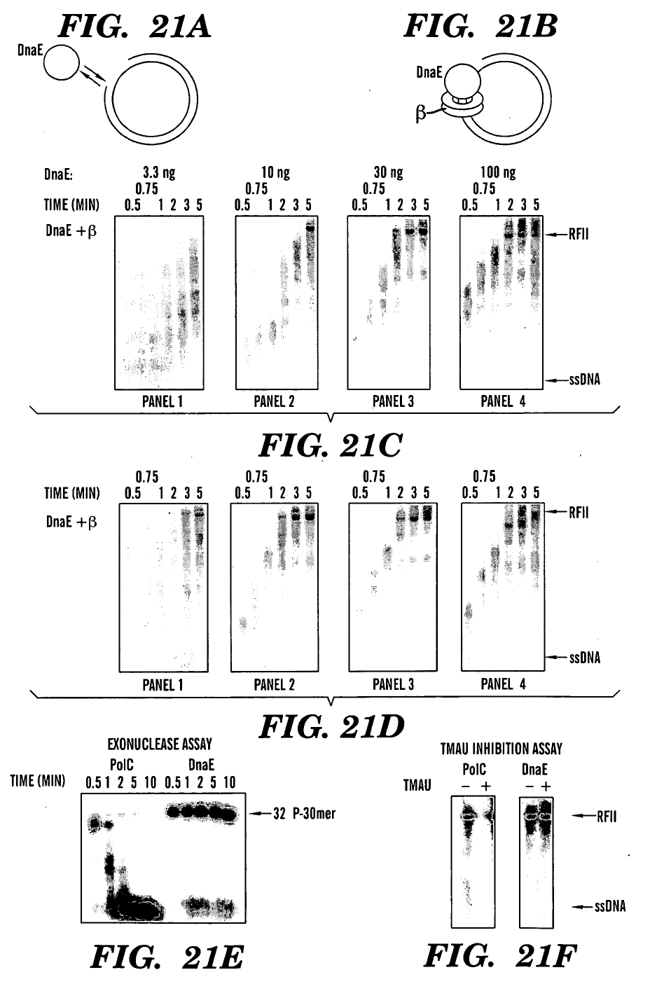

The S. pyogenes DnaE (α-small) polymerase is more homologous to E. coli α than S. pyogenes PolC. Thus, it seems reasonable to expect that the DnaE polymerase may also function with the β clamp (FIGS. 21A-B). To test DnaE for function with τδδ′ and β, replication reactions contained 70 ng (25 fmol) of 30-mer singly primed M13mp18 ssDNA, 0.82 μg of S. pyogenes SSB, and 3.3 ng-300 ng of DnaE (25 fmol-2.3 pmol) in 23.5 μl of 20 mM Tris-HCl (pH 7.5), 4% glycerol, 0.1 mM EDTA, 5 mM dithiothreitol (DTT), 40 μg/ml BSA, 2 mM ATP, 8 mM MgCl₂, and 60 μM each of dGTP and dCTP. When present, reactions included 43.3 ng of β and 10 ng of τδδ′. Reactions were preincubated for 3 min at 37° C., and then NaCl was added to 40 mM followed by another 2 min at 37° C. DNA synthesis was initiated upon addition of 1.5 μl of 1.5 mM dATP, 0.5 mM [α³²P]-dTTP (specific activity 2,000-4,000 cpm/pmol). Aliquots of 25 μl were removed at the indicated times and quenched with an equal volume (25 μl) of 1% SDS, 40 mM EDTA. One-half of the quenched reaction was analyzed for total deoxynucleotide incorporation using DE81 filter paper and the other half was analyzed on a 0.8% neutral agarose gel. The effect of TMAU was also examined, in which 100 μM TMAU in DMSO (2% DMSO final concentration) was present. In this case, replication was allowed to proceed for 1 min before being quenched with 25 μl of 1% SDS, 40 mM EDTA.

At a saturating concentration of DnaE polymerase, the time course of primer extension shows that it completes an M13mp18 primed ssDNA template within 2 minutes for a speed of at least 60 nucleotides/s (FIG. 21C). This rate of synthesis holds true for the highest amount of DnaE in the rightmost panel of the figure. As the DnaE concentration is decreased, a longer time is required to complete the circular template, indicating that the DnaE polymerase is not processive over the entire length of the M13mp18 template. If the DnaE polymerase were fully processive during synthesis of the 7.2 kb ssDNA circle, the product profile over time would be qualitatively similar at all concentrations of enzyme, but the overall intensity of the profile would be diminished. This particular experiment was performed in the absence of β, but presence of τδδ′. When repeated in the presence of but without τδδ′, and in the absence of both β and τδδ′, results similar to those shown in FIG. 21C were observed.

In the presence of β and τδδ′, DnaE polymerase is stimulated in synthesis at low concentration, indicating that β increases the processivity and/or speed of DnaE (FIGS. 21C-D). At higher concentrations of DnaE, the presence of β/τδδ′ has no effect on the rate of synthesis, and thus β does not increase the intrinsic speed of the enzyme (i.e., panels 3 and 4 of FIG. 21D). Hence, the effect of the β clamp on DnaE is primarily due to an increase in processivity. The profile of product length over time remains essentially unchanged at the different DnaE concentrations, and therefore the processivity of DnaE, with β is at least equal to the 7.2 kb length of the M13 mp18 substrate.

The DnaE sequence does not show homology to an exonuclease, implying that it may have no associated nuclease activity. The DnaE preparation was examined for the presence of a 3′-5′ exonuclease (FIG. 21E). The DnaE and PolC polymerases were each incubated with a 5′ 32P-labeled oligonucleotide, followed by analysis in a sequencing gel. The result showed no degradation of the oligonucleotide by DnaE. PolC is a known 3′-5′ exonuclease and it digests the end-labeled oligonucleotide as expected.

Gram positive PolC is known to be inhibited by the antibiotic hydroxyphenylaza-uracil (“HPUra”) and its derivatives. In FIG. 21F, the PolC•τδδ′, β and DnaE were tested for inhibition of synthesis on SSB coated primed M13 mp18 ssDNA by an HPUra derivative, trimethylanilino-uracil (“TMAU”). The PolC•τδδ′β enzyme was prevented from forming the RFII product by TMAU. In contrast, the DnaE polymerase was not affected by TMAU in the presence of τδδ′/β (nor in the absence of τδδ′/β, not shown).

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. An isolated protein or polypeptide from a Gram positive bacterium, wherein the isolated protein or polypeptide is beta.

2. The isolated protein or polypeptide according to claim 1, wherein the Gram positive bacterium is Streptococcus pyogenes.

3. The isolated protein or polypeptide according to claim 2, wherein the beta protein or polypeptide comprises an amino acid sequence of SEQ. ID. No. 28.
